SEQUENCE LISTING (1011 sequences)

SEQ ID NO: 1 - protein, 431 aa, Homo sapiens
MGPVIGMTPDKRAETPGAEKIAGLSQIYKMGSLPEAVDAARPKATLVDSESADDELTNLNWLHESTNLLTNFSLGSEGLPIVSPLYDIEGDDVPSFGPACYQNPEKKSATSKPPYSFSLLIYMAIEHSPNKCLPVKEIYSWILDHFPYFATAPTGWKNSVRHNLSLNKCFQKVERSHGKVNGKGSLWCVDPEYKPNLIQALKKQPFSSASSQNGSLSPHYLSSVIKQNQVRNLKESDIDAAAAMMLLNTSIEQGILECEKPLPLKTALQKKRSYGNAFHHPSAVRLQESDSLATSIDPKEDHNYSASSMAAQRCASRSSVSSLSSVDEVYEFIPKNSHVGSDGSEGFHSEEDTDVDYEDDPLGDSGYASQPCAKISEKGQSGKKMRKQTCQEIDEELKEAAGSLLHLAGIRTCLGSLISTAKTQNQKQRKK

SEQ ID NO: 2 - protein, 456 aa, Homo sapiens
MNKFQGPVTLKDVIVEFTKEEWKLLTPAQRTLYKDVMLENYSHLVSVGYHVNKPNAVFKLKQGKEPWILEVEFPHRGFPEDLWSIHDLEARYQESQAGNSRNGELTKHQKTHTTEKACECKECGKFFCQKSALIVHQHTHSKGKSYDCDKCGKSFSKNEDLIRHQKIHTRDKTYECKECKKIFYHLSSLSRHLRTHAGEKPYECNQCEKSFYQKPHLTEHQKTHTGEKPFECTECGKFFYVKAYLMVHQKTHTGEKPYECKECGKAFSQKSHLTVHQRMHTGEKPYKCKECGKFFSRNSHLKTHQRSHTGEKPYECKECRKCFYQKSALTVHQRTHTGEKPFECNKCGKTFYYKSDLTKHQRKHTGEKPYECTECGKSFAVNSVLRLHQRTHTGEKPYACKECGKSFSQKSHFIIHQRKHTGEKPYECQECGETFIQKSQLTAHQKTHTKKRNAEK

SEQ ID NO: 3 - protein, 410 aa, Homo sapiens
MEQKPSKVECGSDPEENSARSPDGKRKRKNGQCSLKTSMSGYIPSYLDKDEQCVVCGDKATGYHYRCITCEGCKGFFRRTIQKNLHPTYSCKYDSCCVIDKITRNQCQLCRFKKCIAVGMAMDLVLDDSKRVAKRKLIEQNRERRRKEEMIRSLQQRPEPTPEEWDLIHIATEAHRSTNAQGSHWKQRRKFLPDDIGQSPIVSMPDGDKVDLEAFSEFTKIITPAITRVVDFAKKLPMFSELPCEDQIILLKGCCMEIMSLRAAVRYDPESDTLTLSGEMAVKREQLKNGGLGVVSDAIFELGKSLSAFNLDDTEVALLQAVLLMSTDRSGLLCVDKIEKSQEAYLLAFEHYVNHRKHNIPHFWPKLLMKVTDLRMIGACHASRFLHMKVECPTELFPPLFLEVFEDQEV

SEQ ID NO: 4 - protein, 514 aa, Homo sapiens
MDLANHGLILLQQLNAQREFGFLCDCTVAIGDVYFKAHKSVLASFSNYFKMLFVHQTSECVRLKPTDIQPDIFSYLLHLMYTGKMAPQLIDPVRLEQGIKFLHAYPLIQEASLASQGAFSHPDQVFPLASSLYGIQIADHQLRQATKIASAPEKLGRDPRPQTSRISQEQVPEASQLSQLTSNLAQVNRTNMTPSDPLQTSLSPELVSTPVPPPPPGEETNLEASSSDEQPASLTIAHVKPSIMKRNGSFPKYYACHLCGRRFTLRSSLREHLQIHTGVPFTSSQQGESRVPLTLCSNAADLGKDAMEVPEAGMISDSELQHISDSPIIDGQQQSETPPPSDIADIDNLEQADQEREVKRRKYECTICGRKFIQKSHWREHMYIHTGKPFKCSTCDKSFCRANQAARHVCLNQSIDTYTMVDKQTLELCTFEEGSQMDNMLVQTNKPYKCNLCDKTFSTPNEVVKHSCQNQNSDVFALDEGRSILLGSGDSEVTEPDHPVLASIKKEQETVLLD

SEQ ID NO: 5 - protein, 300 aa, Homo sapiens
MSKCARKKYIKTNPRQMTMETVESQHDGSITASLTESKSAHVQTQTGQNSIPALAQVAAIAETDESAESEGVIDSHKRREILSRRPSYRKILNELSSDVPGVPKIEEERSEEEGTPPSIATMAVPTSIYQTSTGQYIAIAQGGTIQISNPGSDGVQGLQALTMTNSGAPPPGATIVQYAAQSADGTQQFFVPGSQVVVQAATGDMPTYQIRAPTAALPQGVVMAASPGSLHSPQQLAEEATRKRELRLMKNREAAKECRRRKKEYVKCLESRVAVLEVQNKKLIEELETLKDICSPKTDY

SEQ ID NO: 6 - protein, 328 aa, Homo sapiens
MDAVLLEHFPGGLDAFPSSYFDEDDFFTDQSSRDPLEDGDELLADEQAEVEFLSHQLHEYCYRDGACLLLQPAPPAAPLALAPPSSGGLGEPDDGGGGGYCCETGAPPGGFPYSPGSPPSCLAYPCAGAAVLSPGARLRGLSGAAAAAARRRRRVRSEAELQQLRQAANVRERRRMQSINDAFEGLRSHIPTLPYEKRLSKVDTLRLAIGYINFLSELVQADLPLRGGGAGGCGGPGGGGRLGGDSPGSQAQKVIICHRGTRSPSPSDPDYGLPPLAGHSLSWTDEKQLKEQNIIRTAKVWTPEDPRKLNSKSSFNNIENEPPFEFVS

SEQ ID NO: 7 - protein, 526 aa, Homo sapiens [sequence omitted]

SEQ ID NO: 8 - protein, 610 aa, Homo sapiens
MQYSHHCEHLLERLNKQREAGFLCDCTIVIGEFQFKAHRNVLASFSEYFGAIYRSTSENNVFLDQSQVKADGFQKLLEFIYTGTLNLDSWNVKEIHQAADYLKVEEVVTKCKIKMEDFAFIANPSSTEISSITGNIELNQQTCLLTLRDYNNREKSEVSTDLIQANPKQGALAKKSSQTKKKKKAFNSPKTGQNKTVQYPSDILENASVELFLDANKLPTPVVEQVAQINDNSELELTSVVENTFPAQDIVHTVTVKRKRGKSQPNCALKEHSMSNIASVKSPYEAENSGEELDQRYSKAKPMCNTCGKVFSEASSLRRHMRIHKGVKPYVCHLCGKAFTQCNQLKTHVRTHTGEKPYKCELCDKGFAQKCQLVFHSRMHHGEEKPYKCDVCNLQFATSSNLKIHARKHSGEKPYVCDRCGQRFAQASTLTYHVRRHTGEKPYVCDTCGKAFAVSSSLITHSRKHTGEKPYICGICGKSFISSGELNKHFRSHTGERPFICELCGNSYTDIKNLKKHKTKVHSGADKTLDSSAEDHTLSEQDSIQKSPLSETMDVKPSDMTLPLALPLGTEDHHMLLPVTDTQSPTSDTLLRSTVNGYSEPQLIFLQQLY

SEQ ID NO: 9 - protein, 841 aa, Homo sapiens
MSVDEKPDSPMYVYESTVHCTNILLGLNDQRKKDILCDVTLIVERKEFRAHRAVLAACSEYFWQALVGQTKNDLVVSLPEEVTARGFGPLLQFAYTAKLLLSRENIREVIRCAEFLRMHNLEDSCFSFLQTQLLNSEDGLFVCRKDAACQRPHEDCENSAGEEEDEEEETMDSETAKMACPRDQMLPEPISFEAAAIPVAEKEEALLPEPDVPTDTKESSEKDALTQYPRYKKYQLACTKNVYNASSHSTSGFASTFREDNSSNSLKPGLARGQIKSEPPSEENEEESITLCLSGDEPDAKDRAGDVEMDRKQPSPAPTPTAPAGAACLERSRSVASPSCLRSLFSITKSVELSGLPSTSQQHFARSPACPFDKGITQGDLKTDYTPFTGNYGQPHVGQKEVSNFTMGSPLRGPGLEALCKQEGELDRRSVIFSSSACDQVSTSVHSYSGVSSLDKDLSEPVPKGLWVGAGQSLPSSQAYSHGGLMADHLPGRMRPNTSCPVPIKVCPRSPPLETRTRTSSSCSSYSYAEDGSGGSPCSLPLCEFSSSPCSQGARFLATEHQEPGLMGDGMYNQVRPQIKCEQSYGTNSSDESGSFSEADSESCPVQDRGQEVKLPFPVDQITDLPRNDFQMMIKMHKLTSEQLEFIHDVRRRSKNRIAAQRCRKRKLDCIQNLECEIRKLVCEKEKLLSERNQLKACMGELLDNFSCLSQEVCRDIQSPEQIQALHRYCPVLRPMDLPTASSINPAPLGAEQNIAASQCAVGENVPCCLEPGAAPPGPPWAPSNTSENCTSGRRLEGTDPGTFSERGPPLEPRSQTVTVDFCQEMTDKCTTDEQPRKDYT

SEQ ID NO: 10 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 11 - protein, 781 aa, Homo sapiens
MTAPEKPVKQEEMAALDVDSGGGGGGGGGHGEYLQQQQQHGNGAVAAAAAAQDTQPSPLALLAATCSKIGPPSPGDDEEEAAAAAGAPAAAGATGDLASAQLGGAPNRWEVLSATPTTIKDEAGNLVQIPSAATSSGQYVLPLQNLQNQQIFSVAPGSDSSNGTVSSVQYQVIPQIQSADGQQVQIGFTGSSDNGGINQESSQIQIIPGSNQTLLASGTPSANIQNLIPQTGQVQVQGVAIGGSSFPGQTQVVANVPLGLPGNITFVPINSVDLDSLGLSGSSQTMTAGINADGHLINTGQAMDSSDNSERTGERVSPDINETNTDTDLFVPTSSSSQLPVTIDSTGILQQNTNSLTTSSGQVHSSDLQGNYIQSPVSEETQAQNIQVSTAQPVVQHLQLQESQQPTSQAQIVQGITPQTIHGVQASGQNISQQALQNLQLQLNPGTFLIQAQTVTPSGQVTWQTFQVQGVQNLQNLQIQNTAAQQITLTPVQTLTLGQVAAGGAFTSTPVSLSTGQLPNLQTVTVNSIDSAGIQLHPGENADSPADIRIKEEEPDPEEWQLSGDSTLNTNDLTHLRVQVVDEEGDQQHQEGKRLRRVACTCPNCKEGGGRGTNLGKKKQHICHIPGCGKVYGKTSHLRAHLRWHSGERPFVCNWMYCGKRFTRSDELQRHRRTHTGEKKFVCPECSKRFMRSDHLAKHIKTHQNKKGIHSSSTVLASVEAARDDTLITAGGTTLILANIQQGSVSGIGTVNTSATSNQDILTNTEIPLQLVTVSGNETME

SEQ ID NO: 12 - protein, 574 aa, Homo sapiens
MLREFSKWGVEASPGKAWERKRSLLRGAVGRYRGATGGDLFWAPFPSWGTMEFPEHSQQLLQSLREQRSQGFLCDCTVMVGSTQFLAHRAVLASCSPFFQLFYKERELDKRDLVCIHNEIVTAPAFGLLLDFMYAGQLTLRGDTPVEDVLAAASYLHMNDIVKVCKRRLQARALAEADSTKKEEETNSQLPSLEFLSSTSRGTQPSLASAETSGHWGKGEWKGSAAPSPTVRPPDEPPMSSGADTTQPGMEVDAPHLRAPHPPVADVSLASPSSSTETIPTNYFSSGISAVSLEPLPSLDVGPESLRVVEPKDPGGPLQGFYPPASAPTSAPAPVSAPVPSQAPAPAEAELVQVKVEAIVISDEETDVSDEQPQGPERAFPSGGAVYGAQPSQPEAFEDPGAAGLEEVGPSDHFLPTDPHLPYHLLPGAGQYHRGLVTSPLPAPASLHEPLYLSSEYEAAPGSFGVFTEDVPTCKTCGKTFSCSYTLRRHATVHTRERPYECRYCLRSYTQSGDLYRHIRKAHNEDLAKRSKPDPEVGPLLGVQPLPGSPTADRQSSSGGGPPKDFVLAPKTNI

SEQ ID NO: 13 - protein, 1097 aa, Homo sapiens
MATQVMGQSSGGGGLFTSSGNIGMALPNDMYDLHDLSKAELAAPQLIMLANVALTGEVNGSCCDYLVGEERQMAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSNSEKFSKTKKSKRKLEVDSHSLHGPVNDEESSTKKKKKVESKSKNNSQEVPKGDSKVEENKKQNTCMKKSTKKKTLKNKSSKKSSKPPQKEPVEKGSAQMDPPQMGPAPTEAVQKGPVQVEPPPPMEHAQMEGAQIRPAPDEPVQMEVVQEGPAQKELLPPVEPAQMVGAQIVLAHMELPPPMETAQTEVAQMGPAPMEPAQMEVAQVESAPMQVVQKEPVQMELSPPMEVVQKEPVQIELSPPMEVVQKEPVKIELSPPIEVVQKEPVQMELSPPMGVVQKEPAQREPPPPREPPLHMEPISKKPPLRKDKKEKSNMQSERARKEQVLIEVGLVPVKDSWLLKESVSTEDLSPPSPPLPKENLREEASGDQKLLNTGEGNKEAPLQKVGAEEADESLPGLAANINESTHISSSGQNLNTPEGETLNGKHQTDSIVCEMKMDTDQNTRENLTGINSTVEEPVSPMLPPSAVEEREAVSKTALASPPATMAANESQEIDEDEGIHSHEGSDLSDNMSEGSDDSGLHGARPVPQESSRKNAKEALAVKAAKGDFVCIFCDRSFRKGKDYSKHLNRHLVNVYYLEEAAQGQE

SEQ ID NO: 14 - protein, 826 aa, Homo sapiens
MVAPVLETSHVFCCPNRVRGVLNWSSGPRGLLAFGTSCSVVLYDPLKRVVVTNLNGHTARVNCIQWICKQDGSPSTELVSGGSDNQVIHWEIEDNQLLKAVHLQGHEGPVYAVHAVYQRRTSDPALCTLIVSAAADSAVRLWSKKGPEVMCLQTLNFGNGFALALCLSFLPNTDVPILACGNDDCRIHIFAQQNDQFQKVLSLCGHEDWIRGVEWAAFGRDLFLASCSQDCLIRIWKLYIKSTSLETQDDDNIRLKENTFTIENESVKIAFAVTLETVLAGHENWVNAVHWQPVFYKDGVLQQPVRLLSASMDKTMILWAPDEESGVWLEQVRVGEVGGNTLGFYDCQFNEDGSMIIAHAFHGALHLWKQNTVNPREWTPEIVISGHFDGVQDLVWDPEGEFIITVGTDQTTRLFAPWKRKDQSQVTWHEIARPQIHGYDLKCLAMINRFQFVSGADEKVLRVFSAPRNFVENFCAITGQSLNHVLCNQDSDLPEGATVPALGLSNKAVFQGDIASQPSDEEELLTSTGFEYQQVAFQPSILTEPPTEDHLLQNTLWPEVQKLYGHGYEIFCVTCNSSKTLLASACKAAKKEHAAIILWNTTSWKQVQNLVFHSLTVTQMAFSPNEKFLLAVSRDRTWSLWKKQDTISPEFEPVFSLFAFTNKITSVHSRIIWSCDWSPDSKYFFTGSRDKKVVVWGECDSTDDCIEHNIGPCSSVLDVGGAVTAVSVCPVLHPSQRYVVAVGLECGKICLYTWKKTDQVPEINDWTHCVETSQSQSHTLAIRKLCWKNCSGKTEQKEAEGAEWLHFASCGEDHTVKIHRVNKCAL

SEQ ID NO: 15 - protein, 847 aa, Homo sapiens
MSLWGLVSKMPPEKVQRLYVDFPQHLRHLLGDWLESQPWEFLVGSDAFCCNLASALLSDTVQHLQASVGEQGEGSTILQHISTLESIYQRDPLKLVATFRQILQGEKKAVMEQFRHLPMPFHWKQEELKFKTGLRRLQHRVGEIHLLREALQKGAEAGQVSLHSLIETPANGTGPSEALAMLLQETTGELEAAKALVLKRIQIWKRQQQLAGNGAPFEESLAPLQERCESLVDIYSQLQQEVGAAGGELEPKTRASLTGRLDEVLRTLVTSCFLVEKQPPQVLKTQTKFQAGVRFLLGLRFLGAPAKPPLVRADMVTEKQARELSVPQGPGAGAESTGEIINNTVPLENSIPGNCCSALFKNLLLKKIKRCERKGTESVTEEKCAVLFSASFTLGPGKLPIQLQALSLPLVVIVHGNQDNNAKATILWPRTFSEMDRVPFVVAERVPWEKMCETLNLKFMAEVGTNRGLLPEHFLFLAQKIFNDNSLSMEAFQHRSVSWSQFNKEILLGRGFTFWQWFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPEQMGKDGRGYVPATIKMTVERDQPLPTPELQMPTMVPSYDLGMAPDSSMSMQLGPDMVPQVYPPHSHSIPPYQGLSPEESVNVLSAFQEPHLQMPPSLGQMSLPFDQPHPQGLLPCQPQEHAVSSPDPLLCSDVTMVEDSCLSQPVTAFPQGTWIGEDIFPPLLPPTEQDLTKLLLEGQGESGGGSLGAQPLLQPSHYGQSGISMSHMDLRANPSW

SEQ ID NO: 16 - protein, 1234 aa, Homo sapiens
MHPQSLAEEEIKTEQEVVEGMDISTRSKDPGSAERTAQKRKFPSPPHSSNGHSPQDTSTSPIKKKKKPGLLNSNNKEQSELRHGPFYYMKQPLTTDPVDVVPQDGRNDFYCWVCHREGQVLCCELCPRVYHAKCLRLTSEPEGDWFCPECEKITVAECIETQSKAMTMLTIEQLSYLLKFAIQKMKQPGTDAFQKPVPLEQHPDYAEYIFHPMDLCTLEKNAKKKMYGCTEAFLADAKWILHNCIIYNGGNHKLTQIAKVVIKICEHEMNEIEVCPECYLAACQKRDNWFCEPCSNPHPLVWAKLKGFPFWPAKALRDKDGQVDARFFGQHDRAWVPINNCYLMSKEIPFSVKKTKSIFNSAMQEMEVYVENIRRKFGVFNYSPFRTPYTPNSQYQMLLDPTNPSAGTAKIDKQEKVKLNFDMTASPKILMSKPVLSGGTGRRISLSDMPRSPMSTNSSVHTGSDVEQDAEKKATSSHFSASEESMDFLDKSTASPASTKTGQAGSLSGSPKPFSPQLSAPITTKTDKTSTTGSILNLNLDRSKAEMDLKELSESVQQQSTPVPLISPKRQIRSRFQLNLDKTIESCKAQLGINEISEDVYTAVEHSDSEDSEKSDSSDSEYISDDEQKSKNEPEDTEDKEGCQMDKEPSAVKKKPKPTNPVEIKEELKSTSPASEKADPGAVKDKASPEPEKDFSEKAKPSPHPIKDKLKGKDETDSPTVHLGLDSDSESELVIDLGEDHSGREGRKNKKEPKEPSPKQDVVGKTPPSTTVGSHSPPETPVLTRSSAQTSAAGATATTSTSSTVTVTAPAPAATGSPVKKQRPLLPKETAPAVQRVVWNSSSKFQTSSQKWHMQKMQRQQQQQQQQNQQQQPQSSQGTRYQTRQAVKAVQQKEITQSPSTSTITLVTSTQSSPLVTSSGSMSTLVSSVNADLPIATASADVAADIAKYTSKMMDAIKGTMTEIYNDLSKNTTGSTIAEIRRLRIEIEKLQWLHQQELSEMKHNLELTMAEMRQSLEQERDRLIAEVKKQLELEKQQAVDETKKKQWCANCKKEAIFYCCWNTSYCDYPCQQAHWPEHMKSCTQSATAPQQEADAEVNTETLNKSSQGSSSSTQSAPSETASASKEKETSAEKSKESGSTLDLSGSRETPSSILLGSNQGSVSKRCDKQPAYAPTTTDHQPHPNYPAQKYHSRSNKSSWSSSDEKRGSTRSDHNTSTSTKSLLPKESRLDTFWD

SEQ ID NO: 17 - protein, 449 aa, Homo sapiens
MEFFISMSETIKYNDDDHKTLFLKTLNEQRLEGEFCDIAIVVEDVKFRAHRCVLAACSTYFKKLFKKLEVDSSSVIEIDFLRSDIFEEVLNYMYTAKISVKKEDVNLMMSSGQILGIRFLDKLCSQKRDVSSPDENNGQSKSKYCLKINRPIGDAADTQDDDVEEIGDQDDSPSDDTVEGTPPSQEDGKSPTTTLRVQEAILKELGSEEVRKVNCYGQEVESMETPESKDLGSQTPQALTFNDGMSEVKDEQTPGWTTAASDMKFEYLLYGHHREQIACQACGKTFSDEGRLRKHEKLHTADRPFVCEMCTKGFTTQAHLKEHLKIHTGYKPYSCEVCGKSFIRAPDLKKHERVHSNERPFACHMCDKAFKHKSHLKDHERRHRGEKPFVCGSCTKAFAKASDLKRHENNMHSERKQVTPSAIQSETEQLQAAAMAAEAEQQLETIACS

SEQ ID NO: 18 - protein, 422 aa, Homo sapiens
MPRSFLVKSKKAHSYHQPRSPGPDYSLRLENVPAPSRADSTSNAGGAKAEPRDRLSPESQLTEAPDRASASPDSCEGSVCERSSEFEDFWRPPSPSASPASEKSMCPSLDEAQPFPLPFKPYSWSGLAGSDLRHLVQSYRPCGALERGAGLGLFCEPAPEPGHPAALYGPKRAAGGAGAGAPGSCSAGAGATAGPGLGLYGDFGSAAAGLYERPTAAAGLLYPERGHGLHADKGAGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK

SEQ ID NO: 19 - protein, 337 aa, Homo sapiens
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 20 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 21 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 22 - protein, 637 aa, Homo sapiens
MHDSIRFGNVDMPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGPRTACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 23 - protein, 637 aa, Homo sapiens; identical to SEQ ID NO: 22

SEQ ID NO: 24 - protein, 399 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 25 - protein, 487 aa, Homo sapiens
MSLPPIRLPSPYGSDRLVQLAARLRPALCDTLITVGSQEFPAHSLVLAGVSQQLGRRGQWALGEGISPSTFAQLLNFVYGESVELQPGELRPLQEAARALGVQSLEEACWRARGDRAKKPDPGLKKHQEEPEKPSRNPERELGDPGEKQKPEQVSRTGGREQEMLHKHSPPRGRPEMAGATQEAQQEQTRSKEKRLQAPVGQRGADGKHGVLTWLRENPGGSEESLRKLPGPLPPAGSLQTSVTPRPSWAEAPWLVGGQPALWSILLMPPRYGIPFYHSTPTTGAWQEVWREQRIPLSLNAPKGLWSQNQLASSSPTPGSLPQGPAQLSPGEMEESDQGHTGALATCAGHEDKAGCPPRPHPPPAPPARSRPYACSVCGKRFSLKHQMETHYRVHTGEKPFSCSLCPQRSRDFSAMTKHLRTHGAAPYRCSLCGAGCPSLASMQAHMRGHSPSQLPPGWTIRSTFLYSSSRPSRPSTSPCCPSSSTT

SEQ ID NO: 26 - protein, 967 aa, Homo sapiens
MAVALDSQIDAPLEVEGCLIMKVEKDPEWASEPILEGSDSSETFRKCFRQFCYEDVTGPHEAFSKLWELCCRWLKPEMRSKEQILELLVIEQFLTILPEKIQAWAQKQCPQSGEEAVALVVHLEKETGRLRQQVSSPVHREKHSPLGAAWEVADFQPEQVETQPRAVSREEPGSLHSGHQEQLNRKRERRPLPKNARPSPWVPALADEWNTLDQEVTTTRLPAGSQEPVKDVHVARGFSYRKSVHQIPAQRDLYRDFRKENVGNVVSLGSAVSTSNKITRLEQRKEPWTLGLHSSNKRSILRSNYVKEKSVHAIQVPARSAGKTWREQQQWGLEDEKIAGVHWSYEETKTFLAILKESRFYETLQACPRNSQVYGAVAEWLRECGFLRTPEQCRTKFKSLQKSYRKVRNGHMLEPCAFFEDMDALLNPAARAPSTDKPKEMIPVPRLKRIAISAKEHISLVEEEEAAEDSDDDEIGIEFIRKSEIHGAPVLFQNLSGVHWGYEETKTFLDILRETRFYEALQACHRKSKLYGAVAEQLRECGFLRTPEQCRTKFKSLQKSYRKVKNGHVLESCAFYKEMDALINSRASAPSPSTPEEVPSPSRQERGGIEVEPQEPTGWEPEETSQEAVIEDSCSERMSEEEIVQEPEFQGPPGLLQSPNDFEIGSSIKEDPTQIVYKDMEQHRALIEKSKRVVSQSTDPSKYRKRECISGRQWENLQGIRQGKPMSQPRDLGKAVVHQRPFVGKRPYRLLKYGESFGRSTRLMCRMTHHKENPYKCGVCGKCFGRSRSLIRHQRIHTGEKPFKCLDCGKSFNDSSNFGAHQRIHTGEKPYRCGECGKCFSQSSSLIIHQRTHTGEKPYQCGECGKSFTNSSHFSAHRRVHTGENPYKCVDCEKSFNNCTRFREHRRIHTGEKPYGCAQCGKRFSKSSVLTKHREVHVREKPLPHPPSLYCPENPHKGKTDEFRKTF

SEQ ID NO: 27 - protein, 594 aa, Homo sapiens
MAAPVKGNRKQSTEGDALDPPASPKPAGKQNGIQNPISLEDSPEAGGEREEEQEREEEQAFLVSLYKFMKERHTPIERVPHLGFKQINLWKIYKAVEKLGAYELVTGRRLWKNVYDELGGSPGSTSAATCTRRHYERLVLPYVRHLKGEDDKPLPTSKPRKQYKMAKENRGDDGATERPKKAKEERRMDQMMPGKTKADAADPAPLPSQEPPRNSTEQQGLASGSSVSFVGASGCPEAYKRLLSSFYCKGTHGIMSPLAKKKLLAQVSKVEALQCQEEGCRHGAEPQASPAVHLPESPQSPKGLTENSRHRLTPQEGLQAPGGSLREEAQAGPCPAAPIFKGCFYTHPTEVLKPVSQHPRDFFSRLKDGVLLGPPGKEGLSVKEPQLVWGGDANRPSAFHKGGSRKGILYPKPKACWVSPMAKVPAESPTLPPTFPSSPGLGSKRSLEEEGAAHSGKRLRAVSPFLKEADAKKCGAKPAGSGLVSCLLGPALGPVPPEAYRGTMLHCPLNFTGTPGPLKGQAALPFSPLVIPAFPAHFLATAGPSPMAAGLMHFPPTSFDSALRHRLCPASSAWHAPPVTTYAAPHFFHLNTKL

SEQ ID NO: 28 - protein, 417 aa, Homo sapiens
MSQDSVTFADVAVNFTKEEWTLLDPAQRNLYRDVMLENSRNLAFIDWATPCKTKDATPQPDILPKRTFPEANRVCLTSISSQHSTLREDWRCPKTEEPHRQGVNNVKPPAVAPEKDESPVSICEDHEMRNHSKPTCRLVPSQGDSIRQCILTRDSSIFKYNPVLNDSQKTHENNEDDGVLGWNIQWVPCGRKTELKSSTWTGSQNTVHHIRDEIDTGANRHQRNPFGKAFREDGSLRAHNTHGREKMYDFTQCENTSRNNSIHAMQMQLYTAETNKKDCQTGATSANAPNSGSHKSHCTGEKTHKCPECGRAFFYQSFLMRHMKIHTGEKPYECGKCGKAFRYSLHLNKHLRKHVVQKKPYECEECGKVIRESSKYTHIRSHTGEKPYKCKTCGKDFAKSSGLKKHLKTHKDEKPCE

SEQ ID NO: 29 - protein, 784 aa, Homo sapiens
MEGAMAVRVTAAHTAEAQAEAGREAGEGAVAAVAAALAPSGFLGLPAPFSEEDEDDVHRCGRCQAEFTALEDFVQHKIQKACQRAPPEALPATPATTALLGQEVVPAAPGPEEPITVAHIVVEAASLAADISHASDLVGGGHIKEVIVAAEAELGDGEMAEAPGSPRQQGLGLAGEGEQAQVKLLVNKDGRYVCALCHKTFKTGSILKAHMVTHSSRKDHECKLCGASFRTKGSLIRHHRRHTDERPYKCSKCGKSFRESGALTRHLKSLTPCTEKIRFSVSKDVVVSKEDARAGSGAGAAGLGTATSSVTGEPIETSPVIHLVTDAKGTVIHEVHVQMQELSLGMKALAPEPPVSQELPCSSEGSRENLLHQAMQNSGIVLERAAGEEGALEPAPAAGSSPQPLAVAAPQLPVLEVQPLETQVASEASAVPRTHPCPQCSETFPTAATLEAHKRGHTGPRPFACAQCGKAFPKAYLLKKHQEVHVRERRFRCGDCGKLYKTIAHVRGHRRVHSDERPYPCPKCGKRYKTKNAQQVHFRTHLEEKPHVCQFCSRGFREKGSLVRHVRHHTGEKPFKCYKCGRGFAEHGTLNRHLRTKGGCLLEVEELLVSEDSPAAATTVLTEDPHTVLVEFSSVVADTQEYIIEATADDAETSEATEIIEGTQTEVDSHIMKVVQQIVHQASAGHQIIVQNVTMDEETALGPEAAAADTITIATPESLTEQVAMTLASAISEGTVLAARAGTSGTEQATVTMVSSEDIEILEHAGELVIASPEGQLEVQTVIV

SEQ ID NO: 30 - protein, 784 aa, Homo sapiens; identical to SEQ ID NO: 29

SEQ ID NO: 31 - protein, 524 aa, Homo sapiens
MHSLDEPLDLKLSITKLRAAREKRERTLGVVRPRALHRELGLVDDSPTPGSPGSPPSGFLLNSKFPEKVEGRFSAAPLVDLSLSPPSGLDSPNGSSSLSPERQGNGDLPPVPSASDFQPLRYLDGVPSSFQFFLPLGSGGALHLPASSFLTPPKDKCLSPDLPLPKQLVCRWAKCNQLFELLQDLVDHVNDYHVKPEKDAGYCCHWEGCARHGRGFNARYKMLIHIRTHTNEKPHRCPTCSKSFSRLENLKIHNRSHTGEKPYVCPYEGCNKRYSNSSDRFKHTRTHYVDKPYYCKMPGCHKRYTDPSSLRKHIKAHGHFVSHEQQELLQLRPPPKPPLPAPDGGPYVSGAQIIIPNPAALFGGPGLPGLPLPLAPGPLDLSALACGNGGGSGGGGGMGPGLPGPVLPLNLAKNPLLPSPFGAGGLGLPVVSLLAGAAGGKAEGEKGRGSVPTRALGMEGHKTPLERTESSCSRPSPDGLPLLPGTVLDLSTGVNSAASSPEALAPGWVVIPPGSVLLKPAVVN

SEQ ID NO: 32 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 33 - protein, 404 aa, Homo sapiens
MAMVTGGWGGPGGDTNGVDKAGGYPRAAEDDSASPPGAASDAEPGDEERPGLQVDCVVCGDKSSGKHYGVFTCEGCKSFFKRSIRRNLSYTCRSNRDCQIDQHHRNQCQYCRLKKCFRVGMRKEAVQRGRIPHSLPGAVAASSGSPPGSALAAVASGGDLFPGQPVSELIAQLLRAEPYPAAAGRFGAGGGAAGAVLGIDNVCELAARLLFSTVEWARHAPFFPELPVADQVALLRLSWSELFVLNAAQAALPLHTAPLLAAAGLHAAPMAAERAVAFMDQVRAFQEQVDKLGRLQVDSAEYGCLKAIALFTPDACGLSDPAHVESLQEKAQVALTEYVRAQYPSQPQRFGRLLLRLPALRAVPASLISQLFFMRLVGKTPIETLIRDMLLSGSTFNWPYGSGQ

SEQ ID NO: 34 - protein, 903 aa, Homo sapiens
MAASRSTRVTRSTVGLNGLDESFCGRTLRNRSIAHPEEISSNSQVRSRSPKKRPEPVPIQKGNNNGRTTDLKQQSTRESWVSPRKRGLSSSEKDNIERQAIENCERRQTEPVSPVLKRIKRCLRSEAPNSSEEDSPIKSDKESVEQRSTVVDNDADFQGTKRACRCLILDDCEKREIKKVNVSEEGPLNSAVVEEITGYLAVNGVDDSDSAVINCDDCQPDGNTKQNSIGSYVLQEKSVAENGDTDTQTSMFLDSRKEDSYIDHKVPCTDSQVQVKLEDHKIVTACLPVEHVNQLTTEPATGPFSETQSSLRDSEEEVDVVGDSSASKEQCKENTNNELDTSLESMPASGEPEPSPVLDCVSAQMMSLSEPQEHRYTLRTSPRRAAPTRGSPTKNSSPYRENGQFEENNLSPNETNATVSDNVSQSPTNPGEISQNEKGICCDSQNNGSEGVSKPPSEARLNIGHLPSAKESASQHITEEEDDDPDVYYFESDHVALKHNKDYQRLLQTIAVLEAQRSQAVQDLESLGRHQREALKNPIGFVEKLQKKADIGLPYPQRVVQLPEIVWDQYTHSLGNFEREFKNRKRHTRRVKLVFDKVGLPARPKSPLDPKKDGESLSYSMLPLSDGPEGSSSRPQMIRGRLCDDTKPETFNQLWTVEEQKKLEQLLIKYPPEEVESRRWQKIADELGNRTAKQVASRVQKYFIKLTKAGIPVPGRTPNLYIYSKKSSTSRRQHPLNKHLFKPSTFMTSHEPPVYMDEDDDRSCFHSHMNTAVEDASDDESIPIMYRNLPEYKELLQFKKLKKQKLQQMQAESGFVQHVGFKCDNCGIEPIQGVRWHCQDCPPEMSLDFCDSCSDCLHETDIHKEDHQLEPIYRSETFLDRDYCVSQGTSYNYLDPNYFPANR

SEQ ID NO: 35 - protein, 512 aa, Homo sapiens
MKAGCSIVEKPEGGGGYQFPDWAYKTESSPGSRQIQLWHFILELLQKEEFRHVIAWQQGEYGEFVIKDPDEVARLWGRRKCKPQMNYDKLSRALRYYYNKRILHKTKGKRFTYKFNFNKLVMPNYPFINIRSSGVVPQSAPPVPTASSRFHFPPLDTHSPTNDVQPGRFSASSLTASGQESSNGTDRKTELSELEDGSAADWRRGVDPVSSRNAIGGGGIGHQKRKPDIMLPLFARPGMYPDPHSPFAVSPIPGRGGVLNVPISPALSLTPTIFSYSPSPGLSPFTSSSCFSFNPEEMKHYLHSQACSVFNYHLSPRTFPRYPGLMVPPLQCQMHPEESTQFSIKLQPPPVGRKNRERVESSEESAPVTTPTMASIPPRIKVEPASEKDPESLRQSAREKEEHTQEEGTVPSRTIEEEKGTIFARPAAPPIWPSVPISTPSGEPLEVTEDSEDRPGKEPSAPEKKEDALMPPKLRLKRRWNDDPEARELSKSGKFLWNGSGPQGLATAAADA

SEQ ID NO: 36 - protein, 473 aa, Homo sapiens
MNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKAVAGADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 37 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 38 - protein, 237 aa, Homo sapiens
MSDSDLGEDEGLLSLAGKRKRRGNLPKESVKILRDWLYLHRYNAYPSEQEKLSLSGQTNLSVLQICNWFINARRRLLPDMLRKDGKDPNQFTISRRGGKASDVALPRGSSPSVLAVSVPAPTNVLSLSVCSMPLHSGQGEKPAAPFPRGELESPKPLVTPGSTLTLLTRAEAGSPTGGLFNTPPPTPPEQDKEDFSSFQLLVEVALQRAAEMELQKQQDPSLPLLHTPIPLVSENPQ

SEQ ID NO: 39 - protein, 896 aa, Homo sapiens
MTEESEETVLYIEHRYVCSECNQLYGSLEEVLMHQNSHVPQQHFELVGVADPGVTVATDTASGTGLYQTLVQESQYQCLECGQLLMSPSQLLEHQELHLKMMAPQEAVPAEPSPKAPPLSSSTIHYECVDCKALFASQELWLNHRQTHLRATPTKAPAPVVLGSPVVLGPPVGQARVAVEHSYRKAEEGGEGATVPSAAATTTEVVTEVELLLYKCSECSQLFQLPADFLEHQATHFPAPVPESQEPALQQEVQASSPAEVPVSQPDPLPASDHSYELRNGEAIGRDRRGRRARRNNSGEAGGAATQELFCSACDQLFLSPHQLQQHLRSHREGVFKCPLCSRVFPSPSSLDQHLGDHSSESHFLCVDCGLAFGTEALLLAHRRAHTPNPLHSCPCGKTFVNLTKFLYHRRTHGVGGVPLPTTPVPPEEPVIGFPEPAPAETGEPEAPEPPVSEETSAGPAAPGTYRCLLCSREFGKALQLTRHQRFVHRLERRHKCSICGKMFKKKSHVRNHLRTHTGERPFPCPDCSKPFNSPANLARHRLTHTGERPYRCGDCGKAFTQSSTLRQHRLVHAQHFPYRCQECGVRFHRPYRLLMHRYHHTGEYPYKCRECPRSFLLRRLLEVHQLVVHAGRQPHRCPSCGAAFPSSLRLREHRCAAAAAQAPRRFECGTCGKKVGSAARLQAHEAAHAAAGPGEVLAKEPPAPRAPRATRAPVASPAALGSTATASPAAPARRRGLECSECKKLFSTETSLQVHRRIHTGERPYPCPDCGKAFRQSTHLKDHRRLHTGERPFACEVCGKAFAISMRLAEHRRIHTGERPYSCPDCGKSYRSFSNLWKHRKTHQQQHQAAVRQQLAEAEAAVGLAVMETAVEALPLVEAIEIYPLAEAEGVQISG

SEQ ID NO: 40 - protein, 388 aa, Homo sapiens
MTAIIKEIVSRNKRRYQEDGFDLDLTYIYPNIIAMGFPAERLEGVYRNNIDDVVSCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 41 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 42 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 43 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 44 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWPRTFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 45 - protein, 794 aa, Homo sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWPRTFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 46 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 47 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 48 - protein, 452 aa, Homo sapiens
MDGTIKEALSVVSDDQSLFDSAYGAAAHLPKADMTASGSPDYGQPHKINPLPPQQEWINQPVRVNVKREYDHMNGSRESPVDCSVSKCSKLVGGGESNPMNYNSYMDEKNGPPPPNMTTNERRVIVPADPTLWTQEHVRQWLEWAIKEYSLMEIDTSFFQNMDGKELCKMNKEDFLRATTLYNTEVLLSHLSYLRESSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 49 - protein, 441 aa, Homo sapiens
MKAAVDLKPTLTIIKTEKVDLELFPSPDMECADVPLLTPSSKEMMSQALKATFSGFTKEQQRLGIPKDPRQWTETHVRDWVMWAVNEFSLKGVDFQKFCMNGAALCALGKDCFLELAPDFVGDILWEHLEILQKEDVKPYQVNGVNPAYPESRYTSDYFISYGIEHAQCVPPSEFSEPSFITESYQTLHPISSEELLSLKYENDYPSVILRDPLQTDTLQNDYFAIKQEVVTPDNMCMGRTSRGKLGGQDSFESIESYDSCDRLTQSWSSQSSFNSLQRVPSYDSFDSEDYPAALPNHKPKGTFKDYVRDRADLNKDKPVIPAAALAGYTGSGPIQLWQFLLELLTDKSCQSFISWTGDGWEFKLSDPDEVARRWGKRKNKPKMNYEKLSRGLRYYYDKNIIHKTAGKRYVYRFVCDLQSLLGYTPEELHAMLDVKPDADE

SEQ ID NO: 50 - protein, 347 aa, Homo sapiens
MKMEEMSLSGLDNSKLEAIAQEIYADLVEDSCLGFCFEVHRAVKCGYFFLDDTDPDSMKDFEIVDQPGLDIFGQVFNQWKSKECVCPNCSRSIAASRFAPHLEKCLGMGRNSSRIANRRIANSNNMNKSESDQEDNDDINDNDWSYGSEKKAKKRKSDKNPNSPRRSKSLKHKNGELSNSDPFKYNNSTGISYETLGPEELRSLLTTQCGVISEHTKKMCTRSLRCPQHTDEQRRTVRIYFLGPSAVLPEVESSLDNDSFDMTDSQALISRLQWDGSSDLSPSDSGSSKTSENQGWGLGTNSSESRKTKKKKSHLSLVGTASGLGSNKKKKPKPPAPPTPSIYDDIN

SEQ ID NO: 51 - protein, 3969 aa, Homo sapiens
MAHSCRWRFPARPGTTGGGGGGGRRGLGGAPRQRVPALLLPPGPPVGGGGPGAPPSPPAVAAAAAAAGSSGAGVPGGAAAASAASSSSASSSSSSSSSASSGPALLRVGPGFDAALQVSAAIGTNLRRFRAVFGESGGGGGSGEDEQFLGFGSDEEVRVRSPTRSPSVKTSPRKPRGRPRSGSDRNSAILSDPSVFSPLNKSETKSGDKIKKKDSKSIEKKRGRPPTFPGVKIKITHGKDISELPKGNKEDSLKKIKRTPSATFQQATKIKKLRAGKLSPLKSKFKTGKLQIGRKGVQIVRRRGRPPSTERIKTPSGLLINSELEKPQKVRKDKEGTPPLTKEDKTVVRQSPRRIKPVRIIPSSKRTDATIAKQLLQRAKKGAQKKIEKEAAQLQGRKVKTQVKNIRQFIMPVVSAISSRIIKTPRRFIEDEDYDPPIKIARLESTPNSRFSAPSCGSSEKSSAASQHSSQMSSDSSRSSSPSVDTSTDSQASEEIQVLPEERSDTPEVHPPLPISQSPENESNDRRSRRYSVSERSFGSRTTKKLSTLQSAPQQQTSSSPPPPLLTPPPPLQPASSISDHTPWLMPPTIPLASPFLPASTAPMQGKRKSILREPTFRWTSLKHSRSEPQYFSSAKYAKEGLIRKPIFDNFRPPPLTPEDVGFASGFSASGTAASARLFSPLHSGTRFDMHKRSPLLRAPRFTPSEAHSRIFESVTLPSNRTSAGTSSSGVSNRKRKRKVFSPIRSEPRSPSHSMRTRSGRLSSSELSPLTPPSSVSSSLSISVSPLATSALNPTFTFPSHSLTQSGESAEKNQRPRKQTSAPAEPFSSSSPTPLFPWFTPGSQTERGRNKDKAPEELSKDRDADKSVEKDKSRERDREREKENKRESRKEKRKKGSEIQSSSALYPVGRVSKEKVVGEDVATSSSAKKATGRKKSSSHDSGTDITSVTLGDTTAVKTKILIKKGRGNLEKTNLDLGPTAPSLEKEKTLCLSTPSSSTVKHSTSSIGSMLAQADKLPMTDKRVASLLKKAKAQLCKIEKSKSLKQTDQPKAQGQESDSSETSVRGPRIKHVCRRAAVALGRKRAVFPDDMPTLSALPWEEREKILSSMGNDDKSSIAGSEDAEPLAPPIKPIKPVTRNKAPQEPPVKKGRRSRRCGQCPGCQVPEDCGVCTNCLDKPKFGGRNIKKQCCKMRKCQNLQWMPSKAYLQKQAKAVKKKEKKSKTSEKKDSKESSVVKNVVDSSQKPTPSAREDPAPKKSSSEPPPRKPVEEKSEEGNVSAPGPESKQATTPASRKSSKQVSQPALVIPPQPPTTGPPRKEVPKTTPSEPKKKQPPPPESGPEQSKQKKVAPRPSIPVKQKPKEKEKPPPVNKQENAGTLNILSTLSNGNSSKQKIPADGVHRIRVDFKEDCEAENVWEMGGLGILTSVPITPRVVCFLCASSGHVEFVYCQVCCEPFHKFCLEENERPLEDQLENWCCRRCKFCHVCGRQHQATKQLLECNKCRNSYHPECLGPNYPTKPTKKKKVWICTKCVRCKSCGSTTPGKGWDAQWSHDFSLCHDCAKLFAKGNFCPLCDKCYDDDDYESKMMQCGKCDRWVHSKCENLSDEMYEILSNLPESVAYTCVNCTERHPAEWRLALEKELQISLKQVLTALLNSRTTSHLLRYRQAAKPPDLNPETEESIPSRSSPEGPDPPVLTEVSKQDDQQPLDLEGVKRKMDQGNYTSVLEFSDDIVKIIQAAINSDGGQPEIKKANSMVKSFFIRQMERVFPWFSVKKSRFWEPNKVSSNSGMLPNAVLPPSLDHNYAQWQEREENSHTEQPPLMKKIIPAPKPKGPGEPDSPTPLHPPTPPILSTDRSREDSPELNPPPGIEDNRQCALCLTYGDDSANDAGRLLYIGQNEWTHVNCALWSAEVFEDDDGSLKNVHMAVIRGKQLRCEFCQKPGATVGCCLTSCTSNYHFMCSRAKNCVFLDDKKVYCQRHRDLIKGEVVPENGFEVFRRVFVDFEGISLRRKFLNGLEPENIHMMIGSMTIDCLGILNDLSDCEDKLFPIGYQCSRVYWSTTDARKRCVYTCKIVECRPPVVEPDINSTVEHDENRTIAHSPTSFTESSSKESQNTAEIISPPSPDRPPHSQTSGSCYYHVISKVPRIRTPSYSPTQRSPGCRPLPSAGSPTPTTHEIVTVGDPLLSSGLRSIGSRRHSTSSLSPQRSKLRIMSPMRTGNTYSRNNVSSVSTTGTATDLESSAKVVDHVLGPLNSSTSLGQNTSTSSNLQRTVVTVGNKNSHLDGSSSSEMKQSSASDLVSKSSSLKGEKTKVLSSKSSEGSAHNVAYPGIPKLAPQVHNTTSRELNVSKIGSFAEPSSVSFSSKEALSFPHLHLRGQRNDRDQHTDSTQSANSSPDEDTEVKTLKLSGMSNRSSIINEHMGSSSRDRRQKGKKSCKETFKEKHSSKSFLEPGQVTTGEEGNLKPEFMDEVLTPEYMGQRPCNNVSSDKIGDKGLSMPGVPKAPPMQVEGSAKELQAPRKRTVKVTLTPLKMENESQSKNALKESSPASPLQIESTSPTEPISASENPGDGPVAQPSPNNTSCQDSQSNNYQNLPVQDRNLMLPDGPKPQEDGSFKRRYPRRSARARSNMFFGLTPLYGVRSYGEEDIPFYSSSTGKKRGKRSAEGQVDGADDLSTSDEDDLYYYNFTRTVISSGGEERLASHNLFREEEQCDLPKISQLDGVDDGTESDTSVTATTRKSSQIPKRNGKENGTENLKIDRPEDAGEKEHVTKSSVGHKNEPKMDNCHSVSRVKTQGQDSLEAQLSSLESSRRVHTSTPSDKNLLDTYNTELLKSDSDNNNSDDCGNILPSDIMDFVLKNTPSMQALGESPESSSSELLNLGEGLGLDSNREKDMGLFEVFSQQLPTTEPVDSSVSSSISAEEQFELPLELPSDLSVLTTRSPTVPSQNPSRLAVISDSGEKRVTITEKSVASSESDPALLSPGVDPTPEGHMTPDHFIQGHMDADHISSPPCGSVEQGHGNNQDLTRNSSTPGLQVPVSPTVPIQNQKYVPNSTDSPGPSQISNAAVQTTPPHLKPATEKLIVVNQNMQPLYVLQTLPNGVTQKIQLTSSVSSTPSVMETNTSVLGPMGGGLTLTTGLNPSLPTSQSLFPSASKGLLPMSHHQHLHSFPAATQSSFPPNISNPPSGLLIGVQPPPDPQLLVSESSQRTDLSTTVATPSSGLKKRPISRLQTRKNKKLAPSSTPSNIAPSDVVSNMTLINFTPSQLPNHPSLLDLGSLNTSSHRTVPNIIKRSKSSIMYFEPAPLLPQSVGGTAATAAGTSTISQDTSHLTSGSVSGLASSSSVLNVVSMQTTTTPTSSASVPGHVTLTNPRLLGTPDIGSISNLLIKASQQSLGIQDQPVALPPSSGMFPQLGTSQTPSTAAITAASSICVLPSTQTTGITAASPSGEADEHYQLQHVNQLLASKTGIHSSQRDLDSASGPQVSNFTQTVDAPNSMGLEQNKALSSAVQASPTSPGGSPSSPSSGQRSASPSVPGPTKPKPKTKRFQLPLDKGNGKKHKVSHLRTSSSEAHIPDQETTSLTSGTGTPGAEAEQQDTASVEQSSQKECGQPAGQVAVLPEVQVTQNPANEQESAEPKTVEEEESNFSSPLMLWLQQEQKRKESITEKKPKKGLVFEISSDDGFQICAESIEDAWKSLTDKVQEARSNARLKQLSFAGVNGLRMLGILHDAVVFLIEQLSGAKHCRNYKFRFHKPEEANEPPLNPHGSARAEVHLRKSAFDMFNFLASKHRQPPEYNPNDEEEEEVQLKSARRATSMDLPMPMRFRHLKKTSKEAVGVYRSPIHGRGLFCKRNIDAGEMVIEYAGNVIRSIQTDKREKYYDSKGIGCYMFRIDDSEVVDATMHGNAARFINHSCEPNCYSRVINIDGQKHIVIFAMRKIYRGEELTYDYKFPIEDASNKLPCNCGAKKCRKFLN

SEQ ID NO: 52 - protein, 1124 aa, Homo sapiens
MADGPRCKRRKQANPRRNNVTNYNTVVETNSDSDDEDKLHIVEEESVTDAADCEGVPEDDLPTDQTVLPGRSSEREGNAKNCWEDDRKEGQEILGPEAQADEAGCTVKDDECESDAENEQNHDPNVEEFLQQQDTAVIFPEAPEEDQRQGTPEASGHDENGTPDAFSQLLTCPYCDRGYKRFTSLKEHIKYRHEKNEDNFSCSLCSYTFAYRTQLERHMTSHKSGRDQRHVTQSGCNRKFKCTECGKAFKYKHHLKEHLRIHSGEKPYECPNCKKRFSHSGSYSSHISSKKCISLIPVNGRPRTGLKTSQCSSPSLSASPGSPTRPQIRQKIENKPLQEQLSVNQIKTEPVDYEFKPIVVASGINCSTPLQNGVFTGGGPLQATSSPQGMVQAVVLPTVGLVSPISINLSDIQNVLKVAVDGNVIRQVLENNQANLASKEQETINASPIQQGGHSVISAISLPLVDQDGTTKIIINYSLEQPSQLQVVPQNLKKENPVATNSCKSEKLPEDLTVKSEKDKSFEGGVNDSTCLLCDDCPGDINALPELKHYDLKQPTQPPPLPAAEAEKPESSVSSATGDGNLSPSQPPLKNLLSLLKAYYALNAQPSAEELSKIADSVNLPLDVVKKWFEKMQAGQISVQSSEPSSPEPGKVNIPAKNNDQPQSANANEPQDSTVNLQSPLKMTNSPVLPVGSTTNGSRSSTPSPSPLNLSSSRNTQGYLYTAEGAQEEPQVEPLDLSLPKQQGELLERSTITSVYQNSVYSVQEEPLNLSCAKKEPQKDSCVTDSEPVVNVIPPSANPINIAIPTVTAQLPTIVAIADQNSVPCLRALAANKQTILIPQVAYTYSTTVSPAVQEPPLKVIQPNGNQDERQDTSSEGVSNVEDQNDSDSTPPKKKMRKTENGMYACDLCDKIFQKSSSLLRHKYEHTGKRPHECGICKKAFKHKHHLIEHMRLHSGEKPYQCDKCGKRFSHSGSYSQHMNHRYSYCKREAEERDSTEQEEAGPEILSNEHVGARASPSQGDSDERESLTREEDEDSEKEEEEEDKEMEELQEEKECEKPQGDEEEEEEEEEVEEEEVEEAENEGEEAKTEGLMKDDRAESQASSLGQKVGESSEQVSEEKTNEA

SEQ ID NO: 53 - protein, 411 aa, Homo sapiens
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 54 - protein, 1608 aa, Homo sapiens
MYSAHRPLMPASSAASRGLGMFVWTNVEPRSVAVFPWHSLVPFLAPSQPDPSVQPSEAQQPASHPVASNQSKEPAESAAVAHERPPGGTGSADPERPPGATCPESPGPGPPHPLGVVESGKGPPPTTEEEASGPPGEPRLDSETESDHDDAFLSIMSPEIQLPLPPGKRRTQSLSALPKERDSSSEKDGRSPNKREKDHIRRPMNAFMIFSKRHRALVHQRHPNQDNRTVSKILGEWWYALGPKEKQKYHDLAFQVKEAHFKAHPDWKWCNKDRKKSSSEAKPTSLGLAGGHKETRERSMSETGTAAAPGVSSELLSVAAQTLLSSDTKAPGSSSCGAERLHTVGGPGSARPRAFSHSGVHSLDGGEVDSQALQELTQMVSGPASYSGPKPSTQYGAPGPFAAPGEGGALAATGRPPLLPTRASRSQRAASEDMTSDEERMVICEEEGDDDVIADDGFGTTDIDLKCKERVTDSESGDSSGEDPEGNKGFGRKVFSPVIRSSFTHCRPPLDPEPPGPPDPPVAFGKGYGSAPSSSASSPASSSASAATSFSLGSGTFKAQESGQGSTAGPLRPPPPGAGGPATPSKATRFLPMDPATFRRKRPESVGGLEPPGPSVIAAPPSGGGNILQTLVLPPNKEEQEGGGARVPSAPAPSLAYGAPAAPLSRPAATMVTNVVRPVSSTPVPIASKPFPTSGRAEASPNDTAGARTEMGTGSRVPGGSPLGVSLVYSDKKSAAATSPAPHLVAGPLLGTVGKAPATVTNLLVGTPGYGAPAPPAVQFIAQGAPGGGTTAGSGAGAGSGPNGPVPLGILQPGALGKAGGITQVQYILPTLPQQLQVAPAPAPAPGTKAAAPSGPAPTTSIRFTLPPGTSTNGKVLAATAPTPGIPILQSVPSAPPPKAQSVSPVQAPPPGGSAQLLPGKVLVPLAAPSMSVRGGGAGQPLPLVSPPFSVPVQNGAQPPSKIIQLTPVPVSTPSGLVPPLSPATLPGPTSQPQKVLLPSSTRITYVQSAGGHALPLGTSPASSQAGTVTSYGPTSSVALGFTSLGPSGPAFVQPLLSAGQAPLLAPGQVGVSPVPSPQLPPACAAPGGPVITAFYSGSPAPTSSAPLAQPSQAPPSLVYTVATSTTPPAATILPKGPPAPATATPAPTSPFPSATAGSMTYSLVAPKAQRPSPKAPQKVKAAIASIPVGSFEAGASGRPGPAPRQPLEPGPVREPTAPESELEGQPTPPAPPPLPETWTPTARSSPPLPPPAEERTSAKGPETMASKFPSSSSDWRVPGQGLENRGEPPTPPSPAPAPAVAPGGSSESSSGRAAGDTPERKEAAGTGKKVKVRPPPLKKTFDSVDNRVLSEVDFEERFAELPEFRPEEVLPSPTLQSLATSPRAILGSYRKKRKNSTDLDSAPEDPTSPKRKMRRRSSCSSEPNTPKSAKCEGDIFTFDRTGTEAEDVLGELEYDKVPYSSLRRTLDQRRALVMQLFQDHGFFPSAQATAAFQARYADIFPSKVCLQLKIREVRQKIMQAATPTEQPPGAEAPLPVPPPTGTAAAPAPTPSPAGGPDPTSPSSDSGTAQAAPPLPPPPESGPGQPGWEGAPQPSPPPPGPSTAATGR

SEQ ID NO: 55 - protein, 589 aa, Homo sapiens
MKRVRTEQIQMAVSCYLKRRQYVDSDGPLKQGLRLSQTAEEMAANLTVQSESGCANIVSAAPCQAEPQQYEVQFGRLRNFLTDSDSQHSHEVMPLLYPLFVYLHLNLVQNSPKSTVESFYSRFHGMFLQNASQKDVIEQLQTTQTIQDILSNFKLRAFLDNKYVVRLQEDSYNYLIRYLQSDNNTALCKVLTLHIHLDVQPAKRTDYQLYASGSSSRSENNGLEPPDMPSPILQNEAALEVLQESIKRVKDGPPSLTTICFYAFYNTEQLLNTAEISPDSKLLAAGFDNSCIKLWSLRSKKLKSEPHQVDVSRIHLACDILEEEDDEDPRTGTEMKILRGHCGPVYSTRFLADSSGLLSCSEDMSIRYWDLGSFTNTVLYQGHAYPVWDLDISPYSLYFASGSHDRTARLWSFDRTYPLRIYAGHLADVDCVKFHPNSNYLATGSTDKTVRLWSAQQGNSVRLFTGHRGPVLSLAFSPNGKYLASAGEDQRLKLWDLASGTLYKELRGHTDNITSLTFSPDSGLIASASMDNSVRVWDIRNTYCSAPADGSSSELVGVYTGQMSNVLSVQFMACNLLLVTGITQENQEH

SEQ ID NO: 56 - protein, 525 aa, Homo sapiens
MVSRPEPEGEAMDAELAVAPPGCSHLGSFKVDNWKQNLRAIYQCFVWSGTAEARKRKAKSCICHVCGVHLNRLHSCLYCVFFGCFTKKHIHEHAKAKRHNLAIDLMYGGIYCFLCQDYIYDKDMEIIAKEEQRKAWKMQGVGEKFSTWEPTKRELELLKHNPKRRKITSNCTIGLRGLINLGNTCFMNCIVQALTHTPLLRDFFLSDRHRCEMQSPSSCLVCEMSSLFQEFYSGHRSPHIPYKLLHLVWTHARHLAGYEQQDAHEFLIAALDVLHRHCKGDDNGKKANNPNHCNCIIDQIFTGGLQSDVTCQVCHGVSTTIDPFWDISLDLPGSSTPFWPLSPGSEGNVVNGESHVSGTTTLTDCLRRFTRPEHLGSSAKIKCSGCHSYQESTKQLTMKKLPIVACFHLKRFEHSAKLRRKITTYVSFPLELDMTPFMASSKESRMNGQYQQPTDSLNNDNKYSLFAVVNHQGTLESGHYTSFIRQHKDQWFKCDDAIITKASIKDVLDSEGYLLFYHKQFLEYE

SEQ ID NO: 57 - protein, 178 aa, Homo sapiens
MSTPPLAASGMAPGPFAGPQAQQAAREVNTASLCRIGQETVQDIVYRTMEIFQLLRNMQLPNGVTYHTGTYQDRLTKLQDNLRQLSVLFRKLRLVYDKCNENCGGMDPIPVEQLIPYVEEDGSKNDDRAGPPRFASEERREIAEVNKKLKQKNQQLKQIMDQLRNLIWDINAMLAMRN

SEQ ID NO: 58 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 59 - protein, 117 aa, Homo sapiens
MATYSLANERLRALEDIEREIGAILQNAGTVILELSKEKTNERLLDRQAAAFTASVQHVEAELSAQIRYLTQVATGQPHEGSSYSSRKDCQMALKRVDYARLKLSDVARTCEQMLEN

SEQ ID NO: 60 - protein, 533 aa, Homo sapiens
MSWAARPPFLPQRHAAGQCGPVGVRKEMHCGVASRWRRRRPWLDPAAAAAAAVAGGEQQTPEPEPGEAGRDGMGDSGRDSRSPDSSSPNPLPQGVPPPSPPGPPLPPSTAPSLGGSGAPPPPPMPPPPLGSPFPVISSSMGSPGLPPPAPPGFSGPVSSPQINSTVSLPGGGSGPPEDVKPPVLGVRGLHCPPPPGGPGAGKRLCAICGDRSSGKHYGVYSCEGCKGFFKRTIRKDLTYSCRDNKDCTVDKRQRNRCQYCRYQKCLATGMKREAVQEERQRGKDKDGDGEGAGGAPEEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPDAKGLSNPSEVEVLREKVYASLETYCKQKYPEQQGRFAKLLLRLPALRSIGLKCLEHLFFFKLIGDTPIDTFLMEMLEAPHQLA

SEQ ID NO: 61 - protein, 509 aa, Homo sapiens
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 62 - protein, 322 aa, Homo sapiens
MADRDSGSEQGGAALGSGGSLGHPGSGSGSGGGGGGGGGGGGSGGGGGGAPGGLQHETQELASKRVDIQNKRFYLDVKQNAKGRFLKIAEVGAGGNKSRLTLSMSVAVEFRDYLGDFIEHYAQLGPSQPPDLAQAQDEPRRALKSEFLVRENRKYYMDLKENQRGRFLRIRQTVNRGPGLGSTQGQTIALPAQGLIEFRDALAKLIDDYGVEEEPAELPEGTSLTVDNKRFFFDVGSNKYGVFMRVSEVKPTYRNSITVPYKVWAKFGHTFCKYSEEMKKIQEKQREKRAACEQLHQQQQQQQEETAAATLLLQGEEEGEED

SEQ ID NO: 63 - protein, 1290 aa, Homo sapiens
MSSLPGCIGLDAATATVESEEIAELQQAVVEELGISMEELRHFIDEELEKMDCVQQRKKQLAELETWVIQKESEVAHVDQLFDDASRAVTNCESLVKDFYSKLGLQYRDSSSEDESSRPTEIIEIPDEDDDVLSIDSGDAGSRTPKDQKLREAMAALRKSAQDVQKFMDAVNKKSSSQDLHKGTLSQMSGELSKDGDLIVSMRILGKKRTKTWHKGTLIAIQTVGPGKKYKVKFDNKGKSLLSGNHIAYDYHPPADKLYVGSRVVAKYKDGNQVWLYAGIVAETPNVKNKLRFLIFFDDGYASYVTQSELYPICRPLKKTWEDIEDISCRDFIEEYVTAYPNRPMVLLKSGQLIKTEWEGTWWKSRVEEVDGSLVRILFLDDKRCEWIYRGSTRLEPMFSMKTSSASALEKKQGQLRTRPNMGAVRSKGPVVQYTQDLTGTGTQFKPVEPPQPTAPPAPPFPPAPPLSPQAGDSDLESQLAQSRKQVAKKSTSFRPGSVGSGHSSPTSPALSENVSGGKPGINQTYRSPLGSTASAPAPSALPAPPAPPVFHGMLERAPAEPSYRAPMEKLFYLPHVCSYTCLSRVRPMRNEQYRGKNPLLVPLLYDFRRMTARRRVNRKMGFHVIYKTPCGLCLRTMQEIERYLFETGCDFLFLEMFCLDPYVLVDRKFQPYKPFYYILDITYGKEDVPLSCVNEIDTTPPPQVAYSKERIPGKGVFINTGPEFLVGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKIRAGTELTWDYNYEVGSVEGKELLCCCGAIECRGRLL

SEQ ID NO: 64 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 65 - protein, 418 aa, Homo sapiens
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 66 - protein, 508 aa, Homo sapiens
MLPTQAGAAAALGRGSALGGSLNRTPTGRPGGGGGTRGANGGRVPGNGAGLGPGRLEREAAAAAATTPAPTAGALYSGSEGDSESGEEEELGAERRGLKRSLSEMEIGMVVGGPEASAAATGGYGPVSGAVSGAKPGKKTRGRVKIKMEFIDNKLRRYTTFSKRKTGIMKKAYELSTLTGTQVLLLVASETGHVYTFATRKLQPMITSETGKALIQTCLNSPDSPPRSDPTTDQRMSATGFEETDLTYQVSESDSSGETKDTLKPAFTVTNLPGTTSTIQTAPSTSTTMQVSSGPSFPITNYLAPVSASVSPSAVSSANGTVLKSTGSGPVSSGGLMQLPTSFTLMPGGAVAQQVPVQAIQVHQAPQQASPSRDSSTDLTQTSSSGTVTLPATIMTSSVPTTVGGHMMYPSPHAVMYAPTSGLGDGSLTVLNAFSQAPSTMQVSHSQVQEPGGVPQVFLTASSGTVQIPVSAVQLHQMAVIGQQAGSSSNLTELQVVNLDTAHSTKSE

SEQ ID NO: 67 - protein, 443 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 68 - protein, 577 aa, Homo sapiens
MEESHFNSNPYFWPSIPTVSGQIENTMFINKMKDQLLPEKGCGLAPPHYPTLLTVPASVSLPSGISMDTESKSDQLTPHSQASVTQNITVVPVPSTGLMTAGVSCSQRWRREGSQSRGPGLVITSPSGSLVTTASSAQTFPISAPMIVSALPPGSQALQVVPDLSKKVASTLTEEGGGGGGGGGSVAPKPPRGRKKKRMLESGLPEMNDPYVLSPEDDDDHQKDGKTYRCRMCSLTFYSKSEMQIHSKSHTETKPHKCPHCSKTFANSSYLAQHIRIHSGAKPYSCNFCEKSFRQLSHLQQHTRIHSKMHTETIKPHKCPHCSKTFANTSYLAQHLRIHSGAKPYNCSYCQKAFRQLSHLQQHTRIHTGDRPYKCAHPGCEKAFTQLSNLQSHRRQHNKDKPFKCHNCHRAYTDAASLEVHLSTHTVKHAKVYTCTICSRAYTSETYLMKHMRKHNPPDLQQQVQAAAAAAAVAQAQAQAQAQAQAQAQAQAQAQASQASQQQQQQQQQQQQQQQQPPPHFQSPGAAPQGGGGGDSNPNPPPQCSFDLTPYKTAEHHKDICLTVTTSTIQVEHLASS

SEQ ID NO: 69 - protein, 794 aa, Homo sapiens
MNIDDKLEGLFLKCGGIDEMQSSRTMVVMGGVSGQSTVSGELQDSVLQDRSMPHQEILAADEVLQESEMRQQDMISHDELMVHEETVKNDEEQMETHERLPQGLQYALNVPISVKQEITFTDVSEQLMRDKKQIREPVDLQKKKKRKQRSPAKILTINEDGSLGLKTPKSHVCEHCNAAFRTNYHLQRHVFIHTGEKPFQCSQCDMRFIQKYLLQRHEKIHTGEKPFRCDECGMRFIQKYHMERHKRTHSGEKPYQCEYCLQYFSRTDRVLKHKRMCHENHDKKLNRCAIKGGLLTSEEDSGFSTSPKDNSLPKKKRQKTEKKSSGMDKESALDKSDLKKDKNDYLPLYSSSTKVKDEYMVAEYAVEMPHSSVGGSHLEDASGEIHPPKLVLKKINSKRSLKQPLEQNQTISPLSTYEESKVSKYAFELVDKQALLDSEGNADIDQVDNLQEGPSKPVHSSTNYDDAMQFLKKKRYLQAASNNSREYALNVGTIASQPSVTQAAVASVIDESTTASILESQALNVEIKSNHDKNVIPDEVLQTLLDHYSHKANGQHEISFSVADTEVTSSISINSSEVPEVTPSENVGSSSQASSSDKANMLQEYSKFLQQALDRTSQNDAYLNSPSLNFVTDNQTLPNQPAFSSIDKQVYATMPINSFRSGMNSPLRTTPDKSHFGLIVGDSQHSFPFSGDETNHASATSTQDFLDQVTSQKKAEAQPVHQAYQMSSFEQPFRAPYHGSRAGIATQFSTANGQVNLRGPGTSAEFSEFPLVNVNDNRAGMTSSPDATTGQTFG

SEQ ID NO: 70 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 71 - protein, 1454 aa, Homo sapiens
MAPVQLENHQLVPPGGGGGGSGGPPSAPAPPPPGAAVAAAAAAAASPGYRLSTLIEFLLHRAYSELMVLTDLLPRKSDVERKIEIVQFASRTRQLFVRLLALVKWANNAGKVEKCAMISSFLDQQAILFVDTADRLASLARDALVHARLPSFAIPYAIDVLTTGSYPRLPTCIRDKIIPPDPITKIEKQATLHQLNQILRHRLVTTDLPPQLANLTVANGRVKFRVEGEFEATLTVMGDDPDVPWRLLKLEILVEDKETGDGRALVHSMQISFIHQLVQSRLFADEKPLQDMYNCLHSFCLSLQLEVLHSQTLMLIRERWGDLVQVERYHAGKCLSLSVWNQQVLGRKTGTASVHKVTIKIDENDVSKPLQIFHDPPLPASDSKLVERAMKIDHLSIEKLLIDSVHARAHQKLQELKAILRGFNANENSSIETALPALVVPILEPCGNSECLHIFVDLHSGMFQLMLYGLDQATLDDMEKSVNDDMKRIIPWIQQLKFWLGQQRCKQSIKHLPTISSETLQLSNYSTHPIGNLSKNKLFIKLTRLPQYYIVVEMLEVPNKPTQLSYKYYFMSVNAADREDSPAMALLLQQFKENIQDLVFRTKTGKQTRTNAKRKLSDDPCPVESKKTKRAGEMCAFNKVLAHFVAMCDTNMPFVGLRLELSNLEIPHQGVQVEGDGFSHAIRLLKIPPCKGITEETQKALDRSLLDCTFRLQGRNNRTWVAELVFANCPLNGTSTREQGPSRHVYLTYENLLSEPVGGRKVVEMFLNDWNSIARLYECVLEFARSLPDIPAHLNIFSEVRVYNYRKLILCYGTTKGSSISIQWNSIHQKFHISLGTVGPNSGCSNCHNTILHQLQEMFNKTPNVVQLLQVLFDTQAPLNAINKLPTVPMLGLTQRTNTAYQCFSILPQSSTHIRLAFRNMYCIDIYCRSRGVVAIRDGAYSLFDNSKLVEGFYPAPGLKTFLNMFVDSNQDARRRSVNEDDNPPSPIGGDMMDSLISQLQPPPQQQPFPKQPGTSGAYPLTSPPTSYHSTVNQSPSMMHTQSPGNLHAASSPSGALRAPSPASFVPTPPPSSHGISIGPGASFASPHGTLDPSSPYTMVSPSGRAGNWPGSPQVSGPSPAARMPGMSPANPSLHSPVPDASHSPRAGTSSQTMPTNMPPPRKLPQRSWAASIPTILTHSALNILLLPSPTPGLVPGLAGSYLCSPLERFLGSVIMRRHLQRIIQQETLQLINSNEPGVIMFKTDALKCRVALSPKTNQTLQLKVTPENAGQWKPDELQVLEKFFETRVAGPPFKANTLIAFTKLLGAPTHILRDCVHIMKLELFPDQATQLKWNVQFCLTIPPSAPPIAPPGTPAVVLKSKMLFFLQLTQKTSVPPQEPVSIIVPIIYDMASGTTQQADIPRQQNSSVAAPMMVSNILKRFAEMNPPRQGECTIFAAVRDLMANLTLPPGGRP

SEQ ID NO: 72 - protein, 436 aa, Homo sapiens
MMATQTLSIDSYQDGQQMQVVTELKTEQDPNCSEPDAEGVSPPPVESQTPMDVDKQAIYRHPLFPLLALLFEKCEQSTQGSEGTTSASFDVDIENFVRKQEKEGKPFFCEDPETDNLMVKAIQVLRIHLLELEKVNELCKDFCSRYIACLKTKMNSETLLSGEPGSPYSPVQSQQIQSAITGTISPQGIVVPASALQQGNVAMATVAGGTVYQPVTVVTPQGQVVTQTLSPGTIRIQNSQLQLQLNQDLSILHQDDGSSKNKRGVLPKHATNVMRSWLFQHIGHPYPTEDEKKQIAAQTNLTLLQVNNWFINARRRILQPMLDSSCSETPKTKKKTAQNRPVQRFWPDSIASGVAQPPPSELTMSEGAVVTITTPVNMNVDSLQSLSSDGATLAVQQVMMAGQSEDESVDSTEEDAGALAPAHISGLVLENSDSLQ

SEQ ID NO: 73 - protein, 346 aa, Homo sapiens
MDMLDPGLDPAASATAAAAASHDKGPEAEEGVELQEGGDGPGAEEQTAVAITSVQQAAFGDHNIQYQFRTETNGGQVTYRVVQVTDGQLDGQGDTAGAVSVVSTAAFAGGQQAVTQVGVDGAAQRPGPAAASVPPGPAAPFPLAVIQNPFSNGGSPAAEAVSGEARFAYFPASSVGDTTAVSVQTTDQSLQAGGQFYVMMTPQDVLQTGTQRTIAPRTHPYSPKIDGTRTPRDERRRAQHNEVERRRRDKINNWIVQLSKIIPDCNADNSKTGASKGGILSKACDYIRELRQTNQRMQETFKEAERLQMDNELLRQQIEELKNENALLRAQLQQHNLEMVGEGTRQ

SEQ ID NO: 74 - protein, 283 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 75 - protein, 1616 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 76 - protein, 428 aa, Homo sapiens
MAENDVDNELLDYEDDEVETAAGGDGAEAPAKKDVKGSYVSIHSSGFRDFLLKPELLRAIVDCGFEHPSEVQHECIPQAILGMDVLCQAKSGMGKTAVFVLATLQQLEPVTGQVSVLVMCHTRELAFQISKEYERFSKYMPNVKVAVFFGGLSIKKDEEVLKKNCPHIVVGTPGRILALARNKSLNLKHIKHFILDECDKMLEQLDMRRDVQEIFRMTPHEKQVMMFSATLSKEIRPVCRKFMQDPMEIFVDDETKLTLHGLQQYYVKLKDNEKNRKLFDLLDVLEFNQVVIFVKSVQRCIALAQLLVEQNFPAIAIHRGMPQEERLSRYQQFKDFQRRILVATNLFGRGMDIERVNIAFNYDMPEDSDTYLHRVARAGRFGTKGLAITFVSDENDAKILNDVQDRFEVNISELPDEIDISSYIEQTR

SEQ ID NO: 77 - protein, 1845 aa, Homo sapiens
MGLCGLLERCWLHHDPDGVLTLNAENTNYAYQVPNFHKCEICLLSFPKESQFQRHMRDHERNDKPHRCDQCPQTFNVEFNLTLHKCTHSGEDPTCPVCNKKFSRVASLKAHIMLHEKEENLICSECGDEFTLQSQLAVHMEEHRQELAGTRQHACKACKKEFETSSELKEHMKTHYKIRVSSTRSYNRNIDRSGFTYSCPHCGKTFQKPSQLTRHIRIHTGERPFKCSECGKAFNQKGALQTHMIKHTGEKPHACAFCPAAFSQKGNLQSHVQRVHSEVKNGPTYNCTECSCVFKSLGSLNTHISKMHMGGPQNSTSSTETAHVLTATLFQTLPLQQTEAQATSASSQPSSQAVSDVIQQLLELSEPAPVESGQSPQPGQQLSITVGINQDILQQALENSGLSSIPAAAHPNDSCHAKTSAPHAQNPDVSSVSNEQTDPTDAEQEKEQESPEKLDKKEKKMIKKKSPFLPGSIREENGVRWHVCPYCAKEFRKPSDLVRHIRIHTHEKPFKCPQCFRAFAVKSTLTAHIKTHTGIKAFKCQYCMKSFSTSGSLKVHIRLHTGVRPFACPHCDKKFRTSGHRKTHIASHFKHTELRKMRHQRKPAKVRVGKTNIPVPDIPLQEPILITDLGLIQPIPKNQFFQSYFNNNFVNEADRPYKCFYCHRAYKKSCHLKQHIRSHTGEKPFKCSQCGRGFVSAGVLKAHIRTHTGLKSFKCLICNGAFTTGGSLRRHMGIHNDLRPYMCPYCQKTFKTSLNCKKHMKTHRYELAQQLQQHQQAASIDDSTVDQQSMQASTQMQVEIESDELPQTAEVVAANPEAMLDLEPQHVVGTEEAGLGQQLADQPLEADEDGFVAPQDPLRGHVDQFEEQSPAQQSFEPAGLPQGFTVTDTYHQQPQFPPVQQLQDSSTLESQALSTSFHQQSLLQAPSSDGMNVTTRLIQESSQEELDLQAQGSQFLEDNEDQSRRSYRCDYCNKGFKKSSHLKQHVRSHTGEKPYKCKLCGRGFVSSGVLKSHEKTHTGVKAFSCSVCNASFTTNGSLTRHMATHMSMKPYKCPFCEEGFRTTVHCKKHMKRHQTVPSAVSATGETEGGDICMEEEEEHSDRNASRKSRPEVITFTEEETAQLAKIRPQESATVSEKVLVQSAAEKDRISELRDKQAELQDEPKHANCCTYCPKSFKKPSDLVRHVRIHTGEKPYKCDECGKSFTVKSTLDCHVKTHTGQKLFSCHVCSNAFSTKGSLKVHMRLHTGAKPFKCPHCELRFRTSGRRKTHMQFHYKPDPKKARKPMTRSSSEGLQPVNLLNSSSTDPNVFIMNNSVLTGQFDQNLLQPGLVGQAILPASVSAGGDLTVSLTDGSLATLEGIQLQLAANLVGPNVQISGIDAASINNITLQIDPSILQQTLQQGNLLAQQLTGEPGLAPQNSSLQTSDSTVPASVVIQPISGLSLQPTVTSANLTIGPLSEQDSVLTTNSSGTQDLTQVMTSQGLVSPSGGPHEITLTINNSSLSQVLAQAAGPTATSSSGSPQEITLTISELNTTSGSLPSTTPMSPSAISTQNLVMSSSGVGGDASVTLTLADTQGMLSGGLDTVTLNITSQGQQFPALLTDPSLSGQGGAGSPQVILVSHTPQSASAACEEIAYQVAGVSGNLAPGNQPEKEGRAHQCLECDRAFSSAAVLMHHSKEVHGRERIHGCPVCRKAFKRATHLKEHMQTHQAGPSLSSQKPRVFKCDTCEKAFAKPSQLERHSRIHTGERPFHCTLCEKAFNQKSALQVHMKKHTGERPYKCAYCVMGFTQKSNMKLHMKRAHSYAGALQESAGHPEQDGEELSRTLHLEEVVQEAAGEWQALTHVF

SEQ ID NO: 78 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 79 - protein, 454 aa, Homo sapiens
MTKREAEELIEIEIDGTEKAECTEESIVEQTYAPAECVSQAIDINEPIGNLKKLLEPRLQCSLDAHEICLQDIQLDPERSLFDQGVKTDGTVQLSVQVISYQGIEPKLNILEIVKPADTVEVVIDPDAHHAESEAHLVEEAQVITLDGTKHITTISDETSEQVTRWAAALEGYRKEQERLGIPYDPIQWSTDQVLHWVVWVMKEFSMTDIDLTTLNISGRELCSLNQEDFFQRVPRGEILWSHLELLRKYVLASQEQQMNEIVTIDQPVQIIPASVQSATPTTIKVINSSAKAAKVQRAPRISGEDRSSPGNRTGNNGQIQLWQFLLELLTDKDARDCISWVGDEGEFKLNQPELVAQKWGQRKNKPTMNYEKLSRALRYYYDGDMICKVQGKRFVYKFVCDLKTLIGYSAAELNRLVTECEQKKLAKMQLHGIAQPVTAVALATASLQTEKDN

SEQ ID NO: 80 - protein, 500 aa, Homo sapiens
MDHTEGSPAEEPPAHAPSPGKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 81 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 82 - protein, 723 aa, Homo sapiens
MAAAATMAAAARELVLRAGTSDMEEEEGPLAGGPGLQEPLQLGELDITSDEFILDEVDVHIQANLEDELVKEALKTGVDLRHYSKQVELELQQIEQKSIRDYIQESENIASLHNQITACDAVLERMEQMLGAFQSDLSSISSEIRTLQEQSGAMNIRLRNRQAVRGKLGELVDGLVVPSALVTAILEAPVTEPRFLEQLQELDAKAAAVREQEARGTAACADVRGVLDRLRVKAVTKIREFILQKIYSFRKPMTNYQIPQTALLKYRFFYQFLLGNERATAKEIRDEYVETLSKIYLSYYRSYLGRLMKVQYEEVAEKDDLMGVEDTAKKGFFSKPSLRSRNTIFTLGTRGSVISPTELEAPILVPHTAQRGEQRYPFEALFRSQHYALLDNSCREYLFICEFFVVSGPAAHDLFHAVMGRTLSMTLKHLDSYLADCYDAIAVFLCIHIVLRFRNIAAKRDVPALDRYWEQVLALLWPRFELILEMNVQSVRSTDPQRLGGLDTRPHYITRRYAEFSSALVSINQTIPNERTMQLLGQLQVEVENFVLRVAAEFSSRKEQLVFLINNYDMMLGVLMERAADDSKEVESFQQLLNARTQEFIEELLSPPFGGLVAFVKEAEALIERGQAERLRGEEARVTQLIRGFGSSWKSSVESLSQDVMRSFTNFRNGTSIIQGALTQLIQLYHRFHRVLSQPQLRALPARAELINIHHLMVELKKHKPNF

SEQ ID NO: 83 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 84 - protein, 894 aa, Homo sapiens
MSRRKQGNPQHLSQRELITPEADHVEAAILEEDEGLEIEEPSGLGLMVGGPDPDLLTCGQCQMNFPLGDILVFIEHKRKQCGGSLGACYDKALDKDSPPPSSRSELRKVSEPVEIGIQVTPDEDDHLLSPTKGICPKQENIAGPCRPAQLPAVAPIAASSHPHSSVITSPLRALGALPPCLPLPCCSARPVSGDGTQGEGQTEAPFGCQCQLSGKDEPSSYICTTCKQPFNSAWFLLQHAQNTHGFRIYLEPGPASSSLTPRLTIPPPLGPEAVAQSPLMNFLGDSNPFNLLRMTGPILRDHPGFGEGRLPGTPPLFSPPPRHHLDPHRLSAEEMGLVAQHPSAFDRVMRLNPMAIDSPAMDFSRRLRELAGNSSTPPPVSPGRGNPMHRLLNPFQPSPKSPFLSTPPLPPMPPGGTPPPQPPAKSKSCEFCGKTFKFQSNLIVHRRSHTGEKPYKCQLCDHACSQASKLKRHMKTHMHKAGSLAGRSDDGLSAASSPEPGTSELAGEGLKAADGDFRHHESDPSLGHEPEEEDEEEEEEEEELLLENESRPESSFSMDSELSRNRENGGGGVPGVPGAGGGAAKALADEKALVLGKVMENVGLGALPQYGELLADKQKRGAFLKRAAGGGDAGDDDDAGGCGDAGAGGAVNGRGGGFAPGTEPFPGLFPRKPAPLPSPGLNSAAKRIKVEKDLELPPAALIPSENVYSQWLVGYAASRHFMKDPFLGFTDARQSPFATSSEHSSENGSLRFSTPPGDLLDGGLSGRSGTASGGSTPHLGGPGPGRPSSKEGRRSDTCEYCGKVFKNCSNLTVHRRSHTGERPYKCELCNYACAQSSKLTRHMKTHGQIGKEVYRCDICQMPFSVYSTLEKHMKKWHGEHLLTNDVKIEQAERS

SEQ ID NO: 85 - protein, 631 aa, Homo sapiens
MFYAHFVLSKRGPLAKIWLAAHWDKKLTKAHVFECNLESSVESIISPKVKMALRTSGHLLLGVVRIYHRKAKYLLADCNEAFIKIKMAFRPGVVDLPEENREAAYNAITLPEEFHDFDQPLPDLDDIDVAQQFSLNQSRVEEITMREEVGNISILQENDFGDFGMDDREIMREGSAFEDDDMLVSTTTSNLLLESEQSTSNLNEKINHLEYEDQYKDDNFGEGNDGGILDDKLISNNDGGIFDDPPALSEAGVMLPEQPAHDDMDEDDNVSMGGPDSPDSVDPVEPMPTMTDQTTLVPNEEEAFALEPIDITVKETKAKRKRKLIVDSVKELDSKTIRAQLSDYSDIVTTLDLAPPTKKLMMWKETGGVEKLFSLPAQPLWNNRLLKLFTRCLTPLVPEDLRKRRKGGEADNLDEFLKEFENPEVPREDQQQQHQQRDVIDEPIIEEPSRLQESVMEASRTNIDESAMPPPPPQGVKRKAGQIDPEPVMPPQQVEQMEIPPVELPPEEPPNICQLIPELELLPEKEKEKEKEKEDDEEEEDEDASGGDQDQEERRWNKRTQQMLHGLQRALAKTGAESISLLELCRNTNRKQAAAKFYSFLVLKKQQAIELTQEEPYSDIIATPGPRFHII

SEQ ID NO: 86 - protein, 2446 aa, Homo sapiens
MDTGDTALGQKATSRSGETDKASGRWRQEQSAVIKMSTFGSHEGQRQPQIEPEQIGNTASAQLFGSGKLASPSEVVQQVAEKQYPPHRPSPYSCQHSLSFPQHSLPQGVMHSTKPHQSLEGPPWLFPGPLPSVASEDLFPFPIHGHSGGYPRKKISSLNPAYSQYSQKSIEQAEEAHKKEHKPKKPGKYICPYCSRACAKPSVLKKHIRSHTGERPYPCIPCGFSFKTKSNLYKHRKSHAHAIKAGLVPFTESAVSKLDLEAGFIDVEAEIHSDGEQSTDTDEESSLFAEASDKMSPGPPIPLDIASRGGYHGSLEESLGGPMKVPILIIPKSGIPLPNESSQYIGPDMLPNPSLNTKADDSHTVKQKLALRLSEKKGQDSEPSLNLLSPHSKGSTDSGYFSRSESAEQQISPPNTNAKSYEEIIFGKYCRLSPRNALSVTTTSQERAAMGRKGIMEPLPHVNTRLDVKMFEDPVSQLIPSKGDVDPSQTSMLKSTKFNSESRQPQIIPSSIRNEGKLYPANFQGSNPVLLEAPVDSSPLIRSNSVPTSSATNLTIPPSLRGSHSFDERMTGSDDVFYPGTVGIPPQRMLRRQAAFELPSVQEGHVEVEHHGRMLKGISSSSLKEKKLSPGDRVGYDYDVCRKPYKKWEDSETPKQNYRDISCLSSLKHGGEYFMDPVVPLQGVPSMFGTTCENRKRRKEKSVGDEEDTPMICSSIVSTPVGIMASDYDPKLQMQEGVRSGFAMAGHENLSHGHTERFDPCRPQLQPGSPSLVSEESPSAIDSDKMSDLGGRKPPGNVISVIQHTNSLSRPNSFERSESAELVACTQDKAPSPSETCDSEISEAPVSPEWAPPGDGAESGGKPSPSQQVQQQSYHTQPRLVRQHNIQVPEIRVTEEPDKPEKEKEAQSKEPEKPVEEFQWPQRSETLSQLPAEKLPPKKKRLRLADMEHSSGESSFESTGTGLSRSPSQESNLSHSSSFSMSFEREETSKLSALPKQDEFGKHSEFLTVPAGSYSLSVPGHHHQKEMRRCSSEQMPCPHPAEVPEVRSKSFDYGNLSHAPVSGAAASTVSPSRERKKCFLVRQASFSGSPEISQGEVGMDQSVKQEQLEHLHAGLRSGWHHGPPAVLPPLQQEDPGKQVAGPCPPLSSGPLHLAQPQIMHMDSQESLRNPLIQPTSYMTSKHLPEQPHLFPHQETIPFSPIQNALFQFQYPTVCMVHLPAQQPPWWQAHFPHPFAQHPQKSYGKPSFQTEIHSSYPLEHVAEHTGKKPAEYAHTKEQTYPCYSGASGLHPKNLLPKFPSDQSSKSTETPSEQVLQEDFASANAGSLQSLPGTVVPVRIQTHVPSYGSVMYTSISQILGQNSPAIVICKVDENMTQRTLVTNAAMQGIGFNIAQVLGQHAGLEKYPIWKAPQTLPLGLESSIPLCLPSTSDSVATLGGSKRMLSPASSLELFMETKQQKRVKEEKMYGQIVEELSAVELTNSDIKKDLSRPQKPQLVRQGCASEPKDGLQSGSSSFSSLSPSSSQDYPSVSPSSREPFLPSKEMLSGSRAPLPGQKSSGPSESKESSDELDIDETASDMSMSPQSSSLPAGDGQLEEEGKGHKRPVGMLVRMASAPSGNVADSTLLLTDMADFQQILQFPSLRTTTTVSWCFLNYTKPNYVQQATFKSSVYASWCISSCNPNPSGLNTKTTLALLRSKQKITAEIYTLAAMHRPGTGKLTSSSAWKQFTQMKPDASFLFGSKLERKLVGNILKERGKGDIHGDKDIGSKQTEPIRIKIFEGGYKSNEDYVYVRGRGRGKYICEECGIRCKKPSMLKKHIRTHTDVRPYVCKLCNFAFKTKGNLTKHMKSKAHMKKCLELGVSMTSVDDTETEEAENLEDLHKAAEKHSMSSISTDHQFSDAEESDGEDGDDNDDDDEDEDDFDDQGDLTPKTRSRSTSPQPPRFSSLPVNVGAVPHGVPSDSSLGHSSLISYLVTLPSIRVTQLMTPSDSCEDTQMTEYQRLFQSKSTDSEPDKDRLDIPSCMDEECMLPSEPSSSPRDFSPSSHHSSPGYDSSPCRDNSPKRYLIPKGDLSPRRHLSPRRDLSPMRHLSPRKEAALRREMSQRDVSPRRHLSPRRPVSPGKDITARRDLSPRRERRYMTTIRAPSPRRALYHNPPLSMGQYLQAEPIVLGPPNLRRGLPQVPYFSLYGDQEGAYEHPGSSLFPEGPNDYVFSHLPLHSQQQVRAPIPMVPVGGIQMVHSMPPALSSLHPSPTLPLPMEGFEEKKGASGESFSKDPYVLSKQHEKRGPHALQSSGPPSTPSSPRLLMKQSTSEDSLNATEREQEENIQTCTKAIASLRIATEEAALLGPDQPARVQEPHQNPLGSAHVSIRHFSRPEPGQPCTSATHPDLHDGEKDNFGTSQTPLAHSTFYSKSCVDDKQLDFHSSKELSSSTEESKDPSSEKSQLH

SEQ ID NO: 87 - protein, 349 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 88 - protein, 338 aa, Homo sapiens
MEYFMVPTQKVPSLQHFRKTEKEVIGGLCSLANIPLTPETQRDQERRIRREIANSNERRRMQSINAGFQSLKTLIPHTDGEKLSKAAILQQTAEYIFSLEQEKTRLLQQNTQLKRFIQELSGSSPKRRRAEDKDEGIGSPDIWEDEKAEDLRREMIELRQQLDKERSVRMMLEEQVRSLEAHMYPEKLKVIAQQVQLQQQQEQVRLLHQEKLEREQQQLRTQLLPPPAPTHHPTVIVPAPPPPPSHHINVVTMGPSSVINSVSTSRQNLDTIVQAIQHIEGTQEKQELEEEQRRAVIVKPVRSCPEAPTSDTASDSEASDSDAMDQSREEPSGDGELP

SEQ ID NO: 89 - protein, 825 aa, Homo sapiens
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISPRTDRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 90 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 91 - protein, 521 aa, Homo sapiens
MGRKKIQIQRITDERNRQVTFTKRKFGLMKKAYELSVLCDCEIALIIFNHSNKLFQYASTDMDKVLLKYTEYNEPHESRTNADIIETLRKKGFNGCDSPEPDGEDSLEQSPLLEDKYRRASEELDGLFRRYGSTVPAPNFAMPVTVPVSNQSSLQFSNPSGSLVTPSLVTSSLTDPRLLSPQQPALQRNSVSPGLPQRPASAGAMLGGDLNSANGACPSPVGNGYVSARASPGLLPVANGNSLNKVIPAKSPPPPTHSTQLGAPSRKPDLRVITSQAGKGLMHHLTEDHLDLNNAQRLGVSQSTHSLTTPVVSVATPSLLSQGLPFSSMPTAYNTDYQLTSAELSSLPAFSSPGGLSLGNVTAWQQPQQPQQPQQPQPPQQQPPQPQQPQPQQPQQPQQPPQQQSHLVPVSLSNLIPGSPLPHVGAALTVTTHPHISIKSEPVSPSRERSPAPPPPAVFPAARPEPGDGLSSPAGGSYETGDRDDGRGDFGPTLGLLRPAPEPEAEGSAVKRMRLDTWTLK

SEQ ID NO: 92 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 93 - protein, 900 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVTKLRANVNARTFAGNTPLHLAAGLGYPTLTRLLLKAGADIHAENEEPLCPLPSPPTSDSDSDSEGPEKDTRSSFRGHTPLDLTCSTKVKTLLLNAAQNTMEPPLTPPSPAGPGLSLGDTALQNLEQLLDGPEAQGSWAELAERLGLRSLVDTYRQTTSPSGSLLRSYELAGGDLAGLLEALSDMGLEEGVRLLRGPETRDKLPSTAEVKEDSAYGSQSVEQEAEKLGPPPEPPGGLCHGHPQPQVH

SEQ ID NO: 94 - protein, 464 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 95 - protein, 753 aa, Homo sapiens
MGEHSPDNNIIYFEAEEDELTPDDKMLRFVDKNGLVPSSSGTVYDRTTVLIEQDPGTLEDEDDDGQCGEHLPFLVGGEEGFHLIDHEAMSQGYVQHIISPDQIHLTINPGSTPMPRNIEGATLTLQSECPETKRKEVKRYQCTFEGCPRTYSTAGNLRTHQKTHRGEYTFVCNQEGCGKAFLTSYSLRIHVRVHTKEKPFECDVQGCEKAFNTLYRLKAHQRLHTGKTFNCESEGCSKYFTTLSDLRKHIRTHTGEKPFRCDHDGCGKAFAASHHLKTHVRTHTGERPFFCPSNGCEKTFSTQYSLKSHMKGHDNKGHSYNALPQHNGSEDTNHSLCLSDLSLLSTDSELRENSSTTQGQDLSTISPAIIFESMFQNSDDTAIQEDPQQTASLTESFNGDAESVSDVPPSTGNSASLSLPLVLQPGLSEPPQPLLPASAPSAPPPAPSLGPGSQQAAFGNPPALLQPPEVPVPHSTQFAANHQEFLPHPQAPQPIVPGLSVVAGASASAAAVASAVAAPAPPQSTTEPLPAMVQTLPLGANSVLTNNPTITITPTPNTAILQSSLVMGEQNLQWILNGATSSPQNQEQIQQASKVEKVFFTTAVPVASSPGSSVQQIGLSVPVIIIKQEEACQCQCACRDSAKERASSRRKGCSSPPPPEPSPQAPDGPSLQLPAQTFSSAPVPGSSSSTLPSSCEQSRQAETPSDPQTETLSAMDVSEFLSLQSLDTPSNLIPIEALLQGEEEMGLTSSFSK

SEQ ID NO: 96 - protein, 845 aa, Homo sapiens
MPKAPKQQPPEPEWIGDGESTSPSDKVVKKGKKDKKIKKTFFEELAVEDKQAGEEEKVLKEKEQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKPQNKFAALDNEEEDKEEEIIKEKEPPKQGKEKAKKAEQGSEEEGEGEEEEEEGGESKADDPYAHLSKKEKKKLKKQMEYERQVASLKAANAAENDFSVSQAEMSSRQAMLENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 97 - protein, 339 aa, Homo sapiens
MDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 98 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 99 - protein, 450 aa, Homo sapiens
MNLEGGGRGGEFGMSAVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMSMSHPYTMTTPYPSLPAQVHNYMMPPLDRSWRDYVPDQPHPEIPYQCPMTFGPRGHHWQGPACENGCQVTGTFYACAPPESQAPGVPTEPSIRSAEALAFSDCRLHICLYYREILVKELTTSSPEGCRISHGHTYDASNLDQVLFPYPEDNGQRKNIEKLLSHLERGVVLWMAPDGLYAKRLCQSRIYWDGPLALCNDRPNKLERDQTCKLFDTQQFLSELQAFAHHGRSLPRFQVTLCFGEEFPDPQRQRKLITAHVEPLLARQLYYFAQQNSGHFLRGYDLPEHISNPEDYHRSIRHSSIQE

SEQ ID NO: 100 - protein, 763 aa, Homo sapiens
MDHLNEATQGKEHSEMSNNVSDPKGPPAKIARLEQNGSPLGRGRLGSTGAKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQGLLSEILRKEEDPKTASQSLLVNLRAMQNFLQLPEAERDRIYQDERERSLNAASAMGPAPLISTPPSRPPQVKTATIATERNGKPENNTMNINASIYDEIQQEMKRAKVSQALFAKVAATKSQGWLCELLRWKEDPSPENRTLWENLSMIRRFLSLPQPERDAIYEQESNAVHHHGDRPPHIIHVPAEQIQQQQQQQQQQQQQQQAPPPPQPQQQPQTGPRLPPRQPTVASPAESDEENRQKTRPRTKISVEALGILQSFIQDVGLYPDEEAIQTLSAQLDLPKYTIIKFFQNQRYYLKHHGKLKDNSGLEVDVAEYKEEELLKDLEESVQDKNTNTLFSVKLEEELSVEGNTDINTDLKD

SEQ ID NO: 101 - protein, 327 aa, Homo sapiens
MTMESGAENQQSGDAAVTEAENQQMTVQAQPQIATLAQVSMPAAHATSSAPTVTLVQLPNGQTVQVHGVIQAAQPSVIQSPQVQTVQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 102 - protein, 125 aa, Homo sapiens
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 103 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 104 - protein, 182 aa, Homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEVRVSQLLAVTGKKTTRP

SEQ ID NO: 105 - protein, 975 aa, Homo sapiens
MSGIGNKRAAGEPGTSMPPEKKAAVEDSGTTVETIKLGGVSSTEELDIRTLQTKNRKLAEMLDQRQAIEDELREHIEKLERRQATDDASLLIVNRYWSQFDENIRIILKRYDLEQGLGDLLTERKALVVPEPEPDSDSNQERKDDRERGEGQEPAFSFLATLASSSSEEMESQLQERVESSRRAVSQIVTVYDKLQEKVELLSRKLNSGDNLIVEEAVQELNSFLAQENMRLQELTDLLQEKHRTMSQEFSKLQSKVETAESRVSVLESMIDDLQWDIDKIRKREQRLNRHLAEVLERVNSKGYKVYGAGSSLYGGTITINARKFEEMNAELEENKELAQNRLCELEKLRQDFEEVTTQNEKLKVELRSAVEQVVKETPEYRCMQSQFSVLYNESLQLKAHLDEARTLLHGTRGTHQHQVELIERDEVSLHKKLRTEVIQLEDTLAQVRKEYEMLRIEFEQTLAANEQAGPINREMRHLISSLQNHNHQLKGEVLRYKRKLREAQSDLNKTRLRSGSALLQSQSSTEDPKDEPAELKPDSEDLSSQSSASKASQEDANEIKSKRDEEERERERREKEREREREREKEKEREREKQKLKESEKERDSAKDKEKGKHDDGRKKEAEIIKQLKIELKKAQESQKEMKLLLDMYRSAPKEQRDKVQLMAAEKKSKAELEDLRQRLKDLEDKEKKENKKMADEDALRKIRAVEEQIEYLQKKLAMAKQEEEALLSEMDVTGQAFEDMQEQNIRLMQQLREKDDANFKLMSERIKSNQIHKLLKEEKEELADQVLTLKTQVDAQLQVVRKLEEKEHLLQSNIGTGEKELGLRTQALEMNKRKAMEAAQLADDLKAQLELAQKKLHDFQDEIVENSVTKEKDMFNFKRAQEDISRLRRKLETTKKPDNVPKCDEILMEEIKDYKARLTCPCCNMRKKDAVLTKCFHVFCFECVKTRYDTRQRKCPKCNAAFGANDFHRIYIG

SEQ ID NO: 106 - protein, 615 aa, Homo sapiens
MTSPSPRIQIISTDSAVASPQRIQGSEPASGPLSVFTSLNKEKIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 107 - protein, 733 aa, Homo sapiens
MAEVGEDSGARALLALRSAPCSPVLCAAAAAAAFPAAAPPPAPAQPQPPPGPPPPPPPPLPPGAIAGAGSSGGSSGVSGDSAVAGAAPALVAAAAASVRQSPGPALARLEGREFEFLMRQPSVTIGRNSSQGSVDLSMGLSSFISRRHLQLSFQEPHFYLRCLGKNGVFVDGAFQRRGAPALQLPKQCTFRFPSTAIKIQFTSLYHKEEAPASPLRPLYPQISPLKIHIPEPDLRSMVSPVPSPTGTISVPNSCPASPRGAGSSSYRFVQNVTSDLQLAAEFAAKAASEQQADTSGGDSPKDESKPPFSYAQLIVQAISSAQDRQLTLSGIYAHITKHYPYYRTADKGWQNSIRHNLSLNRYFIKVPRSQEEPGKGSFWRIDPASEAKLVEQAFRKRRQRGVSCFRTPFGPLSSRSAPASPTHPGLMSPRSGGLQTPECLSREGSPIPHDPEFGSKLASVPEYRYSQSAPGSPVSAQPVIMAVPPRPSSLVAKPVAYMPASIVTSQQPAGHAIHVVQQAPTVTMVRVVTTSANSANGYILTSQGAAGGSHDAAGAAVLDLGSEARGLEEKPTIAFATIPAAGGVIQTVASQMAPGVPGHTVTILQPATPVTLGQHHLPVRAVTQNGKHAVPTNSLAGNAYALTSPLQLLATQASSSAPVVVTRVCEVGPKEPAAAVAATATTTPATATTASASASSTGEPEVKRSRVEEPSGAVTTPAGVIAAAGPQGPGTGE

SEQ ID NO: 108 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 109 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 110 - protein, 410 aa, Homo sapiens
MAKDAGLIEANGELKVFIDQNLSPGKGVVSLVAVHPSTVNPLGKQLLPKTFGQSNVNIAQQVVIGTPQRPAASNTLVVGSPHTPSTHFASQNQPSDSSPWSAGKRNRKGEKNGKGLRHFSMKVCEKVQRKGTTSYNEVADELVAEFSAADNHILPNESAYDQKNIRRRVYDALNVLMAMNIISKEKKEIKWIGLPTNSAQECQNLEVERQRRLERIKQKQSQLQELILQQIAFKNLVQRNRHAEQQASRPPPPNSVIHLPFIIVNTSKKTVIDCSISNDKFEYLFNFDNTFEIHDDIEVLKRMGMACGLESGSCSAEDLKMARSLVPKALEPYVTEMAQGTVGGVFITTAGSTSNGTRFSASDLTNGADGMLATSSNGSQYSGSRVETPVSYVGEDDEEDDDFNENDEDD

SEQ ID NO: 111 - protein, 2920 aa, Homo sapiens
MRGRRGRPPKQPAAPAAERCAPAPPPPPPPPTSGPIGGLRSRHRGSSRGRWAAAQAEVAPKTRLSSPRGGSSSRRKPPPPPPAPPSTSAPGRGGRGGGGGRTGGGGGGGHLARTTAARRAVNKVVYDDHESEEEEEEEDMVSEEEEEEDGDAEETQDSEDDEEDEMEEDDDDSDYPEEMEDDDDDASYCTESSFRSHSTYSSTPGRRKPRVHRPRSPILEEKDIPPLEFPKSSEDLMVPNEHIMNVIAIYEVLRNFGTVLRLSPFRFEDFCAALVSQEQCTLMAEMHVVLLKAVLREEDTSNTTFGPADLKDSVNSTLYFIDGMTWPEVLRVYCESDKEYHHVLPYQEAEDYPYGPVENKIKVLQFLVDQFLTTNIAREELMSEGVIQYDDHCRVCHKLGDLLCCETCSAVYHLECVKPPLEEVPEDEWQCEVCVAHKVPGVTDCVAEIQKNKPYIRHEPIGYDRSRRKYWFLNRRLIIEEDTENENEKKIWYYSTKVQLAELIDCLDKDYWEAELCKILEEMREEIHRHMDITEDLTNKARGSNKSFLAAANEEILESIRAKKGDIDNVKSPEETEKDKNETENDSKDAEKNREEFEDQSLEKDSDDKTPDDDPEQGKSEVGDFKSEKSNGELSESPGAGKGASGSTRIITRLRNPDSKLSQLKSQQVAAAAHEANKLFKEGKEVLVVNSQGEISRLSTKKEVIMKGNINNYFKLGQEGKYRVYHNQYSTNSFALNKHQHREDHDKRRHLAHKFCLTPAGEFKWNGSVHGSKVLTISTLRLTITQLENNIPSSFLHPNWASHRANWIKAVQMCSKPREFALALAILECAVKPVVMLPIWRESLGHTRLHRMTSIEREEKEKVKKKEKKQEEEETMQQATWVKYTFPVKHQVWKQKGEEYRVTGYGGWSWISKTHVYRFVPKLPGNTNVNYRKSLEGTKNNMDENMDESDKRKCSRSPKKIKIEPDSEKDEVKGSDAAKGADQNEMDISKITEKKDQDVKELLDSDSDKPCKEEPMEVDDDMKTESHVNCQESSQVDVVNVSEGFHLRTSYKKKTKSSKLDGLLERRIKQFTLEEKQRLEKIKLEGGIKGIGKTSTNSSKNLSESPVITKAKEGCQSDSMRQEQSPNANNDQPEDLIQGCSESDSSVLRMSDPSHTTNKLYPKDRVLDDVSIRSPETKCPKQNSIENDIEEKVSDLASRGQEPSKSKTKGNDFFIDDSKLASADDIGTLICKNKKPLIQEESDTIVSSSKSALHSSVPKSTNDRDATPLSRAMDFEGKLGCDSESNSTLENSSDTVSIQDSSEEDMIVQNSNESISEQFRTREQDVEVLEPLKCELVSGESTGNCEDRLPVKGTEANGKKPSQQKKLEERPVNKCSDQIKLKNTTDKKNNENRESEKKGQRTSTFQINGKDNKPKIYLKGECLKEISESRVVSGNVEPKVNNINKIIPENDIKSLTVKESAIRPFINGDVIMEDFNERNSSETKSHLLSSSDAEGNYRDSLETLPSTKESDSTQTTTPSASCPESNSVNQVEDMEIETSEVKKVTSSPITSEEESNLSNDFIDENGLPINKNENVNGESKRKTVITEVTTMTSTVATESKTVIKVEKGDKQTVVSSTENCAKSTVTTTTTTVTKLSTPSTGGSVDIISVKEQSKTVVTTTVTDSLTTTGGTLVTSMTVSKEYSTRDKVKLMKFSRPKKTRSGTALPSYRKFVTKSSKKSIFVLPNDDLKKLARKGGIREVPYFNYNAKPALDIWPYPSPRPTFGITWRYRLQTVKSLAGVSLMLRLLWASLRWDDMAAKAPPGGGTTRTETSETEITTTEIIKRRDVGPYGIRSEYCIRKIICPIGVPETPKETPTPQRKGLRSSALRPKRPETPKQTGPVIIETWVAEEELELWEIRAFAERVEKEKAQAVEQQAKKRLEQQKPTVIATSTTSPTSSTTSTISPAQKVMVAPISGSVTTGTKMVLTTKVGSPATVTFQQNKNFHQTFATWVKQGQSNSGVVQVQQKVLGIIPSSTGTSQQTFTSFQPRTATVTIRPNTSGSGGTTSNSQVITGPQIRPGMTVIRTPLQQSTLGKAIIRTPVMVQPGAPQQVMTQIIRGQPVSTAVSAPNTVSSTPGQKSLTSATSTSNIQSSASQPPRPQQGQVKLTMAQLTQLTQGHGGNQGLTVVIQGQGQTTGQLQLIPQGVTVLPGPGQQLMQAAMPNGTVQRFLFTPLATTATTASTTTTTVSTTAAGTGEQRQSKLSPQMQVHQDKTLPPAQSSSVGPAEAQPQTAQPSAQPQPQTQPQSPAQPEVQTQPEVQTQTTVSSHVPSEAQPTHAQSSKPQVAAQSQPQSNVQGQSPVRVQSPSQTRIRPSTPSQLSPGQQSQVQTTTSQPIPIQPHTSLQIPSQGQPQSQPQVQSSTQTLSSGQTLNQVTVSSPSRPQLQIQQPQPQVIAVPQLQQQVQVLSQIQSQVVAQIQAQQSGVPQQIKLQLPIQIQQSSAVQTHQIQNVVTVQAASVQEQLQRVQQLRDQQQKKKQQQIEIKREHTLQASNQSEIIQKQVVMKHNAVIEHLKQKKSMTPAEREENQRMIVCNQVMKYILDKIDKEEKQAAKKRKREESVEQKRSKQNATKLSALLFKHKEQLRAEILKKRALLDKDLQIEVQEELKRDLKIKKEKDLMQLAQATAVAAPCPPVTPAPPAPPAPPPSPPPPPAVQHTGLLSTPTLPAASQKRKREEEKDSSSKSKKKKMISTTSKETKKDTKLYCICKTPYDESKFYIGCDRCQNWYHGRCVGILQSEAELIDEYVCPQCQSTEDAMTVLTPLTEKDYEGLKRVLRSLQAHKMAWPFLEPVDPNDAPDYYGVIKEPMDLATMEERVQRRYYEKLTEFVADMTKIFDNCRYYNPSDSPFYQCAEVLESFFVQKLKGFKASRSHNNKLQSTAS

SEQ ID NO: 112 - protein, 288 aa, Homo sapiens
MAAAAYVDHFAAECLVSMSSRAVVHGPREGPESRPEGAAVAATPTLPRVEERRDGKDSASLFVVARILADLNQQAPAPAPAERREGAAARKARTPCRLPPPAPEPTSPGAEGAAAAPPSPAWSEPEPEAGLEPEREPGPAGSGEPGLRQRVRRGRSRADLESPQRKHKCHYAGCEKVYGKSSHLKAHLRTHTGERPFACSWQDCNKKFARSDELARHYRTHTGEKKFSCPICEKRFMRSDHLTKHARRHANFHPGMLQRRGGGSRTGSLSDYSRSDASSPTISPASSP

SEQ ID NO: 113 - protein, 640 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTPRTIKNHWNSTMRRKVEQEGYLQESSKASQPAVATSFQKNSHLMGFAQAPPTAQLPATGQPTVNNDYSYYHISEAQNVSSHVPYPVALHVNIVNVPQPAAAAIQRHYNDEDPEKEKRIKELELLLMSTENELKGQQVLPTQNHTCSYPGWHSTTIADHTRPHGDSAPVSCLGEHHSTPSLPADPGSLPEESASPARCMIVHQGTILDNVKNLLEFAETLQFIDSFLNTSSNHENSDLEMPSLTSTPLIGHKLTVTTPFHRDQTVKTQKENTVFRTPAIKRSILESSPRTPTPFKHALAAQEIKYGPLKMLPQTPSHLVEDLQDVIKQESDESGIVAEFQENGPPLLKKIKQEVESPTDKSGNFFCSHHWEGDSLNTQLFTQTSPVADAPNILTSSVLMAPASEDEDNVLKAFTVPKNRSLASPLQPCSSTWEPASCGKMEEQMTSSSQARKYVNAFSARTLVM

SEQ ID NO: 114 - protein, 1048 aa, Homo sapiens
MQSKVTGNMPTQSLLMYMDGPEVIGSSLGSPMEMEDALSMKGTAVVPFRATQEKNVIQIEGYMPLDCMFCSQTFTHSEDLNKHVLMQHRPTLCEPAVLRVEAEYLSPLDKSQVRTEPPKEKNCKENEFSCEVCGQTFRVAFDVEIHMRTHKDSFTYGCNMCGRRFKEPWFLKNHMRTHNGKSGARSKLQQGLESSPATINEVVQVHAAESISSPYKICMVCGFLFPNKESLIEHRKVHTKKTAFGTSSAQTDSPQGGMPSSREDFLQLFNLRPKSHPETGKKPVRCIPQLDPFTTFQAWQLATKGKVAICQEVKESGQEGSTDNDDSSSEKELGETNKGSCAGLSQEKEKCKHSHGEAPSVDADPKLPSSKEKPTHCSECGKAFRTYHQLVLHSRVHKKDRRAGAESPTMSVDGRQPGTCSPDLAAPLDENGAVDRGEGGSEDGSEDGLPEGIHLDKNDDGGKIKHLTSSRECSYCGKFFRSNYYLNIHLRTHTGEKPYKCEFCEYAAAQKTSLRYHLERHHKEKQTDVAAEVKNDGKNQDTEDALLTADSAQTKNLKRFFDGAKDVTGSPPAKQLKEMPSVFQNVLGSAVLSPAHKDTQDFHKNAADDSADKVNKNPTPAYLDLLKKRSAVETQANNLICRTKADVTPPPDGSTTHNLEVSPKEKQTETAADCRYRPSVDCHEKPLNLSVGALHNCPAISLSKSLIPSITCPFCTFKTFYPEVLMMHQRLEHKYNPDVHKNCRNKSLLRSRRTGCPPALLGKDVPPLSSFCKPKPKSAFPAQSKSLPSAKGKQSPPGPGKAPLTSGIDSSTLAPSNLKSHRPQQNVGVQGAATRQQQSEMFPKTSVSPAPDKTKRPETKLKPLPVAPSQPTLGSSNINGSIDYPAKNDSPWAPPGRDYFCNRSASNTAAEFGEPLPKRLKSSVVALDVDQPGANYRRGYDLPKYHMVRGITSLLPQDCVYPSQALPPKPRFLSSSEVDSPNVLTVQKPYGGSGPLYTCVPAGSPASSSTLEGKRPVSYQHLSNSMAQKRNYENFIGNAHYRPNDKKT

SEQ ID NO: 115 - DNA, 20 bp, Synthetic construct
  source 1..20: mol_type = other DNA
agaagactaaaggagtatgg

SEQ ID NO: 116 - DNA, 20 bp, Synthetic construct
  source 1..20: mol_type = other DNA
cataaacactgaccttgcca

SEQ ID NO: 117 - DNA, 20 bp, Synthetic construct
  source 1..20: mol_type = other DNA
tctccctctatgtcatacaa

SEQ ID NO: 118 - DNA, 20 bp, Synthetic construct
  source 1..20: mol_type = other DNA
tgactcactgtccactagag

SEQ ID NO: 119 - DNA, 20 bp, Synthetic construct
  source 1..20: mol_type = other DNA
acatcagagaatgcacacag

SEQ ID NO: 120 - DNA, 20 bp, Synthetic construct
  source 1..20: mol_type = other DNA
agtatgctgatgtactatga

SEQ ID NO: 121 - DNA, 20 bp, Synthetic construct
  source 1..20: mol_type = other DNA
atacagagtcctctgagcag

SEQ ID NO: 122 - DNA, 20 bp, Synthetic construct
  source 1..20: mol_type = other DNA
tagaagtagaatttccacat

SEQ ID NO: 123 - DNA, 20 bp, Synthetic construct
  source 1..20: mol_type = other DNA
gatcttggtaaactcgctga

SEQ ID NO: 124 - DNA, 20 bp, Synthetic construct
  source 1..20: mol_type = other DNA
ggtaactagggatataccct

SEQ ID NO: 125 - DNA, 20 bp, Synthetic construct
  source 1..20: mol_type = other DNA
taccgctgtatcacttgtga

SEQ ID NO: 126 - DNA, 20 bp, Synthetic construct
  source 1..20: mol_type = other DNA
tggttctagatgactcgaag

SEQ ID NO: 127 - DNA, 20 bp, Synthetic construct
  source 1..20: mol_type = other DNA
atgtgaaaggtactcctgtg

SEQ ID NO: 128 - DNA, 20 bp, Synthetic construct
  source 1..20: mol_type = other DNA
cttgacgtgggctattgtga

SEQ ID NO: 129 - DNA, 20 bp, Synthetic construct
  source 1..20: mol_type = other DNA
gacccggttcgattagaaca

SEQ ID NO: 130 - DNA, 20 bp, Synthetic construct
  source 1..20: mol_type = other DNA
gtatgtcagttggtttcaag

SEQ ID NO: 131 - DNA, 20 bp, Synthetic construct
  source 1..20: mol_type = other DNA
acaatccagatttctaaccc

SEQ ID NO: 132 - DNA, 20 bp, Synthetic construct
  source 1..20: mol_type = other DNA
gcatatatcagactagcacg

SEQ ID NO: 133 - DNA, 20 bp, Synthetic construct
  source 1..20: mol_type = other DNA
tactgtacaattgtagcacc

SEQ ID NO: 134 - DNA, 20 bp, Synthetic construct
  source 1..20: mol_type = other DNA
tgcctacctgagctaaagca

SEQ ID NO: 135 - DNA, 20 bp, Synthetic construct
  source 1..20: mol_type = other DNA
cctggcctacccgtgcgccg

SEQ ID NO: 136 - DNA, 20 bp, Synthetic construct
  source 1..20: mol_type = other DNA
ctcggtgagccagacgacgg

SEQ ID NO: 137 - DNA, 20 bp, Synthetic construct
  source 1..20: mol_type = other DNA
gtggctaaggaactccacct

SEQ ID NO: 138 - DNA, 20 bp, Synthetic construct
  source 1..20: mol_type = other DNA
tccttcttcgtacttcgacg

SEQ ID NO: 139 - DNA, 20 bp, Synthetic construct
  source 1..20: mol_type = other DNA
gaacttaacatatgagaagg

SEQ ID NO: 140 - DNA, 20 bp, Synthetic construct
  source 1..20: mol_type = other DNA
gagtaaaagaagctccacac

SEQ ID NO: 141 - DNA, 20 bp, Synthetic construct
  source 1..20: mol_type = other DNA
tagctacgcctgtagaagaa

SEQ ID NO: 142 - DNA, 20 bp, Synthetic construct
  source 1..20: mol_type = other DNA
tgacattctggagatagttg

SEQ ID NO: 143 - DNA, 20 bp, Synthetic construct
  source 1..20: mol_type = other DNA
actcgagttgacatcagttg

SEQ ID NO: 144 - DNA, 20 bp, Synthetic construct
  source 1..20: mol_type = other DNA
ctcataaggactcttgacgc

SEQ ID NO: 145 - DNA, 20 bp, Synthetic construct
  source 1..20: mol_type = other DNA
ctgttggagtttatatacac

SEQ ID NO: 146 - DNA, 20 bp, Synthetic construct
  source 1..20: mol_type = other DNA
taaatgcctttccacataag

SEQ ID NO: 147 - DNA, 20 bp, Synthetic construct
  source 1..20: mol_type = other DNA
aattatggacagccccacgt

SEQ ID NO: 148 - DNA, 20 bp, Synthetic construct
  source 1..20: mol_type = other DNA
cccagaataagaatgcaccg

SEQ ID NO: 149 - DNA, 20 bp, Synthetic construct
  source 1..20: mol_type = other DNA
ctcttcctattcctacgcgg

SEQ ID NO: 150 - DNA, 20 bp, Synthetic construct
  source 1..20: mol_type = other DNA
gttcagcataacgaaaagtg

SEQ ID NO: 151 - DNA, 20 bp, Synthetic construct
  source 1..20: mol_type = other DNA
cactaatgcatttgagactg

SEQ ID NO: 152 - DNA, 20 bp, Synthetic construct
  source 1..20: mol_type = other DNA
ctcatgactgcttatctctg

SEQ ID NO: 153 - DNA, 20 bp, Synthetic construct
  source 1..20: mol_type = other DNA
gccaccctccacagtaacga

SEQ ID NO: 154 - DNA, 20 bp, Synthetic construct
  source 1..20: mol_type = other DNA
tgcgtgttccacggacatgg

SEQ ID NO: 155 - DNA, 20 bp, Synthetic construct
  source 1..20: mol_type = other DNA
actgcaggcattaatgccga

SEQ ID NO: 156 - DNA, 20 bp, Synthetic construct
  source 1..20: mol_type = other DNA
aggagcaccaaaccgatggg

SEQ ID NO: 157 - DNA, 20 bp, Synthetic construct
  source 1..20: mol_type = other DNA
atagcttgactacatctagt

SEQ ID NO: 158 - DNA, 20 bp, Synthetic construct
  source 1..20: mol_type = other DNA
gcaaacgtttcaagtacaag

SEQ ID NO: 159 - DNA, 20 bp, Synthetic construct
  source 1..20: mol_type = other DNA
aactccatagtaccccacga

SEQ ID NO: 160 - DNA, 20 bp, Synthetic construct
  source 1..20: mol_type = other DNA
gtttgtaggaatggtctcag

SEQ ID NO: 161 - DNA, 20 bp, Synthetic construct
  source 1..20: mol_type = other DNA
tcgtcaaggtgtgtaagcgg

SEQ ID NO: 162 - DNA, 20 bp, Synthetic construct
  source 1..20: mol_type = other DNA
tgagccaccaatgtctagtg

SEQ ID NO: 163 - DNA, 20 bp, Synthetic construct
  source 1..20: mol_type = other DNA
aactcgaaaatcagtaacag

SEQ ID NO: 164 - DNA, 20 bp, Synthetic construct
  source 1..20: mol_type = other DNA
aatagtcaggaagtgccaaa

SEQ ID NO: 165 - DNA, 20 bp, Synthetic construct
  source 1..20: mol_type = other DNA
cagggcaattgaactgccgt

SEQ ID NO: 166 - DNA, 20 bp, Synthetic construct
  source 1..20: mol_type = other DNA
gattagtattgtagccgcag

SEQ ID NO: 167 - DNA, 20 bp, Synthetic construct
  source 1..20: mol_type = other DNA
aatttcatgccaagtcacct

SEQ ID NO: 168 - DNA, 20 bp, Synthetic construct
  source 1..20: mol_type = other DNA
ccagtaccaatattagcatg

SEQ ID NO: 169 - DNA, 20 bp, Synthetic construct
  source 1..20: mol_type = other DNA
gttattgtacaggttcgagt

SEQ ID NO: 170 - DNA, 20 bp, Synthetic construct
  source 1..20: mol_type = other DNA
tgataatcaagtgattcact

SEQ ID NO: 171 - DNA, 20 bp, Synthetic construct
  source 1..20: mol_type = other DNA
atcaagcggtgtgagcggaa

SEQ ID NO: 172 - DNA, 20 bp, Synthetic construct
  source 1..20: mol_type = other DNA
cagccaccacaaagggcacg

SEQ ID NO: 173 - DNA, 20 bp, Synthetic construct
  source 1..20: mol_type = other DNA
gctggaaggcctccatactg

SEQ ID NO: 174 - DNA, 20 bp, Synthetic construct
  source 1..20: mol_type = other DNA
gctgggaataaatgtccacc

SEQ ID NO: 175 - DNA, 20 bp, Synthetic construct
  source 1..20: mol_type = other DNA
agatgtattccgcatagtca

SEQ ID NO: 176 - DNA, 20 bp, Synthetic construct
  source 1..20: mol_type = other DNA
cacttagcgtgataaacccg

SEQ ID NO: 177 - DNA, 20 bp, Synthetic construct
  source 1..20: mol_type = other DNA
ctcttccgcccaaacttccg

SEQ ID NO: 178 - DNA, 20 bp, Synthetic construct
  source 1..20: mol_type = other DNA
ggagcgcggcatatccgaca

SEQ ID NO: 179 - DNA, 20 bp, Synthetic construct
  source 1..20: mol_type = other DNA
ctaaatgaacaacgcctgga

SEQ ID NO: 180 - DNA, 20 bp, Synthetic construct
  source 1..20: mol_type = other DNA
gtgaaagatgaacagacacc

SEQ ID NO: 181 - DNA, 20 bp, Synthetic construct
  source 1..20: mol_type = other DNA
tgatgatgtagaggaaatcg

SEQ ID NO: 182 - DNA, 20 bp, Synthetic construct
  source 1..20: mol_type = other DNA
ttcctgaaccctgagcgttg

SEQ ID NO: 183 - DNA, 20 bp, Synthetic construct
  source 1..20: mol_type = other DNA
acgtgcacctcgagcccgtg

SEQ ID NO: 184 - DNA, 20 bp, Synthetic construct
  source 1..20: mol_type = other DNA
ccggtacattctctaaacgg

SEQ ID NO: 185 - DNA, 20 bp, Synthetic construct
  source 1..20: mol_type = other DNA
tgggcacattgacttctccg

SEQ ID NO: 186 - DNA, 20 bp, Synthetic construct
  source 1..20: mol_type = other DNA
ttcaaaccgtactcatggag

SEQ ID NO: 187 - DNA, 20 bp, Synthetic construct
  source 1..20: mol_type = other DNA
ggccgacatcatgtccatag

SEQ ID NO: 188 - DNA, 20 bp, Synthetic construct
  source 1..20: mol_type = other DNA
tggccctgagcaatagcacg

SEQ ID NO: 189 - DNA, 20 bp, Synthetic construct
  source 1..20: mol_type = other DNA
ttccgatagtgttgaaacca

SEQ ID NO: 190 - DNA, 20 bp, Synthetic construct
  source 1..20: mol_type = other DNA
ttgtaaacagtacactctgg

SEQ ID NO: 191 - DNA, 20 bp, Synthetic construct
  source 1..20: mol_type = other DNA
aagggatgaagatcctcaca

SEQ ID NO: 192 - DNA, 20 bp, Synthetic construct
  source 1..20: mol_type = other DNA
cagtgcggataactatacac

SEQ ID NO: 193 - DNA, 20 bp, Synthetic construct
  source 1..20: mol_type = other DNA
cctggattattatagccacg

SEQ ID NO: 194 - DNA, 20 bp, Synthetic construct
  source 1..20: mol_type = other DNA
gggttgcctgcgatgacgag

SEQ ID NO: 195 - DNA, 20 bp, Synthetic construct
  source 1..20: mol_type = other DNA
cagggtgacgatgtcaactg

SEQ ID NO: 196 - DNA, 20 bp, Synthetic construct
  source 1..20: mol_type = other DNA
ccacagatgcaacataagct

SEQ ID NO: 197 - DNA, 20 bp, Synthetic construct
  source 1..20: mol_type = other DNA
gctgggaaaagaacttcgag

SEQ ID NO: 198 - DNA, 20 bp, Synthetic construct
  source 1..20: mol_type = other DNA
ttgggaaggacaccattccg

SEQ ID NO: 199 - DNA, 20 bp, Synthetic construct
  source 1..20: mol_type = other DNA
cctgcgtgtaccaaatgcag

SEQ ID NO: 200 - DNA, 20 bp, Synthetic construct
  source 1..20: mol_type = other DNA
cgggtggctctcaagcgcgg

SEQ ID NO: 201 - DNA, 20 bp, Synthetic construct
  source 1..20: mol_type = other DNA
gaaatcgacagtactgacat

SEQ ID NO: 202 - DNA, 20 bp, Synthetic construct
  source 1..20: mol_type = other DNA
tggtcagcttggtgtagtcg

SEQ ID NO: 203 - DNA, 20 bp, Synthetic construct
  source 1..20: mol_type = other DNA
cctgtatatcccatcacggg

SEQ ID NO: 204 - DNA, 20 bp, Synthetic construct
  source 1..20: mol_type = other DNA
gcagtcaaataaagtgcccg

SEQ ID NO: 205 - DNA, 20 bp, Synthetic construct
  source 1..20: mol_type = other DNA
gtacccggaataactcgagt

SEQ ID NO: 206 - DNA, 20 bp, Synthetic construct
  source 1..20: mol_type = other DNA
gtgttacaatagctggatga

SEQ ID NO: 207 - DNA, 20 bp, Synthetic construct
  source 1..20: mol_type = other DNA
cacaagcactcgccaccaag

SEQ ID NO: 208 - DNA, 20 bp, Synthetic construct
  source 1..20: mol_type = other DNA
cgtggttgagggaaaatcca

SEQ ID NO: 209 - DNA, 20 bp, Synthetic construct
  source 1..20: mol_type = other DNA
gtgatactctgatcaccgta

SEQ ID NO: 210 - DNA, 20 bp, Synthetic construct
  source 1..20: mol_type = other DNA
tgtccctaaatgcccccaaa

SEQ ID NO: 211 - DNA, 20 bp, Synthetic construct
  source 1..20: mol_type = other DNA
agatgcccgagagttaatca

SEQ ID NO: 212 - DNA, 20 bp, Synthetic construct
  source 1..20: mol_type = other DNA
cataaaaccgagtctcacgg

SEQ ID NO: 213 - DNA, 20 bp, Synthetic construct
  source 1..20: mol_type = other DNA
ccatacacttggctatttcg

SEQ ID NO: 214 - DNA, 20 bp, Synthetic construct
  source 1..20: mol_type = other DNA
tgaaccgaaagcgagaacgt

SEQ ID NO: 215 - DNA, 20 bp, Synthetic construct
  source 1..20: mol_type = other DNA
ccccgccgtacctctcgtag

SEQ ID NO: 216 - DNA, 20 bp, Synthetic construct
  source 1..20: mol_type = other DNA
cttctctagacttaaagatg

SEQ ID NO: 217 - DNA, 20 bp, Synthetic construct
  source 1..20: mol_type = other DNA
gcgacacacgcccatcgaga

SEQ ID NO: 218 - DNA, 20 bp, Synthetic construct
  source 1..20: mol_type = other DNA
gctgtcctgaggcctacaag

SEQ ID NO: 219 - DNA, 20 bp, Synthetic construct
  source 1..20: mol_type = other DNA
acgaagacgatggagtcttg

SEQ ID NO: 220 - DNA, 20 bp, Synthetic construct
  source 1..20: mol_type = other DNA
ccaaaacagaggaaccacac

SEQ ID NO: 221 - DNA, 20 bp, Synthetic construct
  source 1..20: mol_type = other DNA
cttatggaatctccctgtga

SEQ ID NO: 222 - DNA, 20 bp, Synthetic construct
  source 1..20: mol_type = other DNA
tacgtgatgaaattgatacg

SEQ ID NO: 223 - DNA, 20 bp, Synthetic construct
  source 1..20: mol_type = other DNA
aggtggtgggcacatcaaag

SEQ ID NO: 224 - DNA, 20 bp, Synthetic construct
  source 1..20: mol_type = other DNA
ctgaacaaaatcctccaagg

SEQ ID NO: 225 - DNA, 20 bp, Synthetic construct
  source 1..20: mol_type = other DNA
ggcacagcgcacacacatag

SEQ ID NO: 226 - DNA, 20 bp, Synthetic construct
  source 1..20: mol_type = other DNA
gggaaggtctcactgcactg

SEQ ID NO: 227 - DNA, 20 bp, Synthetic construct
  source 1..20: mol_type = other DNA
acaccatccaaatagcgcag

SEQ ID NO: 228 - DNA, 20 bp, Synthetic construct
  source 1..20: mol_type = other DNA
gacaggctgaggtccacgag

SEQ ID NO: 229 - DNA, 20 bp, Synthetic construct
  source 1..20: mol_type = other DNA
gggtgcgcgtgtgcttaaag

SEQ ID NO: 230 - DNA, 20 bp, Synthetic construct
  source 1..20: mol_type = other DNA
tgacatggtaatcgttgaca

SEQ ID NO: 231 - DNA, 20 bp, Synthetic construct
  source 1..20: mol_type = other DNA
cacagctacgggatacggcc

SEQ ID NO: 232 - DNA, 20 bp, Synthetic construct
  source 1..20: mol_type = other DNA
ccggagtctcaaagcgcctg

SEQ ID NO: 233 - DNA, 20 bp, Synthetic construct
  source 1..20: mol_type = other DNA
ctgaggttggtgtaaacggg

SEQ ID NO: 234 - DNA, 20 bp, Synthetic construct
  source 1..20: mol_type = other DNA
gggtaaaagtactgtcccgg

SEQ ID NO: 235 - DNA, 20 bp, Synthetic construct
  source 1..20: mol_type = other DNA
aagtcgagcggcaagcatta

SEQ ID NO: 236 - DNA, 20 bp, Synthetic construct
  source 1..20: mol_type = other DNA
cgagctcttcgtgctgaacg

SEQ ID NO: 237 - DNA, 20 bp, Synthetic construct
  source 1..20: mol_type = other DNA
gcggcggagacctcttcccg

SEQ ID NO: 238 - DNA, 20 bp, Synthetic construct
  source 1..20: mol_type = other DNA
gctggtcgatctggcagtca

SEQ ID NO: 239 - DNA, 20 bp, Synthetic construct
  source 1..20: mol_type = other DNA
acagacgattgctgtactcg

SEQ ID NO: 240 - DNA, 20 bp, Synthetic construct
  source 1..20: mol_type = other DNA
agagtataacgatgttcttg

SEQ ID NO: 241 - DNA, 20 bp, Synthetic construct
  source 1..20: mol_type = other DNA
gcagagtacccgagaatcat

SEQ ID NO: 242 - DNA, 20 bp, Synthetic construct
  source 1..20: mol_type = other DNA
tcaattgcctgagatcgtat

SEQ ID NO: 243 - DNA, 20 bp, Synthetic construct
  source 1..20: mol_type = other DNA
aggacacgggatccacaccc

SEQ ID NO: 244 - DNA, 20 bp, Synthetic construct
  source 1..20: mol_type = other DNA
atccctggcctagcaaacag

SEQ ID NO: 245 - DNA, 20 bp, Synthetic construct
  source 1..20: mol_type = other DNA
gagaatgggtgtccagaggt

SEQ ID NO: 246 - DNA, 20 bp, Synthetic construct
  source 1..20: mol_type = other DNA
gggtaacggggaaaagtccg

SEQ ID NO: 247 - DNA, 20 bp, Synthetic construct
  source 1..20: mol_type = other DNA
cggaagtggctaccacccga

SEQ ID NO: 248 - DNA, 20 bp, Synthetic construct
  source 1..20: mol_type = other DNA
ctgagcccgatgtaccccag

SEQ ID NO: 249 - DNA, 20 bp, Synthetic construct
  source 1..20: mol_type = other DNA
gaccggccagaggcgacaca

SEQ ID NO: 250 - DNA, 20 bp, Synthetic construct
  source 1..20: mol_type = other DNA
ggaaggcacccagatccgga

SEQ ID NO: 251 - DNA, 20 bp, Synthetic construct
  source 1..20: mol_type = other DNA
cacttcgaccgacaaacctg

SEQ ID NO: 252 - DNA, 20 bp, Synthetic construct
  source 1..20: mol_type = other DNA
ctgatcgtaggaccacggtg

SEQ ID NO: 253 - DNA, 20 bp, Synthetic construct
  source 1..20: mol_type = other DNA
ggcagtggagtggttcaggg

SEQ ID NO: 254 - DNA, 20 bp, Synthetic construct
  source 1..20: mol_type = other DNA
tagatgatcagaccaagccc

SEQ ID NO: 255 - DNA, 20 bp, Synthetic construct
  source 1..20: mol_type = other DNA
acaggttggtctgtccagaa

SEQ ID NO: 256 - DNA, 20 bp, Synthetic construct
  source 1..20: mol_type = other DNA
gcaagtacagccagtcccgg

SEQ ID NO: 257 - DNA, 20 bp, Synthetic construct
  source 1..20: mol_type = other DNA
ggagtcggtgaagatcctcc

SEQ ID NO: 258 - DNA, 20 bp, Synthetic construct
  source 1..20: mol_type = other DNA
ggcgggcaaaaggaagcgca

SEQ ID NO: 259 - DNA, 20 bp, Synthetic construct
  source 1..20: mol_type = other DNA
acatgaatgcagaggattcg

SEQ ID NO: 260 - DNA, 20 bp, Synthetic construct
  source 1..20: mol_type = other DNA
agggcccggaggaacaacag

SEQ ID NO: 261 - DNA, 20 bp, Synthetic construct
  source 1..20: mol_type = other DNA
ccagccgatgcacaaaacgt

SEQ ID NO: 262 - DNA, 20 bp, Synthetic construct
  source 1..20: mol_type = other DNA
tgtggagcactcataccgaa

SEQ ID NO: 263 - DNA, 20 bp, Synthetic construct
  source 1..20: mol_type = other DNA
agagcgtgcagataatgaca

SEQ ID NO: 264 - DNA, 20 bp, Synthetic construct
  source 1..20: mol_type = other DNA
agctggcagaccacaaactg

SEQ ID NO: 265 - DNA, 20 bp, Synthetic construct
  source 1..20: mol_type = other DNA
attcttcataccaggaccag

SEQ ID NO: 266 - DNA, 20 bp, Synthetic construct
  source 1..20: mol_type = other DNA
ccaattcaggacccacacga

SEQ ID NO: 267 - DNA, 20 bp, Synthetic construct
  source 1..20: mol_type = other DNA
ctgcagggaacctccaccat

SEQ ID NO: 268 - DNA, 20 bp, Synthetic construct
  source 1..20: mol_type = other DNA
ggatacagggctctacacag

SEQ ID NO: 269 - DNA, 20 bp, Synthetic construct
  source 1..20: mol_type = other DNA
tggctttgaatgtggcgtgt

SEQ ID NO: 270 - DNA, 20 bp, Synthetic construct
  source 1..20: mol_type = other DNA
ttgtttcgttgtgttccgag

SEQ ID NO: 271 - DNA, 20 bp, Synthetic construct
  source 1..20: mol_type = other DNA
acaggttgccccacgcgttg

SEQ ID NO: 272 - DNA, 20 bp, Synthetic construct
  source 1..20: mol_type = other DNA
ggagtttagccagtccaact

SEQ ID NO: 273 - DNA, 20 bp, Synthetic construct
  source 1..20: mol_type = other DNA
ggtggcgcaaacgagtagca

SEQ ID NO: 274 - DNA, 20 bp, Synthetic construct
  source 1..20: mol_type = other DNA
tagcatttgagctagttcga

SEQ ID NO: 275 - DNA, 20 bp, Synthetic construct
  source 1..20: mol_type = other DNA
agaggaggagacacatgtcg

SEQ ID NO: 276 - DNA, 20 bp, Synthetic construct
  source 1..20: mol_type = other DNA
catacaccatgtccatagag

SEQ ID NO: 277 - DNA, 20 bp, Synthetic construct
  source 1..20: mol_type = other DNA
gccttctgacaattcagccc

SEQ ID NO: 278 - DNA, 20 bp, Synthetic construct
  source 1..20: mol_type = other DNA
gttctgtagacttcacatgc

SEQ ID NO: 279 - DNA, 20 bp, Synthetic construct
  source 1..20: mol_type = other DNA
cagccaggacaacaatgcga

SEQ ID NO: 280 - DNA, 20 bp, Synthetic construct
  source 1..20: mol_type = other DNA
gtggccttaatgttctcctg

SEQ ID NO: 281 - DNA, 20 bp, Synthetic construct
  source 1..20: mol_type = other DNA
gttcattgtacaatatatgg

SEQ ID NO: 282 - DNA, 20 bp, Synthetic construct
  source 1..20: mol_type = other DNA
taagaggtcagaccgtcgtg

SEQ ID NO: 283 - DNA, 20 bp, Synthetic construct
  source 1..20: mol_type = other DNA
acattctgtacaatgaacag

SEQ ID NO: 284 - DNA, 20 bp, Synthetic construct
  source 1..20: mol_type = other DNA
atcaagcgtgctgaccggcg

SEQ ID NO: 285 - DNA, 20 bp, Synthetic construct
  source 1..20: mol_type = other DNA
cagccaggaccacaatgcca

SEQ ID NO: 286 - DNA, 20 bp, Synthetic construct
  source 1..20: mol_type = other DNA
cgtgcacatgaatccccccc

SEQ ID NO: 287 - DNA, 20 bp, Synthetic construct
  source 1..20: mol_type = other DNA
acatcgactgctggacaatg

SEQ ID NO: 288 - DNA, 20 bp, Synthetic construct
  source 1..20: mol_type = other DNA
cagtgagtagtgccaaacca

SEQ ID NO: 289 - DNA, 20 bp, Synthetic construct
  source 1..20: mol_type = other DNA
gtggcgtactgcacgtgtcg

SEQ ID NO: 290 - DNA, 20 bp, Synthetic construct
  source 1..20: mol_type = other DNA
ttcacattatgaccaacacc

SEQ ID NO: 291 - DNA, 20 bp, Synthetic construct
  source 1..20: mol_type = other DNA
acccaggcatcatccgacaa

SEQ ID NO: 292 - DNA, 20 bp, Synthetic construct
  source 1..20: mol_type = other DNA
cccacccacagggatcaacg

SEQ ID NO: 293 - DNA, 20 bp, Synthetic construct
  source 1..20: mol_type = other DNA
cctacttaggcactgccagg

SEQ ID NO: 294 - DNA, 20 bp, Synthetic construct
  source 1..20: mol_type = other DNA
gagggtgccaccatgactag

SEQ ID NO: 295 - DNA, 20 bp, Synthetic construct
  source 1..20: mol_type = other DNA
actcaatcgtgaggattggt

SEQ ID NO: 296 - DNA, 20 bp, Synthetic construct
  source 1..20: mol_type = other DNA
actgtgtaaaatgaacaagg

SEQ ID NO: 297 - DNA, 20 bp, Synthetic construct
  source 1..20: mol_type = other DNA
ccaacatgaccaccaacgag

SEQ ID NO: 298 - DNA, 20 bp, Synthetic construct
  source 1..20: mol_type = other DNA
cttacctggatccattcatg

SEQ ID NO: 299 - DNA, 20 bp, Synthetic construct
  source 1..20: mol_type = other DNA
cttactaatgaagtaatccg

SEQ ID NO: 300 - DNA, 20 bp, Synthetic construct
  source 1..20: mol_type = other DNA
gagaaagcagtctttaccca

SEQ ID NO: 301 - DNA, 20 bp, Synthetic construct
  source 1..20: mol_type = other DNA
ggtctcggagaatgaccgag

SEQ ID NO: 302 - DNA, 20 bp, Synthetic construct
  source 1..20: mol_type = other DNA
tgcatggggaggaccagtcg

SEQ ID NO: 303 - DNA, 20 bp, Synthetic construct
  source 1..20: mol_type = other DNA
cacggaccctgatagcatga

SEQ ID NO: 304 - DNA, 20 bp, Synthetic construct
  source 1..20: mol_type = other DNA
catcgctcaggagatatacg

SEQ ID NO: 305 - DNA, 20 bp, Synthetic construct
  source 1..20: mol_type = other DNA
gcagccgaatcgccaaccgc

SEQ ID NO: 306 - DNA, 20 bp, Synthetic construct
  source 1..20: mol_type = other DNA
gcttcgcagcctgctaacca

SEQ ID NO: 307 - DNA, 20 bp, Synthetic construct
  source 1..20: mol_type = other DNA
aagatcagtagcggtcccgg

SEQ ID NO: 308 - DNA, 20 bp, Synthetic construct
  source 1..20: mol_type = other DNA
agaaaggacgtcgatcgagg

SEQ ID NO: 309 - DNA, 20 bp, Synthetic construct
  source 1..20: mol_type = other DNA
aggggtcttaatgatccgcg

SEQ ID NO: 310 - DNA, 20 bp, Synthetic construct
  source 1..20: mol_type = other DNA
ttgaccataattatgctcag

SEQ ID NO: 311 - DNA, 20 bp, Synthetic construct
  source 1..20: mol_type = other DNA
aatgcttcacccatacaaca

SEQ ID NO: 312 - DNA, 20 bp, Synthetic construct
  source 1..20: mol_type = other DNA
cagaccagacagtgttacca

SEQ ID NO: 313 - DNA, 20 bp, Synthetic construct
  source 1..20: mol_type = other DNA
gaaggacaaaagctttgaag

SEQ ID NO: 314 - DNA, 20 bp, Synthetic construct
  source 1..20: mol_type = other DNA
ggttacttgtacacagctga

SEQ ID NO: 315 - DNA, 20 bp, Synthetic construct
  source 1..20: mol_type = other DNA
agccggtccctttcccgtcg

SEQ ID NO: 316 - DNA, 20 bp, Synthetic construct
  source 1..20: mol_type = other DNA
cctcagttggcaaggtacct

SEQ ID NO: 317 - DNA, 20 bp, Synthetic construct
  source 1..20: mol_type = other DNA
cctctcaatcaaaataaggt

SEQ ID NO: 318 - DNA, 20 bp, Synthetic construct
  source 1..20: mol_type = other DNA
cgaaaatctgggctaagtgc

SEQ ID NO: 319 - DNA, 20 bp, Synthetic construct
  source 1..20: mol_type = other DNA
actgtcactaacctactggt

SEQ ID NO: 320 - DNA, 20 bp, Synthetic construct
  source 1..20: mol_type = other DNA
cccgcccgctgactgcacat

SEQ ID NO: 321 - DNA, 20 bp, Synthetic construct
  source 1..20: mol_type = other DNA
ctctaccgcccggaaaacgt

SEQ ID NO: 322 - DNA, 20 bp, Synthetic construct
  source 1..20: mol_type = other DNA
ttgggccagagtacgatgca

SEQ ID NO: 323 - DNA, 20 bp, Synthetic construct
  source 1..20: mol_type = other DNA
cgggacacgtctacttggtg

SEQ ID NO: 324 - DNA, 20 bp, Synthetic construct
  source 1..20: mol_type = other DNA
gcagaacgaggctgccctag

SEQ ID NO: 325 - DNA, 20 bp, Synthetic construct
  source 1..20: mol_type = other DNA
gcggaccagtgtacagcacg

SEQ ID NO: 326 - DNA, 20 bp, Synthetic construct
  source 1..20: mol_type = other DNA
taaggtgaggactttgcaca

SEQ ID NO: 327 - DNA, 20 bp, Synthetic construct
  source 1..20: mol_type = other DNA
acctggtgtggacccacgcg

SEQ ID NO: 328 - DNA, 20 bp, Synthetic construct
  source 1..20: mol_type = other DNA
cctcgaactgcaccataggt

SEQ ID NO: 329 - DNA, 20 bp, Synthetic construct
  source 1..20: mol_type = other DNA
gccattgatctgatgtacgg

SEQ ID NO: 330 - DNA, 20 bp, Synthetic construct
  source 1..20: mol_type = other DNA
tggggctctgcatctcacag

SEQ ID NO: 331 - DNA, 20 bp, Synthetic construct
  source 1..20: mol_type = other DNA
acactggaacatatcaagac

SEQ ID NO: 332 - DNA, 20 bp, Synthetic construct
  source 1..20: mol_type = other DNA
gacaaatgcaatgaaaactg

SEQ ID NO: 333 - DNA, 20 bp, Synthetic construct
  source 1..20: mol_type = other DNA
ggacatcgtgtaccgcacca

SEQ ID NO: 334 - DNA, 20 bp, Synthetic construct
  source 1..20: mol_type = other DNA
ggccgcccgggaagtcaaca

SEQ ID NO: 335 - DNA, 20 bp, Synthetic construct
  source 1..20: mol_type = other DNA
gaaaatgaatggctcccaca

SEQ ID NO: 336 - DNA, 20 bp, Synthetic construct
  source 1..20: mol_type = other DNA
gatggcttggtccatcacgt

SEQ ID NO: 337 - DNA, 20 bp, Synthetic construct
  source 1..20: mol_type = other DNA
ggggcctcattcacccagaa

SEQ ID NO: 338 - DNA, 20 bp, Synthetic construct
  source 1..20: mol_type = other DNA
tccaagagtgacagagtcgt

SEQ ID NO: 339 - DNA, 20 bp, Synthetic construct
  source 1..20: mol_type = other DNA
atcccgaacctgcattctga

SEQ ID NO: 340 - DNA, 20 bp, Synthetic construct
  source 1..20: mol_type = other DNA
caccgcttcagtgcaacacg

SEQ ID NO: 341 - DNA, 20 bp, Synthetic construct
  source 1..20: mol_type = other DNA
ccaaggaaaaaactaacgag

SEQ ID NO: 342 - DNA, 20 bp, Synthetic construct
  source 1..20: mol_type = other DNA
gcggctcctagaccggcagg

SEQ ID NO: 343 - DNA, 20 bp, Synthetic construct
  source 1..20: mol_type = other DNA
acggctatgtgcaatctgcg

SEQ ID NO: 344 - DNA, 20 bp, Synthetic construct
  source 1..20: mol_type = other DNA
gccctggctggatcccgcag

SEQ ID NO: 345 - DNA, 20 bp, Synthetic construct
  source 1..20: mol_type = other DNA
ggacaacaaagactgcacag

SEQ ID NO: 346 - DNA, 20 bp, Synthetic construct
  source 1..20: mol_type = other DNA
gtggcttcacatcttcaggg

SEQ ID NO: 347 - DNA, 20 bp, Synthetic construct
  source 1..20: mol_type = other DNA
aagatgaactgcgatgtgtg

SEQ ID NO: 348 - DNA, 20 bp, Synthetic construct
  source 1..20: mol_type = other DNA
caagcagagaagttcccttg

SEQ ID NO: 349 - DNA, 20 bp, Synthetic construct
  source 1..20: mol_type = other DNA
gctcatacagacccgcatga

SEQ ID NO: 350 - DNA, 20 bp, Synthetic construct
  source 1..20: mol_type = other DNA
ggacagattagcaagcaatg

SEQ ID NO: 351 - DNA, 20 bp, Synthetic construct
  source 1..20: mol_type = other DNA
ccttactctctccatgtcag

SEQ ID NO: 352 - DNA, 20 bp, Synthetic construct
  source 1..20: mol_type = other DNA
gaactcgatgagcccctgcg

SEQ ID NO: 353 - DNA, 20 bp, Synthetic construct
  source 1..20: mol_type = other DNA
gctcatcgacgactacggag

SEQ ID NO: 354 - DNA, 20 bp, Synthetic construct
  source 1..20: mol_type = other DNA
tccgccagacggtcaaccgg

SEQ ID NO: 355 - DNA, 20 bp, Synthetic construct
  source 1..20: mol_type = other DNA
aaggaaagagtctactgtcg

SEQ ID NO: 356 - DNA, 20 bp, Synthetic construct
  source 1..20: mol_type = other DNA
agatgtgagtggatctatcg

SEQ ID NO: 357 - DNA, 20 bp, Synthetic construct
  source 1..20: mol_type = other DNA
ccttacctgaatcaatactg

SEQ ID NO: 358 - DNA, 20 bp, Synthetic construct
  source 1..20: mol_type = other DNA
gttatctataagacaccttg

SEQ ID NO: 359 - DNA, 20 bp, Synthetic construct
  source 1..20: mol_type = other DNA
actacgacctgaatgctgtg

SEQ ID NO: 360 - DNA, 20 bp, Synthetic construct
  source 1..20: mol_type = other DNA
gcttccgctacaagtgcgag

SEQ ID NO: 361 - DNA, 20 bp, Synthetic construct
  source 1..20: mol_type = other DNA
ggaagatctcatccccaccg

SEQ ID NO: 362 - DNA, 20 bp, Synthetic construct
  source 1..20: mol_type = other DNA
tcaatggctacacaggacca

SEQ ID NO: 363 - DNA, 20 bp, Synthetic construct
  source 1..20: mol_type = other DNA
aatcagatgacaatgagtca

SEQ ID NO: 364 - DNA, 20 bp, Synthetic construct
  source 1..20: mol_type = other DNA
caatacaatatgccacaggg

SEQ ID NO: 365 - DNA, 20 bp, Synthetic construct
  source 1..20: mol_type = other DNA
cctaaccatatgcctatgca

SEQ ID NO: 366 - DNA, 20 bp, Synthetic construct
  source 1..20: mol_type = other DNA
ggcatgttgtgagagcgtgg

SEQ ID NO: 367 - DNA, 20 bp, Synthetic construct
  source 1..20: mol_type = other DNA
aggttggtgactgtgaacgc

SEQ ID NO: 368 - DNA, 20 bp, Synthetic construct
  source 1..20: mol_type = other DNA
agttcatcgacaacaagctg

SEQ ID NO: 369 - DNA, 20 bp, Synthetic construct
  source 1..20: mol_type = other DNA
gggctgacactagcagacac

SEQ ID NO: 370 - DNA, 20 bp, Synthetic construct
  source 1..20: mol_type = other DNA
tctgttgtggggtctgaacg

SEQ ID NO: 371 - DNA, 20 bp, Synthetic construct
  source 1..20: mol_type = other DNA
aggtaccctccgacccacca

SEQ ID NO: 372 - DNA, 20 bp, Synthetic construct
  source 1..20: mol_type = other DNA
cagggagtgtgtgaactgtg

SEQ ID NO: 373 - DNA, 20 bp, Synthetic construct
  source 1..20: mol_type = other DNA
ggagctgtactcgggcacgt

SEQ ID NO: 374 - DNA, 20 bp, Synthetic construct
  source 1..20: mol_type = other DNA
tccaagacgtccatccacca

SEQ ID NO: 375 - DNA, 20 bp, Synthetic construct
  source 1..20: mol_type = other DNA
cgttacccagaatatcacgg

SEQ ID NO: 376 - DNA, 20 bp, Synthetic construct
  source 1..20: mol_type = other DNA
gcacatccgtatacactcag

SEQ ID NO: 377 - DNA, 20 bp, Synthetic construct
  source 1..20: mol_type = other DNA
ggtagcatcgaccctaaccg

SEQ ID NO: 378 - DNA, 20 bp, Synthetic construct
  source 1..20: mol_type = other DNA
tggcccccgagtggatacgg

SEQ ID NO: 379 - DNA, 20 bp, Synthetic construct
  source 1..20: mol_type = other DNA
agatcgaagtatgcctcacc

SEQ ID NO: 380 - DNA, 20 bp, Synthetic construct
  source 1..20: mol_type = other DNA
agtgcatactgtagtccttg

SEQ ID NO: 381 - DNA, 20 bp, Synthetic construct
  source 1..20: mol_type = other DNA
gagcccccaactgacgaatg

SEQ ID NO: 382 - DNA, 20 bp, Synthetic construct
  source 1..20: mol_type = other DNA
taattagtactactatgcac

SEQ ID NO: 383 - DNA, 20 bp, Synthetic construct
  source 1..20: mol_type = other DNA
actctccaggcttaacacag

SEQ ID NO: 384 - DNA, 20 bp, Synthetic construct
  source 1..20: mol_type = other DNA
agctctcgaagactgctgtg

SEQ ID NO: 385 - DNA, 20 bp, Synthetic construct
  source 1..20: mol_type = other DNA
gtgtacaaattcctgcacca

SEQ ID NO: 386 - DNA, 20 bp, Synthetic construct
  source 1..20: mol_type = other DNA
tgacgcggaggcgtattcgg

SEQ ID NO: 387 - DNA, 20 bp, Synthetic construct
  source 1..20: mol_type = other DNA
atcacacatagcgacgaagt

SEQ ID NO: 388 - DNA, 20 bp, Synthetic construct
  source 1..20: mol_type = other DNA
cagagcatctctagctaacg

SEQ ID NO: 389 - DNA, 20 bp, Synthetic construct
  source 1..20: mol_type = other DNA
ctaactctgctacccaagtg

SEQ ID NO: 390 - DNA, 20 bp, Synthetic construct
  source 1..20: mol_type = other DNA
taatgttaatccgagaacgg

SEQ ID NO: 391 - DNA, 20 bp, Synthetic construct
  source 1..20: mol_type = other DNA
aacagtgaaactctgttgag

SEQ ID NO: 392 - DNA, 20 bp, Synthetic construct
  source 1..20: mol_type = other DNA
gggaaacgtagccatggcga

SEQ ID NO: 393 - DNA, 20 bp, Synthetic construct
  source 1..20: mol_type = other DNA
ggttcatctaagaacaagag

SEQ ID NO: 394 - DNA, 20 bp, Synthetic construct
  source 1..20: mol_type = other DNA
tgctctgaacccgatgcaga

SEQ ID NO: 395 - DNA, 20 bp, Synthetic construct
  source 1..20: mol_type = other DNA
agccgtagtatctcccacac

SEQ ID NO: 396 - DNA, 20 bp, Synthetic construct
  source 1..20: mol_type = other DNA
ccacgacaagggacccgagg

SEQ ID NO: 397 - DNA, 20 bp, Synthetic construct
  source 1..20: mol_type = other DNA
cctgcacatacggagagtaa

SEQ ID NO: 398 - DNA, 20 bp, Synthetic construct
  source 1..20: mol_type = other DNA
ttccgcacagagacaaatgg

SEQ ID NO: 399 - DNA, 20 bp, Synthetic construct
  source 1..20: mol_type = other DNA
tcggaggtaaacttggccgt

SEQ ID NO: 400 - DNA, 20 bp, Synthetic construct
  source 1..20: mol_type = other DNA
tctaagttgtaacaaaagct

SEQ ID NO: 401 - DNA, 20 bp, Synthetic construct
  source 1..20: mol_type = other DNA
tctgaggctgaaacatagca

SEQ ID NO: 402 - DNA, 20 bp, Synthetic construct
  source 1..20: mol_type = other DNA
tgccagtactcctccagaga

SEQ ID NO: 403 - DNA, 20 bp, Synthetic construct
  source 1..20: mol_type = other DNA
gaggcaaaaagaaatcccca

SEQ ID NO: 404 - DNA, 20 bp, Synthetic construct
  source 1..20: mol_type = other DNA
gatttctgatgaaaaagacg

SEQ ID NO: 405 - DNA, 20 bp, Synthetic construct
  source 1..20: mol_type = other DNA
gctctactggagcgacgagg

SEQ ID NO: 406 - DNA, 20 bp, Synthetic construct
  source 1..20: mol_type = other DNA
tcacccaaaaaaatgcacca

SEQ ID NO: 407 - DNA, 20 bp, Synthetic construct
  source 1..20: mol_type = other DNA
cctgccaagaaggatgtcaa

SEQ ID NO: 408 - DNA, 20 bp, Synthetic construct
  source 1..20: mol_type = other DNA
ctcaaagccacagtcgacaa

SEQ ID NO: 409 - DNA, 20 bp, Synthetic construct
  source 1..20: mol_type = other DNA
gaggctcttatttcgagcca

SEQ ID NO: 410 - DNA, 20 bp, Synthetic construct
  source 1..20: mol_type = other DNA
gataagatgcttgaacagct

SEQ ID NO: 411 - DNA, 20 bp, Synthetic construct
  source 1..20: mol_type = other DNA
atcacggaagtctcgtcctg

SEQ ID NO: 412 - DNA, 20 bp, Synthetic construct
  source 1..20: mol_type = other DNA
gcatgtgcggtcgcacaccg

SEQ ID NO: 413 - DNA, 20 bp, Synthetic construct
  source 1..20: mol_type = other DNA
ggcagcttacggcgacacat

SEQ ID NO: 414 - DNA, 20 bp, Synthetic construct
  source 1..20: mol_type = other DNA
tggtctacgtgccctcgcag

SEQ ID NO: 415 - DNA, 20 bp, Synthetic construct
  source 1..20: mol_type = other DNA
acctactgaaccctcctccg

SEQ ID NO: 416 - DNA, 20 bp, Synthetic construct
  source 1..20: mol_type = other DNA
gcggatcagaaaggtgccgg

SEQ ID NO: 417 - DNA, 20 bp, Synthetic construct
  source 1..20: mol_type = other DNA
tcagcgtcaagacccagtct

SEQ ID NO: 418 - DNA, 20 bp, Synthetic construct
  source 1..20: mol_type = other DNA
ttgagcacgcagtcgaagcg

SEQ ID NO: 419 - DNA, 20 bp, Synthetic construct
  source 1..20: mol_type = other DNA
aagcagagtgcacagaagaa

SEQ ID NO: 420 - DNA, 20 bp, Synthetic construct
  source 1..20: mol_type = other DNA
ccgaaatgttgagtgtggtg

SEQ ID NO: 421 - DNA, 20 bp, Synthetic construct
  source 1..20: mol_type = other DNA
ctccagagaatttctccccg

SEQ ID NO: 422 - DNA, 20 bp, Synthetic construct
  source 1..20: mol_type = other DNA
tctttctagatcccatacag

SEQ ID NO: 423 - DNA, 20 bp, Synthetic construct
  source 1..20: mol_type = other DNA
aaagaacaaatggaacgcga

SEQ ID NO: 424 - DNA, 20 bp, Synthetic construct
  source 1..20: mol_type = other DNA
cacctagtaagtcgtttagg

SEQ ID NO: 425 - DNA, 20 bp, Synthetic construct
  source 1..20: mol_type = other DNA
catgccagttcacagcagtg

SEQ ID NO: 426 - DNA, 20 bp, Synthetic construct
  source 1..20: mol_type = other DNA
cattgcctcaggaacaaagg

SEQ ID NO: 427 - DNA, 20 bp, Synthetic construct
  source 1..20: mol_type = other DNA
aacatggaagaatccaacaa

SEQ ID NO: 428 - DNA, 20 bp, Synthetic construct
  source 1..20: mol_type = other DNA
acgccggtcttgatgacgag

SEQ ID NO: 429 - DNA, 20 bp, Synthetic construct
  source 1..20: mol_type = other DNA
gagaccgaggtgtatcacca

SEQ ID NO: 430 - DNA, 20 bp, Synthetic construct
  source 1..20: mol_type = other DNA
tcggccggtgctgtacaatg

SEQ ID NO: 431 - DNA, 20 bp, Synthetic construct
  source 1..20: mol_type = other DNA
caatgaacgagcaacagcaa

SEQ ID NO: 432 - DNA, 20 bp, Synthetic construct
  source 1..20: mol_type = other DNA
ccgtacactcagcatgaccc

SEQ ID NO: 433 - DNA, 20 bp, Synthetic construct
  source 1..20: mol_type = other DNA
gaaatcgccaggcagttcgg

SEQ ID NO: 434 - DNA, 20 bp, Synthetic construct
  source 1..20: mol_type = other DNA
tccaggatcagttcaaaccg

SEQ ID NO: 435 - DNA, 20 bp, Synthetic construct
  source 1..20: mol_type = other DNA
aaacttaaatgtgagcatcc

SEQ ID NO: 436 - DNA, 20 bp, Synthetic construct
  source 1..20: mol_type = other DNA
acaactggagcatttactgc

SEQ ID NO: 437 - DNA, 20 bp, Synthetic construct
  source 1..20: mol_type = other DNA
agaagaagaactcaaacctc

SEQ ID NO: 438 - DNA, 20 bp, Synthetic construct
  source 1..20: mol_type = other DNA
ttctttgtagaacttgaagt

SEQ ID NO: 439 - DNA, 20 bp, Synthetic construct
  source 1..20: mol_type = other DNA
agcaagtcgtgcgagttctg

SEQ ID NO: 440 - DNA, 20 bp, Synthetic construct
  source 1..20: mol_type = other DNA
ccagcagctcgctcacgccg

SEQ ID NO: 441 - DNA, 20 bp, Synthetic construct
  source 1..20: mol_type = other DNA
ccgccatggacttctcgcgg

SEQ ID NO: 442 - DNA, 20 bp, Synthetic construct
  source 1..20: mol_type = other DNA
tcagggtgagggtcagacgg

SEQ ID NO: 443 - DNA, 20 bp, Synthetic construct
  source 1..20: mol_type = other DNA
aagtgttgtttgatcagtca

SEQ ID NO: 444 - DNA, 20 bp, Synthetic construct
  source 1..20: mol_type = other DNA
acatactctaagtcaggcag

SEQ ID NO: 445 - DNA, 20 bp, Synthetic construct
  source 1..20: mol_type = other DNA
gtgtaatttagagagcagcg

SEQ ID NO: 446 - DNA, 20 bp, Synthetic construct
  source 1..20: mol_type = other DNA
tctgttcagactctaatagg

SEQ ID NO: 447 - DNA, 20 bp, Synthetic construct
  source 1..20: mol_type = other DNA
ccctggatagaatacatcgt

SEQ ID NO: 448 - DNA, 20 bp, Synthetic construct
  source 1..20: mol_type = other DNA
gacaagatgtcagacctagg

SEQ ID NO: 449 - DNA, 20 bp, Synthetic construct
  source 1..20: mol_type = other DNA
gaggtggaaggtaaacacaa

SEQ ID NO: 450 - DNA, 20 bp, Synthetic construct
  source 1..20: mol_type = other DNA
ttctaggataaccaccactg

SEQ ID NO: 451 - DNA, 20 bp, Synthetic construct
  source 1..20: mol_type = other DNA
acctgatcccaaaacatgga

SEQ ID NO: 452 - DNA, 20 bp, Synthetic construct
  source 1..20: mol_type = other DNA
cagcattcggtagaccctga

SEQ ID NO: 453 - DNA, 20 bp, Synthetic construct
  source 1..20: mol_type = other DNA
ggatgcatgcggctagacat

SEQ ID NO: 454 - DNA, 20 bp, Synthetic construct
  source 1..20: mol_type = other DNA
tacctgcataggaagacacg

SEQ ID NO: 455 - DNA, 20 bp, Synthetic construct
  source 1..20: mol_type = other DNA
acagctcaagcgcttcatcc

SEQ ID NO: 456 - DNA, 20 bp, Synthetic construct
  source 1..20: mol_type = other DNA
aggctccccggacatctggg

SEQ ID NO: 457 - DNA, 20 bp, Synthetic construct
  source 1..20: mol_type = other DNA
cactaacccccgagactcag

SEQ ID NO: 458 - DNA, 20 bp, Synthetic construct
  source 1..20: mol_type = other DNA
cgcatgcagagcatcaacgc

SEQ ID NO: 459 - DNA, 20 bp, Synthetic construct
  source 1..20: mol_type = other DNA
aggatgcggatatgactctg

SEQ ID NO: 460 - DNA, 20 bp, Synthetic construct
  source 1..20: mol_type = other DNA
ggacgcgttcaagtaagcgt

SEQ ID NO: 461 - DNA, 20 bp, Synthetic construct
  source 1..20: mol_type = other DNA
ggggagcgagtgatgtacgt

SEQ ID NO: 462 - DNA, 20 bp, Synthetic construct
  source 1..20: mol_type = other DNA
tttggacagatctattccag

SEQ ID NO: 463 - DNA, 20 bp, Synthetic construct
  source 1..20: mol_type = other DNA
ccacttgttaacagagaccg

SEQ ID NO: 464 - DNA, 20 bp, Synthetic construct
  source 1..20: mol_type = other DNA
cttgtggcgctgaaaacgaa

SEQ ID NO: 465 - DNA, 20 bp, Synthetic construct
  source 1..20: mol_type = other DNA
tatgccatgagtgctcagag

SEQ ID NO: 466 - DNA, 20 bp, Synthetic construct
  source 1..20: mol_type = other DNA
tgagagactccagttgccag

SEQ ID NO: 467 - DNA, 20 bp, Synthetic construct
  source 1..20: mol_type = other DNA
caagtaccgacgcgccagcg

SEQ ID NO: 468 - DNA, 20 bp, Synthetic construct
  source 1..20: mol_type = other DNA
catcatccctcacggacccg

SEQ ID NO: 469 - DNA, 20 bp, Synthetic construct
  source 1..20: mol_type = other DNA
gatgactgcactcaccaaca

SEQ ID NO: 470 - DNA, 20 bp, Synthetic construct
  source 1..20: mol_type = other DNA
ggtgagcgaatgagtagact

SEQ ID NO: 471 - DNA, 20 bp, Synthetic construct
  source 1..20: mol_type = other DNA
gagaacctcggaacatacgg

SEQ ID NO: 472 - DNA, 20 bp, Synthetic construct
  source 1..20: mol_type = other DNA
gcagatcgagtcctacccca

SEQ ID NO: 473 - DNA, 20 bp, Synthetic construct
  source 1..20: mol_type = other DNA
tcttcttgtctcggcccatg

SEQ ID NO: 474 - DNA, 20 bp, Synthetic construct
  source 1..20: mol_type = other DNA
tgagaacgcatctcagcccg

SEQ ID NO: 475 - DNA, 20 bp, Synthetic construct
  source 1..20: mol_type = other DNA
actcgactacggcgtcaccg

SEQ ID NO: 476 - DNA, 20 bp, Synthetic construct
  source 1..20: mol_type = other DNA
cccactccatagaatctccg

SEQ ID NO: 477 - DNA, 20 bp, Synthetic construct
  source 1..20: mol_type = other DNA
ctgcaactgaaacgcaagcg

SEQ ID NO: 478 - DNA, 20 bp, Synthetic construct
  source 1..20: mol_type = other DNA
gggaccagccaagatcgagg

SEQ ID NO: 479 - DNA, 20 bp, Synthetic construct
  source 1..20: mol_type = other DNA
atgatgtttgatgaccgtcg

SEQ ID NO: 480 - DNA, 20 bp, Synthetic construct
  source 1..20: mol_type = other DNA
ctgttgggacataccgctcg

SEQ ID NO: 481 - DNA, 20 bp, Synthetic construct
  source 1..20: mol_type = other DNA
gatgatatgagccctcgtcg

SEQ ID NO: 482 - DNA, 20 bp, Synthetic construct
  source 1..20: mol_type = other DNA
taaaatcaaagaacttcgag

SEQ ID NO: 483 - DNA, 20 bp, Synthetic construct
  source 1..20: mol_type = other DNA
aatgcacttccacaacacaa

SEQ ID NO: 484 - DNA, 20 bp, Synthetic construct
  source 1..20: mol_type = other DNA
aatgtgctgcacataaccct

SEQ ID NO: 485 - DNA, 20 bp, Synthetic construct
  source 1..20: mol_type = other DNA
ccacgtgcgagtgcacacga

SEQ ID NO: 486 - DNA, 20 bp, Synthetic construct
  source 1..20: mol_type = other DNA
gcacattcgaactcatacag

SEQ ID NO: 487 - DNA, 20 bp, Synthetic construct
  source 1..20: mol_type = other DNA
ctgcagaggccaaagcacgg

SEQ ID NO: 488 - DNA, 20 bp, Synthetic construct
  source 1..20: mol_type = other DNA
gatggagtatgagcgccaag

SEQ ID NO: 489 - DNA, 20 bp, Synthetic construct
  source 1..20: mol_type = other DNA
gcaacacatcaatgttggga

SEQ ID NO: 490 - DNA, 20 bp, Synthetic construct
  source 1..20: mol_type = other DNA
tgtaattgcccctatagtag

SEQ ID NO: 491 - DNA, 20 bp, Synthetic construct
  source 1..20: mol_type = other DNA
acgtcccagcaggcaacaca

SEQ ID NO: 492 - DNA, 20 bp, Synthetic construct
  source 1..20: mol_type = other DNA
ccaatgatgccttatggcac

SEQ ID NO: 493 - DNA, 20 bp, Synthetic construct
  source 1..20: mol_type = other DNA
gataagagagccacgaacca

SEQ ID NO: 494 - DNA, 20 bp, Synthetic construct
  source 1..20: mol_type = other DNA
gtttcgggcacgaagtgcaa

SEQ ID NO: 495 - DNA, 20 bp, Synthetic construct
  source 1..20: mol_type = other DNA
agacgctatccacgtcgtag

SEQ ID NO: 496 - DNA, 20 bp, Synthetic construct
  source 1..20: mol_type = other DNA
gcaaatgttacaggtcctgg

SEQ ID NO: 497 - DNA, 20 bp, Synthetic construct
  source 1..20: mol_type = other DNA
ggcggggacaagaaggtcat

SEQ ID NO: 498 - DNA, 20 bp, Synthetic construct
  source 1..20: mol_type = other DNA
gtcttgcaggaatgacacca

SEQ ID NO: 499 - DNA, 20 bp, Synthetic construct
  source 1..20: mol_type = other DNA
cagacccgtacaaagtgtac

SEQ ID NO: 500 - DNA, 20 bp, Synthetic construct
  source 1..20: mol_type = other DNA
cccatgacgtttggaccccg

SEQ ID NO: 501 - DNA, 20 bp, Synthetic construct
  source 1..20: mol_type = other DNA
ctaccgggaaatcctcgtga

SEQ ID NO: 502 - DNA, 20 bp, Synthetic construct
  source 1..20: mol_type = other DNA
ctgatcgaccagatcgacag

SEQ ID NO: 503 - DNA, 20 bp, Synthetic construct
  source 1..20: mol_type = other DNA
atgctaagtacctgtgaaag

SEQ ID NO: 504 - DNA, 20 bp, Synthetic construct
  source 1..20: mol_type = other DNA
cattgaatatgattgcaagg

SEQ ID NO: 505 - DNA, 20 bp, Synthetic construct
  source 1..20: mol_type = other DNA
taggtgttgatacgagccca

SEQ ID NO: 506 - DNA, 20 bp, Synthetic construct
  source 1..20: mol_type = other DNA
tattcatagatctactgaca

SEQ ID NO: 507 - DNA, 20 bp, Synthetic construct
  source 1..20: mol_type = other DNA
agctgtactagagttacggt

SEQ ID NO: 508 - DNA, 20 bp, Synthetic construct
  source 1..20: mol_type = other DNA
ggctaacaatggtaccgatg

SEQ ID NO: 509 - DNA, 20 bp, Synthetic construct
  source 1..20: mol_type = other DNA
tggagttggcaccgttacag

SEQ ID NO: 510 - DNA, 20 bp, Synthetic construct
  source 1..20: mol_type = other DNA
tgtggagactgaataactga

SEQ ID NO: 511 - DNA, 20 bp, Synthetic construct
  source 1..20: mol_type = other DNA
acagaacgcggctctacgca

SEQ ID NO: 512 - DNA, 20 bp, Synthetic construct
  source 1..20: mol_type = other DNA
aggactctacctgtttgcca

SEQ ID NO: 513 - DNA, 20 bp, Synthetic construct
  source 1..20: mol_type = other DNA
atgtgagaagagttcagagg

SEQ ID NO: 514 - DNA, 20 bp, Synthetic construct
  source 1..20: mol_type = other DNA
ggagatcaagcagctcacag

SEQ ID NO: 515 - DNA, 20 bp, Synthetic construct
  source 1..20: mol_type = other DNA
gaactccctgccagatatcg

SEQ ID NO: 516 - DNA, 20 bp, Synthetic construct
  source 1..20: mol_type = other DNA
tctaggccgatacaaagcag

SEQ ID NO: 517 - DNA, 20 bp, Synthetic construct
  source 1..20: mol_type = other DNA
tctccctcgacagtcatgtg

SEQ ID NO: 518 - DNA, 20 bp, Synthetic construct
  source 1..20: mol_type = other DNA
ttaattccaaccaaatcccg

SEQ ID NO: 519 - DNA, 20 bp, Synthetic construct
  source 1..20: mol_type = other DNA
aagtcgacatactctcggct

SEQ ID NO: 520 - DNA, 20 bp, Synthetic construct
  source 1..20: mol_type = other DNA
cctgcctcacctcacactcg

SEQ ID NO: 521 - DNA, 20 bp, Synthetic construct
  source 1..20: mol_type = other DNA
gccgacttacgatttccgag

SEQ ID NO: 522 - DNA, 20 bp, Synthetic construct
  source 1..20: mol_type = other DNA
ggagtctgtgttatctggaa

SEQ ID NO: 523 - DNA, 20 bp, Synthetic construct
  source 1..20: mol_type = other DNA
acttcggcaagactttgagg

SEQ ID NO: 524 - DNA, 20 bp, Synthetic construct
  source 1..20: mol_type = other DNA
gcatcgcaccatgtctcagg

SEQ ID NO: 525 - DNA, 20 bp, Synthetic construct
  source 1..20: mol_type = other DNA
ggagggcactaccactacgc

SEQ ID NO: 526 - DNA, 20 bp, Synthetic construct
  source 1..20: mol_type = other DNA
tcggttgacaatcaatagtg

SEQ ID NO: 527 - DNA, 20 bp, Synthetic construct
  source 1..20: mol_type = other DNA
ccagtcgacacccatcattg

SEQ ID NO: 528 - DNA, 20 bp, Synthetic construct
  source 1..20: mol_type = other DNA
ccccagtaaacgctccacag

SEQ ID NO: 529 - DNA, 20 bp, Synthetic construct
  source 1..20: mol_type = other DNA
gaacgtcaccttagaatccg

SEQ ID NO: 530 - DNA, 20 bp, Synthetic construct
  source 1..20: mol_type = other DNA
tctttgtctgccacaaacgt

SEQ ID NO: 531 - DNA, 20 bp, Synthetic construct
  source 1..20: mol_type = other DNA
aacaggcattccggaaacgg

SEQ ID NO: 532 - DNA, 20 bp, Synthetic construct
  source 1..20: mol_type = other DNA
aggtcacgttctgcacaaag

SEQ ID NO: 533 - DNA, 20 bp, Synthetic construct
  source 1..20: mol_type = other DNA
ctaacttggacccaaactca

SEQ ID NO: 534 - DNA, 20 bp, Synthetic construct
  source 1..20: mol_type = other DNA
gcattacccctactaccgga

SEQ ID NO: 535 - DNA, 20 bp, Synthetic construct
  source 1..20: mol_type = other DNA
agagtgcatcgacccctcgg

SEQ ID NO: 536 - DNA, 20 bp, Synthetic construct
  source 1..20: mol_type = other DNA
ctgcggggaggactccgtcg

SEQ ID NO: 537 - DNA, 20 bp, Synthetic construct
  source 1..20: mol_type = other DNA
cttcggggagacaacgacgg

SEQ ID NO: 538 - DNA, 20 bp, Synthetic construct
  source 1..20: mol_type = other DNA
gctgcaccgagtcgtagtcg

SEQ ID NO: 539 - DNA, 20 bp, Synthetic construct
  source 1..20: mol_type = other DNA
aaaccagggccaccgaaagg

SEQ ID NO: 540 - DNA, 20 bp, Synthetic construct
  source 1..20: mol_type = other DNA
ccctcgcgcttgaggccgcg

SEQ ID NO: 541 - DNA, 20 bp, Synthetic construct
  source 1..20: mol_type = other DNA
cttcggtctcttcgacgacg

SEQ ID NO: 542 - DNA, 20 bp, Synthetic construct
  source 1..20: mol_type = other DNA
tcggggtaatagaacgcagg

SEQ ID NO: 543 - DNA, 20 bp, Synthetic construct
  source 1..20: mol_type = other DNA
accggcagcgtcaaacaccc

SEQ ID NO: 544 - DNA, 20 bp, Synthetic construct
  source 1..20: mol_type = other DNA
atgaccagaaaaacataaga

SEQ ID NO: 545 - DNA, 20 bp, Synthetic construct
  source 1..20: mol_type = other DNA
ccttcatggagaaatgccgt

SEQ ID NO: 546 - DNA, 20 bp, Synthetic construct
  source 1..20: mol_type = other DNA
ggtgcagagaaaccggcatg

SEQ ID NO: 547 - DNA, 20 bp, Synthetic construct
  source 1..20: mol_type = other DNA
aaaggagcagagcaaaaccg

SEQ ID NO: 548 - DNA, 20 bp, Synthetic construct
  source 1..20: mol_type = other DNA
actgctatcctgaatagaca

SEQ ID NO: 549 - DNA, 20 bp, Synthetic construct
  source 1..20: mol_type = other DNA
gaggacttgggaaattcaag

SEQ ID NO: 550 - DNA, 20 bp, Synthetic construct
  source 1..20: mol_type = other DNA
gccttcgatcaagtgcactg

SEQ ID NO: 551 - DNA, 20 bp, Synthetic construct
  source 1..20: mol_type = other DNA
ccgacctcgagtccccgcag

SEQ ID NO: 552 - DNA, 20 bp, Synthetic construct
  source 1..20: mol_type = other DNA
cgtggtggcgcggatcctag

SEQ ID NO: 553 - DNA, 20 bp, Synthetic construct
  source 1..20: mol_type = other DNA
cgtgtccatgtcgagccgcg

SEQ ID NO: 554 - DNA, 20 bp, Synthetic construct
  source 1..20: mol_type = other DNA
gagttctcaggtgcgccttg

SEQ ID NO: 555 - DNA, 20 bp, Synthetic construct
  source 1..20: mol_type = other DNA
accaggcacacaagagactg

SEQ ID NO: 556 - DNA, 20 bp, Synthetic construct
  source 1..20: mol_type = other DNA
agaaatacggtccgaaacgt

SEQ ID NO: 557 - DNA, 20 bp, Synthetic construct
  source 1..20: mol_type = other DNA
agtctggaaagcgtcacttg

SEQ ID NO: 558 - DNA, 20 bp, Synthetic construct
  source 1..20: mol_type = other DNA
tatttacatgtaacgctaca

SEQ ID NO: 559 - DNA, 20 bp, Synthetic construct
  source 1..20: mol_type = other DNA
caaaatctcaccctgaaacg

SEQ ID NO: 560 - DNA, 20 bp, Synthetic construct
  source 1..20: mol_type = other DNA
ccacggcgaagcgccctccg

SEQ ID NO: 561 - DNA, 20 bp, Synthetic construct
  source 1..20: mol_type = other DNA
ggacacataatggcaaatcg

SEQ ID NO: 562 - DNA, 20 bp, Synthetic construct
  source 1..20: mol_type = other DNA
tgggtggtactgccatccgg

SEQ ID NO: 563 - DNA, 30 bp, Synthetic construct
  source 1..30: mol_type = other DNA
aatgagaagactaaaggagtatgggggctt

SEQ ID NO: 564 - DNA, 30 bp, Synthetic construct
  source 1..30: mol_type = other DNA
tgttcataaacactgaccttgccatggctt

SEQ ID NO: 565 - DNA, 30 bp, Synthetic construct
  source 1..30: mol_type = other DNA
atcatctccctctatgtcatacaatggact

SEQ ID NO: 566 - DNA, 30 bp, Synthetic construct
  source 1..30: mol_type = other DNA
ctgctgactcactgtccactagagtggcct

SEQ ID NO: 567 - DNA, 30 bp, Synthetic construct
  source 1..30: mol_type = other DNA
cagtacatcagagaatgcacacaggggaga

SEQ ID NO: 568 - DNA, 30 bp, Synthetic construct
  source 1..30: mol_type = other DNA
agtgagtatgctgatgtactatgagggcag

SEQ ID NO: 569 - DNA, 30 bp, Synthetic construct
  source 1..30: mol_type = other DNA
ccttatacagagtcctctgagcaggggtca

SEQ ID NO: 570 - DNA, 30 bp, Synthetic construct
  source 1..30: mol_type = other DNA
atattagaagtagaatttccacatcggggc

SEQ ID NO: 571 - DNA, 30 bp, Synthetic construct
  source 1..30: mol_type = other DNA
tgatgatcttggtaaactcgctgaaggctt

SEQ ID NO: 572 - DNA, 30 bp, Synthetic construct
  source 1..30: mol_type = other DNA
tccaggtaactagggatataccctgggagg

SEQ ID NO: 573 - DNA, 30 bp, Synthetic construct
  source 1..30: mol_type = other DNA
tcactaccgctgtatcacttgtgagggctg

SEQ ID NO: 574 - DNA, 30 bp, Synthetic construct
  source 1..30: mol_type = other DNA
acagtggttctagatgactcgaagcgggtg

SEQ ID NO: 575 - DNA, 30 bp, Synthetic construct
  source 1..30: mol_type = other DNA
ctagatgtgaaaggtactcctgtgtggatc

SEQ ID NO: 576 - DNA, 30 bp, Synthetic construct
  source 1..30: mol_type = other DNA
ttggcttgacgtgggctattgtgagggacg

SEQ ID NO: 577 - DNA, 30 bp, Synthetic construct
  source 1..30: mol_type = other DNA
catcgacccggttcgattagaacagggcat

SEQ ID NO: 578 - DNA, 30 bp, Synthetic construct
  source 1..30: mol_type = other DNA
ggctgtatgtcagttggtttcaagcggaca

SEQ ID NO: 579 - DNA, 30 bp, Synthetic construct
  source 1..30: mol_type = other DNA
tggaacaatccagatttctaacccaggatc

SEQ ID NO: 580 - DNA, 30 bp, Synthetic construct
  source 1..30: mol_type = other DNA
actagcatatatcagactagcacggggcaa

SEQ ID NO: 581 - DNA, 30 bp, Synthetic construct
  source 1..30: mol_type = other DNA
tgcgtactgtacaattgtagcacctggtgg

SEQ ID NO: 582 - DNA, 30 bp, Synthetic construct
  source 1..30: mol_type = other DNA
ctattgcctacctgagctaaagcagggatt

SEQ ID NO: 583 - DNA, 30 bp, Synthetic construct
  source 1..30: mol_type = other DNA
cgtgcctggcctacccgtgcgccggggcgg

SEQ ID NO: 584 - DNA, 30 bp, Synthetic construct
  source 1..30: mol_type = other DNA
gggcctcggtgagccagacgacggcggcgg

SEQ ID NO: 585 - DNA, 30 bp, Synthetic construct
  source 1..30: mol_type = other DNA
gctggtggctaaggaactccacctcggcct

SEQ ID NO: 586 - DNA, 30 bp, Synthetic construct
  source 1..30: mol_type = other DNA
cctttccttcttcgtacttcgacgaggacg

SEQ ID NO: 587 - DNA, 30 bp, Synthetic construct
  source 1..30: mol_type = other DNA
atatgaacttaacatatgagaaggaggctg

SEQ ID NO: 588 - DNA, 30 bp, Synthetic construct
  source 1..30: mol_type = other DNA
ttctgagtaaaagaagctccacactggtta

SEQ ID NO: 589 - DNA, 30 bp, Synthetic construct
  source 1..30: mol_type = other DNA
tctgtagctacgcctgtagaagaagggacg

SEQ ID NO: 590 - DNA, 30 bp, Synthetic construct
  source 1..30: mol_type = other DNA
atgctgacattctggagatagttgtggcag

SEQ ID NO: 591 - DNA, 30 bp, Synthetic construct
  source 1..30: mol_type = other DNA
cagaactcgagttgacatcagttgtggaaa

SEQ ID NO: 592 - DNA, 30 bp, Synthetic construct
  source 1..30: mol_type = other DNA
ccgcctcataaggactcttgacgctggcta

SEQ ID NO: 593 - DNA, 30 bp, Synthetic construct
  source 1..30: mol_type = other DNA
gaaactgttggagtttatatacacaggaac

SEQ ID NO: 594 - DNA, 30 bp, Synthetic construct
  source 1..30: mol_type = other DNA
tgggtaaatgcctttccacataagtggcag

SEQ ID NO: 595 - DNA, 30 bp, Synthetic construct
  source 1..30: mol_type = other DNA
agggaattatggacagccccacgtgggcca

SEQ ID NO: 596 - DNA, 30 bp, Synthetic construct
  source 1..30: mol_type = other DNA
tcaccccagaataagaatgcaccgaggtgc

SEQ ID NO: 597 - DNA, 30 bp, Synthetic construct
  source 1..30: mol_type = other DNA
cctgctcttcctattcctacgcggaggacg

SEQ ID NO: 598 - DNA, 30 bp, Synthetic construct
  source 1..30: mol_type = other DNA
ctctgttcagcataacgaaaagtgtggagc

SEQ ID NO: 599 - DNA, 30 bp, Synthetic construct
  source 1..30: mol_type = other DNA
tactcactaatgcatttgagactgggggtt

SEQ ID NO: 600 - DNA, 30 bp, Synthetic construct
  source 1..30: mol_type = other DNA
aggactcatgactgcttatctctgtggttc

SEQ ID NO: 601 - DNA, 30 bp, Synthetic construct
  source 1..30: mol_type = other DNA
cagcgccaccctccacagtaacgacggcag

SEQ ID NO: 602 - DNA, 30 bp, Synthetic construct
  source 1..30: mol_type = other DNA
tgtctgcgtgttccacggacatggggggag

SEQ ID NO: 603 - DNA, 30 bp, Synthetic construct
  source 1..30: mol_type = other DNA
aatgactgcaggcattaatgccgacggaca

SEQ ID NO: 604 - DNA, 30 bp, Synthetic construct
  source 1..30: mol_type = other DNA
taggaggagcaccaaaccgatgggaggttt

SEQ ID NO: 605 - DNA, 30 bp, Synthetic construct
  source 1..30: mol_type = other DNA
acaaatagcttgactacatctagtgggcag

SEQ ID NO: 606 - DNA, 30 bp, Synthetic construct
  source 1..30: mol_type = other DNA
cttggcaaacgtttcaagtacaaggggtcc

SEQ ID NO: 607 - DNA, 30 bp, Synthetic construct
  source 1..30: mol_type = other DNA
tgggaactccatagtaccccacgaaggaaa

SEQ ID NO: 608 - DNA, 30 bp, Synthetic construct
  source 1..30: mol_type = other DNA
agtagtttgtaggaatggtctcagtggagc

SEQ ID NO: 609 - DNA, 30 bp, Synthetic construct
  source 1..30: mol_type = other DNA
gacatcgtcaaggtgtgtaagcggcggctt

SEQ ID NO: 610 - DNA, 30 bp, Synthetic construct
  source 1..30: mol_type = other DNA
cagatgagccaccaatgtctagtggggctg

SEQ ID NO: 611 - DNA, 30 bp, Synthetic construct
  source 1..30: mol_type = other DNA
ccagaactcgaaaatcagtaacagaggtga

SEQ ID NO: 612 - DNA, 30 bp, Synthetic construct
  source 1..30: mol_type = other DNA
aaataatagtcaggaagtgccaaagggtga

SEQ ID NO: 613 - DNA, 30 bp, Synthetic construct
  source 1..30: mol_type = other DNA
catacagggcaattgaactgccgtgggttc

SEQ ID NO: 614 - DNA, 30 bp, Synthetic construct
  source 1..30: mol_type = other DNA
tatcgattagtattgtagccgcagcggtca

SEQ ID NO: 615 - DNA, 30 bp, Synthetic construct
  source 1..30: mol_type = other DNA
ttgcaatttcatgccaagtcacctgggtaa

SEQ ID NO: 616 - DNA, 30 bp, Synthetic construct
  source 1..30: mol_type = other DNA
tcccccagtaccaatattagcatgtggcaa

SEQ ID NO: 617 - DNA, 30 bp, Synthetic construct
  source 1..30: mol_type = other DNA
gtctgttattgtacaggttcgagtaggtga

SEQ ID NO: 618 - DNA, 30 bp, Synthetic construct
  source 1..30: mol_type = other DNA
gatctgataatcaagtgattcactgggaaa

SEQ ID NO: 619 - DNA, 30 bp, Synthetic construct
  source 1..30: mol_type = other DNA
gaagatcaagcggtgtgagcggaagggcac

SEQ ID NO: 620 - DNA, 30 bp, Synthetic construct
  source 1..30: mol_type = other DNA
cgctcagccaccacaaagggcacgcggtcc

SEQ ID NO: 621 - DNA, 30 bp, Synthetic construct
  source 1..30: mol_type = other DNA
cggtgctggaaggcctccatactgaggctg

SEQ ID NO: 622 - DNA, 30 bp, Synthetic construct
  source 1..30: mol_type = other DNA
tgtagctgggaataaatgtccaccaggctt

SEQ ID NO: 623 - DNA, 30 bp, Synthetic construct
  source 1..30: mol_type = other DNA
tggaagatgtattccgcatagtcagggtgc

SEQ ID NO: 624 - DNA, 30 bp, Synthetic construct
  source 1..30: mol_type = other DNA
cagacacttagcgtgataaacccggggaca

SEQ ID NO: 625 - DNA, 30 bp, Synthetic construct
  source 1..30: mol_type = other DNA
cccgctcttccgcccaaacttccgcggctg

SEQ ID NO: 626 - DNA, 30 bp, Synthetic construct
  source 1..30: mol_type = other DNA
tgggggagcgcggcatatccgacaaggaaa

SEQ ID NO: 627 - DNA, 30 bp, Synthetic construct
  source 1..30: mol_type = other DNA
aacactaaatgaacaacgcctggaaggaga

SEQ ID NO: 628 - DNA, 30 bp, Synthetic construct
  source 1..30: mol_type = other DNA
tgaagtgaaagatgaacagacaccaggctg

SEQ ID NO: 629 - DNA, 30 bp, Synthetic construct
  source 1..30: mol_type = other DNA
aggatgatgatgtagaggaaatcggggatc

SEQ ID NO: 630 - DNA, 30 bp, Synthetic construct
  source 1..30: mol_type = other DNA
tcgcttcctgaaccctgagcgttgtggtgg

SEQ ID NO: 631 - DNA, 30 bp, Synthetic construct
  source 1..30: mol_type = other DNA
gcgcacgtgcacctcgagcccgtgcggcgt

SEQ ID NO: 632 - DNA, 30 bp, Synthetic construct
  source 1..30: mol_type = other DNA
ggcgccggtacattctctaaacggagggaa

SEQ ID NO: 633 - DNA, 30 bp, Synthetic construct
  source 1..30: mol_type = other DNA
gcgatgggcacattgacttctccgaggctg

SEQ ID NO: 634 - DNA, 30 bp, Synthetic construct
  source 1..30: mol_type = other DNA
gcctttcaaaccgtactcatggagcggcct

SEQ ID NO: 635 - DNA, 30 bp, Synthetic construct
  source 1..30: mol_type = other DNA
tcacggccgacatcatgtccataggggtga

SEQ ID NO: 636 - DNA, 30 bp, Synthetic construct
  source 1..30: mol_type = other DNA
ccagtggccctgagcaatagcacgaggcct

SEQ ID NO: 637 - DNA, 30 bp, Synthetic construct
  source 1..30: mol_type = other DNA
accattccgatagtgttgaaaccaaggcat

SEQ ID NO: 638 - DNA, 30 bp, Synthetic construct
  source 1..30: mol_type = other DNA
ggacttgtaaacagtacactctggagggtt

SEQ ID NO: 639 - DNA, 30 bp, Synthetic construct
  source 1..30: mol_type = other DNA
tgaaaagggatgaagatcctcacaaggctg

SEQ ID NO: 640 - DNA, 30 bp, Synthetic construct
  source 1..30: mol_type = other DNA
tggtcagtgcggataactatacactggacc

SEQ ID NO: 641 - DNA, 30 bp, Synthetic construct
  source 1..30: mol_type = other DNA
gcatcctggattattatagccacgaggcgg

SEQ ID NO: 642 - DNA, 30 bp, Synthetic construct
  source 1..30: mol_type = other DNA
agcagggttgcctgcgatgacgaggggcgt

SEQ ID NO: 643 - DNA, 30 bp, Synthetic construct
  source 1..30: mol_type = other DNA
ccctcagggtgacgatgtcaactgtggtca

SEQ ID NO: 644 - DNA, 30 bp, Synthetic construct
  source 1..30: mol_type = other DNA
agacccacagatgcaacataagctgggaaa

SEQ ID NO: 645 - DNA, 30 bp, Synthetic construct
  source 1..30: mol_type = other DNA
ctcagctgggaaaagaacttcgaggggtct

SEQ ID NO: 646 - DNA, 30 bp, Synthetic construct
  source 1..30: mol_type = other DNA
gcccttgggaaggacaccattccgtggctc

SEQ ID NO: 647 - DNA, 30 bp, Synthetic construct
  source 1..30: mol_type = other DNA
ccttcctgcgtgtaccaaatgcagcggccc

SEQ ID NO: 648 - DNA, 30 bp, Synthetic construct
  source 1..30: mol_type = other DNA
cgtacgggtggctctcaagcgcggcggcct

SEQ ID NO: 649 - DNA, 30 bp, Synthetic construct
  source 1..30: mol_type = other DNA
ttctgaaatcgacagtactgacatcggttt

SEQ ID NO: 650 - DNA, 30 bp, Synthetic construct
  source 1..30: mol_type = other DNA
tccatggtcagcttggtgtagtcggggttc

SEQ ID NO: 651 - DNA, 30 bp, Synthetic construct
  source 1..30: mol_type = other DNA
tcagcctgtatatcccatcacgggtggatt

SEQ ID NO: 652 - DNA, 30 bp, Synthetic construct
  source 1..30: mol_type = other DNA
ctttgcagtcaaataaagtgcccgtggtgc

SEQ ID NO: 653 - DNA, 30 bp, Synthetic construct
  source 1..30: mol_type = other DNA
ttatgtacccggaataactcgagtaggagg

SEQ ID NO: 654 - DNA, 30 bp, Synthetic construct
  source 1..30: mol_type = other DNA
tgaggtgttacaatagctggatgagggatg

SEQ ID NO: 655 - DNA, 30 bp, Synthetic construct
  source 1..30: mol_type = other DNA
gttgcacaagcactcgccaccaagaggcag

SEQ ID NO: 656 - DNA, 30 bp, Synthetic construct
  source 1..30: mol_type = other DNA
ctcacgtggttgagggaaaatccagggggc

SEQ ID NO: 657 - DNA, 30 bp, Synthetic construct
  source 1..30: mol_type = other DNA
ctctgtgatactctgatcaccgtagggagc

SEQ ID NO: 658 - DNA, 30 bp, Synthetic construct
  source 1..30: mol_type = other DNA
ccactgtccctaaatgcccccaaagggctc

SEQ ID NO: 659 - DNA, 30 bp, Synthetic construct
  source 1..30: mol_type = other DNA
gagcagatgcccgagagttaatcagggcat

SEQ ID NO: 660 - DNA, 30 bp, Synthetic construct
  source 1..30: mol_type = other DNA
gcttcataaaaccgagtctcacggaggata

SEQ ID NO: 661 - DNA, 30 bp, Synthetic construct
  source 1..30: mol_type = other DNA
agcaccatacacttggctatttcggggaca

SEQ ID NO: 662 - DNA, 30 bp, Synthetic construct
  source 1..30: mol_type = other DNA
cagctgaaccgaaagcgagaacgtcggccc

SEQ ID NO: 663 - DNA, 30 bp, Synthetic construct
  source 1..30: mol_type = other DNA
cccgccccgccgtacctctcgtagtggcgg

SEQ ID NO: 664 - DNA, 30 bp, Synthetic construct
  source 1..30: mol_type = other DNA
acttcttctctagacttaaagatggggtgc

SEQ ID NO: 665 - DNA, 30 bp, Synthetic construct
  source 1..30: mol_type = other DNA
aggagcgacacacgcccatcgagagggtgc

SEQ ID NO: 666 - DNA, 30 bp, Synthetic construct
  source 1..30: mol_type = other DNA
agcggctgtcctgaggcctacaagcggctc

SEQ ID NO: 667 - RNA, 30 nt, Synthetic construct
  source 1..30: mol_type = other RNA
aacaacgaagacgatggagtcttggggtgg

SEQ ID NO: 668 - DNA, 30 bp, Synthetic construct
  source 1..30: mol_type = other DNA
tgccccaaaacagaggaaccacacaggcag

SEQ ID NO: 669 - DNA, 30 bp, Synthetic construct
  source 1..30: mol_type = other DNA
ttgtcttatggaatctccctgtgaaggcac

SEQ ID NO: 670 - DNA, 30 bp, Synthetic construct
  source 1..30: mol_type = other DNA
catatacgtgatgaaattgatacgggggcc

SEQ ID NO: 671 - DNA, 30 bp, Synthetic construct
  source 1..30: mol_type = other DNA
tcctaggtggtgggcacatcaaagaggtca

SEQ ID NO: 672 - DNA, 30 bp, Synthetic construct
  source 1..30: mol_type = other DNA
tgtgctgaacaaaatcctccaaggcggtga

SEQ ID NO: 673 - DNA, 30 bp, Synthetic construct
  source 1..30: mol_type = other DNA
ttgtggcacagcgcacacacatagcggcca

SEQ ID NO: 674 - DNA, 30 bp, Synthetic construct
  source 1..30: mol_type = other DNA
tgtcgggaaggtctcactgcactgaggaca

SEQ ID NO: 675 - DNA, 30 bp, Synthetic construct
  source 1..30: mol_type = other DNA
ggggacaccatccaaatagcgcagtggctg

SEQ ID NO: 676 - DNA, 30 bp, Synthetic construct
  source 1..30: mol_type = other DNA
tggtgacaggctgaggtccacgagaggggc

SEQ ID NO: 677 - DNA, 30 bp, Synthetic construct
  source 1..30: mol_type = other DNA
tagtgggtgcgcgtgtgcttaaagcggtca

SEQ ID NO: 678 - DNA, 30 bp, Synthetic construct
  source 1..30: mol_type = other DNA
ggcttgacatggtaatcgttgacatggtcc

SEQ ID NO: 679 - DNA, 30 bp, Synthetic construct
  source 1..30: mol_type = other DNA
catacacagctacgggatacggccgggccc

SEQ ID NO: 680 - DNA, 30 bp, Synthetic construct
  source 1..30: mol_type = other DNA
cctaccggagtctcaaagcgcctggggctc

SEQ ID NO: 681 - DNA, 30 bp, Synthetic construct
  source 1..30: mol_type = other DNA
gctgctgaggttggtgtaaacgggaggtgg

SEQ ID NO: 682 - DNA, 30 bp, Synthetic construct
  source 1..30: mol_type = other DNA
gcgggggtaaaagtactgtcccgggggtgt

SEQ ID NO: 683 - DNA, 30 bp, Synthetic construct
  source 1..30: mol_type = other DNA
ggacaagtcgagcggcaagcattacggtgt

SEQ ID NO: 684 - DNA, 30 bp, Synthetic construct
  source 1..30: mol_type = other DNA
ggagcgagctcttcgtgctgaacgcggcgc

SEQ ID NO: 685 - DNA, 30 bp, Synthetic construct
  source 1..30: mol_type = other DNA
gcgagcggcggagacctcttcccggggcag

SEQ ID NO: 686 - DNA, 30 bp, Synthetic construct
  source 1..30: mol_type = other DNA
tggtgctggtcgatctggcagtcacggttg

SEQ ID NO: 687 - DNA, 30 bp, Synthetic construct
  source 1..30: mol_type = other DNA
tattacagacgattgctgtactcgaggctc

SEQ ID NO: 688 - DNA, 30 bp, Synthetic construct
  source 1..30: mol_type = other DNA
tctcagagtataacgatgttcttgaggttc

SEQ ID NO: 689 - DNA, 30 bp, Synthetic construct
  source 1..30: mol_type = other DNA
aacagcagagtacccgagaatcatgggtaa

SEQ ID NO: 690 - DNA, 30 bp, Synthetic construct
  source 1..30: mol_type = other DNA
ttgttcaattgcctgagatcgtatgggacc

SEQ ID NO: 691 - DNA, 30 bp, Synthetic construct
  source 1..30: mol_type = other DNA
ctggaggacacgggatccacaccccggcgc

SEQ ID NO: 692 - DNA, 30 bp, Synthetic construct
  source 1..30: mol_type = other DNA
gtacatccctggcctagcaaacagaggaag

SEQ ID NO: 693 - DNA, 30 bp, Synthetic construct
  source 1..30: mol_type = other DNA
gttggagaatgggtgtccagaggtgggaaa

SEQ ID NO: 694 - DNA, 30 bp, Synthetic construct
  source 1..30: mol_type = other DNA
ccctgggtaacggggaaaagtccgaggact

SEQ ID NO: 695 - DNA, 30 bp, Synthetic construct
  source 1..30: mol_type = other DNA
gaagcggaagtggctaccacccgacggcac

SEQ ID NO: 696 - DNA, 30 bp, Synthetic construct
  source 1..30: mol_type = other DNA
cacgctgagcccgatgtaccccagtggtgg

SEQ ID NO: 697 - DNA, 30 bp, Synthetic construct
  source 1..30: mol_type = other DNA
ccaggaccggccagaggcgacacagggaag

SEQ ID NO: 698 - DNA, 30 bp, Synthetic construct
  source 1..30: mol_type = other DNA
cctcggaaggcacccagatccggagggccg

SEQ ID NO: 699 - DNA, 30 bp, Synthetic construct
  source 1..30: mol_type = other DNA
cttccacttcgaccgacaaacctgaggtca

SEQ ID NO: 700 - DNA, 30 bp, Synthetic construct
  source 1..30: mol_type = other DNA
aggactgatcgtaggaccacggtggggatg

SEQ ID NO: 701 - DNA, 30 bp, Synthetic construct
  source 1..30: mol_type = other DNA
taaaggcagtggagtggttcagggaggcac

SEQ ID NO: 702 - DNA, 30 bp, Synthetic construct
  source 1..30: mol_type = other DNA
aaactagatgatcagaccaagcccgggagc

SEQ ID NO: 703 - DNA, 30 bp, Synthetic construct
  source 1..30: mol_type = other DNA
actgacaggttggtctgtccagaaaggctc

SEQ ID NO: 704 - DNA, 30 bp, Synthetic construct
  source 1..30: mol_type = other DNA
cggtgcaagtacagccagtcccggaggatc

SEQ ID NO: 705 - DNA, 30 bp, Synthetic construct
  source 1..30: mol_type = other DNA
ccaaggagtcggtgaagatcctccgggact

SEQ ID NO: 706 - DNA, 30 bp, Synthetic construct
  source 1..30: mol_type = other DNA
ccctggcgggcaaaaggaagcgcaggggga

SEQ ID NO: 707 - DNA, 30 bp, Synthetic construct
  source 1..30: mol_type = other DNA
atggacatgaatgcagaggattcggggtgt

SEQ ID NO: 708 - DNA, 30 bp, Synthetic construct
  source 1..30: mol_type = other DNA
gcgcagggcccggaggaacaacagtggaga

SEQ ID NO: 709 - DNA, 30 bp, Synthetic construct
  source 1..30: mol_type = other DNA
cgctccagccgatgcacaaaacgttggtgc

SEQ ID NO: 710 - DNA, 30 bp, Synthetic construct
  source 1..30: mol_type = other DNA
tggctgtggagcactcataccgaaaggcag

SEQ ID NO: 711 - DNA, 30 bp, Synthetic construct
  source 1..30: mol_type = other DNA
gtatagagcgtgcagataatgacaaggaat

SEQ ID NO: 712 - DNA, 30 bp, Synthetic construct
  source 1..30: mol_type = other DNA
ctttagctggcagaccacaaactgaggatc

SEQ ID NO: 713 - DNA, 30 bp, Synthetic construct
  source 1..30: mol_type = other DNA
atacattcttcataccaggaccagaggaaa

SEQ ID NO: 714 - DNA, 30 bp, Synthetic construct
  source 1..30: mol_type = other DNA
tcctccaattcaggacccacacgacgggaa

SEQ ID NO: 715 - DNA, 30 bp, Synthetic construct
  source 1..30: mol_type = other DNA
gtcactgcagggaacctccaccatgggaaa

SEQ ID NO: 716 - DNA, 30 bp, Synthetic construct
  source 1..30: mol_type = other DNA
ccagggatacagggctctacacagaggtcc

SEQ ID NO: 717 - DNA, 30 bp, Synthetic construct
  source 1..30: mol_type = other DNA
gccatggctttgaatgtggcgtgtgggatc

SEQ ID NO: 718 - DNA, 30 bp, Synthetic construct
  source 1..30: mol_type = other DNA
tcacttgtttcgttgtgttccgagtggcta

SEQ ID NO: 719 - DNA, 30 bp, Synthetic construct
  source 1..30: mol_type = other DNA
taggacaggttgccccacgcgttgcggcgg

SEQ ID NO: 720 - DNA, 30 bp, Synthetic construct
  source 1..30: mol_type = other DNA
ggccggagtttagccagtccaactcggcca

SEQ ID NO: 721 - DNA, 30 bp, Synthetic construct
  source 1..30: mol_type = other DNA
gtttggtggcgcaaacgagtagcacggcgt

SEQ ID NO: 722 - DNA, 30 bp, Synthetic construct
  source 1..30: mol_type = other DNA
gtactagcatttgagctagttcgagggcga

SEQ ID NO: 723 - DNA, 30 bp, Synthetic construct
  source 1..30: mol_type = other DNA
gtgcagaggaggagacacatgtcgtggtca

SEQ ID NO: 724 - DNA, 30 bp, Synthetic construct
  source 1..30: mol_type = other DNA
cttgcatacaccatgtccatagagaggatg

SEQ ID NO: 725 - DNA, 30 bp, Synthetic construct
  source 1..30: mol_type = other DNA
caaggccttctgacaattcagcccgggcag

SEQ ID NO: 726 - DNA, 30 bp, Synthetic construct
  source 1..30: mol_type = other DNA
ttgggttctgtagacttcacatgcaggtgg

SEQ ID NO: 727 - DNA, 30 bp, Synthetic construct
  source 1..30: mol_type = other DNA
atggcagccaggacaacaatgcgacggcca

SEQ ID NO: 728 - DNA, 30 bp, Synthetic construct
  source 1..30: mol_type = other DNA
ctgggtggccttaatgttctcctgtggatt

SEQ ID NO: 729 - DNA, 30 bp, Synthetic construct
  source 1..30: mol_type = other DNA
ctctgttcattgtacaatatatggcggatg

SEQ ID NO: 730 - DNA, 30 bp, Synthetic construct
  source 1..30: mol_type = other DNA
gaattaagaggtcagaccgtcgtggggcag

SEQ ID NO: 731 - DNA, 30 bp, Synthetic construct
  source 1..30: mol_type = other DNA
cggcacattctgtacaatgaacagaggctg

SEQ ID NO: 732 - DNA, 30 bp, Synthetic construct
  source 1..30: mol_type = other DNA
gaggatcaagcgtgctgaccggcggggtgc

SEQ ID NO: 733 - DNA, 30 bp, Synthetic construct
  source 1..30: mol_type = other DNA
acggcagccaggaccacaatgccacggcta

SEQ ID NO: 734 - DNA, 30 bp, Synthetic construct
  source 1..30: mol_type = other DNA
tgaacgtgcacatgaatcccccccaggtga

SEQ ID NO: 735 - DNA, 30 bp, Synthetic construct
  source 1..30: mol_type = other DNA
atccacatcgactgctggacaatgaggatg

SEQ ID NO: 736 - DNA, 30 bp, Synthetic construct
  source 1..30: mol_type = other DNA
cagtcagtgagtagtgccaaaccaaggcac

SEQ ID NO: 737 - DNA, 30 bp, Synthetic construct
  source 1..30: mol_type = other DNA
atgggtggcgtactgcacgtgtcgtggctg

SEQ ID NO: 738 - DNA, 30 bp, Synthetic construct
  source 1..30: mol_type = other DNA
acctttcacattatgaccaacaccaggtca

SEQ ID NO: 739 - DNA, 30 bp, Synthetic construct
  source 1..30: mol_type = other DNA
cctcacccaggcatcatccgacaagggctc

SEQ ID NO: 740 - DNA, 30 bp, Synthetic construct
  source 1..30: mol_type = other DNA
tgtccccacccacagggatcaacgtggcca

SEQ ID NO: 741 - DNA, 30 bp, Synthetic construct
  source 1..30: mol_type = other DNA
tctccctacttaggcactgccaggcggacc

SEQ ID NO: 742 - DNA, 30 bp, Synthetic construct
  source 1..30: mol_type = other DNA
cccggagggtgccaccatgactaggggcag

SEQ ID NO: 743 - DNA, 30 bp, Synthetic construct
  source 1..30: mol_type = other DNA
tgacactcaatcgtgaggattggtcggtgt

SEQ ID NO: 744 - DNA, 30 bp, Synthetic construct
  source 1..30: mol_type = other DNA
aggaactgtgtaaaatgaacaaggaggact

SEQ ID NO: 745 - DNA, 30 bp, Synthetic construct
  source 1..30: mol_type = other DNA
cctcccaacatgaccaccaacgagaggaga

SEQ ID NO: 746 - DNA, 30 bp, Synthetic construct
  source 1..30: mol_type = other DNA
tgagcttacctggatccattcatgtggtca

SEQ ID NO: 747 - DNA, 30 bp, Synthetic construct
  source 1..30: mol_type = other DNA
tgaacttactaatgaagtaatccgaggtat

SEQ ID NO: 748 - DNA, 30 bp, Synthetic construct
  source 1..30: mol_type = other DNA
gctcgagaaagcagtctttacccagggcgc

SEQ ID NO: 749 - DNA, 30 bp, Synthetic construct
  source 1..30: mol_type = other DNA
agagggtctcggagaatgaccgaggggtag

SEQ ID NO: 750 - DNA, 30 bp, Synthetic construct
  source 1..30: mol_type = other DNA
catgtgcatggggaggaccagtcgtggtag

SEQ ID NO: 751 - DNA, 30 bp, Synthetic construct
  source 1..30: mol_type = other DNA
acgacacggaccctgatagcatgaaggatt

SEQ ID NO: 752 - DNA, 30 bp, Synthetic construct
  source 1..30: mol_type = other DNA
aggccatcgctcaggagatatacgcggacc

SEQ ID NO: 753 - DNA, 30 bp, Synthetic construct
  source 1..30: mol_type = other DNA
aacagcagccgaatcgccaaccgccggtga

SEQ ID NO: 754 - DNA, 30 bp, Synthetic construct
  source 1..30: mol_type = other DNA
aggagcttcgcagcctgctaaccacggtga

SEQ ID NO: 755 - DNA, 30 bp, Synthetic construct
  source 1..30: mol_type = other DNA
attcaagatcagtagcggtcccggtggtgg

SEQ ID NO: 756 - DNA, 30 bp, Synthetic construct
  source 1..30: mol_type = other DNA
gtaaagaaaggacgtcgatcgaggcggtgt

SEQ ID NO: 757 - DNA, 30 bp, Synthetic construct
  source 1..30: mol_type = other DNA
gccgaggggtcttaatgatccgcgaggaga

SEQ ID NO: 758 - DNA, 30 bp, Synthetic construct
  source 1..30: mol_type = other DNA
tcacttgaccataattatgctcagtggcag

SEQ ID NO: 759 - DNA, 30 bp, Synthetic construct
  source 1..30: mol_type = other DNA
aatcaatgcttcacccatacaacaaggtgg

SEQ ID NO: 760 - DNA, 30 bp, Synthetic construct
  source 1..30: mol_type = other DNA
ccaacagaccagacagtgttaccagggagg

SEQ ID NO: 761 - DNA, 30 bp, Synthetic construct
  source 1..30: mol_type = other DNA
ctgagaaggacaaaagctttgaaggggggg

SEQ ID NO: 762 - DNA, 30 bp, Synthetic construct
  source 1..30: mol_type = other DNA
acagggttacttgtacacagctgagggtgc

SEQ ID NO: 763 - DNA, 30 bp, Synthetic construct
  source 1..30: mol_type = other DNA
cgggagccggtccctttcccgtcggggagc

SEQ ID NO: 764 - DNA, 30 bp, Synthetic construct
  source 1..30: mol_type = other DNA
caagcctcagttggcaaggtacctgggaaa

SEQ ID NO: 765 - DNA, 30 bp, Synthetic construct
  source 1..30: mol_type = other DNA
cgatcctctcaatcaaaataaggttggtta

SEQ ID NO: 766 - DNA, 30 bp, Synthetic construct
  source 1..30: mol_type = other DNA
gatccgaaaatctgggctaagtgctggcaa

SEQ ID NO: 767 - DNA, 30 bp, Synthetic construct
  source 1..30: mol_type = other DNA
tgccactgtcactaacctactggtgggcac

SEQ ID NO: 768 - DNA, 30 bp, Synthetic construct
  source 1..30: mol_type = other DNA
cgtgcccgcccgctgactgcacataggtga

SEQ ID NO: 769 - DNA, 30 bp, Synthetic construct
  source 1..30: mol_type = other DNA
ttgcctctaccgcccggaaaacgtcggacc

SEQ ID NO: 770 - DNA, 30 bp, Synthetic construct
  source 1..30: mol_type = other DNA
ggctttgggccagagtacgatgcagggcca

SEQ ID NO: 771 - DNA, 30 bp, Synthetic construct
  source 1..30: mol_type = other DNA
gatgcgggacacgtctacttggtggggctc

SEQ ID NO: 772 - DNA, 30 bp, Synthetic construct
  source 1..30: mol_type = other DNA
ttctgcagaacgaggctgccctagaggtct

SEQ ID NO: 773 - DNA, 30 bp, Synthetic construct
  source 1..30: mol_type = other DNA
cactgcggaccagtgtacagcacgaggttc

SEQ ID NO: 774 - DNA, 30 bp, Synthetic construct
  source 1..30: mol_type = other DNA
tatgtaaggtgaggactttgcacagggcag

SEQ ID NO: 775 - DNA, 30 bp, Synthetic construct
  source 1..30: mol_type = other DNA
ctgcacctggtgtggacccacgcgaggcac

SEQ ID NO: 776 - DNA, 30 bp, Synthetic construct
  source 1..30: mol_type = other DNA
atcacctcgaactgcaccataggtgggtgg

SEQ ID NO: 777 - DNA, 30 bp, Synthetic construct
  source 1..30: mol_type = other DNA
ctcagccattgatctgatgtacggaggcat

SEQ ID NO: 778 - DNA, 30 bp, Synthetic construct
  source 1..30: mol_type = other DNA
gagctggggctctgcatctcacagcggtgc

SEQ ID NO: 779 - DNA, 30 bp, Synthetic construct
  source 1..30: mol_type = other DNA
taccacactggaacatatcaagaccggtta

SEQ ID NO: 780 - DNA, 30 bp, Synthetic construct
  source 1..30: mol_type = other DNA
atatgacaaatgcaatgaaaactgtggtgg

SEQ ID NO: 781 - DNA, 30 bp, Synthetic construct
  source 1..30: mol_type = other DNA
tgcaggacatcgtgtaccgcaccatggaga

SEQ ID NO: 782 - DNA, 30 bp, Synthetic construct
  source 1..30: mol_type = other DNA
agcaggccgcccgggaagtcaacacggcgt

SEQ ID NO: 783 - DNA, 30 bp, Synthetic construct
  source 1..30: mol_type = other DNA
gagagaaaatgaatggctcccacagggacc

SEQ ID NO: 784 - DNA, 30 bp, Synthetic construct
  source 1..30: mol_type = other DNA
tgttgatggcttggtccatcacgtgggact

SEQ ID NO: 785 - DNA, 30 bp, Synthetic construct
  source 1..30: mol_type = other DNA
gtgcggggcctcattcacccagaagggcaa

SEQ ID NO: 786 - DNA, 30 bp, Synthetic construct
  source 1..30: mol_type = other DNA
aagctccaagagtgacagagtcgtgggtaa

SEQ ID NO: 787 - DNA, 30 bp, Synthetic construct
  source 1..30: mol_type = other DNA
tcgcatcccgaacctgcattctgaaggatg

SEQ ID NO: 788 - DNA, 30 bp, Synthetic construct
  source 1..30: mol_type = other DNA
ccttcaccgcttcagtgcaacacgtggagg

SEQ ID NO: 789 - DNA, 30 bp, Synthetic construct
  source 1..30: mol_type = other DNA
ttgtccaaggaaaaaactaacgagcggctc

SEQ ID NO: 790 - DNA, 30 bp, Synthetic construct
  source 1..30: mol_type = other DNA
acgagcggctcctagaccggcaggcggcgg

SEQ ID NO: 791 - DNA, 30 bp, Synthetic construct
  source 1..30: mol_type = other DNA
gcaaacggctatgtgcaatctgcggggaca

SEQ ID NO: 792 - DNA, 30 bp, Synthetic construct
  source 1..30: mol_type = other DNA
gacggccctggctggatcccgcagcggcgg

SEQ ID NO: 793 - DNA, 30 bp, Synthetic construct
  source 1..30: mol_type = other DNA
gccgggacaacaaagactgcacagtggaca

SEQ ID NO: 794 - DNA, 30 bp, Synthetic construct
  source 1..30: mol_type = other DNA
actggtggcttcacatcttcaggggggcca

SEQ ID NO: 795 - DNA, 30 bp, Synthetic construct
  source 1..30: mol_type = other DNA
tggaaagatgaactgcgatgtgtgtggatt

SEQ ID NO: 796 - DNA, 30 bp, Synthetic construct
  source 1..30: mol_type = other DNA
gttacaagcagagaagttcccttgaggagc

SEQ ID NO: 797 - DNA, 30 bp, Synthetic construct
  source 1..30: mol_type = other DNA
gtgagctcatacagacccgcatgatggacc

SEQ ID NO: 798 - DNA, 30 bp, Synthetic construct
  source 1..30: mol_type = other DNA
tactggacagattagcaagcaatgtggcaa

SEQ ID NO: 799 - DNA, 30 bp, Synthetic construct
  source 1..30: mol_type = other DNA
gccgccttactctctccatgtcagtggccg

SEQ ID NO: 800 - DNA, 30 bp, Synthetic construct
  source 1..30: mol_type = other DNA
cacggaactcgatgagcccctgcgcgggca

SEQ ID NO: 801 - DNA, 30 bp, Synthetic construct
  source 1..30: mol_type = other DNA
ccaagctcatcgacgactacggagtggagg

SEQ ID NO: 802 - DNA, 30 bp, Synthetic construct
  source 1..30: mol_type = other DNA
cgcatccgccagacggtcaaccgggggcct

SEQ ID NO: 803 - DNA, 30 bp, Synthetic construct
  source 1..30: mol_type = other DNA
aacaaaggaaagagtctactgtcggggaac

SEQ ID NO: 804 - DNA, 30 bp, Synthetic construct
  source 1..30: mol_type = other DNA
caaaagatgtgagtggatctatcgaggctc

SEQ ID NO: 805 - DNA, 30 bp, Synthetic construct
  source 1..30: mol_type = other DNA
catcccttacctgaatcaatactgaggaca

SEQ ID NO: 806 - DNA, 30 bp, Synthetic construct
  source 1..30: mol_type = other DNA
tcatgttatctataagacaccttgtggtct

SEQ ID NO: 807 - DNA, 30 bp, Synthetic construct
  source 1..30: mol_type = other DNA
ggggactacgacctgaatgctgtgcggctc

SEQ ID NO: 808 - DNA, 30 bp, Synthetic construct
  source 1..30: mol_type = other DNA
atgcgcttccgctacaagtgcgaggggcgc

SEQ ID NO: 809 - DNA, 30 bp, Synthetic construct
  source 1..30: mol_type = other DNA
agtaggaagatctcatccccaccgaggcag

SEQ ID NO: 810 - DNA, 30 bp, Synthetic construct
  source 1..30: mol_type = other DNA
cagatcaatggctacacaggaccagggaca

SEQ ID NO: 811 - DNA, 30 bp, Synthetic construct
  source 1..30: mol_type = other DNA
acagaatcagatgacaatgagtcagggaca

SEQ ID NO: 812 - DNA, 30 bp, Synthetic construct
  source 1..30: mol_type = other DNA
tcagcaatacaatatgccacagggaggcgg

SEQ ID NO: 813 - DNA, 30 bp, Synthetic construct
  source 1..30: mol_type = other DNA
agggcctaaccatatgcctatgcagggacc

SEQ ID NO: 814 - DNA, 30 bp, Synthetic construct
  source 1..30: mol_type = other DNA
tgaaggcatgttgtgagagcgtggaggtgg

SEQ ID NO: 815 - DNA, 30 bp, Synthetic construct
  source 1..30: mol_type = other DNA
cggcaggttggtgactgtgaacgccggctt

SEQ ID NO: 816 - DNA, 30 bp, Synthetic construct
  source 1..30: mol_type = other DNA
atggagttcatcgacaacaagctgcggcgc

SEQ ID NO: 817 - DNA, 30 bp, Synthetic construct
  source 1..30: mol_type = other DNA
actggggctgacactagcagacactggtgc

SEQ ID NO: 818 - DNA, 30 bp, Synthetic construct
  source 1..30: mol_type = other DNA
ctggtctgttgtggggtctgaacggggtgg

SEQ ID NO: 819 - DNA, 30 bp, Synthetic construct
  source 1..30: mol_type = other DNA
gcagaggtaccctccgacccaccacggtga

SEQ ID NO: 820 - DNA, 30 bp, Synthetic construct
  source 1..30: mol_type = other DNA
aaggcagggagtgtgtgaactgtggggcaa

SEQ ID NO: 821 - DNA, 30 bp, Synthetic construct
  source 1..30: mol_type = other DNA
gtccggagctgtactcgggcacgtagggcg

SEQ ID NO: 822 - DNA, 30 bp, Synthetic construct
  source 1..30: mol_type = other DNA
cttctccaagacgtccatccaccacggctc

SEQ ID NO: 823 - DNA, 30 bp, Synthetic construct
  source 1..30: mol_type = other DNA
cgtccgttacccagaatatcacggtggtcc

SEQ ID NO: 824 - DNA, 30 bp, Synthetic construct
  source 1..30: mol_type = other DNA
cccagcacatccgtatacactcaggggcta

SEQ ID NO: 825 - DNA, 30 bp, Synthetic construct
  source 1..30: mol_type = other DNA
agaaggtagcatcgaccctaaccgaggaag

SEQ ID NO: 826 - DNA, 30 bp, Synthetic construct
  source 1..30: mol_type = other DNA
ggcttggcccccgagtggatacggaggtgc

SEQ ID NO: 827 - DNA, 30 bp, Synthetic construct
  source 1..30: mol_type = other DNA
ttcaagatcgaagtatgcctcaccaggaga

SEQ ID NO: 828 - DNA, 30 bp, Synthetic construct
  source 1..30: mol_type = other DNA
attaagtgcatactgtagtccttgaggaag

SEQ ID NO: 829 - DNA, 30 bp, Synthetic construct
  source 1..30: mol_type = other DNA
atgcgagcccccaactgacgaatgtggcat

SEQ ID NO: 830 - DNA, 30 bp, Synthetic construct
  source 1..30: mol_type = other DNA
atcataattagtactactatgcacaggttt

SEQ ID NO: 831 - DNA, 30 bp, Synthetic construct
  source 1..30: mol_type = other DNA
acttactctccaggcttaacacagcggggc

SEQ ID NO: 832 - DNA, 30 bp, Synthetic construct
  source 1..30: mol_type = other DNA
caggagctctcgaagactgctgtggggtcg

SEQ ID NO: 833 - DNA, 30 bp, Synthetic construct
  source 1..30: mol_type = other DNA
ccaggtgtacaaattcctgcaccatggtgc

SEQ ID NO: 834 - DNA, 30 bp, Synthetic construct
  source 1..30: mol_type = other DNA
ttcgtgacgcggaggcgtattcggaggacg

SEQ ID NO: 835 - DNA, 30 bp, Synthetic construct
  source 1..30: mol_type = other DNA
ttgtatcacacatagcgacgaagtgggcta

SEQ ID NO: 836 - DNA, 30 bp, Synthetic construct
  source 1..30: mol_type = other DNA
ggaccagagcatctctagctaacgaggcca

SEQ ID NO: 837 - DNA, 30 bp, Synthetic construct
  source 1..30: mol_type = other DNA
aacactaactctgctacccaagtgcggtta

SEQ ID NO: 838 - DNA, 30 bp, Synthetic construct
  source 1..30: mol_type = other DNA
actctaatgttaatccgagaacggtgggga

SEQ ID NO: 839 - DNA, 30 bp, Synthetic construct
  source 1..30: mol_type = other DNA
aatgaacagtgaaactctgttgagtggaga

SEQ ID NO: 840 - DNA, 30 bp, Synthetic construct
  source 1..30: mol_type = other DNA
agcagggaaacgtagccatggcgacggtgg

SEQ ID NO: 841 - DNA, 30 bp, Synthetic construct
  source 1..30: mol_type = other DNA
tgatggttcatctaagaacaagaggggcgt

SEQ ID NO: 842 - DNA, 30 bp, Synthetic construct
  source 1..30: mol_type = other DNA
aaactgctctgaacccgatgcagaaggagt

SEQ ID NO: 843 - DNA, 30 bp, Synthetic construct
  source 1..30: mol_type = other DNA
acacagccgtagtatctcccacactggacg

SEQ ID NO: 844 - DNA, 30 bp, Synthetic construct
  source 1..30: mol_type = other DNA
gcagccacgacaagggacccgaggcggagg

SEQ ID NO: 845 - DNA, 30 bp, Synthetic construct
  source 1..30: mol_type = other DNA
gtcccctgcacatacggagagtaagggtgt

SEQ ID NO: 846 - DNA, 30 bp, Synthetic construct
  source 1..30: mol_type = other DNA
ccagttccgcacagagacaaatggaggaca

SEQ ID NO: 847 - DNA, 30 bp, Synthetic construct
  source 1..30: mol_type = other DNA
ggggtcggaggtaaacttggccgtgggaga

SEQ ID NO: 848 - DNA, 30 bp, Synthetic construct
  source 1..30: mol_type = other DNA
ggtctctaagttgtaacaaaagctcgggct

SEQ ID NO: 849 - DNA, 30 bp, Synthetic construct
  source 1..30: mol_type = other DNA
gatttctgaggctgaaacatagcagggctc

SEQ ID NO: 850 - DNA, 30 bp, Synthetic construct
  source 1..30: mol_type = other DNA
ctgttgccagtactcctccagagacggcag

SEQ ID NO: 851 - DNA, 30 bp, Synthetic construct
  source 1..30: mol_type = other DNA
agatgaggcaaaaagaaatccccagggtcc

SEQ ID NO: 852 - DNA, 30 bp, Synthetic construct
  source 1..30: mol_type = other DNA
cacagatttctgatgaaaaagacgaggatg

SEQ ID NO: 853 - DNA, 30 bp, Synthetic construct
  source 1..30: mol_type = other DNA
acctgctctactggagcgacgaggaggccg

SEQ ID NO: 854 - DNA, 30 bp, Synthetic construct
  source 1..30: mol_type = other DNA
gccgtcacccaaaaaaatgcaccaggggaa

SEQ ID NO: 855 - DNA, 30 bp, Synthetic construct
  source 1..30: mol_type = other DNA
ggcccctgccaagaaggatgtcaagggctc

SEQ ID NO: 856 - DNA, 30 bp, Synthetic construct
  source 1..30: mol_type = other DNA
gatgctcaaagccacagtcgacaatggccc

SEQ ID NO: 857 - DNA, 30 bp, Synthetic construct
  source 1..30: mol_type = other DNA
ggttgaggctcttatttcgagccagggcta

SEQ ID NO: 858 - DNA, 30 bp, Synthetic construct
  source 1..30: mol_type = other DNA
atgtgataagatgcttgaacagctcggtga

SEQ ID NO: 859 - DNA, 30 bp, Synthetic construct
  source 1..30: mol_type = other DNA
atgcatcacggaagtctcgtcctgaggtca

SEQ ID NO: 860 - DNA, 30 bp, Synthetic construct
  source 1..30: mol_type = other DNA
agcagcatgtgcggtcgcacaccggggaaa

SEQ ID NO: 861 - DNA, 30 bp, Synthetic construct
  source 1..30: mol_type = other DNA
tggtggcagcttacggcgacacatgggtat

SEQ ID NO: 862 - DNA, 30 bp, Synthetic construct
  source 1..30: mol_type = other DNA
aaactggtctacgtgccctcgcagagggtc

SEQ ID NO: 863 - DNA, 30 bp, Synthetic construct
  source 1..30: mol_type = other DNA
cgccacctactgaaccctcctccgaggtgc

SEQ ID NO: 864 - DNA, 30 bp, Synthetic construct
  source 1..30: mol_type = other DNA
tgtcgcggatcagaaaggtgccggcgggct

SEQ ID NO: 865 - DNA, 30 bp, Synthetic construct
  source 1..30: mol_type = other DNA
acgctcagcgtcaagacccagtctgggacc

SEQ ID NO: 866 - DNA, 30 bp, Synthetic construct
  source 1..30: mol_type = other DNA
cagcttgagcacgcagtcgaagcggggcac

SEQ ID NO: 867 - DNA, 30 bp, Synthetic construct
  source 1..30: mol_type = other DNA
gagaaagcagagtgcacagaagaaaggttg

SEQ ID NO: 868 - DNA, 30 bp, Synthetic construct
  source 1..30: mol_type = other DNA
ctccccgaaatgttgagtgtggtgaggtct

SEQ ID NO: 869 - DNA, 30 bp, Synthetic construct
  source 1..30: mol_type = other DNA
atgactccagagaatttctccccgaggaac

SEQ ID NO: 870 - DNA, 30 bp, Synthetic construct
  source 1..30: mol_type = other DNA
tttttctttctagatcccatacagtggtcc

SEQ ID NO: 871 - DNA, 30 bp, Synthetic construct
  source 1..30: mol_type = other DNA
aaaaaaagaacaaatggaacgcgatggttg

SEQ ID NO: 872 - DNA, 30 bp, Synthetic construct
  source 1..30: mol_type = other DNA
tactcacctagtaagtcgtttaggtggagg

SEQ ID NO: 873 - DNA, 30 bp, Synthetic construct
  source 1..30: mol_type = other DNA
ttttcatgccagttcacagcagtggggagc

SEQ ID NO: 874 - DNA, 30 bp, Synthetic construct
  source 1..30: mol_type = other DNA
tatgcattgcctcaggaacaaaggtggctc

SEQ ID NO: 875 - DNA, 30 bp, Synthetic construct
  source 1..30: mol_type = other DNA
gatgaacatggaagaatccaacaacggcag

SEQ ID NO: 876 - DNA, 30 bp, Synthetic construct
  source 1..30: mol_type = other DNA
ctggacgccggtcttgatgacgaggggccg

SEQ ID NO: 877 - DNA, 30 bp, Synthetic construct
  source 1..30: mol_type = other DNA
ctttgagaccgaggtgtatcaccaaggcct

SEQ ID NO: 878 - DNA, 30 bp, Synthetic construct
  source 1..30: mol_type = other DNA
agcatcggccggtgctgtacaatggggtcc

SEQ ID NO: 879 - DNA, 30 bp, Synthetic construct
  source 1..30: mol_type = other DNA
tgggcaatgaacgagcaacagcaaaggaga

SEQ ID NO: 880 - DNA, 30 bp, Synthetic construct
  source 1..30: mol_type = other DNA
tgggccgtacactcagcatgaccctggtaa

SEQ ID NO: 881 - DNA, 30 bp, Synthetic construct
  source 1..30: mol_type = other DNA
cttcgaaatcgccaggcagttcgggggaaa

SEQ ID NO: 882 - DNA, 30 bp, Synthetic construct
  source 1..30: mol_type = other DNA
catctccaggatcagttcaaaccgtggcca

SEQ ID NO: 883 - DNA, 30 bp, Synthetic construct
  source 1..30: mol_type = other DNA
tgtaaaacttaaatgtgagcatcctggtga

SEQ ID NO: 884 - DNA, 30 bp, Synthetic construct
  source 1..30: mol_type = other DNA
agctacaactggagcatttactgctggatt

SEQ ID NO: 885 - DNA, 30 bp, Synthetic construct
  source 1..30: mol_type = other DNA
gtctagaagaagaactcaaacctctggagg

SEQ ID NO: 886 - DNA, 30 bp, Synthetic construct
  source 1..30: mol_type = other DNA
tgttttctttgtagaacttgaagtaggtgc

SEQ ID NO: 887 - DNA, 30 bp, Synthetic construct
  source 1..30: mol_type = other DNA
caagagcaagtcgtgcgagttctgcggcaa

SEQ ID NO: 888 - DNA, 30 bp, Synthetic construct
  source 1..30: mol_type = other DNA
ccggccagcagctcgctcacgccgcggctc

SEQ ID NO: 889 - DNA, 30 bp, Synthetic construct
  source 1..30: mol_type = other DNA
tcgcccgccatggacttctcgcggcggctc

SEQ ID NO: 890 - DNA, 30 bp, Synthetic construct
  source 1..30: mol_type = other DNA
ggactcagggtgagggtcagacggaggctc

SEQ ID NO: 891 - DNA, 30 bp, Synthetic construct
  source 1..30: mol_type = other DNA
gaacaagtgttgtttgatcagtcatggttg

SEQ ID NO: 892 - DNA, 30 bp, Synthetic construct
  source 1..30: mol_type = other DNA
agacacatactctaagtcaggcagtggctg

SEQ ID NO: 893 - DNA, 30 bp, Synthetic construct
  source 1..30: mol_type = other DNA
tcgagtgtaatttagagagcagcgtggaga

SEQ ID NO: 894 - DNA, 30 bp, Synthetic construct
  source 1..30: mol_type = other DNA
gtgctctgttcagactctaataggaggtta

SEQ ID NO: 895 - DNA, 30 bp, Synthetic construct
  source 1..30: mol_type = other DNA
cggtccctggatagaatacatcgtcggaac

SEQ ID NO: 896 - DNA, 30 bp, Synthetic construct
  source 1..30: mol_type = other DNA
ttcagacaagatgtcagacctagggggcag

SEQ ID NO: 897 - DNA, 30 bp, Synthetic construct
  source 1..30: mol_type = other DNA
gtcagaggtggaaggtaaacacaaggggat

SEQ ID NO: 898 - DNA, 30 bp, Synthetic construct
  source 1..30: mol_type = other DNA
ttttttctaggataaccaccactgtggcca

SEQ ID NO: 899 - DNA, 30 bp, Synthetic construct
  source 1..30: mol_type = other DNA
ataaacctgatcccaaaacatggaaggcga

SEQ ID NO: 900 - DNA, 30 bp, Synthetic construct
  source 1..30: mol_type = other DNA
ggggcagcattcggtagaccctgaaggcat

SEQ ID NO: 901 - DNA, 30 bp, Synthetic construct
  source 1..30: mol_type = other DNA
ccctggatgcatgcggctagacatgggtgg

SEQ ID NO: 902 - DNA, 30 bp, Synthetic construct
  source 1..30: mol_type = other DNA
cgcttacctgcataggaagacacgggggag

SEQ ID NO: 903 - DNA, 30 bp, Synthetic construct
  source 1..30: mol_type = other DNA
acacacagctcaagcgcttcatccaggtgc

SEQ ID NO: 904 - DNA, 30 bp, Synthetic construct
  source 1..30: mol_type = other DNA
gcataggctccccggacatctgggaggacg

SEQ ID NO: 905 - DNA, 30 bp, Synthetic construct
  source 1..30: mol_type = other DNA
attccactaacccccgagactcagcgggac

SEQ ID NO: 906 - DNA, 30 bp, Synthetic construct
  source 1..30: mol_type = other DNA
gagacgcatgcagagcatcaacgcgggatt

SEQ ID NO: 907 - DNA, 30 bp, Synthetic construct
  source 1..30: mol_type = other DNA
atggaggatgcggatatgactctgtggaca

SEQ ID NO: 908 - DNA, 30 bp, Synthetic construct
  source 1..30: mol_type = other DNA
cgtaggacgcgttcaagtaagcgtaggagt

SEQ ID NO: 909 - DNA, 30 bp, Synthetic construct
  source 1..30: mol_type = other DNA
aatgggggagcgagtgatgtacgtgggtct

SEQ ID NO: 910 - DNA, 30 bp, Synthetic construct
  source 1..30: mol_type = other DNA
ctcttttggacagatctattccagagggga

SEQ ID NO: 911 - DNA, 30 bp, Synthetic construct
  source 1..30: mol_type = other DNA
tgttccacttgttaacagagaccggggaag

SEQ ID NO: 912 - DNA, 30 bp, Synthetic construct
  source 1..30: mol_type = other DNA
gaagcttgtggcgctgaaaacgaacggtaa

SEQ ID NO: 913 - DNA, 30 bp, Synthetic construct
  source 1..30: mol_type = other DNA
cctttatgccatgagtgctcagagaggcgg

SEQ ID NO: 914 - DNA, 30 bp, Synthetic construct
  source 1..30: mol_type = other DNA
gatttgagagactccagttgccagcggaat

SEQ ID NO: 915 - DNA, 30 bp, Synthetic construct
  source 1..30: mol_type = other DNA
aggacaagtaccgacgcgccagcgaggagc

SEQ ID NO: 916 - DNA, 30 bp, Synthetic construct
  source 1..30: mol_type = other DNA
gtgacatcatccctcacggacccgcggctc

SEQ ID NO: 917 - DNA, 30 bp, Synthetic construct
  source 1..30: mol_type = other DNA
gaaggatgactgcactcaccaacagggctg

SEQ ID NO: 918 - DNA, 30 bp, Synthetic construct
  source 1..30: mol_type = other DNA
gggtggtgagcgaatgagtagactgggaga

SEQ ID NO: 919 - DNA, 30 bp, Synthetic construct
  source 1..30: mol_type = other DNA
tacagagaacctcggaacatacggaggtag

SEQ ID NO: 920 - DNA, 30 bp, Synthetic construct
  source 1..30: mol_type = other DNA
gacagcagatcgagtcctaccccacggaca

SEQ ID NO: 921 - DNA, 30 bp, Synthetic construct
  source 1..30: mol_type = other DNA
gttctcttcttgtctcggcccatgcggttc

SEQ ID NO: 922 - DNA, 30 bp, Synthetic construct
  source 1..30: mol_type = other DNA
tccatgagaacgcatctcagcccgaggtgc

SEQ ID NO: 923 - DNA, 30 bp, Synthetic construct
  source 1..30: mol_type = other DNA
ccctactcgactacggcgtcaccgcggacg

SEQ ID NO: 924 - DNA, 30 bp, Synthetic construct
  source 1..30: mol_type = other DNA
atatcccactccatagaatctccgggggca

SEQ ID NO: 925 - DNA, 30 bp, Synthetic construct
  source 1..30: mol_type = other DNA
gtttctgcaactgaaacgcaagcgaggagg

SEQ ID NO: 926 - DNA, 30 bp, Synthetic construct
  source 1..30: mol_type = other DNA
acgagggaccagccaagatcgaggtggacc

SEQ ID NO: 927 - DNA, 30 bp, Synthetic construct
  source 1..30: mol_type = other DNA
tacaatgatgtttgatgaccgtcgcggacg

SEQ ID NO: 928 - DNA, 30 bp, Synthetic construct
  source 1..30: mol_type = other DNA
taaactgttgggacataccgctcggggcca

SEQ ID NO: 929 - DNA, 30 bp, Synthetic construct
  source 1..30: mol_type = other DNA
ttatgatgatatgagccctcgtcgaggacc

SEQ ID NO: 930 - DNA, 30 bp, Synthetic construct
  source 1..30: mol_type = other DNA
gtgctaaaatcaaagaacttcgagaggtaa

SEQ ID NO: 931 - DNA, 30 bp, Synthetic construct
  source 1..30: mol_type = other DNA
atacaatgcacttccacaacacaatggatc

SEQ ID NO: 932 - DNA, 30 bp, Synthetic construct
  source 1..30: mol_type = other DNA
agataatgtgctgcacataaccctgggaca

SEQ ID NO: 933 - DNA, 30 bp, Synthetic construct
  source 1..30: mol_type = other DNA
ggatccacgtgcgagtgcacacgaaggaga

SEQ ID NO: 934 - DNA, 30 bp, Synthetic construct
  source 1..30: mol_type = other DNA
ggaagcacattcgaactcatacaggggaaa

SEQ ID NO: 935 - DNA, 30 bp, Synthetic construct
  source 1..30: mol_type = other DNA
gcagctgcagaggccaaagcacggcggatc

SEQ ID NO: 936 - DNA, 30 bp, Synthetic construct
  source 1..30: mol_type = other DNA
ctcagatggagtatgagcgccaagtggctt

SEQ ID NO: 937 - DNA, 30 bp, Synthetic construct
  source 1..30: mol_type = other DNA
cacagcaacacatcaatgttgggagggatg

SEQ ID NO: 938 - DNA, 30 bp, Synthetic construct
  source 1..30: mol_type = other DNA
ttactgtaattgcccctatagtagtggagc

SEQ ID NO: 939 - DNA, 30 bp, Synthetic construct
  source 1..30: mol_type = other DNA
gtcaacgtcccagcaggcaacacagggaac

SEQ ID NO: 940 - DNA, 30 bp, Synthetic construct
  source 1..30: mol_type = other DNA
tagtccaatgatgccttatggcactggact

SEQ ID NO: 941 - DNA, 30 bp, Synthetic construct
  source 1..30: mol_type = other DNA
tgaggataagagagccacgaaccacggcac

SEQ ID NO: 942 - DNA, 30 bp, Synthetic construct
  source 1..30: mol_type = other DNA
cggcgtttcgggcacgaagtgcaatggtct

SEQ ID NO: 943 - DNA, 30 bp, Synthetic construct
  source 1..30: mol_type = other DNA
ttatagacgctatccacgtcgtaggggtcc

SEQ ID NO: 944 - DNA, 30 bp, Synthetic construct
  source 1..30: mol_type = other DNA
ggcagcaaatgttacaggtcctggtggtgt

SEQ ID NO: 945 - DNA, 30 bp, Synthetic construct
  source 1..30: mol_type = other DNA
tgccggcggggacaagaaggtcatcggtga

SEQ ID NO: 946 - DNA, 30 bp, Synthetic construct
  source 1..30: mol_type = other DNA
ttctgtcttgcaggaatgacaccaaggaag

SEQ ID NO: 947 - DNA, 30 bp, Synthetic construct
  source 1..30: mol_type = other DNA
atctcagacccgtacaaagtgtacaggatt

SEQ ID NO: 948 - DNA, 30 bp, Synthetic construct
  source 1..30: mol_type = other DNA
atgtcccatgacgtttggaccccgcggcca

SEQ ID NO: 949 - DNA, 30 bp, Synthetic construct
  source 1..30: mol_type = other DNA
tgtactaccgggaaatcctcgtgaaggagc

SEQ ID NO: 950 - DNA, 30 bp, Synthetic construct
  source 1..30: mol_type = other DNA
gtggctgatcgaccagatcgacagcggcaa

SEQ ID NO: 951 - DNA, 30 bp, Synthetic construct
  source 1..30: mol_type = other DNA
ttctatgctaagtacctgtgaaagggggca

SEQ ID NO: 952 - DNA, 30 bp, Synthetic construct
  source 1..30: mol_type = other DNA
acgccattgaatatgattgcaaggaggagc

SEQ ID NO: 953 - DNA, 30 bp, Synthetic construct
  source 1..30: mol_type = other DNA
ctgataggtgttgatacgagcccagggtgc

SEQ ID NO: 954 - DNA, 30 bp, Synthetic construct
  source 1..30: mol_type = other DNA
ggcttattcatagatctactgacaggggga

SEQ ID NO: 955 - DNA, 30 bp, Synthetic construct
  source 1..30: mol_type = other DNA
gggcagctgtactagagttacggtgggagc

SEQ ID NO: 956 - DNA, 30 bp, Synthetic construct
  source 1..30: mol_type = other DNA
agctggctaacaatggtaccgatggggtac

SEQ ID NO: 957 - DNA, 30 bp, Synthetic construct
  source 1..30: mol_type = other DNA
aaattggagttggcaccgttacagtggtga

SEQ ID NO: 958 - DNA, 30 bp, Synthetic construct
  source 1..30: mol_type = other DNA
gacttgtggagactgaataactgatggctg

SEQ ID NO: 959 - DNA, 30 bp, Synthetic construct
  source 1..30: mol_type = other DNA
agaaacagaacgcggctctacgcaaggaga

SEQ ID NO: 960 - DNA, 30 bp, Synthetic construct
  source 1..30: mol_type = other DNA
aaggaggactctacctgtttgccaggggga

SEQ ID NO: 961 - DNA, 30 bp, Synthetic construct
  source 1..30: mol_type = other DNA
gatgatgtgagaagagttcagaggagggag

SEQ ID NO: 962 - DNA, 30 bp, Synthetic construct
  source 1..30: mol_type = other DNA
gcaaggagatcaagcagctcacagaggaac

SEQ ID NO: 963 - DNA, 30 bp, Synthetic construct
  source 1..30: mol_type = other DNA
ccatgaactccctgccagatatcgaggagg

SEQ ID NO: 964 - DNA, 30 bp, Synthetic construct
  source 1..30: mol_type = other DNA
gccctctaggccgatacaaagcaggggaaa

SEQ ID NO: 965 - DNA, 30 bp, Synthetic construct
  source 1..30: mol_type = other DNA
gctttctccctcgacagtcatgtggggatt

SEQ ID NO: 966 - DNA, 30 bp, Synthetic construct
  source 1..30: mol_type = other DNA
cagattaattccaaccaaatcccggggctc

SEQ ID NO: 967 - DNA, 30 bp, Synthetic construct
  source 1..30: mol_type = other DNA
ttctaagtcgacatactctcggctaggtgt

SEQ ID NO: 968 - DNA, 30 bp, Synthetic construct
  source 1..30: mol_type = other DNA
cccgcctgcctcacctcacactcgcggctc

SEQ ID NO: 969 - DNA, 30 bp, Synthetic construct
  source 1..30: mol_type = other DNA
gccagccgacttacgatttccgagcggccg

SEQ ID NO: 970 - DNA, 30 bp, Synthetic construct
  source 1..30: mol_type = other DNA
gaatggagtctgtgttatctggaaaggctg

SEQ ID NO: 971 - DNA, 30 bp, Synthetic construct
  source 1..30: mol_type = other DNA
agaaacttcggcaagactttgaggaggtca

SEQ ID NO: 972 - DNA, 30 bp, Synthetic construct
  source 1..30: mol_type = other DNA
aaaagcatcgcaccatgtctcaggaggtac

SEQ ID NO: 973 - DNA, 30 bp, Synthetic construct
  source 1..30: mol_type = other DNA
tgcaggagggcactaccactacgcaggcgt

SEQ ID NO: 974 - DNA, 30 bp, Synthetic construct
  source 1..30: mol_type = other DNA
agtatcggttgacaatcaatagtgaggcat

SEQ ID NO: 975 - DNA, 30 bp, Synthetic construct
  source 1..30: mol_type = other DNA
gagaccagtcgacacccatcattgaggttg

SEQ ID NO: 976 - DNA, 30 bp, Synthetic construct
  source 1..30: mol_type = other DNA
tcttccccagtaaacgctccacagaggcag

SEQ ID NO: 977 - DNA, 30 bp, Synthetic construct
  source 1..30: mol_type = other DNA
tagagaacgtcaccttagaatccgtggcca

SEQ ID NO: 978 - DNA, 30 bp, Synthetic construct
  source 1..30: mol_type = other DNA
tccatctttgtctgccacaaacgtgggagt

SEQ ID NO: 979 - DNA, 30 bp, Synthetic construct
  source 1..30: mol_type = other DNA
gtggaacaggcattccggaaacggaggcag

SEQ ID NO: 980 - DNA, 30 bp, Synthetic construct
  source 1..30: mol_type = other DNA
tccgaggtcacgttctgcacaaagcggtaa

SEQ ID NO: 981 - DNA, 30 bp, Synthetic construct
  source 1..30: mol_type = other DNA
gaagctaacttggacccaaactcagggtcg

SEQ ID NO: 982 - DNA, 30 bp, Synthetic construct
  source 1..30: mol_type = other DNA
ccaagcattacccctactaccggacggccg

SEQ ID NO: 983 - DNA, 30 bp, Synthetic construct
  source 1..30: mol_type = other DNA
cctcagagtgcatcgacccctcggtggtct

SEQ ID NO: 984 - DNA, 30 bp, Synthetic construct
  source 1..30: mol_type = other DNA
tgccctgcggggaggactccgtcgaggaga

SEQ ID NO: 985 - DNA, 30 bp, Synthetic construct
  source 1..30: mol_type = other DNA
ctcccttcggggagacaacgacggcggtgg

SEQ ID NO: 986 - DNA, 30 bp, Synthetic construct
  source 1..30: mol_type = other DNA
tacggctgcaccgagtcgtagtcgaggtca

SEQ ID NO: 987 - DNA, 30 bp, Synthetic construct
  source 1..30: mol_type = other DNA
gccgaaaccagggccaccgaaaggcggcgg

SEQ ID NO: 988 - DNA, 30 bp, Synthetic construct
  source 1..30: mol_type = other DNA
ggcgccctcgcgcttgaggccgcgcggtcc

SEQ ID NO: 989 - DNA, 30 bp, Synthetic construct
  source 1..30: mol_type = other DNA
cagccttcggtctcttcgacgacgcggccg

SEQ ID NO: 990 - DNA, 30 bp, Synthetic construct
  source 1..30: mol_type = other DNA
gggttcggggtaatagaacgcaggcggcgg

SEQ ID NO: 991 - DNA, 30 bp, Synthetic construct
  source 1..30: mol_type = other DNA
agagaccggcagcgtcaaacaccctggtgg

SEQ ID NO: 992 - DNA, 30 bp, Synthetic construct
  source 1..30: mol_type = other DNA
gcttatgaccagaaaaacataagacggcgc

SEQ ID NO: 993 - DNA, 30 bp, Synthetic construct
  source 1..30: mol_type = other DNA
cagaccttcatggagaaatgccgtaggccc

SEQ ID NO: 994 - DNA, 30 bp, Synthetic construct
  source 1..30: mol_type = other DNA
acctggtgcagagaaaccggcatgcggagc

SEQ ID NO: 995 - DNA, 30 bp, Synthetic construct
  source 1..30: mol_type = other DNA
ctgtaaaggagcagagcaaaaccgtggtca

SEQ ID NO: 996 - DNA, 30 bp, Synthetic construct
  source 1..30: mol_type = other DNA
cttcactgctatcctgaatagacacggtat

SEQ ID NO: 997 - DNA, 30 bp, Synthetic construct
  source 1..30: mol_type = other DNA
ctcagaggacttgggaaattcaaggggcgg

SEQ ID NO: 998 - DNA, 30 bp, Synthetic construct
  source 1..30: mol_type = other DNA
aaaggccttcgatcaagtgcactgcggcca

SEQ ID NO: 999 - DNA, 30 bp, Synthetic construct
  source 1..30: mol_type = other DNA
cgcgccgacctcgagtccccgcagaggaag

SEQ ID NO: 1000 - DNA, 30 bp, Synthetic construct
  source 1..30: mol_type = other DNA
tcttcgtggtggcgcggatcctagcggacc

SEQ ID NO: 1001 - DNA, 30 bp, Synthetic construct
  source 1..30: mol_type = other DNA
gcctcgtgtccatgtcgagccgcgcggtcg

SEQ ID NO: 1002 - DNA, 30 bp, Synthetic construct
  source 1..30: mol_type = other DNA
gtgtgagttctcaggtgcgccttgaggtgc

SEQ ID NO: 1003 - DNA, 30 bp, Synthetic construct
  source 1..30: mol_type = other DNA
atttaccaggcacacaagagactggggaac

SEQ ID NO: 1004 - DNA, 30 bp, Synthetic construct
  source 1..30: mol_type = other DNA
gtacagaaatacggtccgaaacgttggtct

SEQ ID NO: 1005 - DNA, 30 bp, Synthetic construct
  source 1..30: mol_type = other DNA
cccaagtctggaaagcgtcacttggggaaa

SEQ ID NO: 1006 - DNA, 30 bp, Synthetic construct
  source 1..30: mol_type = other DNA
actatatttacatgtaacgctacagggtat

SEQ ID NO: 1007 - DNA, 30 bp, Synthetic construct
  source 1..30: mol_type = other DNA
agaccaaaatctcaccctgaaacggggaag

SEQ ID NO: 1008 - DNA, 30 bp, Synthetic construct
  source 1..30: mol_type = other DNA
actcccacggcgaagcgccctccgtggacg

SEQ ID NO: 1009 - DNA, 30 bp, Synthetic construct
  source 1..30: mol_type = other DNA
atgcggacacataatggcaaatcgggggcc

SEQ ID NO: 1010 - DNA, 30 bp, Synthetic construct
  source 1..30: mol_type = other DNA
gttatgggtggtactgccatccggaggagg

SEQ ID NO: 1011 - DNA, 100 bp, Synthetic construct
  source 1..100: mol_type = other DNA
ttagctctgtttacgtcccagcgggcatgagagtaacaagagggtgtggtaatattacggtaccgagcactatcgatacaatatgtgtcatacggacacg

(19) United States
(12) Patent Application Publication
(10) Pub. No.: US 2026/0347884 A1
(43) Pub. Date: Nov. 5, 2026

(54) GENE TARGETS FOR MANIPULATING T CELL BEHAVIOR

(71) Applicants: The Regents of the University of California, Oakland, CA (US); The Board of Trustees of the Leland Stanford Junior University, Stanford, CA (US); The J. David Gladstone Institutes, A Testamentary Trust Established Under the Will of J. David Glads, San Francisco, CA (US)

(72) Inventors: Alexander Marson, Oakland, CA (US); Maya Arce, San Francisco, CA (US); Jacob Freimer, San Francisco, CA (US); Jennifer Umhoefer, San Francisco, CA (US); Jonathan Pritchard, San Francisco, CA (US)

(21) Appl. No.: 19/111,493

(22) PCT Filed: Sep. 13, 2023
(86) PCT No.: PCT/US2023/074081

§ 371 Date: Mar. 13, 2025

Related U.S. Application Data
(60) Provisional application No. 63/406,192, filed on Sep. 13, 2022.

(51) Int. Cl. C12N 15/90; A61K 35/17; C07K 14/715; C12N 5/0783; C12N 9/22; C12N 15/11

(52) U.S. Cl. CPC C12N 15/907; A61K 35/17; C07K 14/7155; C12N 5/0637; C12N 9/226; C12N 15/11; C12N 2310/20; C12N 2510/00

(57) ABSTRACT

Provided herein are compositions and methods for modifying T cells.

### PRIOR RELATED APPLICATION

This application claims the benefit of and priority to U.S. Provisional Application No. 63/406,192, filed on Sep. 13, 2022, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. R01 HG008140 awarded by the National Institutes of Health. The government has certain rights in the invention.

### BACKGROUND OF THE INVENTION

Decades of work in animal models and cell lines have identified regulators of T cell suppression and activation, but systematic strategies to comprehensively analyze the function of genes that regulate human T cell responses are still lacking. T cells play a role in regulating the immune response in cancer as well as other diseases, for example, autoimmune diseases. Methods of modifying T cells for the treatment of autoimmune diseases or cancer have great therapeutic potential.

The disclosure is based, in part, on the use of sgRNA lentiviral infection with Cas9 protein electroporation (SLICE), to identify regulators of IL2RA in regulatory T cells and effector T cells. Screens were performed in resting effector T cells and regulatory T cells, as well as stimulated CD4+T effectors, which revealed both cell subset and stimulation dependent regulators of IL2RA. Regulators of IL2RA, that positively or negatively control its expression across both cell types and stimulation conditions, as well as the cell type and stimulation dependent regulators of IL2RA were identified. IL2RA is a key gene in immune regulation that has been implicated in autoimmune disease and cancer. Therefore, modulating expression of IL2RA in T cells, for example, effector T cells or regulatory T cells, could have therapeutic applications.

The present invention is directed to compositions and methods for modifying T cells. The inventors have identified nuclear factors that influence expression of IL2RA. T cells can be modified by inhibiting and/or overexpressing one or more of these nuclear factors to manipulate immune cell activity. In some examples, modified T cells are used to treat autoimmune disorders, assist in organ transplantation, to treat graft versus host disease, or inflammation. Examples of autoimmune/inflammatory diseases include but are not limited to: type 1 diabetes, rheumatoid arthritis, inflammatory bowel disease, multiple sclerosis, and multi-organ autoimmune syndromes. In other examples, modified T cells are used to treat cancer. For example, in some embodiments, T cells can be used to target hematological malignancies or solid tumors. Examples of such cancers include but are not limited to, ovarian cancer breast cancers, colon cancers, lung cancers, prostate cancers, liver cancers, bone and soft tissue cancers, head and neck cancers, melanomas and other skin cancers, brain cancers, leukemias, lymphomas.

Provided herein is a T cell comprising: (a) a genetic modification or heterologous polynucleotide that inhibits expression of a nuclear factor set forth in Table 1 and/or (b) a heterologous polynucleotide that encodes a nuclear factor set forth in Table 1.

In some embodiments, the T cell comprises (a) a genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574; and/or (b) a heterologous polynucleotide that encodes FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574

In some embodiments, the T cell comprises (a) a genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is increased in the T cell relative to expression of IL2RA in a T cell not comprising the genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574; and/or (b) a heterologous polynucleotide that encodes FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32, wherein expression of IL2RA is increased in the T cell relative to expression of IL2RA in a T cell not comprising the heterologous polynucleotide that encodes FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32.

In some embodiments, the T cell comprises (a) a genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32, wherein expression of IL2RA is decreased in the T cell relative to expression of IL2RA in a T cell not comprising the genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32; and/or (b) a heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the T cell relative to expression of IL2RA in a T cell not comprising heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574.

In some embodiments, the T cell is a human T cell. In some embodiments, the T cell is a regulatory T cell (Treg), an effector T cell (e.g., a CD4+ T cell) or a stimulated T cell (e.g., a stimulated CD4+ T cell). In some embodiments, the T cell is a resting T cell. Populations comprising any of the genetically modified T cells described herein are also provided.

In some embodiments, the regulatory T cell comprises: (a) a genetic modification or heterologous polynucleotide that inhibits expression ZKSCAN2, ZNF114, E4F1, GLIS2, JUNB, NR2F6, ZZZ3, ETV3, ERF, RUNX1, TGIF2, or ZNF574, wherein expression of IL2RA is increased in the Treg cell relative to expression of IL2RA in a Treg cell not comprising the genetic modification or heterologous polynucleotide that inhibits expression of; and/or (b) a heterologous polynucleotide that encodes FOXN2, ZNF25, THRA, ZBTB2, CREM, PTF1A, IKZF2, MYNN, BACH2, IL15RA, SP3, or ZBTB3, wherein expression of IL2RA is increased in the Treg cell relative to expression of IL2RA in a Treg cell not comprising the heterologous polynucleotide that encodes FOXN2, ZNF25, THRA, ZBTB2, CREM, PTF1A, IKZF2, MYNN, BACH2, IL15RA, SP3, or ZBTB3.

In some embodiments, the Treg cell comprises (a) a genetic modification or heterologous polynucleotide that inhibits expression FOXN2, ZNF25, THRA, ZBTB2, CREM, PTF1A, IKZF2, MYNN, BACH2, IL15RA, SP3, or ZBTB3, wherein expression of IL2RA is decreased in the Treg cell relative to expression of IL2RA in a Treg cell not comprising the genetic modification or heterologous polynucleotide that inhibits expression of FOXN2, ZNF25, THRA, ZBTB2, CREM, PTF1A, IKZF2, MYNN, BACH2, IL15RA, S3, or ZBTB3; and/or (b) a heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the Treg cell relative to expression of IL2RA in a Treg cell not comprising the heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574.

In some embodiments, the CD4+ T cell comprises: (a) a genetic modification or heterologous polynucleotide that inhibits expression of BACH2, ZBTB3, ABTB14, GFI1, or IL2RB, wherein expression of IL2RA is decreased in the CD4+ T cell relative to expression of IL2RA in a CD4+ T cell not comprising the genetic modification or heterologous polynucleotide that inhibits expression of BACH2, REST, ZBTB3, ABTB14, GFI1, or IL2RB.

In some embodiments, the stimulated CD4+ T cell comprises: (a) a genetic modification or heterologous polynucleotide that inhibits expression of REST, ELP2, IL2RB, NR4A3, or ZBTB32, wherein expression of IL2RA is decreased in the stimulated CD4+ T cell relative to expression of IL2RA in a stimulated CD4+ T cell not comprising the genetic modification or heterologous polynucleotide that inhibits expression of REST, ELP2, IL2RB, NR4A3, or ZBTB32; and/or (b) a heterologous polynucleotide that encodes BACH2, STAT6, ZMYND8, IFNGR2, IL4R, LEF1, or ARID5A, wherein expression of IL2RA is decreased in the stimulated CD4+ T cell relative to expression of IL2RA in a stimulated CD4+ T cell not comprising the heterologous polynucleotide that encodes BACH2, STAT6, ZMYND8, IFNGR2, IL4R, LEF1, or ARID5A,

In some embodiments, the stimulated CD4+ T cell comprises: the stimulated CD4+ T cell comprises: (a) a genetic modification or heterologous polynucleotide that inhibits expression of BACH2, STAT6, ZMYND8, IFNGR2, IL4R, LEF1, or ARID5A, wherein expression of IL2RA is increased in the stimulated CD4+ T cell relative to expression of IL2RA in a stimulated CD4+ T cell not comprising the genetic modification or heterologous polynucleotide that inhibits expression of BACH2, STAT6, ZMYND8, IFNGR2, IL4R, LEF1, or ARID5A; and/or (b) a heterologous polynucleotide that encodes REST, ELP2, IL2RB, NR4A3, or ZBTB32, wherein expression of IL2RA is increased in the stimulated CD4+ T cell relative to expression of IL2RA in a stimulated CD4+ T cell not comprising the heterologous polynucleotide that encodes REST, ELP2, IL2RB, NR4A3, or ZBTB32.

Also provided is a method of making a modified T cell, the method comprising: (a) inhibiting expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2 and ZNF574; and/or (b) overexpressing one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574.

In some embodiments, the inhibiting comprises reducing expression of the nuclear factor, or reducing expression of a polynucleotide encoding the nuclear factor. In some embodiments, the inhibiting comprises contacting a polynucleotide encoding the nuclear factor with a targeted nuclease, a guide RNA (gRNA), an siRNA, an antisense RNA, microRNA (miRNA), or short hairpin RNA (shRNA). In some embodiments, the inhibiting comprises mutating the polynucleotide encoding the nuclear factor.

In some embodiments, the inhibiting comprises contacting the polynucleotide with a targeted nuclease. In some embodiments, the targeted nuclease introduces a double-stranded break in a target region in the polynucleotide. In some embodiments, the targeted nuclease is an RNA-guided nuclease. In some embodiments, the RNA-guided nuclease is a Cpf1 nuclease or a Cas9 nuclease and the method further comprises introducing into a T cell a gRNA that specifically hybridizes to a target region in the polynucleotide. In some embodiments, the Cpf1 nuclease or the Cas9 nuclease and the gRNA are introduced into the T cell as a ribonucleoprotein (RNP) complex. In some embodiments, the inhibiting comprises performing clustered regularly interspaced short palindromic repeats (CRISPR)/Cas genome editing.

Any of the methods of making a T cell or population of T cells described herein can further comprise administering the T cell to a human following the inhibiting. In some embodiments, the T cell is obtained from a human prior to treating the T cell to inhibit expression of the nuclear factor, and the treated T cell is reintroduced into a human. In some methods, the T cells is a human cell. In some methods, the T cell is a Treg cell, an effector T cell (e.g. a CD4+ T cell or CD8+ T cell) or a stimulated T cell (e.g. a stimulated CD4+ T cell). Also provided are T cells made by any of the methods provided herein.

In some embodiments, expression of one or more nuclear factors selected from the group consisting of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574 is inhibited in the T cell.

In some embodiments, expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32 is inhibited in the T cell.

In some embodiments, the T cell is a regulatory T cell and expression of one or more nuclear factors selected from the group consisting of ZKSCAN2, ARID5A, ZNF114, E4F1, GLIS2, JUNB, NR2F6, ZZZ3, ETV3, ERF, RUNX1, TGIF2, or ZNF574 is inhibited in the Treg cell.

In some embodiments, the T cell is a regulatory T cell and expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32 is inhibited to decrease IL2RA expression in the regulatory T cell, and the subject has cancer.

In some embodiments, the T cell is a CD4+ T cell and expression of one or more nuclear factors selected from the group consisting of BACH2, ZBTB3, ABTB14, GFI1, IL2RB, REST, ELP2, IL2RB, NR4A3, or ZBTB32 is inhibited to decrease IL2RA expression in the CD4+ T cell, and wherein the subject has an autoimmune disorder.

In some embodiments, the T cell is a CD4+ T cell and expression of one or more nuclear factors selected from the group consisting of BACH2, STAT6, ZMYND8, IFNGR2, IL4R, LEF1, or ARID5A is inhibited to increase IL2RA expression in the CD4+ T cell, and wherein the subject has cancer.

Also provided is a method of modifying T cells in a subject in need thereof, comprising inhibiting expression of a one or more nuclear factors or overexpressing one or more factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IAR, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574 in the human T cells of the subject.

In some embodiments, inhibiting expression of one or more nuclear factors or overexpression of one or more nuclear factors occurs in vivo. In some embodiments, the method comprises: a) obtaining T cells from the subject; b) modifying the T cells by inhibiting expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574; and c) administering the T cells to the subject.

In some embodiments, the method comprises: a) obtaining T cells from the subject; b) modifying the T cells by overexpressing one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574; and c) administering the T cells to the subject. In some embodiments, the subject has cancer or an autoimmune disorder.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the results of a CD25 (IL2RA) screen performed on resting regulatory T cells (Treg). Positive regulators are depicted in green (left portion of graph (low bin) from 0 to −3 log 2fold change in sgRNA abundance) and negative regulators in pink (right portion of graph (high bin) from 0 to 3 log 2fold change in sgRNA abundance).

FIG. 2 shows the results of a CD25 screen performed on resting effector T cells (Teff). Positive regulators are depicted in green (left portion of graph (low bin) from 0 to −3 log 2fold change in sgRNA abundance) and negative regulators in pink (right portion of graph (high bin) from 0 to 3 log 2fold change in sgRNA abundance).

FIG. 3 shows the results of a CD25 screen performed on stimulated effector T cells at peak CD25 expression. Positive regulators are depicted in green (left portion of graph (low bin) from 0 to −3 log 2fold change in sgRNA abundance) and negative regulators in pink (right portion of graph (high bin) from 0 to 3 log 2fold change in sgRNA abundance).

FIG. 4 shows a Treg and resting Teff IL2RA regulator comparison. Shared hits that were both significant (FDR<0.05) in the same direction are depicted in green. Hits that were only significant in one of the two screens are shown in blue (light blue for Tregs, dark blue for Teffs). Hits that were significant in both screens but with the opposite direction of effect are in orange.

FIG. 5 shows a stimulated and resting Teff IL2RA regulator comparison. Shared hits that were both significant (FDR<0.05) in the same direction are depicted in green. Hits that were only significant in one of the two screens are shown in blue (light blue for resting, dark blue for stimulated). Hits that were significant in both screens but with the opposite direction of effect are in orange.

FIG. 6 shows a Stimulated Teff and Treg IL2RA regulator comparison. Shared hits that were both significant (FDR<0.05) in the same direction are depicted in green. Hits that were only significant in one of the two screens are shown in blue (light blue for resting Treg, dark blue for stimulated Teff). Hits that were significant in both screens but with the opposite direction of effect are in orange.

FIG. 7 shows an arrayed validation of select hits from the screen described in the Examples, in both resting Tregs and Teffs. CD25 screen hits are represented as Log 2FC guide abundance in the high bin/low bin of CD25 expression. Arrayed validation of hits are represented as the log 2FC CD25 MFI of gene KO compared to AAVS1 control KOs.

FIG. 8 shows the effect of several regulators on CD25 expression compared to AAVS1 control KO guides, over the course of stimulation (from 0-144 hours) in effector T cells and regulatory T cells.

FIG. 9 shows the effect of several regulators on cell count, compared to AAVS1 control KO guides, over the course of stimulation (from 0-144 hours) in effector T cells and regulatory T cells.

FIG. 10 shows the effect of several regulators on Granzyme B expression, compared to AAVS1 control KO guides, over the course of stimulation (from 0-144 hours) in effector T cells and regulatory T cells.

FIGS. 11A-G show the results of pooled CRISPR KO trans regulator screens. (A) Schematic of trans regulator screens using T effectors and Tregs from human donors. (B) Primary human Tregs (CD4+CD25hiCD127low) and Teffs (CD4+CD25low) were isolated, edited and expanded to screen for regulators of IL2RA in resting Teffs (IL2RA low) and restimulated Teffs near their highest expression levels, 72 hours following stimulation (IL2RA high) as well as in resting Tregs (IL2RA high). (C) CD25 expression levels of cell conditions included in pooled screens. Representative histogram of CD25 expression levels in resting Teffs (light blue), stimulated Teffs (dark blue) and resting Tregs (lilac). (D) A stimulated and resting Teff IL2RA regulator comparison (E) Resting Treg vs resting Teff comparison (left) and resting Treg vs. stimulated Teff IL2RA comparison (right). (F) Overview of context specific trans-regulatory CRISPR screens. (G) Effect of several regulators on CD25 expression compared to AAVS1 control KO guides, over the course of stimulation (from 0-144 hours) in effector T cells and regulatory T cells.

FIG. 12A shows differentially acetylated H3K27ac regions following ablation of MED12. All points represent significantly differentially acetylated regions in the MED12 KO conditions compared to AAVS1 KO (padj<0.05). Gold points indicate regions where the nearest gene is a regulator of IL2RA identified in any of the screens. (n=2 donors per KO).

FIG. 12B shows that MED12 KO effects super enhancer maintenance across cell subsets and stimulation conditions. Super enhancer ranks generated with H3K27ac CUT&RUN from a representative donor in Teffs and Tregs with AAVS1 KO (grey, left panel of each enhancer rank pair) or MED12 KO (red or pink (right panel of each enhancer rank pair).

FIGS. 13A-D show regulators of IL2RA (A) Regulators of IL2RA are enriched in the MED12 downstream network. Fisher's exact test was used for regulators of IL2RA in the differentially expressed genes downstream of MED12 (RNAseq: n=3, padj<0.05). Tests were performed using screen results from the matched cell type and stimulation conditions and genes were subset to those targeted for knock-out in the trans regulator library used in the screens (1350 gene total). (B) Regulators of IL2RA in the downstream network of MED12. Differentially expressed genes are displayed as the log 2FoldChange gene expression in the MED12 KO samples/AAVS1 KO samples for the respective condition (padj<0.05, n=3 donors per KO). The horizontal annotation bars on the top of the figure represent the stimulation condition (dark grey for resting and light for stimulated) and the vertical annotation bars represent the effect of the regulator in the IL2RA KO screens (colored boxes=statistically significant FDR<0.05, navy=positive regulator of IL2RA and gold=negative regulator of IL2RA). (C) Directed network plots depicting select downstream trans regulators of MED12 across conditions. The log 2FoldChange gene expression, as described in B, is depicted with solid lines and the log 2FoldChange guide abundance (CD25 high bin/low bin) as described in 1C-E is depicted with dashed lines. (D) Comparison of the downstream effects on transcription of MED12 and core Mediator subunits. Log 2FoldChange gene expression of significantly differentially expressed genes (padj<0.05, n=3 donors per KO) is depicted between MED12 and MED11 as well as MED12 and MED14. Both MED11 and ME 14 belong to the core Mediator complex while MED12 is part of the kinase domain.

FIGS. 14A-E show the effects of MED12 KO. (A) Stimulation responsive genes are dysregulated in MED12 KO samples. Genes differentially expressed (padj<0.05) downstream in the MED12 KO samples/AAVS1 KO samples are grouped according to stimulation response category. The Bonferroni adjusted p value resulting from a two-tailed T test is displayed comparing each of the stimulation responsive groups to the non-stimulation responsive group (n=3 donors per KO). (B) Activation scores for perturbed regulators across resting and stimulated states. Each point represents the median activation score of cells targeted for knock-down of the respective gene. Dashed grey lines indicate the activation score for non-targeting control cells within the respective conditions. The color of each point indicates in which stimulation condition the activation score for the knock-down is significantly different than non-targeting controls as determined by a Wilcoxon rank sum test with continuity correction (padj<0.01). (C) Clustering of resting and stimulated perturb-seq populations. UMAP representing clusters defined within resting Teff cells and stimulated Teff cells (far left plots). UMAP density plots represent the localization of cells with the respective gene perturbation within the two conditions. Non-targeting guide cell distribution is shown in the background as grey. (D) Regulatory connections among activation suppressing trans regulators in resting Teffs. Differentially expressed genes identified by pseudo-bulking are represented as edges (padj<0.05, n=2 donors per target gene). Light grey nodes are activation suppressors in resting Teffs as identified in B. (E) Regulatory connections among activation promoting trans regulators in stimulated Teffs. Edge data as described in D. Dark grey nodes indicate regulators without significantly different activation scores in stimulated Teffs and white nodes indicate activation promoters.

FIGS. 15A-G show the effects of MED12 KO. (A) MED12 KO CAR-T gene expression activation scores. RNAseq data from Freitas et al. GEO series GSE174279. The GSVA activation score for each group of samples is compared using a two-tailed T test and the Bonferroni adjusted p value displayed for significantly different groups (n=3 donors per KO). (B) Rank plot of total cell abundance within the perturb-seq pool for each gene target. Cell abundance was normalized using the sgRNA distribution in the library plasmid and is represented as the log 2FoldChange compared to non-targeting control cells. The dashed line indicates the total abundance of non-targeting control cells within the respective condition. (C) Proliferative cell distribution within the perturb-seq pool. The ratio of G2M/G1 cells within each condition is represented on the x axis as the log 2FoldChange compared to non-targeting control cells. (D) Top downstream pathways affected by MED12 KO. Significantly enriched pathways within the differentially expressed genes in bulk RNAseq data from MED12 KO cells. (E) Proportion of apoptotic cells following stimulation of Teffs. % apoptotic cells is shown on the y axis as quantified by the percentage of lymphocytes positive for caspase-3/7 activity and negative for SYTOX nucleic acid stain. The dose of anti-CD3/CD28/CD2 is displayed above each grid as the proportion of the recommended dose. Stars indicate significantly different percentage compared to AAVS1 KO using a two-tailed T test (:p<=0.05, : p<=0.01:p<=0.001; n=4 donors×2 guides per target gene). (F) Total live cell count per well of activation induced cell death assay. Stars indicate significantly different percentage compared to AAVS1 KO using a two-tailed T test (:p<=0.05, : p<=0.01; n=4 donors×2 guides per target gene). (G) Summary of activation and resting state core regulatory networks coordinated by MED12.

### DEFINITIONS

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” can refer to the segment of DNA involved in producing or encoding a polypeptide chain. It may include regions preceding and following the coding region (leader and trailer) as well as intervening sequences (introns) between individual coding segments (exons).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “inhibiting expression” refers to inhibiting or reducing the expression of a gene product, e.g., RNA or protein. As used throughout, the term “nuclear factor” refers to a protein that directly or indirectly alters expression of IL2RA, for example, a transcription factor. To inhibit or reduce the expression of a gene, the sequence and/or structure of the gene may be modified such that the gene would not be transcribed (for DNA) or translated (for RNA), or would not be transcribed or translated to produce a functional protein, for example, a polypeptide or protein encoded by a gene set forth in Table 1. Various methods for inhibiting or reducing expression are described in detail further herein. Some methods may introduce nucleic acid substitutions, additions, and/or deletions into the wild-type gene. Some methods may also introduce single or double strand breaks into the gene. To inhibit or reduce the expression of a protein, one may inhibit or reduce the expression of the gene or polynucleotide encoding the protein. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%/6, or at least about 300%, or at least about 40%, or at least about 50%, or at least about 60%, or at least about 70%, or at least about 80%, or at least about 90% or up to and including a 100% decrease (i.e., absent level as compared to a reference sample). It is understood that one or more nuclear factors set forth in Table 1 can be inhibited in a T cell. It is also understood that two or more nuclear factors inhibited in a T cell can be selected from one or more of Table 1, Table 2, Table 3, Table 4, Table 5, Table 6, or Table 7.

The term “overexpressing” or “overexpression” refers to increasing the expression of a gene or protein. “Overexpression” refers to an increase in expression, for example, in increase in the amount of mRNA or protein expressed in a T cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for overexpression are known to those of skill in the art, and include, but are not limited to, stably or transiently introducing a heterologous polynucleotide encoding a protein (i.e., a nuclear factor set forth in Table 1) to be overexpressed into the cell or inducing overexpression of an endogenous gene encoding the protein in the cell. It is understood that one or more nuclear factors set forth in Table 1 can be overexpressed in a T cell. It is also understood that two or more nuclear factors overexpressed in a T cell can be selected from one or more of Table 1, Table 2, Table 3, Table 4, Table 5, Table 6, or Table 7.

As used herein the phrase “heterologous” refers to what is not found in nature. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

As used throughout, the term “targeted nuclease” refers to nuclease that is targeted to a specific DNA sequence in the genome of a cell to produce a strand break at that specific DNA sequence. The strand break can be single-stranded or double-stranded. Targeted nucleases include, but are not limited to, a Cas nuclease, a TAL-effector nuclease and a zinc finger nuclease.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlrofexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. Table 2 shows exemplary gRNA sequences used in methods of the disclosure. A sequence comprising any of the gRNA sequences set forth in Table 2 can be used in the methods provided herein to target an RNA-mediated nuclease to a specific site in the genome of a cell.

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163. Issue 3, p 759-771, 22 Oct. 2015) and homologs thereof. Similarly, as used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” refers to inducing a structural change in the sequence of the genome at a target genomic region in a T cell. For example, the modifying can take the form of inserting a nucleotide sequence into the genome of the cell. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region. “Modifying” can also refer to altering the expression of a nuclear factor in a T cell, for example inhibiting expression of a nuclear factor or overexpressing a nuclear factor in a T cell.

As used herein, the phrase “T cell” refers to a lymphoid cell that expresses a T cell receptor molecule. T cells include human alpha beta (ap) T cells and human gamma delta (76) T cells. T cells include, but are not limited to, naïve T cells, effector T cells, stimulated T cells, resting T cells, primary T cells (e.g., uncultured), cultured T cells, immortalized T cells, helper T cells, cytotoxic T cells, memory T cells, regulatory T cells, natural killer T cells, combinations thereof, or sub-populations thereof. As used throughout, a “resting T cell” is a quiescent, non-proliferating T cell. In some embodiments, T cells can be stimulated to increase proliferation, increase the the production of effector molecules, and/or polarize the cells towards different subtypes. In some embodiments, stimulation conditions include engagement of the TCR with or without signal 2 activation through co-stimulatory receptors. T cells can also be stimulated by anti-CD3/CD28 or antigen presentation. Additionally, cytokines, chemokines, or antibodies including but not limited to IL2, can be added to increase proliferation or induce a specific cell state. In some embodiments, the stimulation period lasts for hours, or days (e.g., 1, 2, 3, 4, 5, 6, 7, 8, 9, 10 days or more) following activation, during which the cell remains proliferative. In some embodiments, for example when using soluble anti-CD3/CD28/CD2 tetramers the stimulation period can last for approximately 6-8 days.

T cells can be CD4⁺, CD8⁺, or CD4⁺ and CD8⁺. T cells can also be CD4⁻, CD8⁻, or CD4⁻ and CD8⁻-T cells can be helper cells, for example helper cells of type T_{H}1, T_{H}2, T_{H}3, T_{H}9, T_{H}17, or T_{FH}. T cells can be cytotoxic T cells. T cells can also be regulatory T cells. Regulatory T cells (Tregs) can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated or stimulated by contact with (e.g., culturing in the presence of) CD3, CD28 agonists, IL2, IFN-γ, or a combination thereof.

As used herein, the phrase “introducing” in the context of introducing a nucleic acid or a complex comprising a nucleic acid, for example, an RNP complex, refers to the translocation of the nucleic acid sequence or the RNP complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Compositions and Methods

As described herein, the disclosure provides compositions and methods directed to modifying T cells by inhibiting the expression of one or more nuclear factors and/or overexpressing one or more nuclear factors in a T cell. The disclosure also features compositions comprising the genetically modified T cells described herein. A population of modified T cells may provide therapeutic benefits in treating diseases with altered immune responses, for example, cancer or treating autoimmune diseases.

The inventors have discovered that by inhibiting the expression of one or more nuclear factors and/or overexpressing one or more nuclear factors, T cells may be altered to modulate T cell function.

Examples of nuclear factors whose expression may be altered to modify the stability of T cells in the methods described herein include but are not limited to the nuclear factors set forth in Table 1. In some embodiments, the present invention provides a method of modifying a T cells, the method comprising inhibiting expression of one or more nuclear factors set forth in Table 1. In some embodiments, the present invention provides a method of modifying a T cells, the method comprising overexpressing one or more nuclear factors set forth in Table 1. Several of the genes listed in Table 1 have T cell subset specific effects which results in a selective change of IL2RA expression on either regulatory T cells or effector T cells. This allows selective regulation of IL2RA expression in specific T cell types for therapeutic uses. For example, BACH2 is a positive regulator (i.e., inhibition of BACH2 decreases IL2RA expression) in regulatory T cells and a negative regulator (i.e., inhibition of BACH 2 increases IL2RA expression) in stimulated CD4+ T cells. One of skill in the art could inhibit BACH2 to decrease IL2RA expression in regulatory T cells for administration to a subject having an autoimmune disorder, or inhibit BACH2 to increase IL2RA expression in stimulated CD4+ T cells for administration to a subject having cancer. This example is merely illustrative as one of skill in the art would know, based on Table 1, which genes to inhibit or overexpress to obtain the desired regulatory effect(s) in one or more specific types of T cells.

In Table 1, “sig_dir.Treg” refers to the direction of the IL2RA expression (i.e., positive or negative), in the resting Treg screen described in the Examples. A positive regulator decreases IL2RA expression in a Treg cell when inhibited (e.g., knocked out). A negative regulator increases IL2RA expression in a Treg cell when inhibited. In some embodiments, a positive regulator is inhibited to decrease expression of IL2RA in a T cell. In some embodiments, a negative regulator is inhibited to increased expression of IL2RA in a T cell. In some embodiments, a positive regulator is overexpressed to increase expression of IL2RA in a T cell. In some embodiments, a negative regulator is overexpressed to decrease expression of IL2RA in a T cell.

In Table 1, “sig_dir.Teff” refers to the direction of the IL2RA expression (i.e., positive or negative), in the resting Teff screen described in the Examples. A positive regulator decreases IL2RA expression in an effector T cell when inhibited (e.g., knocked out). A negative regulator increases IL2RA expression in an effector T cell when inhibited. In Table 1, “sig_dir.Teff_Stim” refers to the direction of the IL2RA expression (i.e., positive or negative), in the stimulated Teff screen described in the Examples. A positive regulator decreases IL2RA expression in a stimulated effector T cell when inhibited (e.g., knocked out). A negative regulator increases IL2RA expression in a stimulated effector T cell when inhibited. As used in Table 1, “NS” refers to an effect that was not significant in a particular T cell screen. Table 2 provides the nuclear factors listed in Table 1, with additional information identifying the sgRNA target sequence, target context sequence and exon targeted for each nuclear factor.

TABLE 1
Nuclear Factors that can be inhibited or overexpressed to modulate IL-12 expression in T cells.

| | | Target | | | | | | | |
|---|---|---|---|---|---|---|---|---|---|
| | Screen | Gene | Target | Genomic | | SEQ ID | sig_dir. | sig_dir | sig_dir |
| Gene ID | Target | ID | Transcript | Sequence | Amino Acid sequence | NO: | Treg | Teff | Teff_Stim |
| FOXN2 | IL2RA | 3344 | NM_002158.3 | NC_000002.12 | MGPVIGMTPDKRAETPGAEKIAGLSQIYK | 1 | Positive | NS | NS |
| | | | | | MGSLPEAVDAARPKATLVDSESADDELT | | Regulator | | |
| | | | | | NLNWLHESTNLLTNFSLGSEGLPIVSPLYD | | | | |
| | | | | | IEGDDVPSFGPACYQNPEKKSATSKPPYSF | | | | |
| | | | | | SLLIYMAIEHSPNKCLPVKEIYSWILDHFP | | | | |
| | | | | | YFATAPTGWKNSVRHNLSLNKCFQKVER | | | | |
| | | | | | SHGKVNGKGSLWCVDPEYKPNLIQALKK | | | | |
| | | | | | QPFSSASSQNGSLSPHYLSSVIKQNQVRNL | | | | |
| | | | | | KESDIDAAAAMMLLNTSIEQGILECEKPLP | | | | |
| | | | | | LKTALQKKRSYGNAFHHPSAVRLQESDSL | | | | |
| | | | | | ATSIDPKEDHNYSASSMAAQRCASRSSVS | | | | |
| | | | | | SLSSVDEVYEFIPKNSHVGSDGSEGFHSEE | | | | |
| | | | | | DTDVDYEDDPLGDSGYASQPCAKISEKG | | | | |
| | | | | | QSGKKMRKQTCQEIDEELKEAAGSLLHL | | | | |
| | | | | | AGIRTCLGSLISTAKTQNQKQRKK | | | | |
| ZNF25 | IL2RA | 219749 | NM_145011.2 | NC_000010.11 | MNKFQGPVTLKDVIVEFTKEEWKLLTPA | 2 | Positive | NS | NS |
| | | | | | QRTLYKDVMLENYSHLVSVGYHVNKPN | | Regulator | | |
| | | | | | AVFKLKQGKEPWILEVEFPHRGFPEDLWS | | | | |
| | | | | | IHDLEARYQESQAGNSRNGELTKHQKTH | | | | |
| | | | | | TTEKACECKECGKFFCQKSALIVHQHTHS | | | | |
| | | | | | KGKSYDCDKCGKSFSKNEDLIRHQKIHTR | | | | |
| | | | | | DKTYECKECKKIFYHLSSLSRHLRTHAGE | | | | |
| | | | | | KPYECNQCEKSFYQKPHLTEHQKTHTGE | | | | |
| | | | | | KPFECTECGKFFYVKAYLMVHQKTHTGE | | | | |
| | | | | | KPYECKECGKAFSQKSHLTVHQRMHTGE | | | | |
| | | | | | KPYKCKECGKFFSRNSHLKTHQRSHTGEK | | | | |
| | | | | | PYECKECRKCFYQKSALTVHQRTHTGEK | | | | |
| | | | | | PFECNKCGKTFYYKSDLTKHQRKHTGEK | | | | |
| | | | | | PYECTECGKSFAVNSVLRLHQRTHTGEKP | | | | |
| | | | | | YACKECGKSFSQKSHFIIHQRKHTGEKPY | | | | |
| | | | | | ECQECGETFIQKSQLTAHQKTHTKKRNAE | | | | |
| | | | | | K | | | | |
| THRA | IL2RA | 7067 | NM_199334.3 | NC_000017.11 | MEQKPSKVECGSDPEENSARSPDGKRKR | 3 | Positive | NS | NS |
| | | | | | KNGQCSLKTSMSGYIPSYLDKDEQCVVC | | Regulator | | |
| | | | | | GDKATGYHYRCITCEGCKGFFRRTIQKNL | | | | |
| | | | | | HPTYSCKYDSCCVIDKITRNQCQLCRFKK | | | | |
| | | | | | CIAVGMAMDLVLDDSKRVAKRKLIEQNR | | | | |
| | | | | | ERRRKEEMIRSLQQRPEPTPEEWDLIHIAT | | | | |
| | | | | | EAHRSTNAQGSHWKQRRKFLPDDIGQSPI | | | | |
| | | | | | VSMPDGDKVDLEAFSEFTKIITPAITRVVD | | | | |
| | | | | | FAKKLPMFSELPCEDQIILLKGCCMEIMSL | | | | |
| | | | | | RAAVRYDPESDTLTLSGEMAVKREQLKN | | | | |
| | | | | | GGLGVVSDAIFELGKSLSAFNLDDTEVAL | | | | |
| | | | | | LQAVLLMSTDRSGLLCVDKIEKSQEAYLL | | | | |
| | | | | | AFEHYVNHRKHNIPHFWPKLLMKVTDLR | | | | |
| | | | | | MIGACHASRFLHMKVECPTELFPPLFLEV | | | | |
| | | | | | FEDQEV | | | | |
| ZBTB2 | IL2RA | 57621 | NM_020861.2 | NC_000006.12 | MDLANHGLILLQQLNAQREFGFLCDCTV | 4 | Positive | NS | NS |
| | | | | | AIGDVYFKAHKSVLASFSNYFKMLFVHQ | | Regulator | | |
| | | | | | TSECVRLKPTDIQPDIFSYLLHLMYTGKM | | | | |
| | | | | | APQLIDPVRLEQGIKFLHAYPLIQEASLAS | | | | |
| | | | | | QGAFSHPDQVFPLASSLYGIQIADHQLRQ | | | | |
| | | | | | ATKIASAPEKLGRDPRPQTSRISQEQVPEA | | | | |
| | | | | | SQLSQLTSNLAQVNRTNMTPSDPLQTSLS | | | | |
| | | | | | PELVSTPVPPPPPGEETNLEASSSDEQPASL | | | | |
| | | | | | TIAHVKPSIMKRNGSFPKYYACHLCGRRF | | | | |
| | | | | | TLRSSLREHLQIHTGVPFTSSQQGESRVPL | | | | |
| | | | | | TLCSNAADLGKDAMEVPEAGMISDSELQ | | | | |
| | | | | | HISDSPIIDGQQQSETPPPSDIADIDNLEQA | | | | |
| | | | | | DQEREVKRRKYECTICGRKFIQKSHWREH | | | | |
| | | | | | MYIHTGKPFKCSTCDKSFCRANQAARHV | | | | |
| | | | | | CLNQSIDTYTMVDKQTLELCTFEEGSQMD | | | | |
| | | | | | NMLVQTNKPYKCNLCDKTFSTPNEVVKH | | | | |
| | | | | | SCQNQNSDVFALDEGRSILLGSGDSEVTE | | | | |
| | | | | | PDHPVLASIKKEQETVLLD | | | | |
| CREM | IL2RA | 1390 | NM_181571.2 | NC_000010.11 | MSKCARKKYIKTNPRQMTMETVESQHDG | 5 | Positive | NS | NS |
| | | | | | SITASLTESKSAHVQTQTGQNSIPALAQVA | | Regulator | | |
| | | | | | AIAETDESAESEGVIDSHKRREILSRRPSYR | | | | |
| | | | | | KILNELSSDVPGVPKIEEERSEEEGTPPSIA | | | | |
| | | | | | TMAVPTSIYQTSTGQYIAIAQGGTIQISNP | | | | |
| | | | | | GSDGVQGLQALTMTNSGAPPPGATIVQY | | | | |
| | | | | | AAQSADGTQQFFVPGSQVVVQAATGDMP | | | | |
| | | | | | TYQIRAPTAALPQGVVMAASPGSLHSPQQ | | | | |
| | | | | | LAEEATRKRELRLMKNREAAKECRRRKK | | | | |
| | | | | | EYVKCLESRVAVLEVQNKKLIEELETLKD | | | | |
| | | | | | ICSPKTDY | | | | |
| PTF1A | IL2RA | 256297 | NM_178161.2 | NC_000010.11 | MDAVLLEHFPGGLDAFPSSYFDEDDFFTD | 6 | Positive | NS | NS |
| | | | | | QSSRDPLEDGDELLADEQAEVEFLSHQLH | | Regulator | | |
| | | | | | EYCYRDGACLLLQPAPPAAPLALAPPSSG | | | | |
| | | | | | GLGEPDDGGGGGYCCETGAPPGGFPYSP | | | | |
| | | | | | GSPPSCLAYPCAGAAVLSPGARLRGLSGA | | | | |
| | | | | | AAAAARRRRRVRSEAELQQLRQAANVRE | | | | |
| | | | | | RRRMQSINDAFEGLRSHIPTLPYEKRLSKV | | | | |
| | | | | | DTLRLAIGYINFLSELVQADLPLRGGGAG | | | | |
| | | | | | GCGGPGGGGRLGGDSPGSQAQKVIICHRG | | | | |
| | | | | | TRSPSPSDPDYGLPPLAGHSLSWTDEKQL | | | | |
| | | | | | KEQNIIRTAKVWTPEDPRKLNSKSSFNNIE | | | | |
| | | | | | NEPPFEFVS | | | | |
| IKZF2 | IL2RA | 22807 | NM_016260.2 | NC_000002.12 | METEAIDGYITCDNELSPEREHSNMAIDLT | 7 | Positive | NS | NS |
| | | | | | SSTPNGQHASPSHMTSTNSVKLEMQSDEE | | Regulator | | |
| | | | | | CDRKPLSREDEIRGHDEGSSLEEPLIESSEV | | | | |
| | | | | | ADNRKVQELQGEGGIRLPNGKLKCDVCG | | | | |
| | | | | | MVCIGPNVLMVHKRSHTGERPFHCNQCG | | | | |
| | | | | | ASFTQKGNLLRHIKLHSGEKPFKCPFCSY | | | | |
| | | | | | ACRRRDALTGHLRTHSVGKPHKCNYCGR | | | | |
| | | | | | SYKQRSSLEEHKERCHNYLQNVSMEAAG | | | | |
| | | | | | QVMSHHVPPMEDCKEQEPIMDNNISLVPF | | | | |
| | | | | | ERPAVIEKLTGNMGKRKSSTPQKFVGEKL | | | | |
| | | | | | MRFSYPDIHFDMNLTYEKEAELMQSHMM | | | | |
| | | | | | DQAINNAITYLGAEALHPLMQHPPSTIAE | | | | |
| | | | | | VAPVISSAYSQVYHPNRIERPISRETADSH | | | | |
| | | | | | ENNMDGPISLIRPKSRPQEREASPSNSCLD | | | | |
| | | | | | STDSESSHDDHQSYQGHPALNPKRKQSPA | | | | |
| | | | | | YMKEDVKALDTTKAPKGSLKDIYKVENG | | | | |
| | | | | | EGEQIRAFKCEHCRVLFLDHVMYTIHMG | | | | |
| | | | | | CHGYRDPLECNICGYRSQDRYEFSSHIVR | | | | |
| | | | | | GEHTFH | | | | |
| MYNN | IL2RA | 55892 | NM_018657.4 | NC_000003.12 | MQYSHHCEHLLERLNKQREAGFLCDCTI | 8 | Positive | NS | NS |
| | | | | | VIGEFQFKAHRNVLASFSEYFGAIYRSTSE | | Regulator | | |
| | | | | | NNVELDQSQVKADGFQKLLEFIYTGTLNL | | | | |
| | | | | | DSWNVKEIHQAADYLKVEEVVTKCKIKM | | | | |
| | | | | | EDFAFIANPSSTEISSITGNIELNQQTCLLTL | | | | |
| | | | | | RDYNNREKSEVSTDLIQANPKQGALAKK | | | | |
| | | | | | SSQTKKKKKAFNSPKTGQNKTVQYPSDIL | | | | |
| | | | | | ENASVELFLDANKLPTPVVEQVAQINDNS | | | | |
| | | | | | ELELTSVVENTFPAQDIVHTVTVKRKRGK | | | | |
| | | | | | SQPNCALKEHSMSNIASVKSPYEAENSGE | | | | |
| | | | | | ELDQRYSKAKPMCNTCGKVFSEASSLRR | | | | |
| | | | | | HMRIHKGVKPYVCHLCGKAFTQCNQLKT | | | | |
| | | | | | HVRTHTGEKPYKCELCDKGFAQKCQLVF | | | | |
| | | | | | HSRMHHGEEKPYKCDVCNLQFATSSNLKI | | | | |
| | | | | | HARKHSGEKPYVCDRCGQRFAQASTLTY | | | | |
| | | | | | HVRRHTGEKPYVCDTCGKAFAVSSSLITH | | | | |
| | | | | | SRKHTGEKPYICGICGKSFISSGELNKHFR | | | | |
| | | | | | SHTGERPFICELCGNSYTDIKNLKKHKTK | | | | |
| | | | | | VHSGADKTLDSSAEDHTLSEQDSIQKSPLS | | | | |
| | | | | | ETMDVKPSDMTLPLALPLGTEDHHMLLP | | | | |
| | | | | | VTDTQSPTSDTLLRSTVNGYSEPQLIFLQQ | | | | |
| | | | | | LY | | | | |
| BACH2 | IL2RA | 60468 | NM_021813.3 | NC_000006.12 | MSVDEKPDSPMYVYESTVHCTNILLGLN | 9 | Positive | Positive | Negative |
| | | | | | DQRKKDILCDVTLIVERKEFRAHRAVLAA | | Regulator | Regulator | Regulator |
| | | | | | CSEYFWQALVGQTKNDLVVSLPEEVTAR | | | | |
| | | | | | GFGPLLQFAYTAKLLLSRENIREVIRCAEF | | | | |
| | | | | | LRMHNLEDSCFSFLQTQLLNSEDGLFVCR | | | | |
| | | | | | KDAACQRPHEDCENSAGEEEDEEEETMD | | | | |
| | | | | | SETAKMACPRDQMLPEPISFEAAAIPVAE | | | | |
| | | | | | KEEALLPEPDVPTDTKESSEKDALTQYPR | | | | |
| | | | | | YKKYQLACTKNVYNASSHSTSGFASTFRE | | | | |
| | | | | | DNSSNSLKPGLARGQIKSEPPSEENEEESIT | | | | |
| | | | | | LCLSGDEPDAKDRAGDVEMDRKQPSPAP | | | | |
| | | | | | TPTAPAGAACLERSRSVASPSCLRSLFSIT | | | | |
| | | | | | KSVELSGLPSTSQQHFARSPACPFDKGITQ | | | | |
| | | | | | GDLKTDYTPFTGNYGQPHVGQKEVSNFT | | | | |
| | | | | | MGSPLRGPGLEALCKQEGELDRRSVIFSSS | | | | |
| | | | | | ACDQVSTSVHSYSGVSSLDKDLSEPVPKG | | | | |
| | | | | | LWVGAGQSLPSSQAYSHGGLMADHLPGR | | | | |
| | | | | | MRPNTSCPVPIKVCPRSPPLETRTRTSSSCS | | | | |
| | | | | | SYSYAEDGSGGSPCSLPLCEFSSSPCSQGA | | | | |
| | | | | | RFLATEHQEPGLMGDGMYNQVRPQIKCE | | | | |
| | | | | | QSYGTNSSDESGSFSEADSESCPVQDRGQ | | | | |
| | | | | | EVKLPFPVDQITDLPRNDFQMMIKMHKLT | | | | |
| | | | | | SEQLEFIHDVRRRSKNRIAAQRCRKRKLD | | | | |
| | | | | | CIQNLECEIRKLVCEKEKLLSERNQLKAC | | | | |
| | | | | | MGELLDNFSCLSQEVCRDIQSPEQIQALH | | | | |
| | | | | | RYCPVLRPMDLPTASSINPAPLGAEQNIAA | | | | |
| | | | | | SQCAVGENVPCCLEPGAAPPGPPWAPSNT | | | | |
| | | | | | SENCTSGRRLEGTDPGTFSERGPPLEPRSQ | | | | |
| | | | | | TVTVDFCQEMTDKCTTDEQPRKDYT | | | | |
| IL15RA | IL2RA | 3601 | NM_0021893 | NC_000010.11 | MAPRRARGCRTLGLPALLLLLLLRPPATR | 10 | Positive | NS | NS |
| | | | | | GITCPPPMSVEHADIWVKSYSLYSRERYIC | | Regulator | | |
| | | | | | NSGFKRKAGTSSLTECVLNKATNVAHWT | | | | |
| | | | | | TPSLKCIRDPALVHQRPAPPSTVTTAGVTP | | | | |
| | | | | | QPESLSPSGKEPAASSPSSNNTAATTAAIV | | | | |
| | | | | | PGSQLMPSKSPSTGTTEISSHESSHGTPSQT | | | | |
| | | | | | TAKNWELTASASHQPPGVYPQGHSDTTV | | | | |
| | | | | | AISTSTVLLCGLSAVSLLACYLKSRQTPPL | | | | |
| | | | | | ASVEMEAMEALPVTWGTSSRDEDLENCS | | | | |
| | | | | | HHL | | | | |
| SP3 | IL2RA | 6670 | NM_ | NC_000002.12 | MTAPEKPVKQEEMAALDVDSGGGGGGG | 11 | Positive | NS | NS |
| | | | 001172712.1 | | GGHGEYLQQQQQHGNGAVAAAAAAQDT | | Regulator | | |
| | | | | | QPSPLALLAATCSKIGPPSPGDDEEEAAAA | | | | |
| | | | | | AGAPAAAGATGDLASAQLGGAPNRWEV | | | | |
| | | | | | LSATPTTIKDEAGNLVQIPSAATSSGQYVL | | | | |
| | | | | | PLQNLQNQQIFSVAPGSDSSNGTVSSVQY | | | | |
| | | | | | QVIPQIQSADGQQVQIGFTGSSDNGGINQE | | | | |
| | | | | | SSQIQIIPGSNQTLLASGTPSANIQNLIPQTG | | | | |
| | | | | | QVQVQGVAIGGSSFPGQTQVVANVPLGL | | | | |
| | | | | | PGNITFVPINSVDLDSLGLSGSSQTMTAGI | | | | |
| | | | | | NADGHLINTGQAMDSSDNSERTGERVSP | | | | |
| | | | | | DINETNTDTDLFVPTSSSSQLPVTIDSTGIL | | | | |
| | | | | | QQNTNSLITSSGQVHSSDLQGNYIQSPVS | | | | |
| | | | | | EETQAQNIQVSTAQPVVQHLQLQESQQPT | | | | |
| | | | | | SQAQIVQGITPQTIHGVQASGQNISQQALQ | | | | |
| | | | | | NLQLQLNPGTFLIQAQTVTPSGQVTWQTF | | | | |
| | | | | | QVQGVQNLQNLQIQNTAAQQITLTPVQTL | | | | |
| | | | | | TLGQVAAGGAFTSTPVSLSTGQLPNLQTV | | | | |
| | | | | | TVNSIDSAGIQLHPGENADSPADIRIKEEEP | | | | |
| | | | | | DPEEWQLSGDSTLNTNDLTHLRVQVVDE | | | | |
| | | | | | EGDQQHQEGKRLRRVACTCPNCKEGGGR | | | | |
| | | | | | GTNLGKKKQHICHIPGCGKVYGKTSHLR | | | | |
| | | | | | AHLRWHSGERPFVCNWMYCGKRFTRSD | | | | |
| | | | | | ELQRHRRTHTGEKKFVCPECSKRFMRSD | | | | |
| | | | | | HLAKHIKTHQNKKGIHSSSTVLASVEAAR | | | | |
| | | | | | DDTLITAGGTTLILANIQQGSVSGIGTVNT | | | | |
| | | | | | SATSNQDILTNTEIPLQLVTVSGNETME | | | | |
| ZBTB3 | IL2RA | 79842 | NM_024784.3 | NC_000011.10 | MLREFSKWGVEASPGKAWERKRSLLRGA | 12 | Positive | NS | NS |
| | | | | | VGRYRGATGGDLFWAPFPSWGTMEFPEH | | Regulator | | |
| | | | | | SQQLLQSLREQRSQGFLCDCTVMVGSTQF | | | | |
| | | | | | LAHRAVLASCSPFFQLFYKERELDKRDLV | | | | |
| | | | | | CIHNEIVTAPAFGLLLDFMYAGQLTLRGD | | | | |
| | | | | | TPVEDVLAAASYLHMNDIVKVCKRRLQA | | | | |
| | | | | | RALAEADSTKKEEETNSQLPSLEFLSSTSR | | | | |
| | | | | | GTQPSLASAETSGHWGKGEWKGSAAPSP | | | | |
| | | | | | TVRPPDEPPMSSGADTTQPGMEVDAPHLR | | | | |
| | | | | | APHPPVADVSLASPSSSTETIPTNYFSSGIS | | | | |
| | | | | | AVSLEPLPSLDVGPESLRVVEPKDPGGPL | | | | |
| | | | | | QGFYPPASAPTSAPAPVSAPVPSQAPAPAE | | | | |
| | | | | | AELVQVKVEAIVISDEETDVSDEQPQGPE | | | | |
| | | | | | RAFPSGGAVYGAQPSQPEAFEDPGAAGLE | | | | |
| | | | | | EVGPSDHFLPTDPHLPYHLLPGAGQYHRG | | | | |
| | | | | | LVTSPLPAPASLHEPLYLSSEYEAAPGSFG | | | | |
| | | | | | VFTEDVPTCKTCGKTFSCSYTLRRHATVH | | | | |
| | | | | | TRERPYECRYCLRSYTQSGDLYRHIRKAH | | | | |
| | | | | | NEDLAKRSKPDPEVGPLLGVQPLPGSPTA | | | | |
| | | | | | DRQSSSGGGPPKDFVLAPKTNI | | | | |
| REST | IL2RA | 5978 | NM_005612.4 | NC_000004.12 | MATQVMGQSSGGGGLFTSSGNIGMALPN | 13 | NS | Positive | Positive |
| | | | | | DMYDLHDLSKAELAAPQLIMLANVALTG | | | Regulator | Regulator |
| | | | | | EVNGSCCDYLVGEERQMAELMPVGDNN | | | | |
| | | | | | FSDSEEGEGLEESADIKGEPHGLENMELRS | | | | |
| | | | | | LELSVVEPQPVFEASGAPDIYSSNKDLPPE | | | | |
| | | | | | TPGAEDKGKSSKTKPFRCKPCQYEAESEE | | | | |
| | | | | | QFVHHIRVHSAKKFFVEESAEKQAKARES | | | | |
| | | | | | GSSTAEEGDFSKGPIRCDRCGYNTNRYDH | | | | |
| | | | | | YTAHLKHHTRAGDNERVYKCIICTYTTVS | | | | |
| | | | | | EYHWRKHLRNHFPRKVYTCGKCNYFSDR | | | | |
| | | | | | KNNYVQHVRTHTGERPYKCELCPYSSSQ | | | | |
| | | | | | KTHLTRHMRTHSGEKPFKCDQCSYVASN | | | | |
| | | | | | QHEVTRHARQVHNGPKPLNCPHCDYKTA | | | | |
| | | | | | DRSNFKKHVELHVNPRQFNCPVCDYAAS | | | | |
| | | | | | KKCNLQYHFKSKHPTCPNKTMDVSKVKL | | | | |
| | | | | | KKTKKREADLPDNITNEKTEIEQTKIKGD | | | | |
| | | | | | VAGKKNEKSVKAEKRDVSKEKKPSNNVS | | | | |
| | | | | | VIQVTTRTRKSVTEVKEMDVHTGSNSEKF | | | | |
| | | | | | SKTKKSKRKLEVDSHSLHGPVNDEESSTK | | | | |
| | | | | | KKKKVESKSKNNSQEVPKGDSKVEENKK | | | | |
| | | | | | QNTCMKKSTKKKTLKNKSSKKSSKPPQK | | | | |
| | | | | | EPVEKGSAQMDPPQMGPAPTEAVQKGPV | | | | |
| | | | | | QVEPPPPMEHAQMEGAQIRPAPDEPVQM | | | | |
| | | | | | EVVQEGPAQKELLPPVEPAQMVGAQIVL | | | | |
| | | | | | AHMELPPPMETAQTEVAQMGPAPMEPAQ | | | | |
| | | | | | MEVAQVESAPMQVVQKEPVQMELSPPM | | | | |
| | | | | | EVVQKEPVQIELSPPMEVVQKEPVKIELSP | | | | |
| | | | | | PIEVVQKEPVQMELSPPMGVVQKEPAQRE | | | | |
| | | | | | PPPPREPPLHMEPISKKPPLRKDKKEKSNM | | | | |
| | | | | | QSERARKEQVLIEVGLVPVKDSWLLKESV | | | | |
| | | | | | STEDLSPPSPPLPKENLREEASGDQKLLNT | | | | |
| | | | | | GEGNKEAPLQKVGAEEADESLPGLAANIN | | | | |
| | | | | | ESTHISSSGQNLNTPEGETLNGKHQTDSIV | | | | |
| | | | | | CEMKMDTDQNTRENLTGINSTVEEPVSP | | | | |
| | | | | | MLPPSAVEEREAVSKTALASPPATMAANE | | | | |
| | | | | | SQEIDEDEGIHSHEGSDLSDNMSEGSDDS | | | | |
| | | | | | GLHGARPVPQESSRKNAKEALAVKAAKG | | | | |
| | | | | | DFVCIFCDRSFRKGKDYSKHLNRHLVNV | | | | |
| | | | | | YYLEEAAQGQE | | | | |
| ELP2 | IL2RA | 55250 | NM_018255.2 | NC_000018.10 | MVAPVLETSHVFCCPNRVRGVLNWSSGP | 14 | NS | NS | Positive |
| | | | | | RGLLAFGTSCSVVLYDPLKRVVVINLNG | | | | Regulator |
| | | | | | HTARVNCIQWICKQDGSPSTELVSGGSDN | | | | |
| | | | | | QVIHWEIEDNQLLKAVHLQGHEGPVYAV | | | | |
| | | | | | HAVYQRRTSDPALCTLIVSAAADSAVRL | | | | |
| | | | | | WSKKGPEVMCLQTLNFGNGFALALCLSF | | | | |
| | | | | | LPNTDVPILACGNDDCRIHIFAQQNDQFQ | | | | |
| | | | | | KVLSLCGHEDWIRGVEWAAFGRDLFLAS | | | | |
| | | | | | CSQDCLIRIWKLYIKSTSLETQDDDNIRLK | | | | |
| | | | | | ENTFTIENESVKIAFAVTLETVLAGHENW | | | | |
| | | | | | VNAVHWQPVFYKDGVLQQPVRLLSASM | | | | |
| | | | | | DKTMILWAPDEESGVWLEQVRVGEVGG | | | | |
| | | | | | NTLGFYDCQFNEDGSMIIAHAFHGALHL | | | | |
| | | | | | WKQNTVNPREWTPEIVISGHFDGVQDLV | | | | |
| | | | | | WDPEGEFIITVGTDQTTRLFAPWKRKDQS | | | | |
| | | | | | QVTWHEIARPQIHGYDLKCLAMINRFQFV | | | | |
| | | | | | SGADEKVLRVFSAPRNFVENFCAITGQSL | | | | |
| | | | | | NHVLCNQDSDLPEGATVPALGLSNKAVF | | | | |
| | | | | | QGDIASQPSDEEELLTSTGFEYQQVAFQPS | | | | |
| | | | | | ILTEPPTEDHLLQNTLWPEVQKLYGHGYE | | | | |
| | | | | | IFCVTCNSSKTLLASACKAAKKEHAAIIL | | | | |
| | | | | | WNTTSWKQVQNLVFHSLTVTQMAFSPNE | | | | |
| | | | | | KFLLAVSRDRTWSLWKKQDTISPEFEPVF | | | | |
| | | | | | SLFAFTNKITSVHSRIWSCDWSPDSKYFF | | | | |
| | | | | | TGSRDKKVVVWGECDSTDDCIEHNIGPCS | | | | |
| | | | | | SVLDVGGAVTAVSVCPVLHPSQRYVVAV | | | | |
| | | | | | GLECGKICLYTWKKTDQVPEINDWTHCV | | | | |
| | | | | | ETSQSQSHTLAIRKLCWKNCSGKTEQKEA | | | | |
| | | | | | EGAEWLHFASCGEDHTVKIHRVNKCAL | | | | |
| STAT6 | IL2RA | 6778 | NM_ | NC_000012.12 | MSLWGLVSKMPPEKVQRLYVDFPQHLRH | 15 | NS | NS | Negative |
| | | | 001178078.1 | | LLGDWLESQPWEFLVGSDAFCCNLASAL | | | | Regulator |
| | | | | | LSDTVQHLQASVGEQGEGSTILQHISTLES | | | | |
| | | | | | IYQRDPLKLVATFRQILQGEKKAVMEQFR | | | | |
| | | | | | HLPMPFHWKQEELKFKTGLRRLQHRVGE | | | | |
| | | | | | IHLLREALQKGAEAGQVSLHSLIETPANG | | | | |
| | | | | | TGPSEALAMLLQETTGELEAAKALVLKRI | | | | |
| | | | | | QIWKRQQQLAGNGAPFEESLAPLQERCES | | | | |
| | | | | | LVDIYSQLQQEVGAAGGELEPKTRASLTG | | | | |
| | | | | | RLDEVLRTLVTSCFLVEKQPPQVLKTQTK | | | | |
| | | | | | FQAGVRELLGLRFLGAPAKPPLVRADMV | | | | |
| | | | | | TEKQARELSVPQGPGAGAESTGEIINNTVP | | | | |
| | | | | | LENSIPGNCCSALFKNLLLKKIKRCERKGT | | | | |
| | | | | | ESVTEEKCAVLFSASFTLGPGKLPIQLQAL | | | | |
| | | | | | SLPLVVIVHGNQDNNAKATILWDNAFSE | | | | |
| | | | | | MDRVPFVVAERVPWEKMCETLNLKFMA | | | | |
| | | | | | EVGTNRGLLPEHFLFLAQKIFNDNSLSME | | | | |
| | | | | | AFQHRSVSWSQFNKEILLGRGFTFWQWF | | | | |
| | | | | | DGVLDLTKRCLRSYWSDRLIIGFISKQYVT | | | | |
| | | | | | SLLLNEPDGTFLLRFSDSEIGGITIAHVIRG | | | | |
| | | | | | QDGSPQIENIQPFSAKDLSIRSLGDRIRDLA | | | | |
| | | | | | QLKNLYPKKPKDEAFRSHYKPEQMGKDG | | | | |
| | | | | | RGYVPATIKMTVERDQPLPTPELQMPTM | | | | |
| | | | | | VPSYDLGMAPDSSMSMQLGPDMVPQVYP | | | | |
| | | | | | PHSHSIPPYQGLSPEESVNVLSAFQEPHLQ | | | | |
| | | | | | MPPSLGQMSLPFDQPHPQGLLPCQPQEHA | | | | |
| | | | | | VSSPDPLLCSDVTMVEDSCLSQPVTAFPQ | | | | |
| | | | | | GTWIGEDIFPPLLPPTEQDLTKLLLEGQGE | | | | |
| | | | | | SGGGSLGAQPLLQPSHYGQSGISMSHMDL | | | | |
| | | | | | RANPSW | | | | |
| ZMYND8 | IL2RA | 23613 | NM_ | NC_000020.11 | MHPQSLAEEEIKTEQEVVEGMDISTRSKD | 16 | NS | NS | Negative |
| | | | 001281775.2 | | PGSAERTAQKRKFPSPPHSSNGHSPQDTST | | | | Regulator |
| | | | | | SPIKKKKKPGLLNSNNKEQSELRHGPFYY | | | | |
| | | | | | MKQPLTTDPVDVVPQDGRNDFYCWVCH | | | | |
| | | | | | REGQVLCCELCPRVYHAKCLRLTSEPEGD | | | | |
| | | | | | WFCPECEKITVAECIETQSKAMTMLTIEQL | | | | |
| | | | | | SYLLKFAIQKMKQPGTDAFQKPVPLEQHP | | | | |
| | | | | | DYAEYIFHPMDLCTLEKNAKKKMYGCTE | | | | |
| | | | | | AFLADAKWILHNCIIYNGGNHKLTQIAKV | | | | |
| | | | | | VIKICEHEMNEIEVCPECYLAACQKRDNW | | | | |
| | | | | | FCEPCSNPHPLVWAKLKGFPFWPAKALR | | | | |
| | | | | | DKDGQVDARFFGQHDRAWVPINNCYLM | | | | |
| | | | | | SKEIPFSVKKTKSIFNSAMQEMEVYVENIR | | | | |
| | | | | | RKFGVFNYSPFRTPYTPNSQYQMLLDPTN | | | | |
| | | | | | PSAGTAKIDKQEKVKLNFDMTASPKILMS | | | | |
| | | | | | KPVLSGGTGRRISLSDMPRSPMSTNSSVH | | | | |
| | | | | | TGSDVEQDAEKKATSSHFSASEESMDFLD | | | | |
| | | | | | KSTASPASTKTGQAGSLSGSPKPFSPQLSA | | | | |
| | | | | | PITTKTDKTSTTGSILNLNLDRSKAEMDLK | | | | |
| | | | | | ELSESVQQQSTPVPLISPKRQIRSRFQLNLD | | | | |
| | | | | | KTIESCKAQLGINEISEDVYTAVEHSDSED | | | | |
| | | | | | SEKSDSSDSEYISDDEQKSKNEPEDTEDKE | | | | |
| | | | | | GCQMDKEPSAVKKKPKPTNPVEIKEELKS | | | | |
| | | | | | TSPASEKADPGAVKDKASPEPEKDFSEKA | | | | |
| | | | | | KPSPHPIKDKLKGKDETDSPTVHLGLDSD | | | | |
| | | | | | SESELVIDLGEDHSGREGRKNKKEPKEPSP | | | | |
| | | | | | KQDVVGKTPPSTTVGSHSPPETPVLTRSSA | | | | |
| | | | | | QTSAAGATATTSTSSTVTVTAPAPAATGS | | | | |
| | | | | | PVKKQRPLLPKETAPAVQRVVWNSSSKF | | | | |
| | | | | | QTSSQKWHMQKMQRQQQQQQQQNQQQ | | | | |
| | | | | | QPQSSQGTRYQTRQAVKAVQQKEITQSPS | | | | |
| | | | | | TSTITLVTSTQSSPLVTSSGSMSTLVSSVN | | | | |
| | | | | | ADLPIATASADVAADIAKYTSKMMDAIK | | | | |
| | | | | | GTMTEIYNDLSKNTTGSTIABIRRLRIEIEK | | | | |
| | | | | | LQWLHQQELSEMKHNLELTMAEMRQSL | | | | |
| | | | | | EQERDRLIAEVKKQLELEKQQAVDETKK | | | | |
| | | | | | KQWCANCKKEAIFYCCWNTSYCDYPCQ | | | | |
| | | | | | QAHWPEHMKSCTQSATAPQQEADAEVN | | | | |
| | | | | | TETLNKSSQGSSSSTQSAPSETASASKEKE | | | | |
| | | | | | TSAEKSKESGSTLDLSGSRETPSSILLGSNQ | | | | |
| | | | | | GSVSKRCDKQPAYAPTTTDHQPHPNYPA | | | | |
| | | | | | QKYHSRSNKSSWSSSDEKRGSTRSDHNTS | | | | |
| | | | | | TSTKSLLPKESRLDTFWD | | | | |
| ZBTB14 | IL2RA | 7541 | NM_ | NC_000018.10 | MEFFISMSETIKYNDDDHKTLFLKTLNEQ | 17 | NS | Positive | NS |
| | | | 001143823.2 | | RLEGEFCDIAIVVEDVKFRAHRCVLAACS | | | Regulator | |
| | | | | | TYFKKLFKKLEVDSSSVIEIDFLRSDIFEEV | | | | |
| | | | | | LNYMYTAKISVKKEDVNLMMSSGQILGIR | | | | |
| | | | | | FLDKLCSQKRDVSSPDENNGQSKSKYCLK | | | | |
| | | | | | INRPIGDAADTQDDDVEEIGDQDDSPSDD | | | | |
| | | | | | TVEGTPPSQEDGKSPTTTLRVQEAILKELG | | | | |
| | | | | | SEEVRKVNCYGQEVESMETPESKDLGSQT | | | | |
| | | | | | PQALTENDGMSEVKDEQTPGWTTAASDM | | | | |
| | | | | | KFEYLLYGHHREQIACQACGKTESDEGRL | | | | |
| | | | | | RKHEKLHTADRPFVCEMCTKGFTTQAHL | | | | |
| | | | | | KEHLKIHTGYKPYSCEVCGKSFIRAPDLK | | | | |
| | | | | | KHERVHSNERPFACHMCDKAFKHKSHLK | | | | |
| | | | | | DHERRHRGEKPFVCGSCTKAFAKASDLK | | | | |
| | | | | | RHENNMHSERKQVTPSAIQSETEQLQAAA | | | | |
| | | | | | MAAEAEQQLETIACS | | | | |
| GFI1 | IL2RA | 2672 | NM_ | NC_000001.11 | MPRSFLVKSKKAHSYHQPRSPGPDYSLRL | 18 | NS | Positive | NS |
| | | | 001127215.1 | | ENVPAPSRADSTSNAGGAKAEPRDRLSPE | | | Regulator | |
| | | | | | SQLTEAPDRASASPDSCEGSVCERSSEFED | | | | |
| | | | | | FWRPPSPSASPASEKSMCPSLDEAQPFPLP | | | | |
| | | | | | FKPYSWSGLAGSDLRHLVQSYRPCGALE | | | | |
| | | | | | RGAGLGLFCEPAPEPGHPAALYGPKRAA | | | | |
| | | | | | GGAGAGAPGSCSAGAGATAGPGLGLYGD | | | | |
| | | | | | FGSAAAGLYERPTAAAGLLYPERGHGLH | | | | |
| | | | | | ADKGAGVKVESELLCTRLLLGGGSYKCIK | | | | |
| | | | | | CSKVFSTPHGLEVHVRRSHSGTRPFACEM | | | | |
| | | | | | CGKTFGHAVSLEQHKAVHSQERSFDCKIC | | | | |
| | | | | | GKSFKRSSTLSTHLLIHSDTRPYPCQYCGK | | | | |
| | | | | | RFHQKSDMKKHTFIHTGEKPHKCQVCGK | | | | |
| | | | | | AFSQSSNLITHSRKHTGFKPFGCDLCGKGF | | | | |
| | | | | | QRKVDLRRHRETQHGLK | | | | |
| IFNGR2 | IL2RA | 3460 | NM_005534.3 | NC_000021.9 | MRPTLLWSLLLLLGVFAAAAAAPPDPLSQ | 19 | NS | NS | Negative |
| | | | | | LPAPQHPKIRLYNAEQVLSWEPVALSNST | | | | Regulator |
| | | | | | RPVVYQVQFKYTDSKWFTADIMSIGVNC | | | | |
| | | | | | TQITATECDFTAASPSAGFPMDFNVTLRL | | | | |
| | | | | | RAELGALHSAWVTMPWFQHYRNVTVGP | | | | |
| | | | | | PENIEVTPGEGSLIIRFSSPFDIADTSTAFFC | | | | |
| | | | | | YYVHYWEKGGIQQVKGPFRSNSISLDNLK | | | | |
| | | | | | PSRVYCLQVQAQLLWNKSNIFRVGHLSNI | | | | |
| | | | | | SCYETMADASTELQQVILISVGTFSLLSVL | | | | |
| | | | | | AGACFFLVLKYRGLIKYWFHTPPSIPLQIE | | | | |
| | | | | | EYLKDPTQPILEALDKDSSPKDDVWDSVS | | | | |
| | | | | | IISFPEKEQEDVLQTL | | | | |
| IL4R | IL2RA | 3566 | NM_000418.3 | NC_000016.10 | MGWLCSGLLFPVSCLVLLQVASSGNMKV | 20 | NS | NS | Negative |
| | | | | | LQEPTCVSDYMSISTCEWKMNGPTNCSTE | | | | Regulator |
| | | | | | LRLLYQLVFLLSEAHTCIPENNGGAGCVC | | | | |
| | | | | | HLLMDDVVSADNYTLDLWAGQQLLWKG | | | | |
| | | | | | SFKPSEHVKPRAPGNLTVHTNVSDTLLLT | | | | |
| | | | | | WSNPYPPDNYLYNHLTYAVNIWSENDPA | | | | |
| | | | | | DFRIYNVTYLEPSLRIAASTLKSGISYRAR | | | | |
| | | | | | VRAWAQCYNTTWSEWSPSTKWHNSYRE | | | | |
| | | | | | PFEQHLLLGVSVSCIVILAVCLLCYVSITKI | | | | |
| | | | | | KKEWWDQIPNPARSRLVAIIIQDAQGSQW | | | | |
| | | | | | EKRSRGQEPAKCPHWKNCLTKLLPCFLEH | | | | |
| | | | | | NMKRDEDPHKAAKEMPFQGSGKSAWCP | | | | |
| | | | | | VEISKTVLWPESISVVRCVELFEAPVECEE | | | | |
| | | | | | EEEVEEEKGSFCASPESSRDDFQEGREGIV | | | | |
| | | | | | ARLTESLFLDLLGEENGGFCQQDMGESCL | | | | |
| | | | | | LPPSGSTSAHMPWDEFPSAGPKEAPPWGK | | | | |
| | | | | | EQPLHLEPSPPASPTQSPDNLTCTETPLVIA | | | | |
| | | | | | GNPAYRSFSNSLSQSPCPRELGPDPLLARH | | | | |
| | | | | | LEEVEPEMPCVPQLSEPTTVPQPEPETWE | | | | |
| | | | | | QILRRNVLQHGAAAAPVSAPTSGYQEFVH | | | | |
| | | | | | AVEQGGTQASAVVGLGPPGEAGYKAFSS | | | | |
| | | | | | LLASSAVSPEKCGFGASSGEEGYKPFQDLI | | | | |
| | | | | | PGCPGDPAPVPVPLFTFGLDREPPRSPQSS | | | | |
| | | | | | HLPSSSPEHLGLEPGEKVEDMPKPPLPQEQ | | | | |
| | | | | | ATDPLVDSLGSGIVYSALTCHLCGHLKQC | | | | |
| | | | | | HGQEDGGQTPVMASPCCGCCCGDRSSPP | | | | |
| | | | | | TTPLRAPDPSPGGVPLEASLCPASLAPSGIS | | | | |
| | | | | | EKSKSSSSFHPAPGNAQSSSQTPKIVNFVS | | | | |
| | | | | | VGPTYMRVS | | | | |
| IL2RB | IL2RA | 3560 | NM_000878.3 | NC_000022.11 | MAAPALSWRLPLLILLLPLATSWASAAVN | 21 | NS | Positive | Positive |
| | | | | | GTSQFTCFYNSRANISCVWSQDGALQDTS | | | Regulator | Regulator |
| | | | | | CQVHAWPDRRRWNQTCELLPVSQASWA | | | | |
| | | | | | CNLILGAPDSQKLTTVDIVTLRVLCREGV | | | | |
| | | | | | RWRVMAIQDFKPFENLRLMAPISLQVVH | | | | |
| | | | | | VETHRQNISWEISQASHYFERHLEFEARTL | | | | |
| | | | | | SPGHTWEEAPLLTLKQKQEWICLETLTPD | | | | |
| | | | | | TQYEFQVRVKPLQGEFTTWSPWSQPLAFR | | | | |
| | | | | | TKPAALGKDTIPWLGHLLVGLSGAFGFIIL | | | | |
| | | | | | VYLLINCRNTGPWLKKVLKCNTPDPSKFF | | | | |
| | | | | | SQLSSEHGGDVQKWLSSPFPSSSFSPGGLA | | | | |
| | | | | | PEISPLEVLERDKVTQLLLQQDKVPEPASL | | | | |
| | | | | | SSNHSLTSCFTNQGYFFFHLPDALEIEACQ | | | | |
| | | | | | VYFTYDPYSEEDPDEGVAGAPTGSSPQPL | | | | |
| | | | | | QPLSGEDDAYCTFPSRDDLLLFSPSLLGGP | | | | |
| | | | | | SPPSTAPGGSGAGEERMPPSLQERVPRDW | | | | |
| | | | | | DPQPLGPPTPGVPDLVDFQPPPELVLREAG | | | | |
| | | | | | EEVPDAGPREGVSFPWSRPPGQGEFRALN | | | | |
| | | | | | ARLPLNTDAYLSLQELQGQDPTHLV | | | | |
| NR4A3 | IL2RA | 8013 | NM_173200.2 | NC_000009.12 | MHDSIRFGNVDMPCVQAQYSPSPPGSSYA | 22 | NS | NS | Positive |
| | | | | | AQTYSSEYTTEIMNPDYTKLTMDLGSTEI | | | | Regulator |
| | | | | | TATATTSLPSISTFVEGYSSNYELKPSCVY | | | | |
| | | | | | QMQRPLIKVEEGRAPSYHHHHHHHHHHH | | | | |
| | | | | | HHHQQQHQQPSIPPASSPEDEVLPSTSMYF | | | | |
| | | | | | KQSPPSTPTTPAFPPQAGALWDEALPSAP | | | | |
| | | | | | GCIAPGPLLDPPMKAVPTVAGARFPLFHF | | | | |
| | | | | | KPSPPHPPAPSPAGGHHLGYDPTAAAALS | | | | |
| | | | | | LPLGAAAAAGSQAAALESHPYGLPLAKR | | | | |
| | | | | | AAPLAFPPLGLTPSPTASSLLGESPSLPSPP | | | | |
| | | | | | SRSSSSGEGTCAVCGDNAACQHYGVRTC | | | | |
| | | | | | EGCKGFFKRTVQKNAKYVCLANKNCPVD | | | | |
| | | | | | KRRRNRCQYCRFQKCLSVGMVKEVVRT | | | | |
| | | | | | DSLKGRRGRLPSKPKSPLQQEPSQPSPPSP | | | | |
| | | | | | PICMMNALVRALTDSTPRDLDYSRYCPTD | | | | |
| | | | | | QAAAGTDAEHVQQFYNLLTASIDVSRSW | | | | |
| | | | | | AEKIPGFTDLPKEDQTLLIESAFLELFVLRL | | | | |
| | | | | | SIRSNTAEDKFVFCNGLVLHRLQCLRGFG | | | | |
| | | | | | EWLDSIKDFSLNLQSLNLDIQALACLSALS | | | | |
| | | | | | MITERHGLKEPKRVEELCNKITSSLKDHQ | | | | |
| | | | | | SKGQALEPTESKVLGALVELRKICTLGLQ | | | | |
| | | | | | RIFYLKLEDLVSPPSIIDKLFLDTLPF | | | | |
| IL2RA | IL2RA | 8013 | NM_173200.2 | NC_000009.12 | MHDSIRFGNVDMPCVQAQYSPSPPGSSYA | 23 | NS | NS | Positive |
| | | | | | AQTYSSEYTTEIMNPDYTKLIMDLGSTEI | | | | Regulator |
| | | | | | TATATTSLPSISTFVEGYSSNYELKPSCVY | | | | |
| | | | | | QMQRPLIKVEEGRAPSYHHHHHHHHHHH | | | | |
| | | | | | HHHQQQHQQPSIPPASSPEDEVLPSTSMYF | | | | |
| | | | | | KQSPPSTPTTPAFPPQAGALWDEALPSAP | | | | |
| | | | | | GCIAPGPLLDPPMKAVPTVAGARFPLFHF | | | | |
| | | | | | KPSPPHPPAPSPAGGHHLGYDPTAAAALS | | | | |
| | | | | | LPLGAAAAAGSQAAALESHPYGLPLAKR | | | | |
| | | | | | AAPLAFPPLGLTPSPTASSLLGESPSLPSPP | | | | |
| | | | | | SRSSSSGEGTCAVCGDNAACQHYGVRTC | | | | |
| | | | | | EGCKGFFKRTVQKNAKYVCLANKNCPVD | | | | |
| | | | | | KRRRNRCQYCRFQKCLSVGMVKEVVRT | | | | |
| | | | | | DSLKGRRGRLPSKPKSPLQQEPSQPSPPSP | | | | |
| | | | | | PICMMNALVRALTDSTPRDLDYSRYCPTD | | | | |
| | | | | | QAAAGTDAEHVQQFYNLLTASIDVSRSW | | | | |
| | | | | | AEKIPGFTDLPKEDQTLLIESAFLELFVLRL | | | | |
| | | | | | SIRSNTAEDKFVFCNGLVLHRLQCLRGFG | | | | |
| | | | | | EWLDSIKDFSLNLQSLNLDIQALACLSALS | | | | |
| | | | | | MITERHGLKEPKRVEELCNKITSSLKDHQ | | | | |
| | | | | | SKGQALEPTESKVLGALVELRKICTLGLQ | | | | |
| | | | | | RIFYLKLEDLVSPPSIIDKLFLDTLPF | | | | |
| LEF1 | IL2RA | 51176 | NM_016269.4 | NC_000004.12 | MPQLSGGGGGGGGDPELCATDEMIPFKD | 24 | NS | NS | Negative |
| | | | | | EGDPQKEKIFAEISHPEEEGDLADIKSSLV | | | | Regulator |
| | | | | | NESEIPASNGHEVARQAQTSQEPYHDKA | | | | |
| | | | | | REHPDDGKHPDGGLYNKGPSYSSYSGYI | | | | |
| | | | | | MMPNMNNDPYMSNGSLSPPIPRTSNKVP | | | | |
| | | | | | VVQPSHAVHPLTPLITYSDEHFSPGSHPSH | | | | |
| | | | | | IPSDVNSKQGMSRHPPAPDIPTFYPLSPGG | | | | |
| | | | | | VGQITPPLGWQGQPVYPITGGFRQPYPSSL | | | | |
| | | | | | SVDTSMSRFSHHMIPGPPGPHTTGIPHPAI | | | | |
| | | | | | VTPQVKQEHPHTDSDLMHVKPQHEQRKE | | | | |
| | | | | | QEPKRPHIKKPLNAFMLYMKEMRANVVA | | | | |
| | | | | | ECTLKESAAINQILGRRWHALSREEQAKY | | | | |
| | | | | | YELARKERQLHMQLYPGWSARDNYGKK | | | | |
| | | | | | KKRKREKLQESASGTGPRMTAAYI | | | | |
| ZBTB32 | IL2RA | 27033 | NM_014383.1 | NC_000019.10 | MSLPPIRLPSPYGSDRLVQLAARLRPALCD | 25 | NS | NS | Positive |
| | | | | | TLITVGSQEFPAHSLVLAGVSQQLGRRGQ | | | | Regulator |
| | | | | | WALGEGISPSTFAQLLNFVYGESVELQPG | | | | |
| | | | | | ELRPLQEAARALGVQSLEEACWRARGDR | | | | |
| | | | | | AKKPDPGLKKHQEEPEKPSRNPERELGDP | | | | |
| | | | | | GEKQKPEQVSRTGGREQEMLHKHSPPRG | | | | |
| | | | | | RPEMAGATQEAQQEQTRSKEKRLQAPVG | | | | |
| | | | | | QRGADGKHGVLTWLRENPGGSEESLRKL | | | | |
| | | | | | PGPLPPAGSLQTSVTPRPSWAEAPWLVGG | | | | |
| | | | | | QPALWSILLMPPRYGIPFYHSTPTTGAWQ | | | | |
| | | | | | EVWREQRIPLSLNAPKGLWSQNQLASSSP | | | | |
| | | | | | TPGSLPQGPAQLSPGEMEESDQGHTGALA | | | | |
| | | | | | TCAGHEDKAGCPPRPHPPPAPPARSRPYA | | | | |
| | | | | | CSVCGKRFSLKHQMETHYRVHTGEKPFS | | | | |
| | | | | | CSLCPQRSRDFSAMTKHLRTHGAAPYRCS | | | | |
| | | | | | LCGAGCPSLASMQAHMRGHSPSQLPPGW | | | | |
| | | | | | TIRSTFLYSSSRPSRPSTSPCCPSSSTT | | | | |
| ZKSCAN2 | IL2RA | 342357 | NM_ | NC_000016.10 | MAVALDSQIDAPLEVEGCLIMKVEKDPE | 26 | Negative | NS | NS |
| | | | 001012981.4 | | WASEPILEGSDSSETFRKCFRQFCYEDVTG | | Regulator | | |
| | | | | | PHEAFSKLWELCCRWLKPEMRSKEQILEL | | | | |
| | | | | | LVIEQFLTILPEKIQAWAQKQCPQSGEEAV | | | | |
| | | | | | ALVVHLEKETGRLRQQVSSPVHREKHSPL | | | | |
| | | | | | GAAWEVADFQPEQVETQPRAVSREEPGS | | | | |
| | | | | | LHSGHQEQLNRKRERRPLPKNARPSPWVP | | | | |
| | | | | | ALADEWNTLDQEVTTTRLPAGSQEPVKD | | | | |
| | | | | | VHVARGFSYRKSVHQIPAQRDLYRDFRK | | | | |
| | | | | | ENVGNVVSLGSAVSTSNKITRLEQRKEPW | | | | |
| | | | | | TLGLHSSNKRSILRSNYVKEKSVHAIQVP | | | | |
| | | | | | ARSAGKTWREQQQWGLEDEKIAGVHWS | | | | |
| | | | | | GAVAEWLRECGFLRTPEQCRTKFKSLQKS | | | | |
| | | | | | YRKVRNGHMLEPCAFFEDMDALLNPAAR | | | | |
| | | | | | APSTDKPKEMIPVPRLKRIAISAKEHISLVE | | | | |
| | | | | | EEEAAEDSDDDEIGIEFIRKSEIHGAPVLFQ | | | | |
| | | | | | NLSGVHWGYEETKTFLDILRETRFYEALQ | | | | |
| | | | | | ACHRKSKLYGAVAEQLRECGFLRTPEQC | | | | |
| | | | | | RTKFKSLQKSYRKVKNGHVLESCAFYKE | | | | |
| | | | | | MDALINSRASAPSPSTPEEVPSPSRQERGG | | | | |
| | | | | | IEVEPQEPTGWEPEETSQEAVIEDSCSERM | | | | |
| | | | | | SEEEIVQEPEFQGPPGLLQSPNDFEIGSSIK | | | | |
| | | | | | EDPTQIVYKDMEQHRALIEKSKRVVSQST | | | | |
| | | | | | DPSKYRKRECISGRQWENLQGIRQGKPMS | | | | |
| | | | | | QPRDLGKAVVHQRPFVGKRPYRLLKYGE | | | | |
| | | | | | SFGRSTRLMCRMTHHKENPYKCGVCGKC | | | | |
| | | | | | FGRSRSLIRHQRIHTGEKPFKCLDCGKSEN | | | | |
| | | | | | DSSNFGAHQRIHTGEKPYRCGECGKCFSQ | | | | |
| | | | | | SSSLIHQRTHTGEKPYQCGECGKSFINSS | | | | |
| | | | | | HFSAHRRVHTGENPYKCVDCEKSENNCT | | | | |
| | | | | | RFREHRRIHTGEKPYGCAQCGKRFSKSSV | | | | |
| | | | | | LTKHREVHVREKPLPHPPSLYCPENPHKG | | | | |
| | | | | | KTDEFRKTF | | | | |
| ARID5A | IL2RA | 10865 | NM_212481.1 | NC_000002.12 | MAAPVKGNRKQSTEGDALDPPASPKPAG | 27 | NS | NS | Negative |
| | | | | | KQNGIQNPISLEDSPEAGGEREEEQEREEE | | | | Regulator |
| | | | | | QAFLVSLYKFMKERHTPIERVPHLGFKQI | | | | |
| | | | | | NLWKIYKAVEKLGAYELVTGRRLWKNV | | | | |
| | | | | | YDELGGSPGSTSAATCTRRHYERLVLPYV | | | | |
| | | | | | RHLKGEDDKPLPTSKPRKQYKMAKENRG | | | | |
| | | | | | DDGATERPKKAKEERRMDQMMPGKTKA | | | | |
| | | | | | DAADPAPLPSQEPPRNSTEQQGLASGSSV | | | | |
| | | | | | SFVGASGCPEAYKRLLSSFYCKGTHGIMS | | | | |
| | | | | | PLAKKKLLAQVSKVEALQCQEEGCRHGA | | | | |
| | | | | | EPQASPAVHLPESPQSPKGLTENSRHRLTP | | | | |
| | | | | | QEGLQAPGGSLREEAQAGPCPAAPIFKGC | | | | |
| | | | | | FYTHPTEVLKPVSQHPRDFFSRLKDGVLL | | | | |
| | | | | | GPPGKEGLSVKEPQLVWGGDANRPSAFH | | | | |
| | | | | | KGGSRKGILYPKPKACWVSPMAKVPAES | | | | |
| | | | | | PTLPPTFPSSPGLGSKRSLEEEGAAHSGKR | | | | |
| | | | | | LRAVSPFLKEADAKKCGAKPAGSGLVSC | | | | |
| | | | | | LLGPALGPVPPEAYRGTMLHCPLNFTGTP | | | | |
| | | | | | GPLKGQAALPFSPLVIPAFPAHFLATAGPS | | | | |
| | | | | | PMAAGLMHFPPTSFDSALRHRLCPASSA | | | | |
| | | | | | WHAPPVTTYAAPHFFHLNTKL | | | | |
| ZNF114 | IL2RA | 163071 | NM_153608.2 | NC_000019.10 | MSQDSVTFADVAVNFTKEEWILLDPAQR | 28 | Negative | NS | NS |
| | | | | | NLYRDVMLENSRNLAFIDWATPCKTKDA | | Regulator | | |
| | | | | | TPQPDILPKRTFPEANRVCLTSISSQHSTLR | | | | |
| | | | | | EDWRCPKTEEPHRQGVNNVKPPAVAPEK | | | | |
| | | | | | DESPVSICEDHEMRNHSKPTCRLVPSQGD | | | | |
| | | | | | SIRQCILTRDSSIFKYNPVLNDSQKTHENN | | | | |
| | | | | | EDDGVLGWNIQWVPCGRKTELKSSTWTG | | | | |
| | | | | | SQNTVHHIRDEIDTGANRHQRNPFGKAFR | | | | |
| | | | | | EDGSLRAHNTHGREKMYDFTQCENTSRN | | | | |
| | | | | | NSIHAMQMQLYTAETNKKDCQTGATSAN | | | | |
| | | | | | APNSGSHKSHCTGEKTHKCPECGRAFFYQ | | | | |
| | | | | | SFLMRHMKIHTGEKPYECGKCGKAFRYS | | | | |
| | | | | | LHLNKHLRKHVVQKKPYECEECGKVIRE | | | | |
| | | | | | SSKYTHIRSHTGEKPYKCKTCGKDFAKSS | | | | |
| | | | | | GLKKHLKTHKDEKPCE | | | | |
| E4F1 | IL2RA | 1877 | NM_004424.4 | NC_000016.10 | MEGAMAVRVTAAHTAEAQAEAGREAGE | 29 | Negative | NS | NS |
| | | | | | GAVAAVAAALAPSGFLGLPAPFSEEDEDD | | Regulator | | |
| | | | | | VHRCGRCQAEFTALEDFVQHKIQKACQR | | | | |
| | | | | | APPEALPATPATTALLGQEVVPAAPGPEE | | | | |
| | | | | | PITVAHIVVEAASLAADISHASDLVGGGHI | | | | |
| | | | | | KEVIVAAEAELGDGEMAEAPGSPRQQGL | | | | |
| | | | | | GLAGEGEQAQVKLLVNKDGRYVCALCH | | | | |
| | | | | | KTFKTGSILKAHMVTHSSRKDHECKLCG | | | | |
| | | | | | ASFRTKGSLIRHHRRHTDERPYKCSKCGK | | | | |
| | | | | | SFRESGALTRHLKSLTPCTEKIRFSVSKDV | | | | |
| | | | | | VVSKEDARAGSGAGAAGLGTATSSVTGE | | | | |
| | | | | | PIETSPVIHLVTDAKGTVIHEVHVQMQELS | | | | |
| | | | | | LGMKALAPEPPVSQELPCSSEGSRENLLH | | | | |
| | | | | | QAMQNSGIVLERAAGEEGALEPAPAAGSS | | | | |
| | | | | | PQPLAVAAPQLPVLEVQPLETQVASEASA | | | | |
| | | | | | VPRTHPCPQCSETFPTAATLEAHKRGHTG | | | | |
| | | | | | PRPFACAQCGKAFPKAYLLKKHQEVHVR | | | | |
| | | | | | ERRFRCGDCGKLYKTIAHVRGHRRVHSD | | | | |
| | | | | | ERPYPCPKCGKRYKTKNAQQVHFRTHLE | | | | |
| | | | | | EKPHVCQFCSRGFREKGSLVRHVRHHTG | | | | |
| | | | | | EKPFKCYKCGRGFAEHGTLNRHLRTKGG | | | | |
| | | | | | CLLEVEELLVSEDSPAAATTVLTEDPHTV | | | | |
| | | | | | LVEFSSVVADTQEYIIEATADDAETSEATE | | | | |
| | | | | | IIEGTQTEVDSHIMKVVQQIVHQASAGHQI | | | | |
| | | | | | IVQNVTMDEETALGPEAAAADTITIATPES | | | | |
| | | | | | LTEQVAMTLASAISEGTVLAARAGTSGTE | | | | |
| | | | | | QATVTMVSSEDIEILEHAGELVIASPEGQL | | | | |
| | | | | | EVQTVIV | | | | |
| E4F1 | IL2RA | 1877 | NM_004424.4 | NC_000016.10 | MEGAMAVRVTAAHTAEAQAEAGREAGE | 30 | Negative | NS | NS |
| | | | | | GAVAAVAAALAPSGFLGLPAPFSEEDEDD | | Regulator | | |
| | | | | | VHRCGRCQAEFTALEDFVQHKIQKACQR | | | | |
| | | | | | APPEALPATPATTALLGQEVVPAAPGPEE | | | | |
| | | | | | PITVAHIVVEAASLAADISHASDLVGGGHI | | | | |
| | | | | | KEVIVAAEAELGDGEMAEAPGSPRQQGL | | | | |
| | | | | | GLAGEGEQAQVKLLVNKDGRYVCALCH | | | | |
| | | | | | KTFKTGSILKAHMVTHSSRKDHECKLCG | | | | |
| | | | | | ASFRTKGSLIRHHRRHTDERPYKCSKCGK | | | | |
| | | | | | SFRESGALTRHLKSLTPCTEKIRFSVSKDV | | | | |
| | | | | | VVSKEDARAGSGAGAAGLGTATSSVTGE | | | | |
| | | | | | PIETSPVIHLVTDAKGTVIHEVHVQMQELS | | | | |
| | | | | | LGMKALAPEPPVSQELPCSSEGSRENLLH | | | | |
| | | | | | QAMQNSGIVLERAAGEEGALEPAPAAGSS | | | | |
| | | | | | PQPLAVAAPQLPVLEVQPLETQVASEASA | | | | |
| | | | | | VPRTHPCPQCSETFPTAATLEAHKRGHTG | | | | |
| | | | | | PRPFACAQCGKAFPKAYLLKKHQEVHVR | | | | |
| | | | | | ERRFRCGDCGKLYKTIAHVRGHRRVHSD | | | | |
| | | | | | ERPYPCPKCGKRYKTKNAQQVHFRTHLE | | | | |
| | | | | | EKPHVCQFCSRGFREKGSLVRHVRHHTG | | | | |
| | | | | | EKPFKCYKCGRGFAEHGTLNRHLRTKGG | | | | |
| | | | | | CLLEVEELLVSEDSPAAATTVLTEDPHTV | | | | |
| | | | | | LVEFSSVVADTQEYIIEATADDAETSEATE | | | | |
| | | | | | IIEGTQTEVDSHIMKVVQQIVHQASAGHQI | | | | |
| | | | | | IVQNVTMDEETALGPEAAAADTITIATPES | | | | |
| | | | | | LTEQVAMTLASAISEGTVLAARAGTSGTE | | | | |
| | | | | | QATVTMVSSEDIEILEHAGELVIASPEGQL | | | | |
| | | | | | EVQTVIV | | | | |
| GLIS2 | IL2RA | 84662 | NM_032575.2 | NC_000016.10 | MHSLDEPLDLKLSITKLRAAREKRERTLG | 31 | Negative | NS | NS |
| | | | | | VVRPRALHRELGLVDDSPTPGSPGSPPSGF | | Regulator | | |
| | | | | | LLNSKFPEKVEGRFSAAPLVDLSLSPPSGL | | | | |
| | | | | | DSPNGSSSLSPERQGNGDLPPVPSASDFQP | | | | |
| | | | | | LRYLDGVPSSFQFFLPLGSGGALHLPASSF | | | | |
| | | | | | LTPPKDKCLSPDLPLPKQLVCRWAKCNQL | | | | |
| | | | | | FELLQDLVDHVNDYHVKPEKDAGYCCH | | | | |
| | | | | | WEGCARHGRGFNARYKMLIHIRTHTNEK | | | | |
| | | | | | PHRCPTCSKSFSRLENLKIHNRSHTGEKPY | | | | |
| | | | | | VCPYEGCNKRYSNSSDRFKHTRTHYVDK | | | | |
| | | | | | PYYCKMPGCHKRYTDPSSLRKHIKAHGH | | | | |
| | | | | | FVSHEQQELLQLRPPPKPPLPAPDGGPYVS | | | | |
| | | | | | GAQIIIPNPAALFGGPGLPGLPLPLAPGPLD | | | | |
| | | | | | LSALACGNGGGSGGGGGMGPGLPGPVLP | | | | |
| | | | | | LNLAKNPLLPSPFGAGGLGLPVVSLLAGA | | | | |
| | | | | | AGGKAEGEKGRGSVPTRALGMEGHKTPL | | | | |
| | | | | | ERTESSCSRPSPDGLPLLPGTVLDLSTGVN | | | | |
| | | | | | SAASSPEALAPGWVVIPPGSVLLKPAVVN | | | | |
| JUNB | IL2RA | 3726 | NM_002229.2 | NC_000019.10 | MCTKMEQPFYHDDSYTATGYGRAPGGLS | 32 | Negative | NS | NS |
| | | | | | LHDYKLLKPSLAVNLADPYRSLKAPGAR | | Regulator | | |
| | | | | | GPGPEGGGGGSYFSGQGSDTGASLKLASS | | | | |
| | | | | | ELERLIVPNSNGVITTTPTPPGQYFYPRGG | | | | |
| | | | | | GSGGGAGGAGGGVTEEQEGFADGFVKAL | | | | |
| | | | | | DDLHKMNHVTPPNVSLGATGGPPAGPGG | | | | |
| | | | | | VYAGPEPPPVYTNLSSYSPASASSGGAGA | | | | |
| | | | | | AVGTGSSYPTTTISYLPHAPPFAGGHPAQL | | | | |
| | | | | | GLGRGASTFKEEPQTVPEARSRDATPPVS | | | | |
| | | | | | PINMEDQERIKVERKRLRNRLAATKCRKR | | | | |
| | | | | | KLERIARLEDKVKILKAENAGLSSTAGLL | | | | |
| | | | | | REQVAQLKQKVMTHVSNGCQLLLGVKG | | | | |
| | | | | | HAF | | | | |
| NR2F6 | IL2RA | 2063 | NM_005234.3 | NC_000019.10 | MAMVTGGWGGPGGDTNGVDKAGGYPR | 33 | Negative | NS | NS |
| | | | | | AAEDDSASPPGAASDAEPGDEERPGLQVD | | Regulator | | |
| | | | | | CVVCGDKSSGKHYGVFTCEGCKSFFKRSI | | | | |
| | | | | | RRNLSYTCRSNRDCQIDQHHRNQCQYCR | | | | |
| | | | | | LKKCFRVGMRKEAVQRGRIPHSLPGAVA | | | | |
| | | | | | ASSGSPPGSALAAVASGGDLFPGQPVSELI | | | | |
| | | | | | AQLLRAEPYPAAAGREGAGGGAAGAVLG | | | | |
| | | | | | IDNVCELAARLLESTVEWARHAPFFPELP | | | | |
| | | | | | VADQVALLRLSWSELFVLNAAQAALPLH | | | | |
| | | | | | TAPLLAAAGLHAAPMAAERAVAFMDQV | | | | |
| | | | | | RAFQEQVDKLGRLQVDSAEYGCLKAIAL | | | | |
| | | | | | FTPDACGLSDPAHVESLQEKAQVALTEYV | | | | |
| | | | | | RAQYPSQPQRFGRLLLRLPALRAVPASLIS | | | | |
| | | | | | QLFFMRLVGKTPIETLIRDMLLSGSTFNWP | | | | |
| | | | | | YGSGQ | | | | |
| ZZZ3 | IL2RA | 26009 | NM_015534.4 | NC_000001.11 | MAASRSTRVTRSTVGLNGLDESFCGRTLR | 34 | Negative | NS | NS |
| | | | | | NRSIAHPEEISSNSQVRSRSPKKRPEPVPIQ | | Regulator | | |
| | | | | | KGNNNGRTTDLKQQSTRESWVSPRKRGL | | | | |
| | | | | | SSSEKDNIERQAIENCERRQTEPVSPVLKRI | | | | |
| | | | | | KRCLRSEAPNSSEEDSPIKSDKESVEQRST | | | | |
| | | | | | VVDNDADFQGTKRACRCLILDDCEKREIK | | | | |
| | | | | | KVNVSEEGPLNSAVVEEITGYLAVNGVD | | | | |
| | | | | | DSDSAVINCDDCQPDGNTKQNSIGSYVLQ | | | | |
| | | | | | EKSVAENGDTDTQTSMELDSRKEDSYIDH | | | | |
| | | | | | KVPCTDSQVQVKLEDHKIVTACLPVEHV | | | | |
| | | | | | NQLTTEPATGPFSETQSSLRDSEEEVDVV | | | | |
| | | | | | GDSSASKEQCKENTNNELDTSLESMPASG | | | | |
| | | | | | EPEPSPVLDCVSAQMMSLSEPQEHRYTLR | | | | |
| | | | | | TSPRRAAPTRGSPTKNSSPYRENGQFEEN | | | | |
| | | | | | NLSPNETNATVSDNVSQSPTNPGEISQNEK | | | | |
| | | | | | GICCDSQNNGSEGVSKPPSEARLNIGHLPS | | | | |
| | | | | | AKESASQHITEEEDDDPDVYYFESDHVAL | | | | |
| | | | | | KHNKDYQRLLQTIAVLEAQRSQAVQDLE | | | | |
| | | | | | SLGRHQREALKNPIGFVEKLQKKADIGLP | | | | |
| | | | | | YPQRVVQLPEIVWDQYTHSLGNFEREFKN | | | | |
| | | | | | RKRHTRRVKLVFDKVGLPARPKSPLDPK | | | | |
| | | | | | KDGESLSYSMLPLSDGPEGSSSRPQMIRG | | | | |
| | | | | | RLCDDTKPETFNQLWTVEEQKKLEQLLIK | | | | |
| | | | | | YPPEEVESRRWQKIADELGNRTAKQVAS | | | | |
| | | | | | RVQKYFIKLTKAGIPVPGRTPNLYIYSKKS | | | | |
| | | | | | STSRRQHPLNKHLFKPSTFMTSHEPPVYM | | | | |
| | | | | | DEDDDRSCFHSHMNTAVEDASDDESIPIM | | | | |
| | | | | | YRNLPEYKELLQFKKLKKQKLQQMQAES | | | | |
| | | | | | GFVQHVGFKCDNCGIEPIQGVRWHCQDC | | | | |
| | | | | | PPEMSLDFCDSCSDCLHETDIHKEDHQLE | | | | |
| | | | | | PIYRSETFLDRDYCVSQGTSYNYLDPNYF | | | | |
| | | | | | PANR | | | | |
| ETV3 | IL2RA | 2117 | NM_ | NC_000001.11 | MKAGCSIVEKPEGGGGYQFPDWAYKTES | 35 | Negative | NS | NS |
| | | | 001145312.1 | | SPGSRQIQLWHFILELLQKEEFRHVIAWQ | | Regulator | | |
| | | | | | QGEYGEFVIKDPDEVARLWGRRKCKPQM | | | | |
| | | | | | NYDKLSRALRYYYNKRILHKTKGKRFTY | | | | |
| | | | | | KFNFNKLVMPNYPFINIRSSGVVPQSAPPV | | | | |
| | | | | | PTASSRFHFPPLDTHSPTNDVQPGRFSASS | | | | |
| | | | | | LTASGQESSNGTDRKTELSELEDGSAADW | | | | |
| | | | | | RRGVDPVSSRNAIGGGGIGHQKRKPDIML | | | | |
| | | | | | PLFARPGMYPDPHSPFAVSPIPGRGGVLN | | | | |
| | | | | | VPISPALSLTPTIFSYSPSPGLSPFTSSSCFSF | | | | |
| | | | | | NPEEMKHYLHSQACSVFNYHLSPRTFPRY | | | | |
| | | | | | PGLMVPPLQCQMHPEESTQFSIKLQPPPV | | | | |
| | | | | | GRKNRERVESSEESAPVTTPTMASIPPRIK | | | | |
| | | | | | VEPASEKDPESLRQSAREKEEHTQEEGTV | | | | |
| | | | | | PSRTIEEEKGTIFARPAAPPIWPSVPISTPSG | | | | |
| | | | | | EPLEVTEDSEDRPGKEPSAPEKKEDALMP | | | | |
| | | | | | PKLRLKRRWNDDPEARELSKSGKFLWNG | | | | |
| | | | | | SGPQGLATAAADA | | | | |
| ERF | IL2RA | 2077 | NM_ | NC_000019.10 | MNYDKLSRALRYYYNKRILHKTKGKRFT | 36 | Negative | NS | NS |
| | | | 001301035.1 | | YKFNFNKLVLVNYPFIDVGLAGGAVPQS | | Regulator | | |
| | | | | | APPVPSGGSHFRFPPSTPSEVLSPTEDPRSP | | | | |
| | | | | | PACSSSSSSLFSAVVARRLGRGSVSDCSDG | | | | |
| | | | | | TSELEEPLGEDPRARPPGPPDLGAFRGPPL | | | | |
| | | | | | ARLPHDPGVFRVYPRPRGGPEPLSPFPVSP | | | | |
| | | | | | LAGPGSLLPPQLSPALPMTPTHLAYTPSPT | | | | |
| | | | | | LSPMYPSGGGGPSGSGGGSHFSFSPEDMK | | | | |
| | | | | | RYLQAHTQSVYNYHLSPRAFLHYPGLVV | | | | |
| | | | | | PQPQRPDKCPLPPMAPETPPVPSSASSSSSS | | | | |
| | | | | | SSSPFKFKLQPPPLGRRQRAAGEKAVAGA | | | | |
| | | | | | DKSGGSAGGLAEGAGALAPPPPPPQIKVE | | | | |
| | | | | | PISEGESEEVEVTDISDEDEEDGEVEKTPR | | | | |
| | | | | | APPAPPKPEPGEAPGASQCMPLKLRFKRR | | | | |
| | | | | | WSEDCRLEGGGGPAGGFEDEGEDKKVRG | | | | |
| | | | | | EGPGEAGGPLTPRRVSSDLQHATAQLSLE | | | | |
| | | | | | HRDS | | | | |
| RUNX1 | IL2RA | 861 | NM_001754.4 | NC_000021.9 | MASDSIFESFPSYPQCFMRECILGMNPSRD | 37 | Negative | NS | NS |
| | | | | | VHDASTSRRFTPPSTALSPGKMSEALPLG | | Regulator | | |
| | | | | | APDAGAALAGKLRSGDRSMVEVLADHPG | | | | |
| | | | | | ELVRTDSPNFLCSVLPTHWRCNKTLPIAF | | | | |
| | | | | | KVVALGDVPDGTLVTVMAGNDENYSAE | | | | |
| | | | | | LRNATAAMKNQVARFNDLRFVGRSGRG | | | | |
| | | | | | KSFTLTITVFTNPPQVATYHRAIKITVDGP | | | | |
| | | | | | REPRRHRQKLDDQTKPGSLSFSERLSELE | | | | |
| | | | | | QLRRTAMRVSPHHPAPTPNPRASLNHSTA | | | | |
| | | | | | FNPQPQSQMQDTRQIQPSPPWSYDQSYQY | | | | |
| | | | | | LGSIASPSVHPATPISPGRASGMTTLSAELS | | | | |
| | | | | | SRLSTAPDLTAFSDPRQFPALPSISDPRMH | | | | |
| | | | | | YPGAFTYSPTPVTSGIGIGMSAMGSATRY | | | | |
| | | | | | HTYLPPPYPGSSQAQGGPFQASSPSYHLY | | | | |
| | | | | | YGASAGSYQFSMVGGERSPPRILPPCTNA | | | | |
| | | | | | STGSALLNPSLPNQSDVVEAEGSHSNSPT | | | | |
| | | | | | NMAPSARLEEAVWRPY | | | | |
| TGIF2 | IL2RA | 60436 | NM_021809.6 | NC_000020.11 | MSDSDLGEDEGLLSLAGKRKRRGNLPKE | 38 | Negative | NS | NS |
| | | | | | SVKILRDWLYLHRYNAYPSEQEKLSLSGQ | | Regulator | | |
| | | | | | TNLSVLQICNWFINARRRLLPDMLRKDGK | | | | |
| | | | | | DPNQFTISRRGGKASDVALPRGSSPSVLA | | | | |
| | | | | | VSVPAPTNVLSLSVCSMPLHSGQGEKPAA | | | | |
| | | | | | PFPRGELESPKPLVTPGSTLTLLTRAEAGS | | | | |
| | | | | | PTGGLFNTPPPTPPEQDKEDFSSFQLLVEV | | | | |
| | | | | | ALQRAAEMELQKQQDPSLPLLHTPIPLVS | | | | |
| | | | | | ENPQ | | | | |
| ZNF574 | IL2RA | 64763 | NM_022752.5 | NC_000019.10 | MTEESEETVLYIEHRYVCSECNQLYGSLE | 39 | Negative | NS | NS |
| | | | | | EVLMHQNSHVPQQHFELVGVADPGVTVA | | Regulator | | |
| | | | | | TDTASGTGLYQTLVQESQYQCLECGQLL | | | | |
| | | | | | MSPSQLLEHQELHLKMMAPQEAVPAEPS | | | | |
| | | | | | PKAPPLSSSTIHYECVDCKALFASQELWL | | | | |
| | | | | | NHRQTHLRATPTKAPAPVVLGSPVVLGPP | | | | |
| | | | | | VGQARVAVEHSYRKAEEGGEGATVPSAA | | | | |
| | | | | | ATTTEVVTEVELLLYKCSECSQLFQLPAD | | | | |
| | | | | | FLEHQATHFPAPVPESQEPALQQEVQASS | | | | |
| | | | | | PAEVPVSQPDPLPASDHSYELRNGEAIGR | | | | |
| | | | | | DRRGRRARRNNSGEAGGAATQELFCSAC | | | | |
| | | | | | DQLFLSPHQLQQHLRSHREGVFKCPLCSR | | | | |
| | | | | | VFPSPSSLDQHLGDHSSESHFLCVDCGLAF | | | | |
| | | | | | GTEALLLAHRRAHTPNPLHSCPCGKTFVN | | | | |
| | | | | | LTKFLYHRRTHGVGGVPLPTTPVPPEEPVI | | | | |
| | | | | | GFPEPAPAETGEPEAPEPPVSEETSAGPAA | | | | |
| | | | | | PGTYRCLLCSREFGKALQLTRHQRFVHRL | | | | |
| | | | | | ERRHKCSICGKMFKKKSHVRNHLRTHTG | | | | |
| | | | | | ERPFPCPDCSKPFNSPANLARHRLTHTGER | | | | |
| | | | | | PYRCGDCGKAFTQSSTLRQHRLVHAQHF | | | | |
| | | | | | PYRCQECGVRFHRPYRLLMHRYHHTGEY | | | | |
| | | | | | PYKCRECPRSFLLRRLLEVHQLVVHAGRQ | | | | |
| | | | | | PHRCPSCGAAFPSSLRLREHRCAAAAAQA | | | | |
| | | | | | PRRFECGTCGKKVGSAARLQAHEAAHAA | | | | |
| | | | | | AGPGEVLAKEPPAPRAPRATRAPVASPAA | | | | |
| | | | | | LGSTATASPAAPARRRGLECSECKKLFST | | | | |
| | | | | | ETSLQVHRRIHTGERPYPCPDCGKAFRQS | | | | |
| | | | | | THLKDHRRLHTGERPFACEVQGKAFAISM | | | | |
| | | | | | RLAEHRRIHTGERPYSCPDCGKSYRSFSNL | | | | |
| | | | | | WKHRKTHQQQHQAAVRQQLAEAEAAVG | | | | |
| | | | | | LAVMETAVEALPLVEAIEIYPLAEAEGVQI | | | | |
| | | | | | SG | | | | |
| PTEN | IL2RA | 5728 | NM_ | NC_000010.11 | MTAIIKEIVSRNKRRYQEDGFDLDLTYIYP | 40 | Positive | Positive | Positive |
| | | | 001304718.1 | | NIIAMGFPAERLEGVYRNNIDDVVSCAER | | Regulator | Regulator | Regulator |
| | | | | | HYDTAKFNCRVAQYPFEDHNPPQLELIKP | | | | |
| | | | | | FCEDLDQWLSEDDNHVAAIHCKAGKGRT | | | | |
| | | | | | GVMICAYLLHRGKFLKAQEALDFYGEVR | | | | |
| | | | | | TRDKKGVTIPSQRRYVYYYSYLLKNHLD | | | | |
| | | | | | YRPVALLFHKMMFETIPMFSGGTCNPQFV | | | | |
| | | | | | VCQLKVKIYSSNSGPTRREDKFMYFEFPQ | | | | |
| | | | | | PLPVCGDIKVEFFHKQNKMLKKDKMFHF | | | | |
| | | | | | WVNTFFIPGPEETSEKVENGSLCDQEIDSI | | | | |
| | | | | | CSIERADNDKEYLVLTLTKNDLDKANKD | | | | |
| | | | | | KANRYFSPNFKVKLYFTKTVEEPSNPEAS | | | | |
| | | | | | SSTSVTPDVSDNEPDHYRYSDTTDSDPEN | | | | |
| | | | | | EPFDEDQHTQITKV | | | | |
| IL2RA | IL2RA | 3559 | NM_000417.2 | NC_000010.11 | MDSYLLMWGLLTFIMVPGCQAELCDDDP | 41 | Positive | Positive | Positive |
| | | | | | PEIPHATFKAMAYKEGTMLNCECKRGFR | | Regulator | Regulator | Regulator |
| | | | | | RIKSGSLYMLCTGNSSHSSWDNQCQCTSS | | | | |
| | | | | | ATRNTTKQVTPQPEEQKERKTTEMQSPM | | | | |
| | | | | | QPVDQASLPGHCREPPPWENEATERIYHF | | | | |
| | | | | | VVGQMVYYQCVQGYRALHRGPAESVCK | | | | |
| | | | | | MTHGKTRWTQPQLICTGEMETSQFPGEE | | | | |
| | | | | | KPQASPEGRPESETSCLVITTDFQIQTEMA | | | | |
| | | | | | ATMETSIFTTEYQVAVAGCVFLLISVLLLS | | | | |
| | | | | | GLTWQRRQRKSRRTI | | | | |
| FOXO1 | IL2RA | 2308 | NM_002015.3 | NC_000013.11 | MAEAPQVVEIDPDFEPLPRPRSCTWPLPRP | 42 | Positive | NS | NS |
| | | | | | EFSQSNSATSSPAPSGSAAANPDAAAGLPS | | Regulator | | |
| | | | | | ASAAAVSADFMSNLSLLEESEDFPQAPGS | | | | |
| | | | | | VAAAVAAAAAAAATGGLCGDFQGPEAG | | | | |
| | | | | | CLHPAPPQPPPPGPLSQHPPVPPAAAGPLA | | | | |
| | | | | | GQPRKSSSSRRNAWGNLSYADLITKAJESS | | | | |
| | | | | | AEKRLTLSQIYEWMVKSVPYFKDKGDSN | | | | |
| | | | | | SSAGWKNSIRHNLSLHSKFIRVQNEGTGK | | | | |
| | | | | | SSWWMLNPEGGKSGKSPRRRAASMDNN | | | | |
| | | | | | SKFAKSRSRAAKKKASLQSGQEGAGDSP | | | | |
| | | | | | GSQFSKWPASPGSHSNDDFDNWSTFRPRT | | | | |
| | | | | | SSNASTISGRLSPIMTEQDDLGEGDVHSM | | | | |
| | | | | | VYPPSAAKMASTLPSLSEISNPENMENLL | | | | |
| | | | | | DNLNLLSSPTSLTVSTQSSPGTMMQQTPC | | | | |
| | | | | | YSFAPPNTSLNSPSPNYQKYTYGQSSMSP | | | | |
| | | | | | LPQMPIQTLQDNKSSYGGMSQYNCAPGL | | | | |
| | | | | | LKELLTSDSPPHNDIMTPVDPGVAQPNSR | | | | |
| | | | | | VLGQNVMMGPNSVMSTYGSQASHNKM | | | | |
| | | | | | MNPSSHTHPGHAQQTSAVNGRPLPHTVST | | | | |
| | | | | | MPHTSGMNRLTQVKTPVQVPLPHPMQMS | | | | |
| | | | | | ALGGYSSVSSCNGYGRMGLLHQEKLPSD | | | | |
| | | | | | LDGMFIERLDCDMESIIRNDLMDGDTLDF | | | | |
| | | | | | NFDNVLPNQSFPHSVKTTTHSWVSG | | | | |
| FOXP1 | IL2RA | 27086 | NM_032682.5 | NC_000003.12 | MMQESGTETKSNGSAIQNGSGGSNHLLE | 43 | Positive | Positive | Positive |
| | | | | | CGGLREGRSNGETPAVDIGAADLAHAQQ | | Regulator | Regulator | Regulator |
| | | | | | QQQQALQVARQLLLQQQQQQQVSGLKSP | | | | |
| | | | | | KRNDKQPALQVPVSVAMMTPQVITPQQM | | | | |
| | | | | | QQILQQQVLSPQQLQVLLQQQQALMLQQ | | | | |
| | | | | | QQLQEFYKKQQEQLQLQLLQQQHAGKQP | | | | |
| | | | | | KEQQQVATQQLAPQQQLLQMQQLQQQH | | | | |
| | | | | | LLSLQRQGLLTIQPGQPALPLQPLAQGMIP | | | | |
| | | | | | TELQQLWKEVTSAHTAEETTGNNHSSLD | | | | |
| | | | | | LTTTCVSSSAPSKTSLIMNPHASINGQLSV | | | | |
| | | | | | HTPKRESLSHEEHPHSHPLYGHGVCKWP | | | | |
| | | | | | GCEAVCEDFQSFLKHLNSEHALDDRSTAQ | | | | |
| | | | | | CRVQMQVVQQLELQLAKDKERLQAMMT | | | | |
| | | | | | HLHVKSTEPKAAPQPLNLVSSVTLSKSAS | | | | |
| | | | | | EASPQSLPHTPTTPTAPLTPVTQGPSVITTT | | | | |
| | | | | | SMHTVGPIRRRYSDKYNVPISSADIAQNQ | | | | |
| | | | | | EFYKNAEVRPPFTYASLIRQAILESPEKQL | | | | |
| | | | | | TLNEIYNWFTRMFAYFRRNAATWKNAVR | | | | |
| | | | | | HNLSLHKCFVRVENVKGAVWTVDEVEFQ | | | | |
| | | | | | KRRPQKISGNPSLIKNMQSSHAYCTPLNA | | | | |
| | | | | | ALQASMAENSIPLYTTASMGNPTLGNLAS | | | | |
| | | | | | AIREELNGAMEHTNSNESDSSPGRSPMQA | | | | |
| | | | | | VHPVHVKEEPLDPEEAEGPLSLVTTANHS | | | | |
| | | | | | PDFDHDRDYEDEPVNEDME | | | | |
| STAT5B | IL2RA | 6777 | NM_012448.3 | NC_000017.11 | MAVWIQAQQLQGEALHQMQALYGQHFP | 44 | Positive | Positive | Positive |
| | | | | | IEVRHYLSQWIESQAWDSVDLDNPQENIK | | Regulator | Regulator | Regulator |
| | | | | | ATQLLEGLVQELQKKAEHQVGEDGFLLKI | | | | |
| | | | | | KLGHYATQLQNTYDRCPMELVRCIRHILY | | | | |
| | | | | | NEQRLVREANNGSSPAGSLADAMSQKHL | | | | |
| | | | | | QINQTFEELRLVTQDTENELKKLQQTQEY | | | | |
| | | | | | FUQYQESLRIQAQFGPLAQLSPQERLSRET | | | | |
| | | | | | ALQQKQVSLEAWLQREAQTLQQYRVELA | | | | |
| | | | | | EKHQKTLQLLRKQQTIILDDELIQWKRRQ | | | | |
| | | | | | QLAGNGGPPEGSLDVLQSWCEKLAEIIWQ | | | | |
| | | | | | NRQQIRRAEHLCQQLPIPGPVEEMLAEVN | | | | |
| | | | | | ATITDIISALVTSTFIIEKQPPQVLKTQTKFA | | | | |
| | | | | | ATVRLLVGGKLNVHMNPPQVKATIISEQQ | | | | |
| | | | | | AKSLLKNENTRNDYSGEILNNCCVMEYH | | | | |
| | | | | | QATGTLSAHFRNMSLKRIKRSDRRGAESV | | | | |
| | | | | | TEEKFTILFESQFSVGGNELVFQVKTLSLP | | | | |
| | | | | | VVVIVHGSQDNNATATVLWDNAFAEPGR | | | | |
| | | | | | VPFAVPDKVLWPQLCEALNMKFKAEVQS | | | | |
| | | | | | NRGLTKENLVFLAQKLFNNSSSHLEDYSG | | | | |
| | | | | | LSVSWSQFNRENLPGRNYTFWQWFDGV | | | | |
| | | | | | MEVLKKHLKPHWNDGAILGFVNKQQAH | | | | |
| | | | | | DLLINKPDGTFLLRFSDSEIGGITIAWKFDS | | | | |
| | | | | | QERMFWNLMPFTTRDFSIRSLADRLGDLN | | | | |
| | | | | | YLIYVFPDRPKDEVYSKYYTPVPCESATA | | | | |
| | | | | | KAVDGYVKPQIKQVVPEFVNASADAGGG | | | | |
| | | | | | SATYMDQAPSPAVCPQAHYNMYPQNPDS | | | | |
| | | | | | VLDTDGDFDLEDTMDVARRVEELLGRPM | | | | |
| | | | | | DSQWIPHAQS | | | | |
| STAT5A | IL2RA | 6776 | NM_003152.3 | NC_000017.11 | MAGWIQAQQLQGDALRQMQVLYGQHFP | 45 | Positive | Positive | Positive |
| | | | | | IEVRHYLAQWIESQPWDAIDLDNPQDRAQ | | Regulator | Regulator | Regulator |
| | | | | | ATQLLEGLVQELQKKAEHQVGEDGFLLKI | | | | |
| | | | | | KLGHYATQLQKTYDRCPLELVRCIRHILY | | | | |
| | | | | | NEQRLVREANNCSSPAGILVDAMSQKHL | | | | |
| | | | | | QINQTFEELRLVTQDTENELKKLQQTQEY | | | | |
| | | | | | FIIQYQESLRIQAQFAQLAQLSPQERLSRET | | | | |
| | | | | | ALQQKQVSLEAWLQREAQTLQQYRVELA | | | | |
| | | | | | EKHQKTLQLLRKQQTIILDDELIQWKRRQ | | | | |
| | | | | | QLAGNGGPPEGSLDVLQSWCEKLAEIIWQ | | | | |
| | | | | | NRQQIRRAEHLCQQLPIPGPVEEMLAEVN | | | | |
| | | | | | ATITDIISALVTSTFIIEKQPPQVLKTQTKFA | | | | |
| | | | | | ATVRLLVGGKLNVHMNPPQVKATIISEQQ | | | | |
| | | | | | AKSLLKNENTRNECSGEILNNCCVMEYH | | | | |
| | | | | | QATGTLSAHFRNMSLKRIKRADRRGAES | | | | |
| | | | | | VTEEKFTVLFESQFSVGSNELVFQVKTLSL | | | | |
| | | | | | PVVVIVHGSQDHNATATVLWDNAFAEPG | | | | |
| | | | | | RVPFAVPDKVLWPQLCEALNMKFKAEVQ | | | | |
| | | | | | SNRGLTKENLVFLAQKLFNNSSSHLEDYS | | | | |
| | | | | | GLSVSWSQFNRENLPGWNYTFWQWEDG | | | | |
| | | | | | VMEVLKKHHKPHWNDGAILGFVNKQQA | | | | |
| | | | | | HDLLINKPDGTFLLRFSDSEIGGITIAWKF | | | | |
| | | | | | DSPERNLWNLKPFTTRDFSIRSLADRLGD | | | | |
| | | | | | LSYLIYVFPDRPKDEVFSKYYTPVLAKAV | | | | |
| | | | | | DGYVKPQIKQVVPEFVNASADAGGSSAT | | | | |
| | | | | | YMDQAPSPAVCPQAPYNMYPQNPDHVL | | | | |
| | | | | | DQDGEFDLDETMDVARHVEELLRRPMDS | | | | |
| | | | | | LDSRLSPPAGLFTSARGSLS | | | | |
| MED12 | IL2RA | 9968 | NM_005120.2 | NC_000023.11 | MAAFGILSYEHRPLKRPRLGPPDVYPQDP | 46 | Positive | Negative | Positive |
| | | | | | KQKEDELTALNVKQGFNNQPAVSGDEHG | | Regulator | Regulator | Regulator |
| | | | | | SAKNVSFNPAKISSNFSSIAEKLRCNTLPD | | | | |
| | | | | | TGRRKPQVNQKDNFWLVTARSQSAINTW | | | | |
| | | | | | FTDLAGTKPLTQLAKKVPIFSKKEEVFGY | | | | |
| | | | | | LAKYTVPVMRAAWLIKMTCAYYAAISET | | | | |
| | | | | | KVKKRHVDPFMEWTQIITKYLWEQLQKM | | | | |
| | | | | | AEYYRPGPAGSGGCGSTIGPLPHDVEVAI | | | | |
| | | | | | RQWDYTEKLAMFMFQDGMLDRHEFLTW | | | | |
| | | | | | VLECFEKIRPGEDELLKLLLPLLLRYSGEF | | | | |
| | | | | | VQSAYLSRRLAYFCTRRLALQLDGVSSHS | | | | |
| | | | | | SHVISAQSTSTLPTTPAPQPPTSSTPSTPFSD | | | | |
| | | | | | LLMCPQHRPLVFGLSCILQTILLCCPSALV | | | | |
| | | | | | WHYSLTDSRIKTGSPLDHLPIAPSNLPMPE | | | | |
| | | | | | GNSAFTQQVRAKLREIEQQIKERGQAVEV | | | | |
| | | | | | RWSFDKCQEATAGFTIGRVLHTLEVLDSH | | | | |
| | | | | | SFERSDFSNSLDSLCNRIFGLGPSKDGHEIS | | | | |
| | | | | | SDDDAVVSLLCEWAVSCKRSGRHRAMV | | | | |
| | | | | | VAKLLEKRQAEIEAERCGESEAADEKGSI | | | | |
| | | | | | ASGSLSAPSAPIFQDVLLQFLDTQAPMLTD | | | | |
| | | | | | PRSESERVEFFNLVLLFCELIRHDVFSHNM | | | | |
| | | | | | YTCTLISRGDLAFGAPGPRPPSPFDDPADD | | | | |
| | | | | | PEHKEAEGSSSSKLEDPGLSESMDIDPSSS | | | | |
| | | | | | VLFEDMEKPDFSLFSPTMPCEGKGSPSPEK | | | | |
| | | | | | PDVEKEVKPPPKEKIEGTLGVLYDQPRHV | | | | |
| | | | | | QYATHFPIPQEESCSHECNQRLVVLFGVG | | | | |
| | | | | | KQRDDARHAIKKITKDILKVLNRKGTAET | | | | |
| | | | | | DQLAPIVPLNPGDLTFLGGEDGQKRRRNR | | | | |
| | | | | | PEAFPTAEDIFAKFQHLSHYDQHQVTAQV | | | | |
| | | | | | SRNVLEQITSFALGMSYHLPLVQHVQFIED | | | | |
| | | | | | LMEYSLSISGLIDFAIQLLNELSVVEAELLL | | | | |
| | | | | | KSSDLVGSYTTSLCLCIVAVLRHYHACLIL | | | | |
| | | | | | NQDQMAQVFEGLCGVVKHGMNRSDGSS | | | | |
| | | | | | AERCILAYLYDLYTSCSHLKNKFGELFSD | | | | |
| | | | | | FCSKVKNTIYCNVEPSESNMRWAPEFMID | | | | |
| | | | | | TLENPAAHTFTYTGLGKSLSENPANRYSF | | | | |
| | | | | | VCNALMHVCVGHHDPDRVNDIAILCAEL | | | | |
| | | | | | TGYCKSLSAEWLGVLKALCCSSNNGTCG | | | | |
| | | | | | FNDLLCNVDVSDLSFHDSLATFVAILIARQ | | | | |
| | | | | | CLLLEDLIRCAAIPSLLNAACSEQDSEPGA | | | | |
| | | | | | RLTCRILLHLFKTPQLNPCQSDGNKPTVGI | | | | |
| | | | | | RSSCDRHLLAASQNRIVDGAVFAVLKAVF | | | | |
| | | | | | VLGDAELKGSGFTVTGGTEELPEEEGGGG | | | | |
| | | | | | SGGRRQGGRNISVETASLDVYAKYVLRSI | | | | |
| | | | | | CQQEWVGERCLKSLCEDSNDLQDPVLSS | | | | |
| | | | | | AQAQRLMQLICYPHRLLDNEDGENPQRQ | | | | |
| | | | | | RIKRILQNLDQWTMRQSSLELQLMIKQTP | | | | |
| | | | | | NNEMNSLLENIAKATIEVFQQSAETGSSSG | | | | |
| | | | | | STASNMPSSSKTKPVLSSLERSGVWLVAP | | | | |
| | | | | | LIAKLPTSVQGHVLKAAGEELEKGQHLGS | | | | |
| | | | | | SSRKERDRQKQKSMSLLSQQPFLSLVLTC | | | | |
| | | | | | LKGQDEQREGLLTSLYSQVHQIVNNWRD | | | | |
| | | | | | DQYLDDCKPKQLMHEALKLRLNLVGGM | | | | |
| | | | | | FDTVQRSTQQTTEWAMLLLEIIISGTVDM | | | | |
| | | | | | QSNNELFTTVLDMLSVLINGTLAADMSSI | | | | |
| | | | | | SQGSMEENKRAYMNLAKKLQKELGERQS | | | | |
| | | | | | DSLEKVRQLLPLPKQTRDVITCEPQGSLID | | | | |
| | | | | | TKGNKIAGEDSIFKKEGLQVSTKQKISPW | | | | |
| | | | | | DLFEGLKPSAPLSWGWFGTVRVDRRVAR | | | | |
| | | | | | GEEQQRLLLYHTHLRPRPRAYYLEPLPLP | | | | |
| | | | | | PEDEEPPAPTLLEPEKKAPEPPKTDKPGAA | | | | |
| | | | | | PPSTEERKKKSTKGKKRSQPATKTEDYG | | | | |
| | | | | | MGPGRSGPYGVTVPPDLLHHPNPGSITHL | | | | |
| | | | | | NYRQGSIGLYTQNQPLPAGGPRVDPYRPV | | | | |
| | | | | | RLPMQKLPTRPTYPGVLPTTMTGVMGLE | | | | |
| | | | | | PSSYKTSVYRQQQPAVPQGQRLRQQLQQ | | | | |
| | | | | | SQGMLGQSSVHQMTPSSSYGLQTSQGYT | | | | |
| | | | | | PYVSHVGLQQHTGPAGTMVPPSYSSQPY | | | | |
| | | | | | QSTHPSTNPTLVDPTRHLQQRPSGYVHQQ | | | | |
| | | | | | APTYGHGLTSTQRFSHQTLQQTPMISTMT | | | | |
| | | | | | PMSAQGVQAGVRSTAILPEQQQQQQQQQ | | | | |
| | | | | | QQHQQQQQQQAAPPQPQPQSQPQFQRQG | | | | |
| | | | | | LQQTQQQQQTAALVRQLQQQLSNTQPQP | | | | |
| | | | | | STNIFGRY | | | | |
| FOXP3 | IL2RA | 50943 | NM_014009.3 | NC_000023.11 | MPNPRPGKPSAPSLALGPSPGASPSWRAA | 47 | Positive | Positive | NS |
| | | | | | PKASDLLGARGPGGTFQGRDLRGGAHAS | | Regulator | Regulator | |
| | | | | | SSSLNPMPPSQLQLPTLPLVMVAPSGARL | | | | |
| | | | | | GPLPHLQALLQDRPHFMHQLSTVDAHAR | | | | |
| | | | | | TPVLQVHPLESPAMISLTPPTTATGVFSLK | | | | |
| | | | | | ARPGLPPGINVASLEWVSREPALLCTFPNP | | | | |
| | | | | | SAPRKDSTLSAVPQSSYPLLANGVCKWPG | | | | |
| | | | | | CEKVFEEPEDFLKHCQADHLLDEKGRAQ | | | | |
| | | | | | CLLQREMVQSLEQQLVLEKEKLSAMQAH | | | | |
| | | | | | LAGKMALTKASSVASSDKGSCCIVAAGS | | | | |
| | | | | | QGPVVPAWSGPREAPDSLFAVRRHLWGS | | | | |
| | | | | | HGNSTFPEFLHNMDYFKFHNMRPPFTYAT | | | | |
| | | | | | LIRWAILEAPEKQRTLNEIYHWFTRMFAF | | | | |
| | | | | | FRNHPATWKNAIRHNLSLHKCFVRVESEK | | | | |
| | | | | | GAVWTVDELEFRKKRSQRPSRCSNPTPGP | | | | |
| FLI1 | IL2RA | 2313 | NM_002017.4 | NC_000011.10 | MDGTIKEALSVVSDDQSLEDSAYGAAAH | 48 | Positive | NS | NS |
| | | | | | LPKADMTASGSPDYGQPHKINPLPPQQEW | | Regulator | | |
| | | | | | INQPVRVNVKREYDHMNGSRESPVDCSV | | | | |
| | | | | | SKCSKLVGGGESNPMNYNSYMDEKNGPP | | | | |
| | | | | | PPNMTTNERRVIVPADPTLWTQEHVRQW | | | | |
| | | | | | LEWAIKEYSLMEIDTSFFQNMDGKELCK | | | | |
| | | | | | MNKEDFLRATTLYNTEVLLSHLSYLRESS | | | | |
| | | | | | LLAYNTTSHTDQSSRLSVKEDPSYDSVRR | | | | |
| | | | | | GAWGNNMNSGLNKSPPLGGAQTISKNTE | | | | |
| | | | | | QRPQPDPYQILGPTSSRLANPGSGQIQLW | | | | |
| | | | | | QFLLELLSDSANASCITWEGINGEFKMTD | | | | |
| | | | | | PDEVARRWGERKSKPNMNYDKLSRALR | | | | |
| | | | | | YYYDKNIMTKVHGKRYAYKFDFHGIAQA | | | | |
| | | | | | LQPHPTESSMYKYPSDISYMPSYHAHQQK | | | | |
| | | | | | VNFVPPHPSSMPVTSSSFFGAASQYWTSP | | | | |
| | | | | | TGGIYPNPNVPRHPNTHVPSHLGSYY | | | | |
| ETS1 | IL2RA | 2113 | NM_005238.3 | NC_000011.10 | MKAAVDLKPTLTIIKTEKVDLELFPSPDM | 49 | Positive | Positive | Positive |
| | | | | | ECADVPLLTPSSKEMMSQALKATFSGFTK | | Regulator | Regulator | Regulator |
| | | | | | EQQRLGIPKDPRQWTETHVRDWVMWAV | | | | |
| | | | | | NEFSLKGVDFQKFCMNGAALCALGKDCF | | | | |
| | | | | | LELAPDFVGDILWEHLEILQKEDVKPYQV | | | | |
| | | | | | NGVNPAYPESRYTSDYFISYGIEHAQCVPP | | | | |
| | | | | | SEFSEPSFITESYQTLHPISSEELLSLKYEND | | | | |
| | | | | | YPSVILRDPLQTDTLQNDYFAIKQEVVTP | | | | |
| | | | | | DNMCMGRTSRGKLGGQDSFESIESYDSC | | | | |
| | | | | | DRLTQSWSSQSSENSLQRVPSYDSEDSED | | | | |
| | | | | | YPAALPNHKPKGTFKDYVRDRADLNKDK | | | | |
| | | | | | PVIPAAALAGYTGSGPIQLWQFLLELLTD | | | | |
| | | | | | KSCQSFISWTGDGWEFKLSDPDEVARRW | | | | |
| | | | | | GKRKNKPKMNYEKLSRGLRYYYDKNIH | | | | |
| | | | | | KTAGKRYVYRFVCDLQSLLGYTPEELHA | | | | |
| | | | | | MLDVKPDADE | | | | |
| ATXN7L3 | IL2RA | 56970 | NM_ | NC_000017.11 | MKMEEMSLSGLDNSKLEAIAQEIYADLVE | 50 | Positive | Negative | NS |
| | | | 001098833.1 | | DSCLGFCFEVHRAVKCGYFFLDDTDPDS | | Regulator | Regulator | |
| | | | | | MKDFEIVDQPGLDIFGQVFNQWKSKECV | | | | |
| | | | | | CPNCSRSIAASRFAPHLEKCLGMGRNSSRI | | | | |
| | | | | | ANRRIANSNNMNKSESDQEDNDDINDND | | | | |
| | | | | | WSYGSEKKAKKRKSDKNPNSPRRSKSLK | | | | |
| | | | | | HKNGELSNSDPFKYNNSTGISYETLGPEEL | | | | |
| | | | | | RSLLTTQCGVISEHTKKMCTRSLRCPQHT | | | | |
| | | | | | DEQRRTVRIYFLGPSAVLPEVESSLDNDSF | | | | |
| | | | | | DMTDSQALISRLQWDGSSDLSPSDSGSSK | | | | |
| | | | | | TSENQGWGLGTNSSESRKTKKKKSHLSL | | | | |
| | | | | | VGTASGLGSNKKKKPKPPAPPTPSIYDDIN | | | | |
| KMT2A | IL2RA | 4297 | NM_005933.3 | NC_000011.10 | MAHSCRWRFPARPGTTGGGGGGGRRGL | 51 | Positive | Positive | NS |
| | | | | | GGAPRQRVPALLLPPGPPVGGGGPGAPPS | | Regulator | Regulator | |
| | | | | | PPAVAAAAAAAGSSGAGVPGGAAAASA | | | | |
| | | | | | ASSSSASSSSSSSSSASSGPALLRVGPGFDA | | | | |
| | | | | | ALQVSAAIGTNLRRFRAVFGESGGGGGSG | | | | |
| | | | | | EDEQFLGFGSDEEVRVRSPTRSPSVKTSPR | | | | |
| | | | | | KPRGRPRSGSDRNSAILSDPSVFSPLNKSE | | | | |
| | | | | | TKSGDKIKKKDSKSIEKKRGRPPTFPGVKI | | | | |
| | | | | | KITHGKDISELPKGNKEDSLKKIKRTPSAT | | | | |
| | | | | | FQQATKIKKLRAGKLSPLKSKFKTGKLQI | | | | |
| | | | | | GRKGVQIVRRRGRPPSTERIKTPSGLLINS | | | | |
| | | | | | ELEKPQKVRKDKEGTPPLTKEDKTVVRQS | | | | |
| | | | | | PRRIKPVRIIPSSKRTDATIAKQLLQRAKK | | | | |
| | | | | | GAQKKIEKEAAQLQGRKVKTQVKNIRQFI | | | | |
| | | | | | MPVVSAISSRIIKTPRRFIEDEDYDPPIKIAR | | | | |
| | | | | | LESTPNSRFSAPSCGSSEKSSAASQHSSQM | | | | |
| | | | | | SSDSSRSSSPSVDTSTDSQASEEIQVLPEER | | | | |
| | | | | | SDTPEVHPPLPISQSPENESNDRRSRRYSV | | | | |
| | | | | | SERSFGSRTTKKLSTLQSAPQQQTSSSPPPP | | | | |
| | | | | | LLTPPPPLQPASSISDHTPWLMPPTIPLASP | | | | |
| | | | | | FLPASTAPMQGKRKSILREPTFRWTSLKH | | | | |
| | | | | | SRSEPQYFSSAKYAKEGLIRKPIFDNFRPPP | | | | |
| | | | | | LTPEDVGFASGFSASGTAASARLFSPLHSG | | | | |
| | | | | | TRFDMHKRSPLLRAPRFTPSEAHSRIFESV | | | | |
| | | | | | TLPSNRTSAGTSSSGVSNRKRKRKVFSPIR | | | | |
| | | | | | SEPRSPSHSMRTRSGRLSSSELSPLTPPSSV | | | | |
| | | | | | SSSLSISVSPLATSALNPTFTFPSHSLTQSG | | | | |
| | | | | | ESAEKNQRPRKQTSAPAEPFSSSSPTPLFP | | | | |
| | | | | | WFTPGSQTERGRNKDKAPEELSKDRDAD | | | | |
| | | | | | KSVEKDKSRERDREREKENKRESRKEKR | | | | |
| | | | | | KKGSEIQSSSALYPVGRVSKEKVVGEDVA | | | | |
| | | | | | TSSSAKKATGRKKSSSHDSGTDITSVTLG | | | | |
| | | | | | DTTAVKTKILIKKGRGNLEKTNLDLGPTA | | | | |
| | | | | | PSLEKEKTLCLSTPSSSTVKHSTSSIGSMLA | | | | |
| | | | | | QADKLPMTDKRVASLLKKAKAQLCKIEK | | | | |
| | | | | | SKSLKQTDQPKAQGQESDSSETSVRGPRI | | | | |
| | | | | | KHVCRRAAVALGRKRAVFPDDMPTLSAL | | | | |
| | | | | | PWEEREKILSSMGNDDKSSIAGSEDAEPL | | | | |
| | | | | | APPIKPIKPVTRNKAPQEPPVKKGRRSRRC | | | | |
| | | | | | GQCPGCQVPEDCGVCTNCLDKPKFGGRN | | | | |
| | | | | | IKKQCCKMRKCQNLQWMPSKAYLQKQA | | | | |
| | | | | | KAVKKKEKKSKTSEKKDSKESSVVKNVV | | | | |
| | | | | | DSSQKPTPSAREDPAPKKSSSEPPPRKPVE | | | | |
| | | | | | EKSEEGNVSAPGPESKQATTPASRKSSKQ | | | | |
| | | | | | VSQPALVIPPQPPTTGPPRKEVPKTTPSEPK | | | | |
| | | | | | KKQPPPPESGPEQSKQKKVAPRPSIPVKQK | | | | |
| | | | | | PKEKEKPPPVNKQENAGTLNILSTLSNGN | | | | |
| | | | | | SSKQKIPADGVHRIRVDFKEDCEAENVWE | | | | |
| | | | | | MGGLGILTSVPITPRVVCFLCASSGHVEFV | | | | |
| | | | | | YCQVCCEPFHKFCLEENERPLEDQLENWC | | | | |
| | | | | | CRRCKFCHVCGRQHQATKQLLECNKCRN | | | | |
| | | | | | SYHPECLGPNYPTKPTKKKKVWICTKCVR | | | | |
| | | | | | CKSCGSTTPGKGWDAQWSHDFSLCHDCA | | | | |
| | | | | | KLFAKGNFCPLCDKCYDDDDYESKMMQ | | | | |
| | | | | | CGKCDRWVHSKCENLSDEMYEILSNLPES | | | | |
| | | | | | VAYTCVNCTERHPAEWRLALEKELQISLK | | | | |
| | | | | | QVLTALLNSRTTSHLLRYRQAAKPPDLNP | | | | |
| | | | | | ETEESIPSRSSPEGPDPPVLTEVSKQDDQQ | | | | |
| | | | | | PLDLEGVKRKMDQGNYTSVLEFSDDIVKI | | | | |
| | | | | | IQAAINSDGGQPEIKKANSMVKSFFIRQME | | | | |
| | | | | | RVFPWFSVKKSRFWEPNKVSSNSGMLPN | | | | |
| | | | | | AVLPPSLDHNYAQWQEREENSHTEQPPL | | | | |
| | | | | | MKKIIPAPKPKGPGEPDSPTPLHPPTPPILS | | | | |
| | | | | | TDRSREDSPELNPPPGIEDNRQCALCLTYG | | | | |
| | | | | | DDSANDAGRLLYIGQNEWTHVNCALWS | | | | |
| | | | | | AEVFEDDDGSLKNVHMAVIRGKQLRCEF | | | | |
| | | | | | CQKPGATVGCCLTSCTSNYHFMCSRAKN | | | | |
| | | | | | CVFLDDKKVYCQRHRDLIKGEVVPENGF | | | | |
| | | | | | EVFRRVFVDFEGISLRRKFLNGLEPENIHM | | | | |
| | | | | | MIGSMTIDCLGILNDLSDCEDKLFPIGYQC | | | | |
| | | | | | SRVYWSTTDARKRCVYTCKIVECRPPVVE | | | | |
| | | | | | PDINSTVEHDENRTIAHSPTSFTESSSKESQ | | | | |
| | | | | | NTAEHSPPSPDRPPHSQTSGSCYYHVISKV | | | | |
| | | | | | PRIRTPSYSPTQRSPGCRPLPSAGSPTPTTH | | | | |
| | | | | | EIVTVGDPLLSSGLRSIGSRRHSTSSLSPQR | | | | |
| | | | | | SKLRIMSPMRTGNTYSRNNVSSVSTTGTA | | | | |
| | | | | | TDLESSAKVVDHVLGPLNSSTSLGQNTST | | | | |
| | | | | | SSNLQRTVVTVGNKNSHLDGSSSSEMKQS | | | | |
| | | | | | SASDLVSKSSSLKGEKTKVLSSKSSEGSAH | | | | |
| | | | | | NVAYPGIPKLAPQVHNTTSRELNVSKIGSF | | | | |
| | | | | | AEPSSVSFSSKEALSFPHLHLRGQRNDRD | | | | |
| | | | | | QHTDSTQSANSSPDEDTEVKTLKLSGMSN | | | | |
| | | | | | RSSIINEHMGSSSRDRRQKGKKSCKETFKE | | | | |
| | | | | | KHSSKSFLEPGQVTTGEEGNLKPEFMDEV | | | | |
| | | | | | LTPEYMGQRPCNNVSSDKIGDKGLSMPG | | | | |
| | | | | | VPKAPPMQVEGSAKELQAPRKRTVKVTL | | | | |
| | | | | | TPLKMENESQSKNALKESSPASPLQIESTS | | | | |
| | | | | | PTEPISASENPGDGPVAQPSPNNTSCQDSQ | | | | |
| | | | | | SNNYQNLPVQDRNLMLPDGPKPQEDGSF | | | | |
| | | | | | KRRYPRRSARARSNMFFGLTPLYGVRSY | | | | |
| | | | | | GEEDIPFYSSSTGKKRGKRSAEGQVDGAD | | | | |
| | | | | | DLSTSDEDDLYYYNFTRTVISSGGEERLAS | | | | |
| | | | | | HNLFREEEQCDLPKISQLDGVDDGTESDT | | | | |
| | | | | | SVTATTRKSSQIPKRNGKENGTENLKIDRP | | | | |
| | | | | | EDAGEKEHVTKSSVGHKNEPKMDNCHSV | | | | |
| | | | | | SRVKTQGQDSLEAQLSSLESSRRVHTSTPS | | | | |
| | | | | | DKNLLDTYNTELLKSDSDNNNSDDCGNIL | | | | |
| | | | | | PSDIMDFVLKNTPSMQALGESPESSSSELL | | | | |
| | | | | | NLGEGLGLDSNREKDMGLFEVFSQQLPTT | | | | |
| | | | | | EPVDSSVSSSISAEEQFELPLELPSDLSVLT | | | | |
| | | | | | TRSPTVPSQNPSRLAVISDSGEKRVTITEKS | | | | |
| | | | | | VASSESDPALLSPGVDPTPEGHMTPDHFIQ | | | | |
| | | | | | GHMDADHISSPPCGSVEQGHGNNQDLTR | | | | |
| | | | | | NSSTPGLQVPVSPTVPIQNQKYVPNSTDSP | | | | |
| | | | | | GPSQISNAAVQTTPPHLKPATEKLIVVNQ | | | | |
| | | | | | NMQPLYVLQTLPNGVTQKIQLTSSVSSTP | | | | |
| | | | | | SVMETNTSVLGPMGGGLTLTTGLNPSLPT | | | | |
| | | | | | SQSLFPSASKGLLPMSHHQHLHSFPAATQ | | | | |
| | | | | | SSFPPNISNPPSGLLIGVQPPPDPQLLVSESS | | | | |
| | | | | | QRTDLSTTVATPSSGLKKRPISRLQTRKNK | | | | |
| | | | | | KLAPSSTPSNIAPSDVVSNMTLINFTPSQLP | | | | |
| | | | | | NHPSLLDLGSLNTSSHRTVPNIIKRSKSSIM | | | | |
| | | | | | YFEPAPLLPQSVGGTAATAAGTSTISQDTS | | | | |
| | | | | | HLTSGSVSGLASSSSVLNVVSMQTTTTPTS | | | | |
| | | | | | SASVPGHVILTNPRLLGTPDIGSISNLLIKA | | | | |
| | | | | | SQQSLGIQDQPVALPPSSGMFPQLGTSQTP | | | | |
| | | | | | STAAITAASSICVLPSTQTTGITAASPSGEA | | | | |
| | | | | | DEHYQLQHVNQLLASKTGIHSSQRDLDSA | | | | |
| | | | | | SGPQVSNFTQTVDAPNSMGLEQNKALSS | | | | |
| | | | | | AVQASPTSPGGSPSSPSSGQRSASPSVPGP | | | | |
| | | | | | TKPKPKTKRFQLPLDKGNGKKHKVSHLR | | | | |
| | | | | | TSSSEAHIPDQETTSLTSGTGTPGAEAEQQ | | | | |
| | | | | | DTASVEQSSQKECGQPAGQVAVLPEVQV | | | | |
| | | | | | TQNPANEQESAEPKTVEEEESNFSSPLML | | | | |
| | | | | | WLQQEQKRKESITEKKPKKGLVFEISSDD | | | | |
| | | | | | GFQICAESIEDAWKSLTDKVQEARSNARL | | | | |
| | | | | | KQLSFAGVNGLRMLGILHDAVVFLIEQLS | | | | |
| | | | | | GAKHCRNYKFRFHKPEEANEPPLNPHGSA | | | | |
| | | | | | RAEVHLRKSAFDMFNFLASKHRQPPEYNP | | | | |
| | | | | | NDEEEEEVQLKSARRATSMDLPMPMRFR | | | | |
| | | | | | HLKKTSKEAVGVYRSPIHGRGLFCKRNID | | | | |
| | | | | | AGEMVIEYAGNVIRSIQTDKREKYYDSKG | | | | |
| | | | | | IGCYMFRIDDSEVVDATMHGNAARFINHS | | | | |
| | | | | | CEPNCYSRVINIDGQKHIVIFAMRKIYRGE | | | | |
| | | | | | ELTYDYKFPIEDASNKLPCNCGAKKCRKF | | | | |
| | | | | | LN | | | | |
| ZEB1 | IL2RA | 6935 | NM_030751.5 | NC_000010.11 | MADGPRCKRRKQANPRRNNVTNYNTVV | 52 | Positive | NS | NS |
| | | | | | ETNSDSDDEDKLHIVEEESVTDAADCEGV | | Regulator | | |
| | | | | | PEDDLPTDQTVLPGRSSEREGNAKNCWE | | | | |
| | | | | | DDRKEGQEILGPEAQADEAGCTVKDDEC | | | | |
| | | | | | ESDAENEQNHDPNVEEFLQQQDTAVIFPE | | | | |
| | | | | | APEEDQRQGTPEASGHDENGTPDAFSQLL | | | | |
| | | | | | TCPYCDRGYKRFTSLKEHIKYRHEKNEDN | | | | |
| | | | | | FSCSLCSYTFAYRTQLERHMTSHKSGRDQ | | | | |
| | | | | | RHVTQSGCNRKFKCTECGKAFKYKHHLK | | | | |
| | | | | | EHLRIHSGEKPYECPNCKKRFSHSGSYSSH | | | | |
| | | | | | ISSKKCISLIPVNGRPRTGLKTSQCSSPSLS | | | | |
| | | | | | ASPGSPTRPQIRQKIENKPLQEQLSVNQIK | | | | |
| | | | | | TEPVDYEFKPIVVASGINCSTPLQNGVFTG | | | | |
| | | | | | GGPLQATSSPQGMVQAVVLPTVGLVSPISI | | | | |
| | | | | | NLSDIQNVLKVAVDGNVIRQVLENNQAN | | | | |
| | | | | | LASKEQETINASPIQQGGHSVISAISLPLVD | | | | |
| | | | | | QDGTTKIIINYSLEQPSQLQVVPQNLKKEN | | | | |
| | | | | | PVATNSCKSEKLPEDLTVKSEKDKSFEGG | | | | |
| | | | | | VNDSTCLLCDDCPGDINALPELKHYDLKQ | | | | |
| | | | | | PTQPPPLPAAEAEKPESSVSSATGDGNLSP | | | | |
| | | | | | SQPPLKNLLSLLKAYYALNAQPSAEELSKI | | | | |
| | | | | | ADSVNLPLDVVKKWFEKMQAGQISVQSS | | | | |
| | | | | | EPSSPEPGKVNIPAKNNDQPQSANANEPQ | | | | |
| | | | | | DSTVNLQSPLKMINSPVLPVGSTTNGSRS | | | | |
| | | | | | STPSPSPLNLSSSRNTQGYLYTAEGAQEEP | | | | |
| | | | | | QVEPLDLSLPKQQGELLERSTITSVYQNSV | | | | |
| | | | | | YSVQEEPLNLSCAKKEPQKDSCVTDSEPV | | | | |
| | | | | | VNVIPPSANPINIAIPTVTAQLPTIVAIADQ | | | | |
| | | | | | NSVPCLRALAANKQTILIPQVAYTYSTTVS | | | | |
| | | | | | PAVQEPPLKVIQPNGNQDERQDTSSEGVS | | | | |
| | | | | | NVEDQNDSDSTPPKKKMRKTENGMYAC | | | | |
| | | | | | DLCDKIFQKSSSLLRHKYEHTGKRPHECGI | | | | |
| | | | | | CKKAFKHKHHLIEHMRLHSGEKPYQCDK | | | | |
| | | | | | CGKRFSHSGSYSQHMNHRYSYCKREAEE | | | | |
| | | | | | RDSTEQEEAGPEILSNEHVGARASPSQGD | | | | |
| | | | | | SDERESLTREEDEDSEKEEEEEDKEMEEL | | | | |
| | | | | | QEEKECEKPQGDEEEEEEEEEVEEEEVEE | | | | |
| | | | | | AENEGEEAKTEGLMKDDRAESQASSLGQ | | | | |
| | | | | | KVGESSEQVSEEKTNEA | | | | |
| MBD2 | IL2RA | 8932 | NM_003927.4 | NC_000018.10 | MRAHPGGGRCCPEQEEGESAAGGSGAGG | 53 | Positive | NS | NS |
| | | | | | DSAIEQGGQGSALAPSPVSGVRREGARGG | | Regulator | | |
| | | | | | GRGRGRWKQAGRGGGVCGRGRGRGRGR | | | | |
| | | | | | GRGRGRGRGRGRPPSGGSGLGGDGGGCG | | | | |
| | | | | | GGGSGGGGAPRREPVPFPSGSAGPGPRGP | | | | |
| | | | | | RATESGKRMDCPALPPGWKKEEVIRKSG | | | | |
| | | | | | LSAGKSDVYYFSPSGKKFRSKPQLARYLG | | | | |
| | | | | | NTVDLSSFDFRTGKMMPSKLQKNKQRLR | | | | |
| | | | | | NDPLNQNKGKPDLNTTLPIRQTASIFKQPV | | | | |
| | | | | | TKVTNHPSNKVKSDPQRMNEQPRQLFWE | | | | |
| | | | | | KRLQGLSASDVTEQIIKTMELPKGLQGVG | | | | |
| | | | | | PGSNDETLLSAVASALHTSSAPITGQVSAA | | | | |
| | | | | | VEKNPAVWLNTSQPLCKAFIVIDEDIRKQ | | | | |
| | | | | | EERVQQVRKKLEEALMADILSRAADTEE | | | | |
| | | | | | MDIEMDSGDEA | | | | |
| CIC | IL2RA | 23152 | NM_015125.4 | NC_000019.10 | MYSAHRPLMPASSAASRGLGMFVWTNV | 54 | Positive | Positive | NS |
| | | | | | EPRSVAVFPWHSLVPFLAPSQPDPSVQPSE | | Regulator | Regulator | |
| | | | | | AQQPASHPVASNQSKEPAESAAVAHERPP | | | | |
| | | | | | GGTGSADPERPPGATCPESPGPGPPHPLGV | | | | |
| | | | | | VESGKGPPPTTEEEASGPPGEPRLDSETES | | | | |
| | | | | | DHDDAFLSIMSPEIQLPLPPGKRRTQSLSA | | | | |
| | | | | | LPKERDSSSEKDGRSPNKREKDHIRRPMN | | | | |
| | | | | | AFMIFSKRHRALVHQRHPNQDNRTVSKIL | | | | |
| | | | | | GEWWYALGPKEKQKYHDLAFQVKEAHF | | | | |
| | | | | | KAHPDWKWCNKDRKKSSSEAKPTSLGLA | | | | |
| | | | | | GGHKETRERSMSETGTAAAPGVSSELLSV | | | | |
| | | | | | AAQTLLSSDTKAPGSSSCGAERLHTVGGP | | | | |
| | | | | | GSARPRAFSHSGVHSLDGGEVDSQALQEL | | | | |
| | | | | | TQMVSGPASYSGPKPSTQYGAPGPFAAPG | | | | |
| | | | | | EGGALAATGRPPLLPTRASRSQRAASEDM | | | | |
| | | | | | TSDEERMVICEEEGDDDVIADDGFGTTDI | | | | |
| | | | | | DLKCKERVTDSESGDSSGEDPEGNKGFGR | | | | |
| | | | | | KVFSPVIRSSFTHCRPPLDPEPPGPPDPPVA | | | | |
| | | | | | FGKGYGSAPSSSASSPASSSASAATSFSLG | | | | |
| | | | | | SGTFKAQESGQGSTAGPLRPPPPGAGGPA | | | | |
| | | | | | TPSKATRFLPMDPATFRRKRPESVGGLEPP | | | | |
| | | | | | GPSVIAAPPSGGGNILQTLVLPPNKEEQEG | | | | |
| | | | | | GGARVPSAPAPSLAYGAPAAPLSRPAATM | | | | |
| | | | | | VTNVVRPVSSTPVPIASKPFPTSGRAEASP | | | | |
| | | | | | NDTAGARTEMGTGSRVPGGSPLGVSLVY | | | | |
| | | | | | SDKKSAAATSPAPHLVAGPLLGTVGKAP | | | | |
| | | | | | ATVTNLLVGTPGYGAPAPPAVQFIAQGAP | | | | |
| | | | | | GGGTTAGSGAGAGSGPNGPVPLGILQPGA | | | | |
| | | | | | LGKAGGITQVQYILPTLPQQLQVAPAPAP | | | | |
| | | | | | APGTKAAAPSGPAPTTSIRFTLPPGTSTNG | | | | |
| | | | | | KVLAATAPTPGIPILQSVPSAPPPKAQSVSP | | | | |
| | | | | | VQAPPPGGSAQLLPGKVLVPLAAPSMSVR | | | | |
| | | | | | GGGAGQPLPLVSPPFSVPVQNGAQPPSKII | | | | |
| | | | | | QLTPVPVSTPSGLVPPLSPATLPGPTSQPQ | | | | |
| | | | | | KVLLPSSTRITYVQSAGGHALPLGTSPASS | | | | |
| | | | | | QAGTVTSYGPTSSVALGFTSLGPSGPAFV | | | | |
| | | | | | QPLLSAGQAPLLAPGQVGVSPVPSPQLPP | | | | |
| | | | | | ACAAPGGPVITAFYSGSPAPTSSAPLAQPS | | | | |
| | | | | | QAPPSLVYTVATSTTPPAATILPKGPPAPA | | | | |
| | | | | | TATPAPTSPFPSATAGSMTYSLVAPKAQR | | | | |
| | | | | | PSPKAPQKVKAAIASIPVGSFEAGASGRPG | | | | |
| | | | | | PAPRQPLEPGPVREPTAPESELEGQPTPPA | | | | |
| | | | | | PPPLPETWTPTARSSPPLPPPAEERTSAKGP | | | | |
| | | | | | ETMASKFPSSSSDWRVPGQGLENRGEPPT | | | | |
| | | | | | PPSPAPAPAVAPGGSSESSSGRAAGDTPER | | | | |
| | | | | | KEAAGTGKKVKVRPPPLKKTFDSVDNRV | | | | |
| | | | | | LSEVDFEERFAELPEFRPEEVLPSPTLQSLA | | | | |
| | | | | | TSPRAILGSYRKKRKNSTDLDSAPEDPTSP | | | | |
| | | | | | KRKMRRRSSCSSEPNTPKSAKCEGDIFTFD | | | | |
| | | | | | RTGTEAEDVLGELEYDKVPYSSLRRTLDQ | | | | |
| | | | | | RRALVMQLFQDHGFFPSAQATAAFQARY | | | | |
| | | | | | ADIFPSKVCLQLKIREVRQKIMQAATPTEQ | | | | |
| | | | | | PPGAEAPLPVPPPTGTAAAPAPTPSPAGGP | | | | |
| | | | | | DPTSPSSDSGTAQAAPPLPPPPESGPGQPG | | | | |
| | | | | | WEGAPQPSPPPPGPSTAATGR | | | | |
| TAF5L | IL2RA | 27097 | NM_014409.3 | NC_000001.11 | MKRVRTEQIQMAVSCYLKRRQYVDSDGP | 55 | Positive | NS | Positive |
| | | | | | LKQGLRLSQTAEEMAANLTVQSESGCANI | | Regulator | | Regulator |
| | | | | | VSAAPCQAEPQQYEVQFGRLRNFLTDSDS | | | | |
| | | | | | QHSHEVMPLLYPLFVYLHLNLVQNSPKST | | | | |
| | | | | | VESFYSRFHGMFLQNASQKDVIEQLQTTQ | | | | |
| | | | | | TIQDILSNFKLRAFLDNKYVVRLQEDSYN | | | | |
| | | | | | YLIRYLQSDNNTALCKVLTLHIHLDVQPA | | | | |
| | | | | | KRTDYQLYASGSSSRSENNGLEPPDMPSPI | | | | |
| | | | | | LQNEAALEVLQESIKRVKDGPPSLTTICFY | | | | |
| | | | | | AFYNTEQLLNTAEISPDSKLLAAGEDNSCI | | | | |
| | | | | | KLWSLRSKKLKSEPHQVDVSRIHLACDIL | | | | |
| | | | | | EEEDDEDDNAGTEMKILRGHCGPVYSTRF | | | | |
| | | | | | LADSSGLLSCSEDMSIRYWDLGSFINTVL | | | | |
| | | | | | YQGHAYPVWDLDISPYSLYFASGSHDRT | | | | |
| | | | | | ARLWSFDRTYPLRIYAGHLADVDCVKFH | | | | |
| | | | | | PNSNYLATGSTDKTVRLWSAQQGNSVRL | | | | |
| | | | | | FTGHRGPVLSLAFSPNGKYLASAGEDQRL | | | | |
| | | | | | KLWDLASGTLYKELRGHTDNITSLTFSPD | | | | |
| | | | | | SGLIASASMDNSVRVWDIRNTYCSAPADG | | | | |
| | | | | | SSSELVGVYTGQMSNVLSVQFMACNLLL | | | | |
| | | | | | VTGITQENQEH | | | | |
| USP22 | IL2RA | 23326 | NM_015276.1 | NC_000017.11 | MVSRPEPEGEAMDAELAVAPPGCSHLGSF | 56 | Positive | NS | NS |
| | | | | | KVDNWKQNLRAIYQCFVWSGTAEARKR | | Regulator | | |
| | | | | | KAKSCICHVCGVHLNRLHSCLYCVFFGCF | | | | |
| | | | | | TKKHIHEHAKAKRHNLAIDLMYGGIYCFL | | | | |
| | | | | | CQDYIYDKDMEIIAKEEQRKAWKMQGVG | | | | |
| | | | | | EKFSTWEPTKRELELLKHNPKRRKITSNC | | | | |
| | | | | | TIGLRGLINLGNTCFMNCIVQALTHTPLLR | | | | |
| | | | | | DFFLSDRHRCEMQSPSSCLVCEMSSLFQE | | | | |
| | | | | | FYSGHRSPHIPYKLLHLVWTHARHLAGYE | | | | |
| | | | | | QQDAHEFLIAALDVLHRHCKGDDNGKKA | | | | |
| | | | | | NNPNHCNCIIDQIFTGGLQSDVTCQVCHG | | | | |
| | | | | | VSTTIDPFWDISLDLPGSSTPFWPLSPGSEG | | | | |
| | | | | | NVVNGESHVSGTTTLTDCLRRFTRPEHLG | | | | |
| | | | | | SSAKIKCSGCHSYQESTKQLTMKKLPIVA | | | | |
| | | | | | CFHLKRFEHSAKLRRKITTYVSFPLELDM | | | | |
| | | | | | TPFMASSKESRMNGQYQQPTDSLNNDNK | | | | |
| | | | | | YSLFAVVNHQGTLESGHYTSFIRQHKDQ | | | | |
| | | | | | WFKCDDANTKASIKDVLDSEGYLLFYHK | | | | |
| | | | | | QFLEYE | | | | |
| MED30 | IL2RA | 90390 | NM_080651.3 | NC_000008.11 | MSTPPLAASGMAPGPFAGPQAQQAAREV | 57 | Positive | NS | NS |
| | | | | | NTASLCRIGQETVQDIVYRTMEIFQLLRN | | Regulator | | |
| | | | | | MQLPNGVTYHTGTYQDRLTKLQDNLRQL | | | | |
| | | | | | SVLFRKLRLVYDKCNENCGGMDPIPVEQL | | | | |
| | | | | | IPYVEEDGSKNDDRAGPPRFASEERREIAE | | | | |
| | | | | | VNKKLKQKNQQLKQIMDQLRNLIWDINA | | | | |
| | | | | | MLAMRN | | | | |
| IKZF1 | IL2RA | 10320 | NM_006060.5 | NC_000007.14 | MDADEGQDMSQVSGKESPPVSDTPDEGD | 58 | Positive | Positive | NS |
| | | | | | EPMPIPEDLSTTSGGQQSSKSDRVVASNV | | Regulator | Regulator | |
| | | | | | KVETQSDEENGRACEMNGEECAEDLRML | | | | |
| | | | | | DASGEKMNGSHRDQGSSALSGVGGIRLP | | | | |
| | | | | | NGKLKCDICGIICIGPNVLMVHKRSHTGE | | | | |
| | | | | | RPFQCNQCGASFTQKGNLLRHIKLHSGEK | | | | |
| | | | | | PFKCHLCNYACRRRDALTGHLRTHSVGK | | | | |
| | | | | | PHKCGYCGRSYKQRSSLEEHKERCHNYL | | | | |
| | | | | | ESMGLPGTLYPVIKEETNHSEMAEDLCKI | | | | |
| | | | | | GSERSLVLDRLASNVAKRKSSMPQKFLG | | | | |
| | | | | | DKGLSDTPYDSSASYEKENEMMKSHVMD | | | | |
| | | | | | QAINNAINYLGAESLRPLVQTPPGGSEVVP | | | | |
| | | | | | VISPMYQLHKPLAEGTPRSNHSAQDSAVE | | | | |
| | | | | | NLLLLSKAKLVPSEREASPSNSCQDSTDTE | | | | |
| | | | | | SNNEEQRSGLIYLTNHIAPHARNGLSLKEE | | | | |
| | | | | | HRAYDLLRAASENSQDALRVVSTSGEQM | | | | |
| | | | | | KVYKCEHCRVLFLDHVMYTIHMGCHGFR | | | | |
| | | | | | DPFECNMCGYHSQDRYEFSSHITRGEHRF | | | | |
| | | | | | HMS | | | | |
| MED11 | IL2RA | 400569 | NM_ | NC_000017.11 | MATYSLANERLRALEDIEREIGAILQNAG | 59 | Positive | Negative | NS |
| | | | 001001683.3 | | TVILELSKEKTNERLLDRQAAAFTASVQH | | Regulator | Regulator | |
| | | | | | VEAELSAQIRYLTQVATGQPHEGSSYSSR | | | | |
| | | | | | KDCQMALKRVDYARLKLSDVARTCEQM | | | | |
| | | | | | LEN | | | | |
| RXRB | IL2RA | 6257 | NM_021976.4 | NC_000006.12 | MSWAARPPFLPQRHAAGQCGPVGVRKE | 60 | Positive | Positive | NS |
| | | | | | MHCGVASRWRRRRPWLDPAAAAAAAVA | | Regulator | Regulator | |
| | | | | | GGEQQTPEPEPGEAGRDGMGDSGRDSRS | | | | |
| | | | | | PDSSSPNPLPQGVPPPSPPGPPLPPSTAPSL | | | | |
| | | | | | GGSGAPPPPPMPPPPLGSPFPVISSSMGSPG | | | | |
| | | | | | LPPPAPPGFSGPVSSPQINSTVSLPGGGSGP | | | | |
| | | | | | PEDVKPPVLGVRGLHCPPPPGGPGAGKRL | | | | |
| | | | | | CAICGDRSSGKHYGVYSCEGCKGFFKRTI | | | | |
| | | | | | RKDLTYSCRDNKDCTVDKRQRNRCQYCR | | | | |
| | | | | | YQKCLATGMKREAVQEERQRGKDKDGD | | | | |
| | | | | | GEGAGGAPEEMPVDRILEAELAVEQKSD | | | | |
| | | | | | QGVEGPGGTGGSGSSPNDPVTNICQAADK | | | | |
| | | | | | QLFTLVEWAKRIPHFSSLPLDDQVILLRAG | | | | |
| | | | | | WNELLIASFSHRSIDVRDGILLATGLHVHR | | | | |
| | | | | | NSAHSAGVGAIFDRVLTELVSKMRDMRM | | | | |
| | | | | | DKTELGCLRAIILFNPDAKGLSNPSEVEVL | | | | |
| | | | | | REKVYASLETYCKQKYPEQQGRFAKLLL | | | | |
| | | | | | RLPALRSIGLKCLEHLFFFKLIGDTPIDTFL | | | | |
| | | | | | MEMLEAPHQLA | | | | |
| IKZF3 | IL2RA | 22806 | NM_012481.4 | NC_000017.11 | MEDIQTNAELKSTQEQSVPAESAAVLNDY | 61 | Positive | Positive | NS |
| | | | | | SLTKSHEMENVDSGEGPANEDEDIGDDS | | Regulator | Regulator | |
| | | | | | MKVKDEYSERDENVLKSEPMGNAEEPEIP | | | | |
| | | | | | YSYSREYNEYENIKLERHVVSFDSSRPTSG | | | | |
| | | | | | KMNCDVCGLSCISFNVLMVHKRSHTGER | | | | |
| | | | | | PFQCNQCGASFTQKGNLLRHIKLHTGEKP | | | | |
| | | | | | FKCHLCNYACQRRDALTGHLRTHSVEKP | | | | |
| | | | | | YKCEFCGRSYKQRSSLEEHKERCRTFLQS | | | | |
| | | | | | TDPGDTASAEARHIKAEMGSERALVLDRL | | | | |
| | | | | | ASNVAKRKSSMPQKFIGEKRHCFDVNYN | | | | |
| | | | | | SSYMYEKESELIQTRMMDQAINNAISYLG | | | | |
| | | | | | AEALRPLVQTPPAPTSEMVPVISSMYPIAL | | | | |
| | | | | | TRAEMSNGAPQELEKKSIHLPEKSVPSER | | | | |
| | | | | | GLSPNNSGHDSTDTDSNHEERQNHIYQQN | | | | |
| | | | | | HMVLSRARNGMPLLKEVPRSYELLKPPPI | | | | |
| | | | | | CPRDSVKVINKEGEVMDVYRCDHCRVLF | | | | |
| | | | | | LDYVMFTIHMGCHGFRDPFECNMCGYRS | | | | |
| | | | | | HDRYEFSSHIARGEHRALLK | | | | |
| PURA | IL2RA | 5813 | NM_005859.4 | NC_000005.10 | MADRDSGSEQGGAALGSGGSLGHPGSGS | 62 | Positive | NS | NS |
| | | | | | GSGGGGGGGGGGGGSGGGGGGAPGGLQ | | Regulator | | |
| | | | | | HETQELASKRVDIQNKRFYLDVKQNAKG | | | | |
| | | | | | RFLKIAEVGAGGNKSRLTLSMSVAVEFRD | | | | |
| | | | | | YLGDFIEHYAQLGPSQPPDLAQAQDEPRR | | | | |
| | | | | | ALKSEFLVRENRKYYMDLKENQRGRFLRI | | | | |
| | | | | | RQTVNRGPGLGSTQGQTIALPAQGLIEFR | | | | |
| | | | | | DALAKLIDDYGVEEEPAELPEGTSLTVDN | | | | |
| | | | | | KRFFFDVGSNKYGVFMRVSEVKPTYRNSI | | | | |
| | | | | | TVPYKVWAKFGHTFCKYSEEMKKIQEKQ | | | | |
| | | | | | REKRAACEQLHQQQQQQQEETAAATLLL | | | | |
| | | | | | QGEEEGEED | | | | |
| SETDB1 | IL2RA | 9869 | NM_012432.3 | NC_000001.11 | MSSLPGCIGLDAATATVESEEIAELQQAV | 63 | Positive | Positive | NS |
| | | | | | VEELGISMEELRHFIDEELEKMDCVQQRK | | Regulator | Regulator | |
| | | | | | KQLAELETWVIQKESEVAHVDQLEDDAS | | | | |
| | | | | | RAVINCESLVKDFYSKLGLQYRDSSSEDE | | | | |
| | | | | | SSRPTEHEIPDEDDDVLSIDSGDAGSRTPK | | | | |
| | | | | | DQKLREAMAALRKSAQDVQKFMDAVNK | | | | |
| | | | | | KSSSQDLHKGTLSQMSGELSKDGDLIVSM | | | | |
| | | | | | RILGKKRTKTWHKGTLIAIQTVGPGKKYK | | | | |
| | | | | | VKFDNKGKSLLSGNHIAYDYHPPADKLY | | | | |
| | | | | | VGSRVVAKYKDGNQVWLYAGIVAETPN | | | | |
| | | | | | VKNKLRFLIFFDDGYASYVTQSELYPICRP | | | | |
| | | | | | LKKTWEDIEDISCRDFIEEYVTAYPNRPM | | | | |
| | | | | | VLLKSGQLIKTEWEGTWWKSRVEEVDGS | | | | |
| | | | | | LVRILFLDDKRCEWIYRGSTRLEPMFSMK | | | | |
| | | | | | TSSASALEKKQGQLRTRPNMGAVRSKGP | | | | |
| | | | | | VVQYTQDLTGTGTQFKPVEPPQPTAPPAP | | | | |
| | | | | | PFPPAPPLSPQAGDSDLESQLAQSRKQVA | | | | |
| | | | | | KKSTSFRPGSVGSGHSSPTSPALSENVSGG | | | | |
| | | | | | KPGINQTYRSPLGSTASAPAPSALPAPPAP | | | | |
| | | | | | PVFHGMLERAPAEPSYRAPMEKLFYLPHV | | | | |
| | | | | | CSYTCLSRVRPMRNEQYRGKNPLLVPLLY | | | | |
| | | | | | DFRRMTARRRVNRKMGFHVIYKTPCGLC | | | | |
| | | | | | LRTMQEIERYLFETGCDFLFLEMFCLDPY | | | | |
| | | | | | VLVDRKFQPYKPFYYILDITYGKEDVPLS | | | | |
| | | | | | CVNEIDTTPPPQVAYSKERIPGKGVFINTG | | | | |
| | | | | | PEFLVGCDCKDGCRDKSKCACHQLTIQAT | | | | |
| | | | | | ACTPGGQINPNSGYQYKRLEECLPTGVYE | | | | |
| | | | | | CNKRCKCDPNMCTNRLVQHGLQVRLQLF | | | | |
| | | | | | KTQNKGWGIRCLDDIAKGSFVCIYAGKIL | | | | |
| | | | | | TDDFADKEGLEMGDEYFANLDHIESVENF | | | | |
| | | | | | KEGYESDAPCSSDSSGVDLKDQEDGNSGT | | | | |
| | | | | | EDPEESNDDSSDDNFCKDEDFSTSSVWRS | | | | |
| | | | | | YATRRQTRGQKENGLSETTSKDSHPPDLG | | | | |
| | | | | | PPHIPVPPSIPVGGCNPPSSEETPKNKVAS | | | | |
| | | | | | WLSCNSVSEGGFADSDSHSSFKTNEGGEG | | | | |
| | | | | | RAGGSRMEAEKASTSGLGIKDEGDIKQAK | | | | |
| | | | | | KEDTDDRNKMSVVTESSRNYGYNPSPVK | | | | |
| | | | | | PEGLRRPPSKTSMHQSRRLMASAQSNPDD | | | | |
| | | | | | VLTLSSSTESEGESGTSRKPTAGQTSATAV | | | | |
| | | | | | DSDDIQTISSGSEGDDFEDKKNMTGPMKR | | | | |
| | | | | | QVAVKSTRGFALKSTHGIAIKSTNMASVD | | | | |
| | | | | | KGESAPVRKNTRQFYDGEESCYIIDAKLE | | | | |
| | | | | | GNLGRYLNHSCSPNLFVQNVFVDTHDLR | | | | |
| | | | | | FPWVAFFASKIRAGTELTWDYNYEVGSV | | | | |
| | | | | | EGKELLCCCGAIECRGRLL | | | | |
| RELA | IL2RA | 5970 | NM_ | NC_000011.10 | MDELFPLIFPAEPAQASGPYVEIIEQPKQR | 64 | Positive | Positive | NS |
| | | | 001243984.1 | | GMRFRYKCEGRSAGSIPGERSTDTTKTHP | | Regulator | Regulator | |
| | | | | | TIKINGYTGPGTVRISLVTKDPPHRPHPHE | | | | |
| | | | | | LVGKDCRDGFYEAELCPDRCIHSFQNLGI | | | | |
| | | | | | QCVKKRDLEQAISQRIQTNNNPFQVPIEEQ | | | | |
| | | | | | RGDYDLNAVRLCFQVTVRDPSGRPLRLPP | | | | |
| | | | | | VLSHPIFDNRAPNTAELKICRVNRNSGSCL | | | | |
| | | | | | GGDEIFLLCDKVQKEDIEVYFTGPGWEAR | | | | |
| | | | | | GSFSQADVHRQVAIVERTPPYADPSLQAP | | | | |
| | | | | | VRVSMQLRRPSDRELSEPMEFQYLPDTDD | | | | |
| | | | | | RHRIEEKRKRTYETFKSIMKKSPFSGPTDP | | | | |
| | | | | | RPPPRRIAVPSRSSASVPKPAPQPYPFTSSL | | | | |
| | | | | | STINYDEFPTMVFPSGQISQASALAPAPPQ | | | | |
| | | | | | VLPQAPAPAPAPAMVSALAQAPAPVPVL | | | | |
| | | | | | APGPPQAVAPPAPKPTQAGEGTLSEALLQ | | | | |
| | | | | | LQFDDEDLGALLGNSTDPAVFTDLASVD | | | | |
| | | | | | NSEFQQLLNQGIPVAPHTTEPMLMEYPEA | | | | |
| | | | | | ITRLVTGAQRPPDPAPAPLGAPGLPNGLLS | | | | |
| | | | | | GDEDFSSIADMDFSALLSQISS | | | | |
| SS18 | IL2RA | 6760 | NM_ | NC_000018.10 | MSVAFAAPRQRGKGEITPAAIQKMLDDN | 65 | Positive | NS | NS |
| | | | 001007559.1 | | NHLIQCIMDSQNKGKTSECSQYQQMLHT | | Regulator | | |
| | | | | | NLVYLATIADSNQNMQSLLPAPPTQNMP | | | | |
| | | | | | MGPGGMNQSGPPPPPRSHNMPSDGMVGG | | | | |
| | | | | | GPPAPHMQNQMNGQMPGPNHMPMQGPG | | | | |
| | | | | | PNQLNMTNSSMNMPSSSHGSMGGYNHSV | | | | |
| | | | | | PSSQSMPVQNQMTMSQGQPMGNYGPRP | | | | |
| | | | | | NMSMQPNQGPMMHQQPPSQQYNMPQGG | | | | |
| | | | | | GQHYQGQQPPMGMMGQVNQGNHMMG | | | | |
| | | | | | QRQIPPYRPPQQGPPQQYSGQEDYYGDQY | | | | |
| | | | | | SHGGQGPPEGMNQQYYPDGHNDYGYQQ | | | | |
| | | | | | PSYPEQGYDRPYEDSSQHYYEGGNSQYG | | | | |
| | | | | | QQQDAYQGPPPQQGYPPQQQQYPGQQG | | | | |
| | | | | | YPGQQQGYGPSQGGPGPQYPNYPQGQGQ | | | | |
| | | | | | QYGGYRPTQPGPPQPPQQRPYGYDQGQY | | | | |
| | | | | | GNYQQ | | | | |
| SRF | IL2RA | 6722 | NM_003131.3 | NC_000006.12 | MLPTQAGAAAALGRGSALGGSLNRTPTG | 66 | Positive | Positive | Positive |
| | | | | | RPGGGGGTRGANGGRVPGNGAGLGPGRL | | Regulator | Regulator | Regulator |
| | | | | | EREAAAAAATTPAPTAGALYSGSEGDSES | | | | |
| | | | | | GEEEELGAERRGLKRSLSEMEIGMVVGGP | | | | |
| | | | | | EASAAATGGYGPVSGAVSGAKPGKKTRG | | | | |
| | | | | | RVKIKMEFIDNKLRRYTTFSKRKTGIMKK | | | | |
| | | | | | AYELSTLTGTQVLLLVASETGHVYTFATR | | | | |
| | | | | | KLQPMITSETGKALIQTCLNSPDSPPRSDP | | | | |
| | | | | | TTDQRMSATGFEETDLTYQVSESDSSGET | | | | |
| | | | | | KDTLKPAFTVTNLPGTTSTIQTAPSTSTTM | | | | |
| | | | | | QVSSGPSFPITNYLAPVSASVSPSAVSSAN | | | | |
| | | | | | GTVLKSTGSGPVSSGGLMQLPTSFTLMPG | | | | |
| | | | | | GAVAQQVPVQAIQVHQAPQQASPSRDSS | | | | |
| | | | | | TDLTQTSSSGTVTLPATIMTSSVPTTVGGH | | | | |
| | | | | | MMYPSPHAVMYAPTSGLGDGSLTVLNAF | | | | |
| | | | | | SQAPSTMQVSHSQVQEPGGVPQVFLTASS | | | | |
| | | | | | GTVQIPVSAVQLHQMAVIGQQAGSSSNLT | | | | |
| | | | | | ELQVVNLDTAHSTKSE | | | | |
| GATA3 | IL2RA | 2625 | NM_002051.2 | NC_000010.11 | MEVTADQPRWVSHHHPAVINGQHPDTH | 67 | Positive | Positive | Positive |
| | | | | | HPGLSHSYMDAAQYPLPEEVDVLFNIDGQ | | Regulator | Regulator | Regulator |
| | | | | | GNHVPPYYGNSVRATVQRYPPTHHGSQV | | | | |
| | | | | | CRPPLLHGSLPWLDGGKALGSHHTASPW | | | | |
| | | | | | NLSPFSKTSIHHGSPGPLSVYPPASSSSLSG | | | | |
| | | | | | GHASPHLFTFPPTPPKDVSPDPSLSTPGSA | | | | |
| | | | | | GSARQDEKECLKYQVPLPDSMKLESSHSR | | | | |
| | | | | | GSMTALGGASSSTHHPITTYPPYVPEYSSG | | | | |
| | | | | | LFPPSSLLGGSPTGFGCKSRPKARSSTGRE | | | | |
| | | | | | CVNCGATSTPLWRRDGTGHYLCNACGLY | | | | |
| | | | | | HKMNGQNRPLIKPKRRLSAARRAGTSCA | | | | |
| | | | | | NCQTTTTTLWRRNANGDPVCNACGLYY | | | | |
| | | | | | KLHNINRPLTMKKEGIQTRNRKMSSKSKK | | | | |
| | | | | | CKKVHDSLEDFPKNSSFNPAALSRHMSSL | | | | |
| | | | | | SHISPFSHSSHMLTTPTPMHPPSSLSFGPHH. | | | | |
| | | | | | PSSMVTAMG | | | | |
| ZNF384 | IL2RA | 171017 | NM_ | NC_000012.12 | MEESHFNSNPYFWPSIPTVSGQIENTMFIN | 68 | Positive | NS | NS |
| | | | 001135734.2 | | KMKDQLLPEKGCGLAPPHYPTLLTVPASV | | Regulator | | |
| | | | | | SLPSGISMDTESKSDQLTPHSQASVTQNIT | | | | |
| | | | | | VVPVPSTGLMTAGVSCSQRWRREGSQSR | | | | |
| | | | | | GPGLVITSPSGSLVTTASSAQTFPISAPMIV | | | | |
| | | | | | SALPPGSQALQVVPDLSKKVASTLTEEGG | | | | |
| | | | | | GGGGGGGSVAPKPPRGRKKKRMLESGLP | | | | |
| | | | | | EMNDPYVLSPEDDDDHQKDGKTYRCRM | | | | |
| | | | | | CSLTFYSKSEMQIHSKSHTETKPHKCPHCS | | | | |
| | | | | | KTFANSSYLAQHIRIHSGAKPYSCNFCEKS | | | | |
| | | | | | FRQLSHLQQHTRIHSKMHTETIKPHKCPH | | | | |
| | | | | | CSKTFANTSYLAQHLRIHSGAKPYNCSYC | | | | |
| | | | | | QKAFRQLSHLQQHTRIHTGDRPYKCAHP | | | | |
| | | | | | GCEKAFTQLSNLQSHRRQHNKDKPFKCH | | | | |
| | | | | | NCHRAYTDAASLEVHLSTHTVKHAKVYT | | | | |
| | | | | | CTICSRAYTSETYLMKHMRKHNPPDLQQ | | | | |
| | | | | | QVQAAAAAAAVAQAQAQAQAQAQAQA | | | | |
| | | | | | QAQAQAQASQASQQQQQQQQQQQQQQQ | | | | |
| | | | | | QPPPHFQSPGAAPQGGGGGDSNPNPPPQC | | | | |
| | | | | | SFDLTPYKTAEHHKDICLTVTTSTIQVEHL | | | | |
| | | | | | ASS | | | | |
| ZNF148 | IL2RA | 7707 | NM_021964.2 | NC_000003.12 | MNIDDKLEGLFLKCGGIDEMQSSRTMVV | 69 | Positive | Positive | NS |
| | | | | | MGGVSGQSTVSGELQDSVLQDRSMPHQE | | Regulator | Regulator | |
| | | | | | ILAADEVLQESEMRQQDMISHDELMVHE | | | | |
| | | | | | ETVKNDEEQMETHERLPQGLQYALNVPIS | | | | |
| | | | | | VKQEITFTDVSEQLMRDKKQIREPVDLQK | | | | |
| | | | | | KKKRKQRSPAKILTINEDGSLGLKTPKSH | | | | |
| | | | | | VCEHCNAAFRTNYHLQRHVFIHTGEKPFQ | | | | |
| | | | | | CSQCDMRFIQKYLLQRHEKIHTGEKPFRC | | | | |
| | | | | | DECGMRFIQKYHMERHKRTHSGEKPYQC | | | | |
| | | | | | EYCLQYFSRTDRVLKHKRMCHENHDKKL | | | | |
| | | | | | NRCAIKGGLLTSEEDSGFSTSPKDNSLPKK | | | | |
| | | | | | KRQKTEKKSSGMDKESALDKSDLKKDKN | | | | |
| | | | | | DYLPLYSSSTKVKDEYMVAEYAVEMPHS | | | | |
| | | | | | SVGGSHLEDASGEIHPPKLVLKKINSKRSL | | | | |
| | | | | | KQPLEQNQTISPLSTYEESKVSKYAFELVD | | | | |
| | | | | | KQALLDSEGNADIDQVDNLQEGPSKPVHS | | | | |
| | | | | | STNYDDAMQFLKKKRYLQAASNNSREYA | | | | |
| | | | | | LNVGTIASQPSVTQAAVASVIDESTTASIL | | | | |
| | | | | | ESQALNVEIKSNHDKNVIPDEVLQTLLDH | | | | |
| | | | | | YSHKANGQHEISFSVADTEVTSSISINSSEV | | | | |
| | | | | | PEVTPSENVGSSSQASSSDKANMLQEYSK | | | | |
| | | | | | FLQQALDRTSQNDAYLNSPSLNFVTDNQT | | | | |
| | | | | | LPNQPAFSSIDKQVYATMPINSFRSGMNSP | | | | |
| | | | | | LRTTPDKSHFGLIVGDSQHSFPFSGDETNH | | | | |
| | | | | | ASATSTQDFLDQVTSQKKAEAQPVHQAY | | | | |
| | | | | | QMSSFEQPFRAPYHGSRAGIATQFSTANG | | | | |
| | | | | | QVNLRGPGTSAEFSEFPLVNVNDNRAGM | | | | |
| | | | | | TSSPDATTGQTFG | | | | |
| JAK3 | IL2RA | 3718 | NM_000215.3 | NC_000019.10 | MAPPSEETPLIPQRSCSLLSTEAGALHVLL | 70 | Positive | Positive | Positive |
| | | | | | PARGPGPPQRLSFSFGDHLAEDLCVQAAK | | Regulator | Regulator | Regulator |
| | | | | | ASGILPVYHSLFALATEDLSCWFPPSHIFS | | | | |
| | | | | | VEDASTQVLLYRIRFYFPNWFGLEKCHRF | | | | |
| | | | | | GLRKDLASAILDLPVLEHLFAQHRSDLVS | | | | |
| | | | | | GRLPVGLSLKEQGECLSLAVLDLARMAR | | | | |
| | | | | | EQAQRPGELLKTVSYKACLPPSLRDLIQG | | | | |
| | | | | | LSFVTRRRIRRTVRRALRRVAACQADRHS | | | | |
| | | | | | LMAKYIMDLERLDPAGAAETFHVGLPGA | | | | |
| | | | | | LGGHDGLGLLRVAGDGGIAWTQGEQEVL | | | | |
| | | | | | QPFCDFPEIVDISIKQAPRVGPAGEHRLVT | | | | |
| | | | | | VTRTDNQILEAEFPGLPEALSFVALVDGY | | | | |
| | | | | | FRLTTDSQHFFCKEVAPPRLLEEVAEQCH | | | | |
| | | | | | GPITLDFAINKLKTGGSRPGSYVLRRSPQD | | | | |
| | | | | | FDSFLLTVCVQNPLGPDYKGCLIRRSPTGT | | | | |
| | | | | | FLLVGLSRPHSSLRELLATCWDGGLHVDG | | | | |
| | | | | | VAVTLTSCCIPRPKEKSNLIVVQRGHSPPT | | | | |
| | | | | | SSLVQPQSQYQLSQMTFHKIPADSLEWHE | | | | |
| | | | | | NLGHGSFTKIYRGCRHEVVDGEARKTEV | | | | |
| | | | | | LLKVMDAKHKNCMESFLEAASLMSQVSY | | | | |
| | | | | | RHLVLLHGVCMAGDSTMVQEFVHLGAID | | | | |
| | | | | | MYLRKRGHLVPASWKLQVVKQLAYALN | | | | |
| | | | | | YLEDKGLPHGNVSARKVLLAREGADGSP | | | | |
| | | | | | PFIKLSDPGVSPAVLSLEMLTDRIPWVAPE | | | | |
| | | | | | CLREAQTLSLEADKWGFGATVWEVFSGV | | | | |
| | | | | | TMPISALDPAKKLQFYEDRQQLPAPKWTE | | | | |
| | | | | | LALLIQQCMAYEPVQRPSFRAVIRDLNSLI | | | | |
| | | | | | SSDYELLSDPTPGALAPRDGLWNGAQLY | | | | |
| | | | | | ACQDPTIFEERHLKYISQLGKGNFGSVELC | | | | |
| | | | | | RYDPLGDNTGALVAVKQLQHSGPDQQRD | | | | |
| | | | | | FQREIQILKALHSDFIVKYRGVSYGPGRQS | | | | |
| | | | | | LRLVMEYLPSGCLRDFLQRHRARLDASRL | | | | |
| | | | | | LLYSSQICKGMEYLGSRRCVHRDLAARNI | | | | |
| | | | | | LVESEAHVKIADFGLAKLLPLDKDYYVV | | | | |
| | | | | | REPGQSPIFWYAPESLSDNIFSRQSDVWSF | | | | |
| | | | | | GVVLYELFTYCDKSCSPSAEFLRMMGCE | | | | |
| | | | | | RDVPALCRLLELLEEGQRLPAPPACPAEV | | | | |
| | | | | | HELMKLCWAPSPQDRPSFSALGPQLDML | | | | |
| | | | | | WSGSRGCETHAFTAHPEGKHHSLSFS | | | | |
| MED14 | IL2RA | 9282 | NM_004229.3 | NC_000023.11 | MAPVQLENHQLVPPGGGGGGSGGPPSAP | 71 | Positive | NS | NS |
| | | | | | APPPPGAAVAAAAAAAASPGYRLSTLIEF | | Regulator | | |
| | | | | | LLHRAYSELMVLTDLLPRKSDVERKIEIV | | | | |
| | | | | | QFASRTRQLFVRLLALVKWANNAGKVEK | | | | |
| | | | | | CAMISSELDQQAILFVDTADRLASLARDA | | | | |
| | | | | | LVHARLPSFAIPYAIDVLTTGSYPRLPTCIR | | | | |
| | | | | | DKIIPPDPITKIEKQATLHQLNQILRHRLVT | | | | |
| | | | | | TDLPPQLANLTVANGRVKFRVEGEFEATL | | | | |
| | | | | | TVMGDDPDVPWRLLKLEILVEDKETGDG | | | | |
| | | | | | RALVHSMQISFIHQLVQSRLFADEKPLQD | | | | |
| | | | | | MYNCLHSFCLSLQLEVLHSQTLMLIRERW | | | | |
| | | | | | GDLVQVERYHAGKCLSLSVWNQQVLGR | | | | |
| | | | | | KTGTASVHKVTIKIDENDVSKPLQIFHDPP | | | | |
| | | | | | LPASDSKLVERAMKIDHLSIEKLLIDSVHA | | | | |
| | | | | | RAHQKLQELKAILRGFNANENSSIETALP | | | | |
| | | | | | ALVVPILEPCGNSECLHIFVDLHSGMFQL | | | | |
| | | | | | MLYGLDQATLDDMEKSVNDDMKRIIPWI | | | | |
| | | | | | QQLKFWLGQQRCKQSIKHLPTISSETLQLS | | | | |
| | | | | | NYSTHPIGNLSKNKLFIKLTRLPQYYIVVE | | | | |
| | | | | | MLEVPNKPTQLSYKYYFMSVNAADREDS | | | | |
| | | | | | PAMALLLQQFKENIQDLVFRTKTGKQTRT | | | | |
| | | | | | NAKRKLSDDPCPVESKKTKRAGEMCAFN | | | | |
| | | | | | KVLAHFVAMCDTNMPFVGLRLELSNLEIP | | | | |
| | | | | | HQGVQVEGDGFSHAIRLLKIPPCKGITEET | | | | |
| | | | | | QKALDRSLLDCTFRLQGRNNRTWVAELV | | | | |
| | | | | | FANCPLNGTSTREQGPSRHVYLTYENLLS | | | | |
| | | | | | EPVGGRKVVEMFLNDWNSIARLYECVLE | | | | |
| | | | | | FARSLPDIPAHLNIFSEVRVYNYRKLILCY | | | | |
| | | | | | GTTKGSSISIQWNSIHQKFHISLGTVGPNS | | | | |
| | | | | | GCSNCHNTILHQLQEMFNKTPNVVQLLQ | | | | |
| | | | | | VLFDTQAPLNAINKLPTVPMLGLTQRTNT | | | | |
| | | | | | AYQCFSILPQSSTHIRLAFRNMYCIDIYCRS | | | | |
| | | | | | RGVVAIRDGAYSLFDNSKLVEGFYPAPGL | | | | |
| | | | | | KTFLNMFVDSNQDARRRSVNEDDNPPSPI | | | | |
| | | | | | GGDMMDSLISQLQPPPQQQPFPKQPGTSG | | | | |
| | | | | | AYPLTSPPTSYHSTVNQSPSMMHTQSPGN | | | | |
| | | | | | LHAASSPSGALRAPSPASFVPTPPPSSHGIS | | | | |
| | | | | | IGPGASFASPHGTLDPSSPYTMVSPSGRAG | | | | |
| | | | | | NWPGSPQVSGPSPAARMPGMSPANPSLHS | | | | |
| | | | | | PVPDASHSPRAGTSSQTMPTNMPPPRKLP | | | | |
| | | | | | QRSWAASIPTILTHSALNILLLPSPTPGLVP | | | | |
| | | | | | GLAGSYLCSPLERFLGSVIMRRHLQRIIQQ | | | | |
| | | | | | ETLQLINSNEPGVIMFKTDALKCRVALSP | | | | |
| | | | | | KTNQTLQLKVTPENAGQWKPDELQVLEK | | | | |
| | | | | | FFETRVAGPPFKANTLIAFTKLLGAPTHIL | | | | |
| | | | | | RDCVHIMKLELFPDQATQLKWNVQFCLTI | | | | |
| | | | | | PPSAPPIAPPGTPAVVLKSKMLFFLQLTQK | | | | |
| | | | | | TSVPPQEPVSIIVPIIYDMASGTTQQADIPR | | | | |
| | | | | | QQNSSVAAPMMVSNILKRFAEMNPPRQG | | | | |
| | | | | | ECTIFAAVRDLMANLTLPPGGRP | | | | |
| PKNOX1 | IL2RA | 5316 | NM_004571.4 | NC_000021.9 | MMATQTLSIDSYQDGQQMQVVTELKTEQ | 72 | Positive | NS | NS |
| | | | | | DPNCSEPDAEGVSPPPVESQTPMDVDKQA | | Regulator | | |
| | | | | | IYRHPLFPLLALLFEKCEQSTQGSEGTTSA | | | | |
| | | | | | SFDVDIENFVRKQEKEGKPFFCEDPETDN | | | | |
| | | | | | LMVKAIQVLRIHLLELEKVNELCKDFCSR | | | | |
| | | | | | YIACLKTKMNSETLLSGEPGSPYSPVQSQ | | | | |
| | | | | | QIQSAITGTISPQGIVVPASALQQGNVAMA | | | | |
| | | | | | TVAGGTVYQPVTVVTPQGQVVTQTLSPG | | | | |
| | | | | | TIRIQNSQLQLQLNQDLSILHQDDGSSKNK | | | | |
| | | | | | RGVLPKHATNVMRSWLFQHIGHPYPTED | | | | |
| | | | | | EKKQIAAQTNLTLLQVNNWFINARRRILQ | | | | |
| | | | | | PMLDSSCSETPKTKKKTAQNRPVQRFWP | | | | |
| | | | | | DSIASGVAQPPPSELTMSEGAVVTITTPVN | | | | |
| | | | | | MNVDSLQSLSSDGATLAVQQVMMAGQS | | | | |
| | | | | | EDESVDSTEEDAGALAPAHISGLVLENSD | | | | |
| | | | | | SLQ | | | | |
| USF2 | IL2RA | 7392 | NM_003367.2 | NC_000019.10 | MDMLDPGLDPAASATAAAAASHDKGPE | 73 | Positive | Positive | NS |
| | | | | | AEEGVELQEGGDGPGAEEQTAVAITSVQ | | Regulator | Regulator | |
| | | | | | QAAFGDHNIQYQFRTETNGGQVTYRVVQ | | | | |
| | | | | | VTDGQLDGQGDTAGAVSVVSTAAFAGG | | | | |
| | | | | | QQAVTQVGVDGAAQRPGPAAASVPPGPA | | | | |
| | | | | | APFPLAVIQNPFSNGGSPAAEAVSGEARF | | | | |
| | | | | | AYFPASSVGDTTAVSVQTTDQSLQAGGQ | | | | |
| | | | | | FYVMMTPQDVLQTGTQRTIAPRTHPYSPK | | | | |
| | | | | | IDGTRTPRDERRRAQHNEVERRRRDKINN | | | | |
| | | | | | WIVQLSKIIPDCNADNSKTGASKGGILSKA | | | | |
| | | | | | CDYIRELRQTNQRMQETFKEAERLQMDN | | | | |
| | | | | | ELLRQQIEELKNENALLRAQLQQHNLEM | | | | |
| | | | | | VGEGTRQ | | | | |
| KLF6 | IL2RA | 1316 | NM_ | NC_000010.11 | MDVLPMCSIFQELQIVHETGYFSALPSLEE | 74 | Positive | NS | NS |
| | | | 001160124.1 | | YWQQTCLELERYLQSEPCYVSASEIKFDS | | Regulator | | |
| | | | | | QEDLWTKIILAREKKEESELKISSSPPEDTL | | | | |
| | | | | | ISPSFCYNLETNSLNSDVSSESSDSSEELSP | | | | |
| | | | | | TAKFTSDPIGEVLVSSGKLSSSVTSTPPSSP | | | | |
| | | | | | ELSREPSQLWGCVPGELPSPGKVRSGTSG | | | | |
| | | | | | KPGDKGNGDASPDGRRRVHRCHFNGCRK | | | | |
| | | | | | VYTKSSHLKAHQRTHTGEKPYRCSWEGC | | | | |
| | | | | | EWRFARSDELTRHFRKHTGAKPFKCSHC | | | | |
| | | | | | DRCFSRSDHLALHMKRHL | | | | |
| DNMT1 | IL2RA | 1786 | NM_001379.2 | NC_000019.10 | MPARTAPARVPTLAVPAISLPDDVRRRLK | 75 | Positive | NS | NS |
| | | | | | DLERDSLTEKECVKEKLNLLHEFLQTEIK | | Regulator | | |
| | | | | | NQLCDLETKLRKEELSEEGYLAKVKSLLN | | | | |
| | | | | | KDLSLENGAHAYNREVNGRLENGNQARS | | | | |
| | | | | | EARRVGMADANSPPKPLSKPRTPRRSKSD | | | | |
| | | | | | GEAKPEPSPSPRITRKSTRQTTITSHFAKGP | | | | |
| | | | | | AKRKPQEESERAKSDESIKEEDKDQDEKR | | | | |
| | | | | | RRVTSRERVARPLPAEEPERAKSGTRTEK | | | | |
| | | | | | EEERDEKEEKRLRSQTKEPTPKQKLKEEP | | | | |
| | | | | | DREARAGVQADEDEDGDEKDEKKHRSQP | | | | |
| | | | | | KDLAAKRRPEEKEPEKVNPQISDEKDEDE | | | | |
| | | | | | KEEKRRKTTPKEPTEKKMARAKTVMNSK | | | | |
| | | | | | THPPKCIQCGQYLDDPDLKYGQHPPDAV | | | | |
| | | | | | DEPQMLTNEKLSIFDANESGFESYEALPQ | | | | |
| | | | | | HKLTCFSVYCKHGHLCPIDTGLIEKNIELF | | | | |
| | | | | | FSGSAKPIYDDDPSLEGGVNGKNLGPINE | | | | |
| | | | | | WWITGFDGGEKALIGFSTSFAEYILMDPSP | | | | |
| | | | | | EYAPIFGLMQEKIYISKIVVEFLQSNSDSTY | | | | |
| | | | | | EDLINKIETTVPPSGLNLNRFTEDSLLRHA | | | | |
| | | | | | QFVVEQVESYDEAGDSDEQPIFLTPCMRD | | | | |
| | | | | | LIKLAGVILGQRRAQARRQTIRHSTREKD | | | | |
| | | | | | RGPTKATTTKLVYQIFDTFFAEQIEKDDRE | | | | |
| | | | | | DKENAFKRRRCGVCEVCQQPECGKCKAC | | | | |
| | | | | | KDMVKFGGSGRSKQACQERRCPNMAMK | | | | |
| | | | | | EADDDEEVDDNIPEMPSPKKMHQGKKKK | | | | |
| | | | | | QNKNRISWVGEAVKTDGKKSYYKKVCID | | | | |
| | | | | | AETLEVGDCVSVIPDDSSKPLYLARVTAL | | | | |
| | | | | | WEDSSNGQMFHAHWFCAGTDTVLGATS | | | | |
| | | | | | DPLELFLVDECEDMQLSYIHSKVKVIYKA | | | | |
| | | | | | PSENWAMEGGMDPESLLEGDDGKTYFYQ | | | | |
| | | | | | LWYDQDYARFESPPKTQPTEDNKFKFCVS | | | | |
| | | | | | CARLAEMRQKEIPRVLEQLEDLDSRVLYY | | | | |
| | | | | | SATKNGILYRVGDGVYLPPEAFTFNIKLSS | | | | |
| | | | | | PVKRPRKEPVDEDLYPEHYRKYSDYIKGS | | | | |
| | | | | | NLDAPEPYRIGRIKEIFCPKKSNGRPNETDI | | | | |
| | | | | | KIRVNKFYRPENTHKSTPASYHADINLLY | | | | |
| | | | | | WSDEEAVVDFKAVQGRCTVEYGEDLPEC | | | | |
| | | | | | VQVYSMGGPNRFYFLEAYNAKSKSFEDP | | | | |
| | | | | | PNHARSPGNKGKGKGKGKGKPKSQACEP | | | | |
| | | | | | SEPEIEIKLPKLRTLDVFSGCGGLSEGFHQ | | | | |
| | | | | | AGISDTLWAIEMWDPAAQAFRLNNPGST | | | | |
| | | | | | VFTEDCNILLKLVMAGETTNSRGQRLPQK | | | | |
| | | | | | GDVEMLCGGPPCQGFSGMNRFNSRTYSK | | | | |
| | | | | | FKNSLVVSFLSYCDYYRPRFFLLENVRNF | | | | |
| | | | | | VSFKRSMVLKLTLRCLVRMGYQCTFGVL | | | | |
| | | | | | QAGQYGVAQTRRRAIILAAAPGEKLPLFP | | | | |
| | | | | | EPLHVFAPRACQLSVVVDDKKFVSNITRL | | | | |
| | | | | | SSGPFRTITVRDTMSDLPEVRNGASALEIS | | | | |
| | | | | | YNGEPQSWFQRQLRGAQYQPILRDHICKD | | | | |
| | | | | | MSALVAARMRHIPLAPGSDWRDLPNIEV | | | | |
| | | | | | RLSDGTMARKLRYTHHDRKNGRSSSGAL | | | | |
| | | | | | RGVCSCVEAGKACDPAARQFNTLIPWCLP | | | | |
| | | | | | HTGNRHNHWAGLYGRLEWDGFFSTTVT | | | | |
| | | | | | NPEPMGKQGRVLHPEQHRVVSVRECARS | | | | |
| | | | | | QGFPDTYRLFGNILDKHRQVGNAVPPPLA | | | | |
| | | | | | KAIGLEIKLCMLAKARESASAKIKEEEAA | | | | |
| | | | | | KD | | | | |
| DDX39B | IL2RA | 7919 | NM_080598.5 | NC_000006.12 | MAENDVDNELLDYEDDEVETAAGGDGA | 76 | NS | Positive | NS |
| | | | | | EAPAKKDVKGSYVSIHSSGFRDFLLKPEL | | | Regulator | |
| | | | | | LRAIVDCGFEHPSEVQHECIPQAILGMDVL | | | | |
| | | | | | CQAKSGMGKTAVFVLATLQQLEPVTGQV | | | | |
| | | | | | SVLVMCHTRELAFQISKEYERFSKYMPNV | | | | |
| | | | | | KVAVFFGGLSIKKDEEVLKKNCPHIVVGT | | | | |
| | | | | | PGRILALARNKSLNLKHIKHFILDECDKM | | | | |
| | | | | | LEQLDMRRDVQEIFRMTPHEKQVMMFSA | | | | |
| | | | | | TLSKEIRPVCRKFMQDPMEIFVDDETKLT | | | | |
| | | | | | LHGLQQYYVKLKDNEKNRKLFDLLDVLE | | | | |
| | | | | | FNQVVIFVKSVQRCIALAQLLVEQNFPAIA | | | | |
| | | | | | IHRGMPQEERLSRYQQFKDFQRRILVATN | | | | |
| | | | | | LFGRGMDIERVNIAFNYDMPEDSDTYLHR | | | | |
| | | | | | VARAGRFGTKGLAITFVSDENDAKILNDV | | | | |
| | | | | | QDRFEVNISELPDEIDISSYIEQTR | | | | |
| ZNF236 | IL2RA | 7776 | NM_007345.3 | NC_000018.10 | MGLCGLLERCWLHHDPDGVLTLNAENTN | 77 | NS | Positive | NS |
| | | | | | YAYQVPNFHKCEICLLSFPKESQFQRHMR | | | Regulator | |
| | | | | | DHERNDKPHRCDQCPQTFNVEFNLTLHK | | | | |
| | | | | | CTHSGEDPTCPVCNKKFSRVASLKAHIML | | | | |
| | | | | | HEKEENLICSECGDEFTLQSQLAVHMEEH | | | | |
| | | | | | RQELAGTRQHACKACKKEFETSSELKEH | | | | |
| | | | | | MKTHYKIRVSSTRSYNRNIDRSGFTYSCP | | | | |
| | | | | | HCGKTFQKPSQLTRHIRIHTGERPFKCSEC | | | | |
| | | | | | GKAFNQKGALQTHMIKHTGEKPHACAFC | | | | |
| | | | | | PAAFSQKGNLQSHVQRVHSEVKNGPTYN | | | | |
| | | | | | CTECSCVFKSLGSLNTHISKMHMGGPQNS | | | | |
| | | | | | TSSTETAHVLTATLFQTLPLQQTEAQATS | | | | |
| | | | | | ASSQPSSQAVSDVIQQLLELSEPAPVESGQ | | | | |
| | | | | | SPQPGQQLSITVGINQDILQQALENSGLSSI | | | | |
| | | | | | PAAAHPNDSCHAKTSAPHAQNPDVSSVS | | | | |
| | | | | | NEQTDPTDAEQEKEQESPEKLDKKEKKMI | | | | |
| | | | | | KKKSPFLPGSIREENGVRWHVCPYCAKEF | | | | |
| | | | | | RKPSDLVRHIRIHTHEKPFKCPQCFRAFAV | | | | |
| | | | | | KSTLTAHIKTHTGIKAFKCQYCMKSFSTS | | | | |
| | | | | | GSLKVHIRLHTGVRPFACPHCDKKFRTSG | | | | |
| | | | | | HRKTHIASHFKHTELRKMRHQRKPAKVR | | | | |
| | | | | | VGKTNIPVPDIPLQEPILITDLGLIQPIPKNQ | | | | |
| | | | | | FFQSYFNNNFVNEADRPYKCFYCHRAYK | | | | |
| | | | | | KSCHLKQHIRSHTGEKPFKCSQCGRGFVS | | | | |
| | | | | | AGVLKAHIRTHTGLKSFKCLICNGAFTTG | | | | |
| | | | | | GSLRRHMGIHNDLRPYMCPYCQKTFKTS | | | | |
| | | | | | LNCKKHMKTHRYELAQQLQQHQQAASID | | | | |
| | | | | | DSTVDQQSMQASTQMQVEIESDELPQTAE | | | | |
| | | | | | VVAANPEAMLDLEPQHVVGTEEAGLGQQ | | | | |
| | | | | | LADQPLEADEDGFVAPQDPLRGHVDQFE | | | | |
| | | | | | EQSPAQQSFEPAGLPQGFTVIDTYHQQPQ | | | | |
| | | | | | FPPVQQLQDSSTLESQALSTSFHQQSLLQA | | | | |
| | | | | | PSSDGMNVTTRLIQESSQEELDLQAQGSQ | | | | |
| | | | | | FLEDNEDQSRRSYRCDYCNKGFKKSSHL | | | | |
| | | | | | KQHVRSHTGEKPYKCKLCGRGFVSSGVL | | | | |
| | | | | | KSHEKTHTGVKAFSCSVCNASFTINGSLT | | | | |
| | | | | | RHMATHMSMKPYKCPFCEEGFRTTVHCK | | | | |
| | | | | | KHMKRHQTVPSAVSATGETEGGDICMEE | | | | |
| | | | | | EEEHSDRNASRKSRPEVITFTEEETAQLAK | | | | |
| | | | | | IRPQESATVSEKVLVQSAAEKDRISELRDK | | | | |
| | | | | | QAELQDEPKHANCCTYCPKSFKKPSDLVR | | | | |
| | | | | | HVRIHTGEKPYKCDECGKSFTVKSTLDCH | | | | |
| | | | | | VKTHTGQKLFSCHVCSNAFSTKGSLKVH | | | | |
| | | | | | MRLHTGAKPFKCPHCELRFRTSGRRKTH | | | | |
| | | | | | MQFHYKPDPKKARKPMTRSSSEGLQPVN | | | | |
| | | | | | LLNSSSTDPNVFIMNNSVLTGQFDQNLLQ | | | | |
| | | | | | PGLVGQAILPASVSAGGDLTVSLTDGSLA | | | | |
| | | | | | TLEGIQLQLAANLVGPNVQISGIDAASINN | | | | |
| | | | | | ITLQIDPSILQQTLQQGNLLAQQLTGEPGL | | | | |
| | | | | | APQNSSLQTSDSTVPASVVIQPISGLSLQPT | | | | |
| | | | | | VTSANLTIGPLSEQDSVLTINSSGTQDLTQ | | | | |
| | | | | | VMTSQGLVSPSGGPHEITLTINNSSLSQVL | | | | |
| | | | | | AQAAGPTATSSSGSPQEITLTISELNTTSGS | | | | |
| | | | | | LPSTTPMSPSAISTQNLVMSSSGVGGDAS | | | | |
| | | | | | VILTLADTQGMLSGGLDTVTLNITSQGQQ | | | | |
| | | | | | FPALLTDPSLSGQGGAGSPQVILVSHTPQS | | | | |
| | | | | | ASAACEEIAYQVAGVSGNLAPGNQPEKE | | | | |
| | | | | | GRAHQCLECDRAFSSAAVLMHHSKEVHG | | | | |
| | | | | | RERIHGCPVCRKAFKRATHLKEHMQTHQ | | | | |
| | | | | | AGPSLSSQKPRVFKCDTCEKAFAKPSQLE | | | | |
| | | | | | RHSRIHTGERPFHCTLCEKAFNQKSALQV | | | | |
| | | | | | HMKKHTGERPYKCAYCVMGFTQKSNMK | | | | |
| | | | | | LHMKRAHSYAGALQESAGHPEQDGEELS | | | | |
| | | | | | RTLHLEEVVQEAAGEWQALTHVE | | | | |
| SOCS3 | IL2RA | 9021 | NM_003955.4 | NC_000017.11 | MVTHSKFPAAGMSRPLDTSLRLKTFSSKS | 78 | NS | NS | Negative |
| | | | | | EYQLVVNAVRKLQESGFYWSAVTGGEA | | | | Regulator |
| | | | | | NLLLSAEPAGTFLIRDSSDQRHFFTLSVKT | | | | |
| | | | | | QSGTKNLRIQCEGGSFSLQSDPRSTQPVPR | | | | |
| | | | | | FDCVLKLVHHYMPPPGAPSFPSPPTEPSSE | | | | |
| | | | | | VPEQPSAQPLPGSPPRRAYYIYSGGEKIPL | | | | |
| | | | | | VLSRPLSSNVATLQHLCRKTVNGHLDSYE | | | | |
| | | | | | KVTQLPGPIREFLDQYDAPL | | | | |
| GABPA | IL2RA | 2551 | NM_ | NC_000021.9 | MTKREAEELIEIEIDGTEKAECTEESIVEQT | 79 | NS | Positive | NS |
| | | | 001197297.1 | | YAPAECVSQAIDINEPIGNLKKLLEPRLQC | | | Regulator | |
| | | | | | SLDAHEICLQDIQLDPERSLFDQGVKTDG | | | | |
| | | | | | TVQLSVQVISYQGIEPKLNILEIVKPADTV | | | | |
| | | | | | EVVIDPDAHHAESEAHLVEEAQVITLDGT | | | | |
| | | | | | KHITTISDETSEQVTRWAAALEGYRKEQE | | | | |
| | | | | | RLGIPYDPIQWSTDQVLHWVVWVMKEFS | | | | |
| | | | | | MTDIDLTTLNISGRELCSLNQEDFFQRVPR | | | | |
| | | | | | GEILWSHLELLRKYVLASQEQQMNEIVTI | | | | |
| | | | | | DQPVQIIPASVQSATPTTIKVINSSAKAAK | | | | |
| | | | | | VQRAPRISGEDRSSPGNRTGNNGQIQLWQ | | | | |
| | | | | | FLLELLTDKDARDCISWVGDEGEFKLNQP | | | | |
| | | | | | ELVAQKWGQRKNKPTMNYEKLSRALRY | | | | |
| | | | | | YYDGDMICKVQGKRFVYKFVCDLKTLIG | | | | |
| | | | | | YSAAELNRLVTECEQKKLAKMQLHGIAQ | | | | |
| | | | | | PVTAVALATASLQTEKDN | | | | |
| RBPJ | IL2RA | 3516 | NM_005349.3 | NC_000004.12 | MDHTEGSPAEEPPAHAPSPGKFGERPPPK | 80 | NS | Positive | Positive |
| | | | | | RLTREAMRNYLKERGDQTVLILHAKVAQ | | | Regulator | Regulator |
| | | | | | KSYGNEKRFFCPPPCVYLMGSGWKKKKE | | | | |
| | | | | | QMERDGCSEQESQPCAFIGIGNSDQEMQQ | | | | |
| | | | | | LNLEGKNYCTAKTLYISDSDKRKHFMLSV | | | | |
| | | | | | KMFYGNSDDIGVFLSKRIKVISKPSKKKQS | | | | |
| | | | | | LKNADLCIASGTKVALFNRLRSQTVSTRY | | | | |
| | | | | | LHVEGGNFHASSQQWGAFFIHLLDDDESE | | | | |
| | | | | | GEEFTVRDGYIHYGQTVKLVCSVTGMAL | | | | |
| | | | | | PRLIIRKVDKQTALLDADDPVSQLHKCAF | | | | |
| | | | | | YLKDTERMYLCLSQERIIQFQATPCPKEPN | | | | |
| | | | | | KEMINDGASWTIISTDKAEYTFYEGMGPV | | | | |
| | | | | | LAPVTPVPVVESLQLNGGGDVAMLELTG | | | | |
| | | | | | QNFTPNLRVWFGDVEAETMYRCGESMLC | | | | |
| | | | | | VVPDISAFREGWRWVRQPVQVPVTLVRN | | | | |
| | | | | | DGIYSTSLTFTYTPEPGPRPHCSAAGAILR | | | | |
| | | | | | ANSSQVPPNESNTNSEGSYTNASTNSTSV | | | | |
| | | | | | TSSTATVVS | | | | |
| STAT3 | IL2RA | 6774 | NM_139276.2 | NC_000017.11 | MAQWNQLQQLDTRYLEQLHQLYSDSFP | 81 | NS | Positive | Positive |
| | | | | | MELRQFLAPWIESQDWAYAASKESHATL | | | Regulator | Regulator |
| | | | | | VFHNLLGEIDQQYSRFLQESNVLYQHNLR | | | | |
| | | | | | RIKQFLQSRYLEKPMEIARIVARCLWEESR | | | | |
| | | | | | LLQTAATAAQQGGQANHPTAAVVTEKQ | | | | |
| | | | | | QMLEQHLQDVRKRVQDLEQKMKVVENL | | | | |
| | | | | | QDDFDFNYKTLKSQGDMQDLNGNNQSV | | | | |
| | | | | | TRQKMQQLEQMLTALDQMRRSIVSELAG | | | | |
| | | | | | LLSAMEYVQKTLTDEELADWKRRQQIAC | | | | |
| | | | | | IGGPPNICLDRLENWITSLAESQLQTRQQI | | | | |
| | | | | | KKLEELQQKVSYKGDPIVQHRPMLEERIV | | | | |
| | | | | | ELFRNLMKSAFVVERQPCMPMHPDRPLVI | | | | |
| | | | | | KTGVQFTTKVRLLVKFPELNYQLKIKVCI | | | | |
| | | | | | DKDSGDVAALRGSRKFNILGTNTKVMNM | | | | |
| | | | | | EESNNGSLSAEFKHLTLREQRCGNGGRAN | | | | |
| | | | | | CDASLIVTEELHLITFETEVYHQGLKIDLE | | | | |
| | | | | | THSLPVVVISNICQMPNAWASILWYNMLT | | | | |
| | | | | | NNPKNVNFFTKPPIGTWDQVAEVLSWQF | | | | |
| | | | | | SSTTKRGLSIEQLTTLAEKLLGPGVNYSGC | | | | |
| | | | | | QITWAKFCKENMAGKGFSFWVWLDNIID | | | | |
| | | | | | LVKKYILALWNEGYIMGFISKERERAILST | | | | |
| | | | | | KPPGTFLLRFSESSKEGGVTFTWVEKDISG | | | | |
| | | | | | KTQIQSVEPYTKQQLNNMSFAEIIMGYKI | | | | |
| | | | | | MDATNILVSPLVYLYPDIPKEEAFGKYCR | | | | |
| | | | | | PESQEHPEADPGSAAPYLKTKFICVTPTTC | | | | |
| | | | | | SNTIDLPMSPRTLDSLMQFGNNGEGAEPS | | | | |
| | | | | | AGGQFESLTFDMELTSECATSPM | | | | |
| VPS52 | IL2RA | 6293 | NM_ | NC_000006.12 | MAAAATMAAAARELVLRAGTSDMEEEE | 82 | NS | Positive | Positive |
| | | | 001289174.1 | | GPLAGGPGLQEPLQLGELDITSDEFILDEV | | | Regulator | Regulator |
| | | | | | DVHIQANLEDELVKEALKTGVDLRHYSK | | | | |
| | | | | | QVELELQQIEQKSIRDYIQESENIASLHNQI | | | | |
| | | | | | TACDAVLERMEQMLGAFQSDLSSISSEIRT | | | | |
| | | | | | LQEQSGAMNIRLRNRQAVRGKLGELVDG | | | | |
| | | | | | LVVPSALVTAILEAPVTEPRFLEQLQELDA | | | | |
| | | | | | KAAAVREQEARGTAACADVRGVLDRLR | | | | |
| | | | | | VKAVTKIREFILQKIYSFRKPMTNYQIPQT | | | | |
| | | | | | ALLKYRFFYQFLLGNERATAKEIRDEYVE | | | | |
| | | | | | TLSKIYLSYYRSYLGRLMKVQYEEVAEK | | | | |
| | | | | | DDLMGVEDTAKKGFFSKPSLRSRNTIFTL | | | | |
| | | | | | GTRGSVISPTELEAPILVPHTAQRGEQRYP | | | | |
| | | | | | FEALFRSQHYALLDNSCREYLFICEFFVVS | | | | |
| | | | | | GPAAHDLFHAVMGRTLSMTLKHLDSYLA | | | | |
| | | | | | DCYDAIAVFLCIHIVLRFRNIAAKRDVPAL | | | | |
| | | | | | DRYWEQVLALLWPRFELILEMNVQSVRS | | | | |
| | | | | | TDPQRLGGLDTRPHYITRRYAEFSSALVSI | | | | |
| | | | | | NQTIPNERTMQLLGQLQVEVENFVLRVA | | | | |
| | | | | | AEFSSRKEQLVFLINNYDMMLGVLMERA | | | | |
| | | | | | ADDSKEVESFQQLLNARTQEFIEELLSPPF | | | | |
| | | | | | GGLVAFVKEAEALIERGQAERLRGEEARV | | | | |
| | | | | | TQLIRGFGSSWKSSVESLSQDVMRSFTNF | | | | |
| | | | | | RNGTSIIQGALTQLIQLYHRFHRVLSQPQL | | | | |
| | | | | | RALPARAELINIHHLMVELKKHKPNF | | | | |
| IL2 | IL2RA | 3558 | NM_000586.3 | NC_000004.12 | MYRMQLLSCIALSLALVTNSAPTSSSTKK | 83 | NS | NS | Negative |
| | | | | | TQLQLEHLLLDLQMILNGINNYKNPKLTR | | | | Regulator |
| | | | | | MLTFKFYMPKKATELKHLQCLEEELKPLE | | | | |
| | | | | | EVLNLAQSKNFHLRPRDLISNINVIVLELK | | | | |
| | | | | | GSETTFMCEYADETATIVEFLNRWITFCQS | | | | |
| | | | | | IISTLT | | | | |
| BCL11B | IL2RA | 64919 | NM_ | NC_000014.9 | MSRRKQGNPQHLSQRELITPEADHVEAAI | 84 | NS | Negative | NS |
| | | | 001282237.1 | | LEEDEGLEIEEPSGLGLMVGGPDPDLLTC | | | Regulator | |
| | | | | | GQCQMNFPLGDILVFIEHKRKQCGGSLGA | | | | |
| | | | | | CYDKALDKDSPPPSSRSELRKVSEPVEIGI | | | | |
| | | | | | QVTPDEDDHLLSPTKGICPKQENIAGPCRP | | | | |
| | | | | | AQLPAVAPIAASSHPHSSVITSPLRALGAL | | | | |
| | | | | | PPCLPLPCCSARPVSGDGTQGEGQTEAPF | | | | |
| | | | | | GCQCQLSGKDEPSSYICTTCKQPFNSAWF | | | | |
| | | | | | LLQHAQNTHGFRIYLEPGPASSSLTPRLTIP | | | | |
| | | | | | PPLGPEAVAQSPLMNFLGDSNPFNLLRMT | | | | |
| | | | | | GPILRDHPGFGEGRLPGTPPLFSPPPRHHL | | | | |
| | | | | | DPHRLSAEEMGLVAQHPSAFDRVMRLNP | | | | |
| | | | | | MAIDSPAMDFSRRLRELAGNSSTPPPVSPG | | | | |
| | | | | | RGNPMHRLLNPFQPSPKSPFLSTPPLPPMP | | | | |
| | | | | | PGGTPPPQPPAKSKSCEFCGKTFKFQSNLI | | | | |
| | | | | | VHRRSHTGEKPYKCQLCDHACSQASKLK | | | | |
| | | | | | RHMKTHMHKAGSLAGRSDDGLSAASSPE | | | | |
| | | | | | PGTSELAGEGLKAADGDFRHHESDPSLGH | | | | |
| | | | | | EPEEEDEEEEEEEEELLLENESRPESSFSM | | | | |
| | | | | | DSELSRNRENGGGGVPGVPGAGGGAAKA | | | | |
| | | | | | LADEKALVLGKVMENVGLGALPQYGELL | | | | |
| | | | | | ADKQKRGAFLKRAAGGGDAGDDDDAGG | | | | |
| | | | | | CGDAGAGGAVNGRGGGFAPGTEPFPGLF | | | | |
| | | | | | PRKPAPLPSPGLNSAAKRIKVEKDLELPPA | | | | |
| | | | | | ALIPSENVYSQWLVGYAASRHFMKDPFL | | | | |
| | | | | | GFTDARQSPFATSSEHSSENGSLRFSTPPG | | | | |
| | | | | | DLLDGGLSGRSGTASGGSTPHLGGPGPGR | | | | |
| | | | | | PSSKEGRRSDTCEYCGKVFKNCSNLTVHR | | | | |
| | | | | | RSHTGERPYKCELCNYACAQSSKLTRHM | | | | |
| | | | | | KTHGQIGKEVYRCDICQMPFSVYSTLEKH | | | | |
| | | | | | MKKWHGEHLLTNDVKIEQAERS | | | | |
| RAD21 | IL2RA | 5885 | NM_006265.2 | NC_000008.11 | MFYAHFVLSKRGPLAKIWLAAHWDKKLT | 85 | Negative | NS | NS |
| | | | | | KAHVFECNLESSVESIISPKVKMALRTSGH | | Regulator | | |
| | | | | | LLLGVVRIYHRKAKYLLADCNEAFIKIKM | | | | |
| | | | | | AFRPGVVDLPEENREAAYNATTLPEEFHD | | | | |
| | | | | | FDQPLPDLDDIDVAQQFSLNQSRVEEITM | | | | |
| | | | | | REEVGNISILQENDFGDFGMDDREIMREG | | | | |
| | | | | | SAFEDDDMLVSTTTSNLLLESEQSTSNLN | | | | |
| | | | | | EKINHLEYEDQYKDDNFGEGNDGGILDD | | | | |
| | | | | | KLISNNDGGIFDDPPALSEAGVMLPEQPA | | | | |
| | | | | | HDDMDEDDNVSMGGPDSPDSVDPVEPMP | | | | |
| | | | | | TMTDQTTLVPNEEEAFALEPIDITVKETKA | | | | |
| | | | | | KRKRKLIVDSVKELDSKTIRAQLSDYSDIV | | | | |
| | | | | | TTLDLAPPTKKLMMWKETGGVEKLFSLP | | | | |
| | | | | | AQPLWNNRLLKLFTRCLTPLVPEDLRKRR | | | | |
| | | | | | KGGEADNLDEFLKEFENPEVPREDQQQQ | | | | |
| | | | | | HQQRDVIDEPHIEEPSRLQESVMEASRTNI | | | | |
| | | | | | DESAMPPPPPQGVKRKAGQIDPEPVMPPQ | | | | |
| | | | | | QVEQMEIPPVELPPEEPPNICQLIPELELLP | | | | |
| | | | | | EKEKEKEKEKEDDEEEEDEDASGGDQDQ | | | | |
| | | | | | EERRWNKRTQQMLHGLQRALAKTGAESI | | | | |
| | | | | | SLLELCRNTNRKQAAAKFYSFLVLKKQQ | | | | |
| | | | | | AIELTQEEPYSDIIATPGPRFHII | | | | |
| HIVEP2 | IL2RA | 3097 | NM_006734.3 | NC_000006.12 | MDTGDTALGQKATSRSGETDKASGRWR | 86 | NS | Negative | NS |
| | | | | | QEQSAVIKMSTFGSHEGQRQPQIEPEQIGN | | | Regulator | |
| | | | | | TASAQLFGSGKLASPSEVVQQVAEKQYPP | | | | |
| | | | | | HRPSPYSCQHSLSFPQHSLPQGVMHSTKP | | | | |
| | | | | | HQSLEGPPWLFPGPLPSVASEDLFPFPIHG | | | | |
| | | | | | HSGGYPRKKISSLNPAYSQYSQKSIEQAEE | | | | |
| | | | | | AHKKEHKPKKPGKYICPYCSRACAKPSVL | | | | |
| | | | | | KKHIRSHTGERPYPCIPCGFSFKTKSNLYK | | | | |
| | | | | | HRKSHAHAIKAGLVPFTESAVSKLDLEAG | | | | |
| | | | | | FIDVEAEIHSDGEQSTDTDEESSLFAEASD | | | | |
| | | | | | KMSPGPPIPLDIASRGGYHGSLEESLGGPM | | | | |
| | | | | | KVPILIIPKSGIPLPNESSQYIGPDMLPNPSL | | | | |
| | | | | | NTKADDSHTVKQKLALRLSEKKGQDSEP | | | | |
| | | | | | SLNLLSPHSKGSTDSGYFSRSESAEQQISPP | | | | |
| | | | | | NTNAKSYEEUFGKYCRLSPRNALSVTTTS | | | | |
| | | | | | QERAAMGRKGIMEPLPHVNTRLDVKMFE | | | | |
| | | | | | DPVSQLIPSKGDVDPSQTSMLKSTKENSES | | | | |
| | | | | | RQPQIIPSSIRNEGKLYPANFQGSNPVLLE | | | | |
| | | | | | APVDSSPLIRSNSVPTSSATNLTIPPSLRGS | | | | |
| | | | | | HSFDERMTGSDDVFYPGTVGIPPQRMLRR | | | | |
| | | | | | QAAFELPSVQEGHVEVEHHGRMLKGISSS | | | | |
| | | | | | SLKEKKLSPGDRVGYDYDVCRKPYKKWE | | | | |
| | | | | | DSETPKQNYRDISCLSSLKHGGEYFMDPV | | | | |
| | | | | | VPLQGVPSMFGTTCENRKRRKEKSVGDE | | | | |
| | | | | | EDTPMICSSIVSTPVGIMASDYDPKLQMQ | | | | |
| | | | | | EGVRSGFAMAGHENLSHGHTERFDPCRP | | | | |
| | | | | | QLQPGSPSLVSEESPSAIDSDKMSDLGGRK | | | | |
| | | | | | PPGNVISVIQHTNSLSRPNSFERSESAELVA | | | | |
| | | | | | CTQDKAPSPSETCDSEISEAPVSPEWAPPG | | | | |
| | | | | | DGAESGGKPSPSQQVQQQSYHTQPRLVR | | | | |
| | | | | | QHNIQVPEIRVTEEPDKPEKEKEAQSKEPE | | | | |
| | | | | | KPVEEFQWPQRSETLSQLPAEKLPPKKKR | | | | |
| | | | | | LRLADMEHSSGESSFESTGTGLSRSPSQES | | | | |
| | | | | | NLSHSSSFSMSFEREETSKLSALPKQDEFG | | | | |
| | | | | | KHSEFLTVPAGSYSLSVPGHHHQKEMRR | | | | |
| | | | | | CSSEQMPCPHPAEVPEVRSKSFDYGNLSH | | | | |
| | | | | | APVSGAAASTVSPSRERKKCFLVRQASFS | | | | |
| | | | | | GSPEISQGEVGMDQSVKQEQLEHLHAGL | | | | |
| | | | | | RSGWHHGPPAVLPPLQQEDPGKQVAGPC | | | | |
| | | | | | PPLSSGPLHLAQPQIMHMDSQESLRNPLIQ | | | | |
| | | | | | PTSYMTSKHLPEQPHLFPHQETIPFSPIQNA | | | | |
| | | | | | LFQFQYPTVCMVHLPAQQPPWWQAHFPH | | | | |
| | | | | | PFAQHPQKSYGKPSFQTEIHSSYPLEHVAE | | | | |
| | | | | | HTGKKPAEYAHTKEQTYPCYSGASGLHP | | | | |
| | | | | | KNLLPKFPSDQSSKSTETPSEQVLQEDFAS | | | | |
| | | | | | ANAGSLQSLPGTVVPVRIQTHVPSYGSVM | | | | |
| | | | | | YTSISQILGQNSPAIVICKVDENMTQRTLV | | | | |
| | | | | | TNAAMQGIGFNIAQVLGQHAGLEKYPIW | | | | |
| | | | | | KAPQTLPLGLESSIPLCLPSTSDSVATLGGS | | | | |
| | | | | | KRMLSPASSLELFMETKQQKRVKEEKMY | | | | |
| | | | | | GQIVEELSAVELTINSDIKKDLSRPQKPQLV | | | | |
| | | | | | RQGCASEPKDGLQSGSSSFSSLSPSSSQDY | | | | |
| | | | | | PSVSPSSREPFLPSKEMLSGSRAPLPGQKS | | | | |
| | | | | | SGPSESKESSDELDIDETASDMSMSPQSSS | | | | |
| | | | | | LPAGDGQLEEEGKGHKRPVGMLVRMAS | | | | |
| | | | | | APSGNVADSTLLLTDMADFQQILQFPSLR | | | | |
| | | | | | TTTTVSWCFLNYTKPNYVQQATFKSSVY | | | | |
| | | | | | ASWCISSCNPNPSGLNTKTTLALLRSKQKI | | | | |
| | | | | | TAEIYTLAAMHRPGTGKLTSSSAWKQFTQ | | | | |
| | | | | | MKPDASFLFGSKLERKLVGNILKERGKGD | | | | |
| | | | | | IHGDKDIGSKQTEPIRIKIFEGGYKSNEDY | | | | |
| | | | | | VYVRGRGRGKYICEECGIRCKKPSMLKK | | | | |
| | | | | | HIRTHTDVRPYVCKLQNFAFKTKGNLTKH | | | | |
| | | | | | MKSKAHMKKCLELGVSMTSVDDTETEEA | | | | |
| | | | | | ENLEDLHKAAEKHSMSSISTDHQFSDAEE | | | | |
| | | | | | SDGEDGDDNDDDDEDEDDFDDQGDLTPK | | | | |
| | | | | | TRSRSTSPQPPRFSSLPVNVGAVPHGVPSD | | | | |
| | | | | | SSLGHSSLISYLVTLPSIRVTQLMIPSDSCE | | | | |
| | | | | | DTQMTEYQRLFQSKSTDSEPDKDRLDIPS | | | | |
| | | | | | CMDEECMLPSEPSSSPRDFSPSSHHSSPGY | | | | |
| | | | | | DSSPCRDNSPKRYLIPKGDLSPRRHLSPRR | | | | |
| | | | | | DLSPMRHLSPRKEAALRREMSQRDVSPRR | | | | |
| | | | | | HLSPRRPVSPGKDITARRDLSPRRERRYM | | | | |
| | | | | | TTIRAPSPRRALYHNPPLSMGQYLQAEPIV | | | | |
| | | | | | LGPPNLRRGLPQVPYFSLYGDQEGAYEHP | | | | |
| | | | | | GSSLFPEGPNDYVFSHLPLHSQQQVRAPIP | | | | |
| | | | | | MVPVGGIQMVHSMPPALSSLHPSPTLPLP | | | | |
| | | | | | MEGFEEKKGASGESFSKDPYVLSKQHEK | | | | |
| | | | | | RGPHALQSSGPPSTPSSPRLLMKQSTSEDS | | | | |
| | | | | | LNATEREQEENIQTCTKAIASLRIATEEAA | | | | |
| | | | | | LLGPDQPARVQEPHQNPLGSAHVSIRHFS | | | | |
| | | | | | RPEPGQPCTSATHPDLHDGEKDNFGTSQT | | | | |
| | | | | | PLAHSTFYSKSCVDDKQLDFHSSKELSSST | | | | |
| | | | | | EESKDPSSEKSQLH | | | | |
| IRF2 | IL2RA | 3660 | NM_002199.3 | NC_000004.12 | MPVERMRMRPWLEEQINSNTIPGLKWLN | 87 | Negative | Negative | Negative |
| | | | | | KEKKIFQIPWMHAARHGWDVEKDAPLFR | | Regulator | Regulator | Regulator |
| | | | | | NWAIHTGKHQPGVDKPDPKTWKANFRC | | | | |
| | | | | | AMNSLPDIEEVKDKSIKKGNNAFRVYRM | | | | |
| | | | | | LPLSERPSKKGKKPKTEKEDKVKHIKQEP | | | | |
| | | | | | VESSLGLSNGVSDLSPEYAVLTSTIKNEVD | | | | |
| | | | | | STVNIIVVGQSHLDSNIENQEIVTNPPDICQ | | | | |
| | | | | | VVEVTTESDEQPVSMSELYPLQISPVSSYA | | | | |
| | | | | | ESETTDSVPSDEESAEGRPHWRKRNIEGK | | | | |
| | | | | | QYLSNMGTRGSYLLPGMASFVTSNKPDL | | | | |
| | | | | | QVTIKEESNPVPYNSSWPPFQDLPLSSSMT | | | | |
| | | | | | PASSSSRPDRETRASVIKKTSDITQARVKS | | | | |
| | | | | | C | | | | |
| TFAP4 | IL2RA | 7023 | NM_003223.2 | NC_000016.10 | MEYFMVPTQKVPSLQHFRKTEKEVIGGLC | 88 | NS | Positive | Positive |
| | | | | | SLANIPLTPETQRDQERRIRREIANSNERR | | | Regulator | Regulator |
| | | | | | RMQSINAGFQSLKTLIPHTDGEKLSKAAIL | | | | |
| | | | | | QQTAEYIFSLEQEKTRLLQQNTQLKRFIQE | | | | |
| | | | | | LSGSSPKRRRAEDKDEGIGSPDIWEDEKA | | | | |
| | | | | | EDLRREMIELRQQLDKERSVRMMLEEQV | | | | |
| | | | | | RSLEAHMYPEKLKVIAQQVQLQQQQEQV | | | | |
| | | | | | RLLHQEKLEREQQQLRTQLLPPPAPTHHP | | | | |
| | | | | | TVIVPAPPPPPSHHINVVTMGPSSVINSVST | | | | |
| | | | | | SRQNLDTIVQAIQHIEGTQEKQELEEEQRR | | | | |
| | | | | | AVIVKPVRSCPEAPTSDTASDSEASDSDA | | | | |
| | | | | | MDQSREEPSGDGELP | | | | |
| PRDM1 | IL2RA | 639 | NM_001198.3 | NC_000006.12 | MLDICLEKRVGTTLAAPKCNSSTVRFQGL | 89 | NS | Negative | Positive |
| | | | | | AEGTKGTMKMDMEDADMTLWTEAEFEE | | | Regulator | Regulator |
| | | | | | KCTYIVNDHPWDSGADGGTSVQAEASLP | | | | |
| | | | | | RNLLFKYATNSEEVIGVMSKEYIPKGTRF | | | | |
| | | | | | GPLIGEIYTNDTVPKNANRKYFWRIYSRG | | | | |
| | | | | | ELHHFIDGFNEEKSNWMRYVNPAHSPRE | | | | |
| | | | | | QNLAACQNGMNIYFYTIKPIPANQELLVW | | | | |
| | | | | | YCRDFAERLHYPYPGELTMMNLTQTQSS | | | | |
| | | | | | LKQPSTEKNELCPKNVPKREYSVKEILKL | | | | |
| | | | | | DSNPSKGKDLYRSNISPLTSEKDLDDFRRR | | | | |
| | | | | | GSPEMPFYPRVVYPIRAPLPEDFLKASLAY | | | | |
| | | | | | GIERPTYITRSPIPSSTTPSPSARSSPDQSLK | | | | |
| | | | | | SSSPHSSPGNTVSPVGPGSQEHRDSYAYL | | | | |
| | | | | | NASYGTEGLGSYPGYAPLPHLPPAFIPSYN | | | | |
| | | | | | AHYPKFLLPPYGMNCNGLSAVSSMNGIN | | | | |
| | | | | | NFGLFPRLCPVYSNLLGGGSLPHPMLNPT | | | | |
| | | | | | SLPSSLPSDGARRLLQPEHPREVLVPAPHS | | | | |
| | | | | | AFSFTGAAASMKDKACSPTSGSPTAGTAA | | | | |
| | | | | | TAEHVVQPKATSAAMAAPSSDEAMNLIK | | | | |
| | | | | | NKRNMTGYKTLPYPLKKQNGKIKYECNV | | | | |
| | | | | | CAKTFGQLSNLKVHLRVHSGERPFKCQTC | | | | |
| | | | | | NKGFTQLAHLQKHYLVHTGEKPHECQVC | | | | |
| | | | | | HKRFSSTSNLKTHLRLHSGEKPYQCKVCP | | | | |
| | | | | | AKFTQFVHLKLHKRLHTRERPHKCSQCH | | | | |
| | | | | | KNYIHLCSLKVHLKGNCAAAPAPGLPLED | | | | |
| | | | | | LTRINEEIEKFDISDNADRLEDVEDDISVIS | | | | |
| | | | | | VVEKEILAVVRKEKEETGLKVSLQRNMG | | | | |
| | | | | | NGLLSSGCSLYESSDLPLMKLPPSNPLPLV | | | | |
| | | | | | PVKVKQETVEPMDP | | | | |
| TNFAIP3 | IL2RA | 7128 | NM_ | NC_000006.12 | MAEQVLPQALYLSNMRKAVKIRERTPEDI | 90 | NS | Negative | NS |
| | | | 001270507.1 | | FKPTNGIIHHFKTMHRYTLEMFRTCQFCP | | | Regulator | |
| | | | | | QFREIIHKALIDRNIQATLESQKKLNWCRE | | | | |
| | | | | | VRKLVALKTNGDGNCLMHATSQYMWG | | | | |
| | | | | | VQDTDLVLRKALFSTLKETDTRNFKFRW | | | | |
| | | | | | QLESLKSQEFVETGLCYDTRNWNDEWDN | | | | |
| | | | | | LIKMASTDTPMARSGLQYNSLEEIHIFVLC | | | | |
| | | | | | NILRRPIIVISDKMLRSLESGSNFAPLKVGG | | | | |
| | | | | | IYLPLHWPAQECYRYPIVLGYDSHHFVPL | | | | |
| | | | | | VTLKDSGPEIRAVPLVNRDRGRFEDLKVH | | | | |
| | | | | | FLTDPENEMKEKLLKEYLMVIEIPVQGWD | | | | |
| | | | | | HGTTHLINAAKLDEANLPKEINLVDDYFE | | | | |
| | | | | | LVQHEYKKWQENSEQGRREGHAQNPME | | | | |
| | | | | | PSVPQLSLMDVKCETPNCPFFMSVNTQPL | | | | |
| | | | | | CHECSERRQKNQNKLPKLNSKPGPEGLPG | | | | |
| | | | | | MALGASRGEAYEPLAWNPEESTGGPHSA | | | | |
| | | | | | PPTAPSPFLFSETTAMKCRSPGCPFTLNVQ | | | | |
| | | | | | HNGFCERCHNARQLHASHAPDHTRHLDP | | | | |
| | | | | | GKCQACLQDVTRTENGICSTCFKRTTAEA | | | | |
| | | | | | SSSLSTSLPPSCHQRSKSDPSRLVRSPSPHS | | | | |
| | | | | | CHRAGNDAPAGCLSQAARTPGDRTGTSK | | | | |
| | | | | | CRKAGCVYFGTPENKGFCTLCFIEYRENK | | | | |
| | | | | | HFAAASGKVSPTASRFQNTIPCLGRECGT | | | | |
| | | | | | LGSTMFEGYCQKCFIEAQNQRFHEAKRTE | | | | |
| | | | | | EQLRSSQRRDVPRTTQSTSRPKCARASCK | | | | |
| | | | | | NILACRSEELCMECQHPNQRMGPGAHRG | | | | |
| | | | | | EPAPEDPPKQRCRAPACDHFGNAKQNGY | | | | |
| | | | | | CNECFQFKQMYG | | | | |
| MEF2D | IL2RA | 4209 | NM_005920.3 | NC_000001.11 | MGRKKIQIQRITDERNRQVTFTKRKFGLM | 91 | Negative | Negative | NS |
| | | | | | KKAYELSVLCDCEIALIIFNHSNKLFQYAS | | Regulator | Regulator | |
| | | | | | TDMDKVLLKYTEYNEPHESRTNADIIETL | | | | |
| | | | | | RKKGFNGCDSPEPDGEDSLEQSPLLEDKY | | | | |
| | | | | | RRASEELDGLFRRYGSTVPAPNFAMPVTV | | | | |
| | | | | | PVSNQSSLQFSNPSGSLVTPSLVTSSLTDP | | | | |
| | | | | | RLLSPQQPALQRNSVSPGLPQRPASAGAM | | | | |
| | | | | | LGGDLNSANGACPSPVGNGYVSARASPG | | | | |
| | | | | | LLPVANGNSLNKVIPAKSPPPPTHSTQLGA | | | | |
| | | | | | PSRKPDLRVITSQAGKGLMHHLTEDHLDL | | | | |
| | | | | | NNAQRLGVSQSTHSLTTPVVSVATPSLLS | | | | |
| | | | | | QGLPFSSMPTAYNTDYQLTSAELSSLPAFS | | | | |
| | | | | | SPGGLSLGNVTAWQQPQQPQQPQQPQPP | | | | |
| | | | | | QQQPPQPQQPQPQQPQQPQQPPQQQSHLV | | | | |
| | | | | | PVSLSNLIPGSPLPHVGAALTVTTHPHISIK | | | | |
| | | | | | SEPVSPSRERSPAPPPPAVFPAARPEPGDG | | | | |
| | | | | | LSSPAGGSYETGDRDDGRGDFGPTLGLLR | | | | |
| | | | | | PAPEPEAEGSAVKRMRLDTWTLK | | | | |
| SMARCB1 | IL2RA | 6598 | NM_003073.3 | NC_000022.11 | MMMMALSKTFGQKPVKFQLEDDGEFYM | 92 | NS | NS | Positive |
| | | | | | IGSEVGNYLRMERGSLYKRYPSLWRRLAT | | | | Regulator |
| | | | | | VEERKKIVASSHGKKTKPNTKDHGYTTL | | | | |
| | | | | | ATSVTLLKASEVEEILDGNDEKYKAVSIST | | | | |
| | | | | | EPPTYLREQKAKRNSQWVPTLPNSSHHLD | | | | |
| | | | | | AVPCSTTINRNRMGRDKKRTFPLCFDDHD | | | | |
| | | | | | PAVIHENASQPEVLVPIRLDMEIDGQKLR | | | | |
| | | | | | DAFTWNMNEKLMTPEMFSEILCDDLDLN | | | | |
| | | | | | PLTFVPAIASAIRQQIESYPTDSILEDQSDQ | | | | |
| | | | | | RVIIKLNIHVGNISLVDQFEWDMSEKENSP | | | | |
| | | | | | EKFALKLCSELGLGGEFVTTIAYSIRGQLS | | | | |
| | | | | | WHQKTYAFSENPLPTVEIAIRNTGDADQ | | | | |
| | | | | | WCPLLETLTDAEMEKKIRDQDRNTRRMR | | | | |
| | | | | | RLANTAPAW | | | | |
| NFKB2 | IL2RA | 4791 | NM_ | NC_000010.11 | MESCYNPGLDGIIEYDDFKLNSSIVEPKEP | 93 | NS | NS | Positive |
| | | | 001077494.3 | | APETADGPYLVIVEQPKQRGFRFRYGCEG | | | | Regulator |
| | | | | | PSHGGLPGASSEKGRKTYPTVKICNYEGP | | | | |
| | | | | | AKIEVDLVTHSDPPRAHAHSLVGKQCSEL | | | | |
| | | | | | GICAVSVGPKDMTAQFNNLGVLHVTKKN | | | | |
| | | | | | MMGTMIQKLQRQRLRSRPQGLTEAEQRE | | | | |
| | | | | | LEQEAKELKKVMDLSIVRLRFSAFLRASD | | | | |
| | | | | | GSFSLPLKPVISQPIHDSKSPGASNLKISRM | | | | |
| | | | | | DKTAGSVRGGDEVYLLCDKVQKDDIEVR | | | | |
| | | | | | FYEDDENGWQAFGDFSPTDVHKQYAIVF | | | | |
| | | | | | RTPPYHKMKIERPVTVFLQLKRKRGGDVS | | | | |
| | | | | | DSKQFTYYPLVEDKEEVQRKRRKALPTFS | | | | |
| | | | | | QPFGGGSHMGGGSGGAAGGYGGAGGGG | | | | |
| | | | | | SLGFFPSSLAYSPYQSGAGPMGCYPGGGG | | | | |
| | | | | | GAQMAATVPSRDSGEEAAEPSAPSRTPQC | | | | |
| | | | | | EPQAPEMLQRAREYNARLFGLAQRSARA | | | | |
| | | | | | LLDYGVTADARALLAGQRHLLTAQDENG | | | | |
| | | | | | DTPLHLAIIHGQTSVIEQIVYVIHHAQDLG | | | | |
| | | | | | VVNLINHLHQTPLHLAVITGQTSVVSFLL | | | | |
| | | | | | RVGADPALLDRHGDSAMHLALRAGAGA | | | | |
| | | | | | PELLRALLQSGAPAVPQLLHMPDFEGLYP | | | | |
| | | | | | VHLAVRARSPECLDLLVDSGAEVEATER | | | | |
| | | | | | QGGRTALHLATEMEELGLVTHLVTKLRA | | | | |
| | | | | | NVNARTFAGNTPLHLAAGLGYPTLTRLLL | | | | |
| | | | | | KAGADIHAENEEPLCPLPSPPTSDSDSDSE | | | | |
| | | | | | GPEKDTRSSFRGHTPLDLTCSTKVKTLLL | | | | |
| | | | | | NAAQNTMEPPLTPPSPAGPGLSLGDTALQ | | | | |
| | | | | | NLEQLLDGPEAQGSWAELAERLGLRSLV | | | | |
| | | | | | DTYRQTTSPSGSLLRSYELAGGDLAGLLE | | | | |
| | | | | | ALSDMGLEEGVRLLRGPETRDKLPSTAEV | | | | |
| | | | | | KEDSAYGSQSVEQEAEKLGPPPEPPGGLC | | | | |
| | | | | | HGHPQPQVH | | | | |
| HNRNPK | IL2RA | 3190 | NM_002140.3 | NC_000009.12 | METEQPEETFPNTETNGEFGKRPAEDMEE | 94 | Negative | Negative | NS |
| | | | | | EQAFKRSRNTDEMVELRILLQSKNAGAVI | | Regulator | Regulator | |
| | | | | | GKGGKNIKALRTDYNASVSVPDSSGPERI | | | | |
| | | | | | LSISADIETIGEILKKIIPTLEEGLQLPSPTAT | | | | |
| | | | | | SQLPLESDAVECLNYQHYKGSDFDCELRL | | | | |
| | | | | | LIHQSLAGGIIGVKGAKIKELRENTQTTIK | | | | |
| | | | | | LFQECCPHSTDRVVLIGGKPDRVVECIKII | | | | |
| | | | | | LDLISESPIKGRAQPYDPNFYDETYDYGGF | | | | |
| | | | | | TMMFDDRRGRPVGFPMRGRGGFDRMPP | | | | |
| | | | | | GRGGRPMPPSRRDYDDMSPRRGPPPPPPG | | | | |
| | | | | | RGGRGGSRARNLPLPPPPPPRGGDLMAYD | | | | |
| | | | | | RRGRPGDRYDGMVGFSADETWDSAIDT | | | | |
| | | | | | WSPSEWQMAYEPQGGSGYDYSYAGGRG | | | | |
| | | | | | SYGDLGGPIITTQVTIPKDLAGSIIGKGGQR | | | | |
| | | | | | IKQIRHESGASIKIDEPLEGSEDRIITITGTQ | | | | |
| | | | | | DQIQNAQYLLQNSVKQYADVEGF | | | | |
| MTF1 | IL2RA | 4520 | NM_005955.2 | NC_000001.11 | MGEHSPDNNIIYFEAEEDELTPDDKMLRF | 95 | NS | Negative | Negative |
| | | | | | VDKNGLVPSSSGTVYDRTTVLIEQDPGTL | | | Regulator | Regulator |
| | | | | | EDEDDDGQCGEHLPFLVGGEEGFHLIDHE | | | | |
| | | | | | AMSQGYVQHIISPDQIHLTINPGSTPMPRN | | | | |
| | | | | | IEGATLTLQSECPETKRKEVKRYQCTFEG | | | | |
| | | | | | CPRTYSTAGNLRTHQKTHRGEYTFVCNQ | | | | |
| | | | | | EGCGKAFLTSYSLRIHVRVHTKEKPFECD | | | | |
| | | | | | VQGCEKAFNTLYRLKAHQRLHTGKTFNC | | | | |
| | | | | | ESEGCSKYFTTLSDLRKHIRTHTGEKPERC | | | | |
| | | | | | DHDGCGKAFAASHHLKTHVRTHTGERPF | | | | |
| | | | | | FCPSNGCEKTFSTQYSLKSHMKGHDNKG | | | | |
| | | | | | HSYNALPQHNGSEDTNHSLCLSDLSLLST | | | | |
| | | | | | DSELRENSSTTQGQDLSTISPAIIFESMFQN | | | | |
| | | | | | SDDTAIQEDPQQTASLTESENGDAESVSD | | | | |
| | | | | | VPPSTGNSASLSLPLVLQPGLSEPPQPLLP | | | | |
| | | | | | ASAPSAPPPAPSLGPGSQQAAFGNPPALLQ | | | | |
| | | | | | PPEVPVPHSTQFAANHQEFLPHPQAPQPIV | | | | |
| | | | | | PGLSVVAGASASAAAVASAVAAPAPPQS | | | | |
| | | | | | TTEPLPAMVQTLPLGANSVLTNNPTITITP | | | | |
| | | | | | TPNTAILQSSLVMGEQNLQWILNGATSSP | | | | |
| | | | | | QNQEQIQQASKVEKVFFTTAVPVASSPGS | | | | |
| | | | | | SVQQIGLSVPVIIIKQEEACQCQCACRDSA | | | | |
| | | | | | KERASSRRKGCSSPPPPEPSPQAPDGPSLQ | | | | |
| | | | | | LPAQTFSSAPVPGSSSSTLPSSCEQSRQAET | | | | |
| | | | | | PSDPQTETLSAMDVSEFLSLQSLDTPSNLI | | | | |
| | | | | | PIEALLQGEEEMGLTSSFSK | | | | |
| ABCF1 | IL2RA | 23 | NM_ | NC_000006.12 | MPKAPKQQPPEPEWIGDGESTSPSDKVVK | 96 | Negative | NS | NS |
| | | | 001025091.1 | | KGKKDKKIKKTFFEELAVEDKQAGEEEK | | Regulator | | |
| | | | | | VLKEKEQQQQQQQQQQKKKRDTRKGRR | | | | |
| | | | | | KKDVDDDGEEKELMERLKKLSVPTSDEE | | | | |
| | | | | | DEVPAPKPRGGKKTKGGNVFAALIQDQS | | | | |
| | | | | | EEEEEEEKHPPKPAKPEKNRINKAVSEEQ | | | | |
| | | | | | QPALKGKKGKEEKSKGKAKPQNKFAALD | | | | |
| | | | | | NEEEDKEEEIIKEKEPPKQGKEKAKKAEQ | | | | |
| | | | | | GSEEEGEGEEEEEEGGESKADDPYAHLSK | | | | |
| | | | | | KEKKKLKKQMEYERQVASLKAANAAEN | | | | |
| | | | | | DFSVSQAEMSSRQAMLENASDIKLEKFSIS | | | | |
| | | | | | AHGKELFVNADLYIVAGRRYGLVGPNGK | | | | |
| | | | | | GKTTLLKHIANRALSIPPNIDVLLCEQEVV | | | | |
| | | | | | ADETPAVQAVLRADTKRLKLLEEERRLQ | | | | |
| | | | | | GQLEQGDDTAAERLEKVYEELRATGAAA | | | | |
| | | | | | AEAKARRILAGLGFDPEMQNRPTQKFSGG | | | | |
| | | | | | WRMRVSLARALFMEPTLLMLDEPTNHLD | | | | |
| | | | | | LNAVIWINNYLQGWRKTLLIVSHDQGFL | | | | |
| | | | | | DDVCTDIIHLDAQRLHYYRGNYMTFKKM | | | | |
| | | | | | YQQKQKELLKQYEKQEKKLKELKAGGKS | | | | |
| | | | | | TKQAEKQTKEALTRKQQKCRRKNQDEES | | | | |
| | | | | | QEAPELLKRPKEYTVRFTFPDPPPLSPPVL | | | | |
| | | | | | GLHGVTFGYQGQKPLFKNLDFGIDMDSRI | | | | |
| | | | | | CIVGPNGVGKSTLLLLLTGKLTPTHGEMR | | | | |
| | | | | | KNHRLKIGFFNQQYAEQLRMEETPTEYLQ | | | | |
| | | | | | RGFNLPYQDARKCLGRFGLESHAHTIQIC | | | | |
| | | | | | KLSGGQKARVVFAELACREPDVLILDEPT | | | | |
| | | | | | NNLDIESIDALGEAINEYKGAVIVVSHDAR | | | | |
| | | | | | LITETNCQLWVVEEQSVSQIDGDFEDYKR | | | | |
| | | | | | EVLEALGEVMVSRPRE | | | | |
| TBP | IL2RA | 6908 | NM_003194.4 | NC_000006.12 | MDQNNSLPPYAQGLASPQGAMTPGIPIFS | 97 | Negative | NS | NS |
| | | | | | PMMPYGTGLTPQPIQNTNSLSILEEQQRQ | | Regulator | | |
| | | | | | QQQQQQQQQQQQQQQQQQQQQQQQQQ | | | | |
| | | | | | QQQQQQQQQQQQAVAAAAVQQSTSQQAT | | | | |
| | | | | | QGTSGQAPQLFHSQTLTTAPLPGTTPLYPS | | | | |
| | | | | | PMTPMTPITPATPASESSGIVPQLQNIVSTV | | | | |
| | | | | | NLGCKLDLKTIALRARNAEYNPKRFAAVI | | | | |
| | | | | | MRIREPRTTALIFSSGKMVCTGAKSEEQSR | | | | |
| | | | | | LAARKYARVVQKLGFPAKFLDFKIQNMV | | | | |
| | | | | | GSCDVKFPIRLEGLVLTHQQFSSYEPELFP | | | | |
| | | | | | GLIYRMIKPRIVLLIFVSGKVVLTGAKVRA | | | | |
| | | | | | EIYEAFENIYPILKGFRKTT | | | | |
| YBX1 | IL2RA | 4904 | NM_004559.3 | NC_000001.11 | MSSEAETQQPPAAPPAAPALSAADTKPGT | 98 | Negative | NS | Positive |
| | | | | | TGSGAGSGGPGGLTSAAPAGGDKKVIAT | | Regulator | | Regulator |
| | | | | | KVLGTVKWFNVRNGYGFINRNDTKEDVF | | | | |
| | | | | | VHQTAIKKNNPRKYLRSVGDGETVEFDV | | | | |
| | | | | | VEGEKGAEAANVTGPGGVPVQGSKYAA | | | | |
| | | | | | DRNHYRRYPRRRGPPRNYQQNYQNSESG | | | | |
| | | | | | EKNEGSESAPEGQAQQRRPYRRRRFPPYY | | | | |
| | | | | | MRRPYGRRPQYSNPPVQGEVMEGADNQ | | | | |
| | | | | | GAGEQGRPVRQNMYRGYRPRFRRGPPRQ | | | | |
| | | | | | RQPREDGNEEDKENQGDETQGQQPPQRR | | | | |
| | | | | | YRRNFNYRRRRPENPKPQDGKETKAADP | | | | |
| | | | | | PAENSSAPEAEQGGAE | | | | |
| IRF4 | IL2RA | 3662 | NM_ | NC_000006.12 | MNLEGGGRGGEFGMSAVSCGNGKLRQW | 99 | Negative | Positive | Positive |
| | | | 001195286.1 | | LIDQIDSGKYPGLVWENEEKSIFRIPWKHA | | Regulator | Regulator | Regulator |
| | | | | | GKQDYNREEDAALFKAWALFKGKFREGI | | | | |
| | | | | | DKPDPPTWKTRLRCALNKSNDFEELVERS | | | | |
| | | | | | QLDISDPYKVYRIVPEGAKKGAKQLTLED | | | | |
| | | | | | PQMSMSHPYTMTTPYPSLPAQVHNYMMP | | | | |
| | | | | | PLDRSWRDYVPDQPHPEIPYQCPMTFGPR | | | | |
| | | | | | GHHWQGPACENGCQVTGTFYACAPPESQ | | | | |
| | | | | | APGVPTEPSIRSAEALAFSDCRLHICLYYR | | | | |
| | | | | | EILVKELTTSSPEGCRISHGHTYDASNLDQ | | | | |
| | | | | | VLFPYPEDNGQRKNIEKLLSHLERGVVLW | | | | |
| | | | | | MAPDGLYAKRLCQSRIYWDGPLALCNDR | | | | |
| | | | | | PNKLERDQTCKLFDTQQFLSELQAFAHHG | | | | |
| | | | | | RSLPRFQVILCFGEEFPDPQRQRKLITAHV | | | | |
| | | | | | EPLLARQLYYFAQQNSGHFLRGYDLPEHI | | | | |
| | | | | | SNPEDYHRSIRHSSIQE | | | | |
| SATB1 | IL2RA | 6304 | NM_002971.4 | NC_000003.12 | MDHLNEATQGKEHSEMSNNVSDPKGPPA | 100 | Negative | Negative | NS |
| | | | | | KIARLEQNGSPLGRGRLGSTGAKMQGVP | | Regulator | Regulator | |
| | | | | | LKHSGHLMKTNLRKGTMLPVFCVVEHYE | | | | |
| | | | | | NAIEYDCKEEHAEFVLVRKDMLFNQLIEM | | | | |
| | | | | | ALLSLGYSHSSAAQAKGLIQVGKWNPVP | | | | |
| | | | | | LSYVTDAPDATVADMLQDVYHVVTLKIQ | | | | |
| | | | | | LHSCPKLEDLPPEQWSHTTVRNALKDLLK | | | | |
| | | | | | DMNQSSLAKECPLSQSMISSIVNSTYYAN | | | | |
| | | | | | VSAAKCQEFGRWYKHFKKTKDMMVEM | | | | |
| | | | | | DSLSELSQQGANHVNFGQQPVPGNTABQ | | | | |
| | | | | | PPSPAQLSHGSQPSVRTPLPNLHPGLVSTPI | | | | |
| | | | | | SPQLVNQQLVMAQLLNQQYAVNRLLAQ | | | | |
| | | | | | QSLNQQYLNHPPPVSRSMNKPLEQQVSTN | | | | |
| | | | | | TEVSSEIYQWVRDELKRAGISQAVFARVA | | | | |
| | | | | | FNRTQGLLSEILRKEEDPKTASQSLLVNLR | | | | |
| | | | | | AMQNFLQLPEAERDRIYQDERERSLNAAS | | | | |
| | | | | | AMGPAPLISTPPSRPPQVKTATIATERNGK | | | | |
| | | | | | PENNTMNINASIYDEIQQEMKRAKVSQAL | | | | |
| | | | | | FAKVAATKSQGWLCELLRWKEDPSPENR | | | | |
| | | | | | TLWENLSMIRRFLSLPQPERDAIYEQESNA | | | | |
| | | | | | VHHHGDRPPHIIHVPAEQIQQQQQQQQQQ | | | | |
| | | | | | QQQQQAPPPPQPQQQPQTGPRLPPRQPTV | | | | |
| | | | | | ASPAESDEENRQKTRPRTKISVEALGILQS | | | | |
| | | | | | FIQDVGLYPDEEAIQTLSAQLDLPKYTIIKF | | | | |
| | | | | | FQNQRYYLKHHGKLKDNSGLEVDVAEY | | | | |
| | | | | | KEEELLKDLEESVQDKNTNTLFSVKLEEE | | | | |
| | | | | | LSVEGNTDINTDLKD | | | | |
| CREB1 | IL2RA | 1385 | NM_004379.3 | NC_000002.12 | MTMESGAENQQSGDAAVTEAENQQMTV | 101 | Negative | NS | NS |
| | | | | | QAQPQIATLAQVSMPAAHATSSAPTVTLV | | Regulator | | |
| | | | | | QLPNGQTVQVHGVIQAAQPSVIQSPQVQT | | | | |
| | | | | | VQISTIAESEDSQESVDSVTDSQKRREILSR | | | | |
| | | | | | RPSYRKILNDLSSDAPGVPRIEEEKSEEETS | | | | |
| | | | | | APAITTVTVPTPIYQTSSGQYIAITQGGAIQ | | | | |
| | | | | | LANNGTDGVQGLQTLTMTNAAATQPGTT | | | | |
| | | | | | ILQYAQTTDGQQILVPSNQVVVQAASGD | | | | |
| | | | | | VQTYQIRTAPTSTIAPGVVMASSPALPTQP | | | | |
| | | | | | AEEAARKREVRLMKNREAARECRRKKKE | | | | |
| | | | | | YVKCLENRVAVLENQNKTLIEELKALKD | | | | |
| | | | | | LYCHKSD | | | | |
| BATF | IL2RA | 10538 | NM_006399.3 | NC_000014.9 | MPHSSDSSDSSFSRSPPPGKQDSSDDVRRV | 102 | Negative | Positive | Positive |
| | | | | | QRREKNRIAAQKSRQRQTQKADTLHLESE | | Regulator | Regulator | Regulator |
| | | | | | DLEKQNAALRKEIKQLTEELKYFTSVLNS | | | | |
| | | | | | HEPLCSVLAASTPSPPEVVYSAHAFHQPH | | | | |
| | | | | | VSSPRFQP | | | | |
| IRF1 | IL2RA | 3659 | NM_002198.2 | NC_000005.10 | MPITRMRMRPWLEMQINSNQIPGLIWINK | 103 | Negative | Negative | Negative |
| | | | | | EEMIFQIPWKHAAKHGWDINKDACLFRS | | Regulator | Regulator | Regulator |
| | | | | | WAIHTGRYKAGEKEPDPKTWKANFRCA | | | | |
| | | | | | MNSLPDIEEVKDQSRNKGSSAVRVYRML | | | | |
| | | | | | PPLTKNQRKERKSKSSRDAKSKAKRKSCG | | | | |
| | | | | | DSSPDTFSDGLSSSTLPDDHSSYTVPGYM | | | | |
| | | | | | QDLEVEQALTPALSPCAVSSTLPDWHIPV | | | | |
| | | | | | EVVPDSTSDLYNFQVSPMPSTSEATTDED | | | | |
| | | | | | EEGKLPEDIMKLLEQSEWQPTNVDGKGY | | | | |
| | | | | | LLNEPGVQPTSVYGDFSCKEEPEIDSPGGD | | | | |
| | | | | | IGLSLQRVFTDLKNMDATWLDSLLTPVRL | | | | |
| | | | | | PSIQAIPCAP | | | | |
| CBFB | IL2RA | 865 | NM_001755.2 | NC_000016.10 | MPRVVPDQRSKFENEEFFRKLSRECEIKY | 104 | Negative | Negative | Positive |
| | | | | | TGFRDRPHEERQARFQNACRDGRSEIAFV | | Regulator | Regulator | Regulator |
| | | | | | ATGTNLSLQFFPASWQGEQRQTPSREYVD | | | | |
| | | | | | LEREAGKVYLKAPMILNGVCVIWKGWID | | | | |
| | | | | | LQRLDGMGCLEFDEERAQQEDALAQQAF | | | | |
| | | | | | EEARRRTREFEDRDRSHREEMEVRVSQLL | | | | |
| | | | | | AVTGKKTTRP | | | | |
| RNF20 | IL2RA | 56254 | NM_019592.6 | NC_000009.12 | MSGIGNKRAAGEPGTSMPPEKKAAVEDS | 105 | Negative | NS | NS |
| | | | | | GTTVETIKLGGVSSTEELDIRTLQTKNRKL | | Regulator | | |
| | | | | | AEMLDQRQAIEDELREHIEKLERRQATDD | | | | |
| | | | | | ASLLIVNRYWSQFDENIRIILKRYDLEQGL | | | | |
| | | | | | GDLLTERKALVVPEPEPDSDSNQERKDDR | | | | |
| | | | | | ERGEGQEPAFSFLATLASSSSEEMESQLQE | | | | |
| | | | | | RVESSRRAVSQIVTVYDKLQEKVELLSRK | | | | |
| | | | | | LNSGDNLIVEEAVQELNSFLAQENMRLQE | | | | |
| | | | | | LTDLLQEKHRTMSQEFSKLQSKVETAESR | | | | |
| | | | | | VSVLESMIDDLQWDIDKIRKREQRLNRHL | | | | |
| | | | | | AEVLERVNSKGYKVYGAGSSLYGGTITIN | | | | |
| | | | | | ARKFEEMNAELEENKELAQNRLCELEKL | | | | |
| | | | | | RQDFEEVTTQNEKLKVELRSAVEQVVKE | | | | |
| | | | | | TPEYRCMQSQFSVLYNESLQLKAHLDEA | | | | |
| | | | | | RTLLHGTRGTHQHQVELIERDEVSLHKKL | | | | |
| | | | | | RTEVIQLEDTLAQVRKEYEMLRIEFEQTL | | | | |
| | | | | | AANEQAGPINREMRHLISSLQNHNHQLKG | | | | |
| | | | | | EVLRYKRKLREAQSDLNKTRLRSGSALLQ | | | | |
| | | | | | SQSSTEDPKDEPAELKPDSEDLSSQSSASK | | | | |
| | | | | | ASQEDANEIKSKRDEEERERERREKERER | | | | |
| | | | | | EREREKEKEREREKQKLKESEKERDSAKD | | | | |
| | | | | | KEKGKHDDGRKKEAEIIKQLKIELKKAQE | | | | |
| | | | | | SQKEMKLLLDMYRSAPKEQRDKVQLMA | | | | |
| | | | | | AEKKSKAELEDLRQRLKDLEDKEKKENK | | | | |
| | | | | | KMADEDALRKIRAVEEQIEYLQKKLAMA | | | | |
| | | | | | KQEEEALLSEMDVTGQAFEDMQEQNIRL | | | | |
| | | | | | MQQLREKDDANFKLMSERIKSNQIHKLL | | | | |
| | | | | | KEEKEELADQVLTLKTQVDAQLQVVRKL | | | | |
| | | | | | EEKEHLLQSNIGTGEKELGLRTQALEMNK | | | | |
| | | | | | RKAMEAAQLADDLKAQLELAQKKLHDF | | | | |
| | | | | | QDEIVENSVTKEKDMFNFKRAQEDISRLR | | | | |
| | | | | | RKLETTKKPDNVPKCDEILMEEIKDYKAR | | | | |
| | | | | | LTCPCCNMRKKDAVLTKCFHVFCFECVK | | | | |
| | | | | | TRYDTRQRKCPKCNAAFGANDFHRIYIG | | | | |
| NR2C2 | IL2RA | 7182 | NM_003298.4 | NC_000003.12 | MTSPSPRIQIISTDSAVASPQRIQGSEPASG | 106 | Negative | NS | NS |
| | | | | | PLSVFTSLNKEKIVTDQQTGQKIQIVTAVD | | Regulator | | |
| | | | | | ASGSPKQQFILTSPDGAGTGKVILASPETS | | | | |
| | | | | | SAKQLIFTTSDNLVPGRIQIVTDSASVERL | | | | |
| | | | | | LGKTDVQRPQVVEYCVVCGDKASGRHY | | | | |
| | | | | | GAVSCEGCKGFFKRSVRKNLTYSCRSNQ | | | | |
| | | | | | DCIINKHHRNRCQFCRLKKCLEMGMKME | | | | |
| | | | | | SVQSERKPFDVQREKPSNCAASTEKTYIRK | | | | |
| | | | | | DLRSPLIATPTFVADKDGARQTGLLDPGM | | | | |
| | | | | | LVNIQQPLIREDGTVLLATDSKAETSQGA | | | | |
| | | | | | LGTLANVVTSLANLSESLNNGDTSEIQPE | | | | |
| | | | | | DQSASEITRAFDTLAKALNTTDSSSSPSLA | | | | |
| | | | | | DGIDTSGGGSIHVISRDQSTPIIEVEGPLLS | | | | |
| | | | | | DTHVTFKLTMPSPMPEYLNVHYICESASR | | | | |
| | | | | | LLFLSMHWARSIPAFQALGQDCNTSLVRA | | | | |
| | | | | | CWNELFTLGLAQCAQVMSLSTILAAIVNH | | | | |
| | | | | | LQNSIQEDKLSGDRIKQVMEHIWKLQEFC | | | | |
| | | | | | NSMAKLDIDGYEYAYLKAIVLFSPDHPGL | | | | |
| | | | | | TSTSQIEKFQEKAQMELQDYVQKTYSEDT | | | | |
| | | | | | YRLARILVRLPALRLMSSNITEELFFTGLIG | | | | |
| | | | | | NVSIDSHIPYILKMETAEYNGQITGASL | | | | |
| FOXK1 | IL2RA | 221937 | NM_ | NC_000007.14 | MAEVGEDSGARALLALRSAPCSPVLCAA | 107 | Negative | Negative | Negative |
| | | | 001037165.1 | | AAAAAFPAAAPPPAPAQPQPPPGPPPPPPP | | Regulator | Regulator | Regulator |
| | | | | | PLPPGAIAGAGSSGGSSGVSGDSAVAGAA | | | | |
| | | | | | PALVAAAAASVRQSPGPALARLEGREFEF | | | | |
| | | | | | LMRQPSVTIGRNSSQGSVDLSMGLSSFISR | | | | |
| | | | | | RHLQLSFQEPHFYLRCLGKNGVFVDGAF | | | | |
| | | | | | QRRGAPALQLPKQCTFRFPSTAIKIQFTSL | | | | |
| | | | | | YHKEEAPASPLRPLYPQISPLKIHIPEPDLR | | | | |
| | | | | | SMVSPVPSPTGTISVPNSCPASPRGAGSSS | | | | |
| | | | | | YRFVQNVTSDLQLAAEFAAKAASEQQAD | | | | |
| | | | | | TSGGDSPKDESKPPFSYAQLIVQAISSAQD | | | | |
| | | | | | RQLTLSGIYAHITKHYPYYRTADKGWQN | | | | |
| | | | | | SIRHNLSLNRYFIKVPRSQEEPGKGSFWRI | | | | |
| | | | | | DPASEAKLVEQAFRKRRQRGVSCFRTPFG | | | | |
| | | | | | PLSSRSAPASPTHPGLMSPRSGGLQTPECL | | | | |
| | | | | | SREGSPIPHDPEFGSKLASVPEYRYSQSAP | | | | |
| | | | | | GSPVSAQPVIMAVPPRPSSLVAKPVAYMP | | | | |
| | | | | | ASIVTSQQPAGHAIHVVQQAPTVTMVRV | | | | |
| | | | | | VTTSANSANGYILTSQGAAGGSHDAAGA | | | | |
| | | | | | AVLDLGSEARGLEEKPTIAFATIPAAGGVI | | | | |
| | | | | | QTVASQMAPGVPGHTVTILQPATPVTLGQ | | | | |
| | | | | | HHLPVRAVTQNGKHAVPTNSLAGNAYAL | | | | |
| | | | | | TSPLQLLATQASSSAPVVVTRVCEVGPKE | | | | |
| | | | | | PAAAVAATATTTPATATTASASASSTGEP | | | | |
| | | | | | EVKRSRVEEPSGAVTTPAGVIAAAGPQGP | | | | |
| | | | | | GTGE | | | | |
| MYC | IL2RA | 4609 | NM_002467.4 | NC_000008.11 | MDFFRVVENQQPPATMPLNVSFTNRNYD | 108 | Negative | NS | NS |
| | | | | | LDYDSVQPYFYCDEEENFYQQQQQSELQ | | Regulator | | |
| | | | | | PPAPSEDIWKKFELLPTPPLSPSRRSGLCSP | | | | |
| | | | | | SYVAVTPFSLRGDNDGGGGSFSTADQLE | | | | |
| | | | | | MVTELLGGDMVNQSFICDPDDETFIKNIII | | | | |
| | | | | | QDCMWSGFSAAAKLVSEKLASYQAARK | | | | |
| | | | | | DSGSPNPARGHSVCSTSSLYLQDLSAAAS | | | | |
| | | | | | ECIDPSVVFPYPLNDSSSPKSCASQDSSAFS | | | | |
| | | | | | PSSDSLLSSTESSPQGSPEPLVLHEETPPTT | | | | |
| | | | | | SSDSEEEQEDEEEIDVVSVEKRQAPGKRSE | | | | |
| | | | | | SGSPSAGGHSKPPHSPLVLKRCHVSTHQH | | | | |
| | | | | | NYAAPPSTRKDYPAAKRVKLDSVRVLRQI | | | | |
| | | | | | SNNRKCTSPRSSDTEENVKRRTHNVLERQ | | | | |
| | | | | | RRNELKRSFFALRDQIPELENNEKAPKVVI | | | | |
| | | | | | LKKATAYILSVQAEEQKLISEEDLLRKRRE | | | | |
| | | | | | QLKHKLEQLRNSCA | | | | |
| KLF2 | IL2RA | 10365 | NM_016270.2 | NC_000019.10 | MALSEPILPSFSTFASPCRERGLQERWPRA | 109 | Negative | Negative | NS |
| | | | | | EPESGGTDDDLNSVLDFILSMGLDGLGAE | | Regulator | Regulator | |
| | | | | | AAPEPPPPPPPPAFYYPEPGAPPPYSAPAG | | | | |
| | | | | | GLVSELLRPELDAPLGPALHGRFLLAPPG | | | | |
| | | | | | RLVKAEPPEADGGGGYGCAPGLTRGPRG | | | | |
| | | | | | LKREGAPGPAASCMRGPGGRPPPPPDTPP | | | | |
| | | | | | LSPDGPARLPAPGPRASFPPPFGGPGFGAP | | | | |
| | | | | | GPGLHYAPPAPPAFGLFDDAAAAAAALG | | | | |
| | | | | | LAPPAARGLLTPPASPLELLEAKPKRGRRS | | | | |
| | | | | | WPRKRTATHTCSYAGCGKTYTKSSHLKA | | | | |
| | | | | | HLRTHTGEKPYHCNWDGCGWKFARSDE | | | | |
| | | | | | LTRHYRKHTGHRPFQCHLCDRAFSRSDHL | | | | |
| | | | | | ALHMKRHM | | | | |
| TFDP1 | IL2RA | 7027 | NM_007111.4 | NC_000013.11 | MAKDAGLIEANGELKVFIDQNLSPGKGV | 110 | Negative | Negative | NS |
| | | | | | VSLVAVHPSTVNPLGKQLLPKTFGQSNVN | | Regulator | Regulator | |
| | | | | | IAQQVVIGTPQRPAASNTLVVGSPHTPSTH | | | | |
| | | | | | FASQNQPSDSSPWSAGKRNRKGEKNGKG | | | | |
| | | | | | LRHFSMKVCEKVQRKGTTSYNEVADELV | | | | |
| | | | | | AEFSAADNHILPNESAYDQKNIRRRVYDA | | | | |
| | | | | | LNVLMAMNIISKEKKEIKWIGLPTNSAQE | | | | |
| | | | | | CQNLEVERQRRLERIKQKQSQLQELILQQI | | | | |
| | | | | | AFKNLVQRNRHAEQQASRPPPPNSVIHLP | | | | |
| | | | | | FIIVNTSKKTVIDCSISNDKFEYLFNFDNTF | | | | |
| | | | | | EIHDDIEVLKRMGMACGLESGSCSAEDLK | | | | |
| | | | | | MARSLVPKALEPYVTEMAQGTVGGVFIT | | | | |
| | | | | | TAGSTSNGTRFSASDLINGADGMLATSSN | | | | |
| | | | | | GSQYSGSRVETPVSYVGEDDEEDDDFNE | | | | |
| | | | | | NDEDD | | | | |
| BPTF | IL2RA | 2186 | NM_182641.3 | NC_000017.11 | MRGRRGRPPKQPAAPAAERCAPAPPPPPP | 111 | Negative | Positive | NS |
| | | | | | PPTSGPIGGLRSRHRGSSRGRWAAAQAEV | | Regulator | Regulator | |
| | | | | | APKTRLSSPRGGSSSRRKPPPPPPAPPSTSA | | | | |
| | | | | | PGRGGRGGGGGRTGGGGGGGHLARTTA | | | | |
| | | | | | ARRAVNKVVYDDHESEEEEEEEDMVSEE | | | | |
| | | | | | EEEEDGDAEETQDSEDDEEDEMEEDDDD | | | | |
| | | | | | SDYPEEMEDDDDDASYCTESSFRSHSTYS | | | | |
| | | | | | STPGRRKPRVHRPRSPILEEKDIPPLEFPKS | | | | |
| | | | | | SEDLMVPNEHIMNVIAIYEVLRNFGTVLR | | | | |
| | | | | | LSPFRFEDFCAALVSQEQCTLMAEMHVV | | | | |
| | | | | | LLKAVLREEDTSNTTFGPADLKDSVNSTL | | | | |
| | | | | | YFIDGMTWPEVLRVYCESDKEYHHVLPY | | | | |
| | | | | | QEAEDYPYGPVENKIKVLQFLVDQFLTTN | | | | |
| | | | | | IAREELMSEGVIQYDDHCRVCHKLGDLLC | | | | |
| | | | | | CETCSAVYHLECVKPPLEEVPEDEWQCEV | | | | |
| | | | | | CVAHKVPGVTDCVAEIQKNKPYIRHEPIG | | | | |
| | | | | | YDRSRRKYWFLNRRLIIEEDTENENEKKI | | | | |
| | | | | | WYYSTKVQLAELIDCLDKDYWEAELCKI | | | | |
| | | | | | LEEMREEIHRHMDITEDLINKARGSNKSF | | | | |
| | | | | | LAAANEEILESIRAKKGDIDNVKSPEETEK | | | | |
| | | | | | DKNETENDSKDAEKNREEFEDQSLEKDS | | | | |
| | | | | | DDKTPDDDPEQGKSEVGDFKSEKSNGELS | | | | |
| | | | | | ESPGAGKGASGSTRIITRLRNPDSKLSQLK | | | | |
| | | | | | SQQVAAAAHEANKLFKEGKEVLVVNSQG | | | | |
| | | | | | EISRLSTKKEVIMKGNINNYFKLGQEGKY | | | | |
| | | | | | RVYHNQYSTNSFALNKHQHREDHDKRRH | | | | |
| | | | | | LAHKFCLTPAGEFKWNGSVHGSKVLTIST | | | | |
| | | | | | LRLTITQLENNIPSSFLHPNWASHRANWIK | | | | |
| | | | | | AVQMCSKPREFALALAILECAVKPVVML | | | | |
| | | | | | PIWRESLGHTRLHRMTSIEREEKEKVKKK | | | | |
| | | | | | EKKQEEEETMQQATWVKYTFPVKHQVW | | | | |
| | | | | | KQKGEEYRVTGYGGWSWISKTHVYRFVP | | | | |
| | | | | | KLPGNTNVNYRKSLEGTKNNMDENMDE | | | | |
| | | | | | SDKRKCSRSPKKIKIEPDSEKDEVKGSDA | | | | |
| | | | | | AKGADQNEMDISKITEKKDQDVKELLDS | | | | |
| | | | | | DSDKPCKEEPMEVDDDMKTESHVNCQES | | | | |
| | | | | | SQVDVVNVSEGFHLRTSYKKKTKSSKLD | | | | |
| | | | | | GLLERRIKQFTLEEKQRLEKIKLEGGIKGI | | | | |
| | | | | | GKTSTNSSKNLSESPVITKAKEGCQSDSM | | | | |
| | | | | | RQEQSPNANNDQPEDLIQGCSESDSSVLR | | | | |
| | | | | | MSDPSHTTNKLYPKDRVLDDVSIRSPETK | | | | |
| | | | | | CPKQNSIENDIEEKVSDLASRGQEPSKSKT | | | | |
| | | | | | KGNDFFIDDSKLASADDIGTLICKNKKPLI | | | | |
| | | | | | QEESDTIVSSSKSALHSSVPKSTNDRDATP | | | | |
| | | | | | LSRAMDFEGKLGCDSESNSTLENSSDTVSI | | | | |
| | | | | | QDSSEEDMIVQNSNESISEQFRTREQDVEV | | | | |
| | | | | | LEPLKCELVSGESTGNCEDRLPVKGTEAN | | | | |
| | | | | | GKKPSQQKKLEERPVNKCSDQIKLKNTTD | | | | |
| | | | | | KKNNENRESEKKGQRTSTFQINGKDNKP | | | | |
| | | | | | KIYLKGECLKEISESRVVSGNVEPKVNNIN | | | | |
| | | | | | KIIPENDIKSLTVKESAIRPFINGDVIMEDF | | | | |
| | | | | | NERNSSETKSHLLSSSDAEGNYRDSLETLP | | | | |
| | | | | | STKESDSTQTTTPSASCPESNSVNQVEDM | | | | |
| | | | | | EIETSEVKKVTSSPITSEEESNLSNDFIDEN | | | | |
| | | | | | GLPINKNENVNGESKRKTVITEVTTMTST | | | | |
| | | | | | VATESKTVIKVEKGDKQTVVSSTENCAKS | | | | |
| | | | | | TVTTTTTTVTKLSTPSTGGSVDIISVKEQS | | | | |
| | | | | | KTVVTTTVTDSLTTTGGTLVTSMTVSKEY | | | | |
| | | | | | STRDKVKLMKFSRPKKTRSGTALPSYRKF | | | | |
| | | | | | VTKSSKKSIFVLPNDDLKKLARKGGIREV | | | | |
| | | | | | PYFNYNAKPALDIWPYPSPRPTFGITWRY | | | | |
| | | | | | RLQTVKSLAGVSLMLRLLWASLRWDDM | | | | |
| | | | | | AAKAPPGGGTTRTETSETEITTTEIIKRRD | | | | |
| | | | | | VGPYGIRSEYCIRKIICPIGVPETPKETPTPQ | | | | |
| | | | | | RKGLRSSALRPKRPETPKQTGPVIIETWVA | | | | |
| | | | | | EEELELWEIRAFAERVEKEKAQAVEQQA | | | | |
| | | | | | KKRLEQQKPTVIATSTTSPTSSTTSTISPAQ | | | | |
| | | | | | KVMVAPISGSVTTGTKMVLTTKVGSPAT | | | | |
| | | | | | VTFQQNKNFHQTFATWVKQGQSNSGVV | | | | |
| | | | | | QVQQKVLGIIPSSTGTSQQTFTSFQPRTAT | | | | |
| | | | | | VTIRPNTSGSGGTTSNSQVITGPQIRPGMT | | | | |
| | | | | | VIRTPLQQSTLGKAIIRTPVMVQPGAPQQV | | | | |
| | | | | | MTQIIRGQPVSTAVSAPNTVSSTPGQKSLT | | | | |
| | | | | | SATSTSNIQSSASQPPRPQQGQVKLTMAQ | | | | |
| | | | | | LTQLTQGHGGNQGLTVVIQGQGQTTGQL | | | | |
| | | | | | QLIPQGVTVLPGPGQQLMQAAMPNGTVQ | | | | |
| | | | | | RFLFTPLATTATTASTTTTTVSTTAAGTGE | | | | |
| | | | | | QRQSKLSPQMQVHQDKTLPPAQSSSVGP | | | | |
| | | | | | AEAQPQTAQPSAQPQPQTQPQSPAQPEVQ | | | | |
| | | | | | TQPEVQTQTTVSSHVPSEAQPTHAQSSKP | | | | |
| | | | | | QVAAQSQPQSNVQGQSPVRVQSPSQTRIR | | | | |
| | | | | | PSTPSQLSPGQQSQVQTTTSQPIPIQPHTSL | | | | |
| | | | | | QIPSQGQPQSQPQVQSSTQTLSSGQTLNQ | | | | |
| | | | | | VTVSSPSRPQLQIQQPQPQVIAVPQLQQQV | | | | |
| | | | | | QVLSQIQSQVVAQIQAQQSGVPQQIKLQL | | | | |
| | | | | | PIQIQQSSAVQTHQIQNVVTVQAASVQEQ | | | | |
| | | | | | LQRVQQLRDQQQKKKQQQIEIKREHTLQ | | | | |
| | | | | | ASNQSEIIQKQVVMKHNAVIEHLKQKKS | | | | |
| | | | | | MTPAEREENQRMIVCNQVMKYILDKIDK | | | | |
| | | | | | EEKQAAKKRKREESVEQKRSKQNATKLS | | | | |
| | | | | | ALLFKHKEQLRAEILKKRALLDKDLQIEV | | | | |
| | | | | | QEELKRDLKIKKEKDLMQLAQATAVAAP | | | | |
| | | | | | CPPVTPAPPAPPAPPPSPPPPPAVQHTGLLS | | | | |
| | | | | | TPTLPAASQKRKREEEKDSSSKSKKKKMI | | | | |
| | | | | | STTSKETKKDTKLYCICKTPYDESKFYIGC | | | | |
| | | | | | DRCQNWYHGRCVGILQSEAELIDEYVCP | | | | |
| | | | | | QCQSTEDAMTVLTPLTEKDYEGLKRVLR | | | | |
| | | | | | SLQAHKMAWPFLEPVDPNDAPDYYGVIK | | | | |
| | | | | | EPMDLATMEERVQRRYYEKLTEFVADMT | | | | |
| | | | | | KIFDNCRYYNPSDSPFYQCAEVLESFFVQ | | | | |
| | | | | | KLKGFKASRSHNNKLQSTAS | | | | |
| KLF13 | IL2RA | 51621 | NM_015995.3 | NC_000015.10 | MAAAAYVDHFAAECLVSMSSRAVVHGP | 112 | Negative | Negative | NS |
| | | | | | REGPESRPEGAAVAATPTLPRVEERRDGK | | Regulator | Regulator | |
| | | | | | DSASLFVVARILADLNQQAPAPAPAERRE | | | | |
| | | | | | GAAARKARTPCRLPPPAPEPTSPGAEGAA | | | | |
| | | | | | AAPPSPAWSEPEPEAGLEPEREPGPAGSGE | | | | |
| | | | | | PGLRQRVRRGRSRADLESPQRKHKCHYA | | | | |
| | | | | | GCEKVYGKSSHLKAHLRTHTGERPFACS | | | | |
| | | | | | WQDCNKKFARSDELARHYRTHTGEKKFS | | | | |
| | | | | | CPICEKRFMRSDHLTKHARRHANFHPGM | | | | |
| | | | | | LQRRGGGSRTGSLSDYSRSDASSPTISPAS | | | | |
| | | | | | SP | | | | |
| MYB | IL2RA | 4602 | NM_005375.2 | NC_000006.12 | MARRPRHSIYSSDEDDEDFEMCDHDYDG | 113 | Negative | Negative | NS |
| | | | | | LLPKSGKRHLGKTRWTREEDEKLKKLVE | | Regulator | Regulator | |
| | | | | | QNGTDDWKVIANYLPNRTDVQCQHRWQ | | | | |
| | | | | | KVLNPELIKGPWTKEEDQRVIELVQKYGP | | | | |
| | | | | | KRWSVIAKHLKGRIGKQCRERWHNHLNP | | | | |
| | | | | | EVKKTSWTEEEDRIIYQAHKRLGNRWAEI | | | | |
| | | | | | AKLLPGRTDNAIKNHWNSTMRRKVEQEG | | | | |
| | | | | | YLQESSKASQPAVATSFQKNSHLMGFAQ | | | | |
| | | | | | APPTAQLPATGQPTVNNDYSYYHISEAQN | | | | |
| | | | | | VSSHVPYPVALHVNIVNVPQPAAAAIQRH | | | | |
| | | | | | YNDEDPEKEKRIKELELLLMSTENELKGQ | | | | |
| | | | | | QVLPTQNHTCSYPGWHSTTIADHTRPHGD | | | | |
| | | | | | SAPVSCLGEHHSTPSLPADPGSLPEESASP | | | | |
| | | | | | ARCMIVHQGTILDNVKNLLEFAETLQFIDS | | | | |
| | | | | | FLNTSSNHENSDLEMPSLTSTPLIGHKLTV | | | | |
| | | | | | TTPFHRDQTVKTQKENTVERTPAIKRSILE | | | | |
| | | | | | SSPRTPTPFKHALAAQEIKYGPLKMLPQTP | | | | |
| | | | | | SHLVEDLQDVIKQESDESGIVAEFQENGPP | | | | |
| | | | | | LLKKIKQEVESPTDKSGNFFCSHHWEGDS | | | | |
| | | | | | LNTQLFTQTSPVADAPNILTSSVLMAPASE | | | | |
| | | | | | DEDNVLKAFTVPKNRSLASPLQPCSSTWE | | | | |
| | | | | | PASCGKMEEQMTSSSQARKYVNAFSART | | | | |
| | | | | | LVM | | | | |
| ZNF217 | IL2RA | 7764 | NM_006526.2 | NC_000020.11 | MQSKVTGNMPTQSLLMYMDGPEVIGSSL | 114 | Negative | Negative | NS |
| | | | | | GSPMEMEDALSMKGTAVVPFRATQEKNV | | Regulator | Regulator | |
| | | | | | IQIEGYMPLDCMFCSQTFTHSEDLNKHVL | | | | |
| | | | | | MQHRPTLCEPAVLRVEAEYLSPLDKSQVR | | | | |
| | | | | | TEPPKEKNCKENEFSCEVCGQTFRVAFDV | | | | |
| | | | | | EIHMRTHKDSFTYGCNMCGRRFKEPWFL | | | | |
| | | | | | KNHMRTHNGKSGARSKLQQGLESSPATI | | | | |
| | | | | | NEVVQVHAAESISSPYKICMVCGFLFPNK | | | | |
| | | | | | ESLIEHRKVHTKKTAFGTSSAQTDSPQGG | | | | |
| | | | | | MPSSREDFLQLFNLRPKSHPETGKKPVRCI | | | | |
| | | | | | PQLDPFTTFQAWQLATKGKVAICQEVKES | | | | |
| | | | | | GQEGSTDNDDSSSEKELGETNKGSCAGLS | | | | |
| | | | | | QEKEKCKHSHGEAPSVDADPKLPSSKEKP | | | | |
| | | | | | THCSECGKAFRTYHQLVLHSRVHKKDRR | | | | |
| | | | | | AGAESPTMSVDGRQPGTCSPDLAAPLDEN | | | | |
| | | | | | GAVDRGEGGSEDGSEDGLPEGIHLDKND | | | | |
| | | | | | DGGKIKHLTSSRECSYCGKFFRSNYYLNI | | | | |
| | | | | | HLRTHTGEKPYKCEFCEYAAAQKTSLRY | | | | |
| | | | | | HLERHHKEKQTDVAAEVKNDGKNQDTE | | | | |
| | | | | | DALLTADSAQTKNLKRFFDGAKDVTGSP | | | | |
| | | | | | PAKQLKEMPSVFQNVLGSAVLSPAHKDT | | | | |
| | | | | | QDFHKNAADDSADKVNKNPTPAYLDLLK | | | | |
| | | | | | KRSAVETQANNLICRTKADVTPPPDGSTT | | | | |
| | | | | | HNLEVSPKEKQTETAADCRYRPSVDCHE | | | | |
| | | | | | KPLNLSVGALHNCPAISLSKSLIPSITCPFC | | | | |
| | | | | | TFKTFYPEVLMMHQRLEHKYNPDVHKNC | | | | |
| | | | | | RNKSLLRSRRTGCPPALLGKDVPPLSSFCK | | | | |
| | | | | | PKPKSAFPAQSKSLPSAKGKQSPPGPGKA | | | | |
| | | | | | PLTSGIDSSTLAPSNLKSHRPQQNVGVQG | | | | |
| | | | | | AATRQQQSEMFPKTSVSPAPDKTKRPETK | | | | |
| | | | | | LKPLPVAPSQPTLGSSNINGSIDYPAKNDS | | | | |
| | | | | | PWAPPGRDYFCNRSASNTAAEFGEPLPKR | | | | |
| | | | | | LKSSVVALDVDQPGANYRRGYDLPKYH | | | | |
| | | | | | MVRGITSLLPQDCVYPSQALPPKPRFLSSS | | | | |
| | | | | | EVDSPNVLTVQKPYGGSGPLYTCVPAGSP | | | | |
| | | | | | ASSSTLEGKRPVSYQHLSNSMAQKRNYE | | | | |
| | | | | | NFIGNAHYRPNDKKT | | | | |

TABLE 2

| | | | SEQ | | SEQ | |
|---|---|---|---|---|---|---|
| Gene | Screen | sgRNA Target | ID | Target Context | ID | Exon |
| ID | Target | Sequence | NO: | Sequence | NO: | Targeted |
| FOXN2 | IL2RA | AGAAGACTAAAGGAGTATGG | 115 | AATGAGAAGACTAAAGGAGTATGGGGGCTT | 563 | 3 |
| FOXN2 | IL2RA | CATAAACACTGACCTTGCCA | 116 | TGTTCATAAACACTGACCTTGCCATGGCTT | 564 | 3 |
| FOXN2 | IL2RA | TCTCCCTCTATGTCATACAA | 117 | ATCATCTCCCTCTATGTCATACAATGGACT | 565 | 3 |
| FOXN2 | IL2RA | TGACTCACTGTCCACTAGAG | 118 | CTGCTGACTCACTGTCCACTAGAGTGGCCT | 566 | 3 |
| ZNF25 | IL2RA | ACATCAGAGAATGCACACAG | 119 | CAGTACATCAGAGAATGCACACAGGGGAGA | 567 | 6 |
| ZNF25 | IL2RA | AGTATGCTGATGTACTATGA | 120 | AGTGAGTATGCTGATGTACTATGAGGGCAG | 568 | 6 |
| ZNF25 | IL2RA | ATACAGAGTCCTCTGAGCAG | 121 | CCTTATACAGAGTCCTCTGAGCAGGGGTCA | 569 | 3 |
| ZNF25 | IL2RA | TAGAAGTAGAATTTCCACAT | 122 | ATATTAGAAGTAGAATTTCCACATCGGGGC | 570 | 4 |
| THRA | IL2RA | GATCTTGGTAAACTCGCTGA | 123 | TGATGATCTTGGTAAACTCGCTGAAGGCTT | 571 | 7 |
| THRA | IL2RA | GGTAACTAGGGATATACCCT | 124 | TCCAGGTAACTAGGGATATACCCTGGGAGG | 572 | 4 |
| THRA | IL2RA | TACCGCTGTATCACTTGTGA | 125 | TCACTACCGCTGTATCACTTGTGAGGGCTG | 573 | 4 |
| THRA | IL2RA | TGGTTCTAGATGÅCTCGAAG | 126 | ACAGTGGTTCTAGATGACTCGAAGCGGGTG | 574 | 6 |
| ZBTB2 | IL2RA | ATGTGAAAGGTACTCCTGTG | 127 | CTAGATGTGAAAGGTACTCCTGTGTGGATC | 575 | 3 |
| ZBTB2 | IL2RA | CTTGACGTGGGCTATTGTGÅ | 128 | TTGGCTTGACGTGGGCTATTGTGAGGGACG | 576 | 3 |
| ZBTB2 | IL2RA | GACCCGGTTCGATTAGAACA | 129 | CATCGACCCGGTTCGATTAGAACAGGGCAT | 577 | 3 |
| ZBTB2 | IL2RA | GTATGTCAGTTGGTTTCAAG | 130 | GGCTGTATGTCAGTTGGTTTCAAGCGGACA | 578 | 3 |
| CREM | IL2RA | ACAATCCAGATTTCTAACCC | 131 | TGGAACAATCCAGATTTCTAACCCAGGATC | 579 | 6 |
| CREM | IL2RA | GCATATATCAGACTAGCACG | 132 | ACTAGCATATATCAGACTAGCACGGGGCAA | 580 | 5 |
| CREM | IL2RA | TACTGTACAATTGTAGCACC | 133 | TGCGTACTGTACAATTGTAGCACCTGGTGG | 581 | 6 |
| CREM | IL2RA | TGCCTACCTGAGCTAAAGCA | 134 | CTATTGCCTACCTGAGCTAAAGCAGGGATT | 582 | 3 |
| PTF1A | IL2RA | CCTGGCCTACCCGTGCGCCG | 135 | CGTGCCTGGCCTACCCGTGCGCCGGGGCGG | 583 | 1 |
| PTF1A | IL2RA | CTCGGTGAGCCAGACGACGG | 136 | GGGCCTCGGTGAGCCAGACGACGGGGGGGG | 584 | 1 |
| PTF1A | IL2RA | GTGGCTAAGGAACTCCACCT | 137 | GCTGGTGGCTAAGGAACTCCACCTCGGCCT | 585 | 1 |
| PTF1A | IL2RA | TCCTTCTTCGTACTTCGACG | 138 | CCTTTCCTTCTTCGTACTTCGACGAGGACG | 586 | 1 |
| IKZF2 | IL2RA | GAACTTAACATATGAGAAGG | 139 | ATATGAACTTAACATATGAGAAGGAGGCTG | 587 | 8 |
| KZF2 | IL2RA | GAGTAAAAGAAGCTCCACAC | 140 | TTCTGAGTAAAAGAAGCTCCACACTGGTTA | 588 | 5 |
| KZF2 | IL2RA | TAGCTACGCCTGTAGAAGAA | 141 | TCTGTAGCTACGCCTGTAGAAGAAGGGACG | 589 | 5 |
| IKZF2 | IL2RA | TGACATTCTGGAGATAGTTG | 142 | ATGCTGACATTCTGGAGATAGTTGTGGCAG | 590 | 6 |
| MYNN | IL2RA | ACTCGAGTTGACATCAGTTG | 143 | CAGAACTCGAGTTGACATCAGTTGTGGAAA | 591 | 3 |
| MYNN | IL2RA | CTCATAAGGACTCTTGACGC | 144 | CCGCCTCATAAGGACTCTTGACGCTGGCTA | 592 | 3 |
| MYNN | IL2RA | CTGTTGGAGTTTATATACAC | 145 | GAAACTGTTGGAGTTTATATACACAGGAAC | 593 | 2 |
| MYNN | IL2RA | TAAATGCCTTTCCACATAAG | 146 | TGGGTAAATGCCTTTCCACATAAGTGGCAG | 594 | 3 |
| BACH2 | IL2RA | AATTATGGACAGCCCCACGT | 147 | AGGGAATTATGGACAGCCCCACGTGGGCCA | 595 | 7 |
| BACH2 | IL2RA | CCCAGAATAAGAATGCACCG | 148 | TCACCCCAGAATAAGAATGCACCGAGGTGC | 596 | 7 |
| BACH2 | IL2RA | CTCTTCCTATTCCTACGCGG | 149 | CCTGCTCTTCCTATTCCTACGCGGAGGACG | 597 | 7 |
| BACH2 | IL2RA | GTTCAGCATAACGAAAAGTG | 150 | CTCTGTTCAGCATAACGAAAAGTGTGGAGC | 598 | 7 |
| IL15RA | IL2RA | CACTAATGCATTTGAGACTG | 151 | TACTCACTAATGCATTTGAGACTGGGGGTT | 599 | 2 |
| IL15RA | IL2RA | CTCATGACTGCTTATCTCTG | 152 | AGGACTCATGACTGCTTATCTCTGTGGTTC | 600 | 4 |
| IL15RA | IL2RA | GCCACCCTCCACAGTAACGA | 153 | CAGCGCCACCCTCCACAGTAACGACGGCAG | 601 | 3 |
| IL15RA | IL2RA | TGCGTGTTCCACGGACATGG | 154 | TGTCTGCGTGTTCCACGGACATGGGGGGAG | 602 | 2 |
| SP3 | IL2RA | ACTGCAGGCATTAATGCCGA | 155 | AATGACTGCAGGCATTAATGCCGACGGACA | 603 | 4 |
| SP3 | IL2RA | AGGAGCACCAAACCGATGGG | 156 | TAGGAGGAGCACCAAACCGATGGGAGGTTT | 604 | 4 |
| SP3 | IL2RA | ATAGCTTGACTACATCTAGT | 157 | ACAAATAGCTTGACTACATCTAGTGGGCAG | 605 | 4 |
| SP3 | IL2RA | GCAAACGTTTCAAGTACAAG | 158 | CTTGGCAAACGTTTCAAGTACAAGGGGTCC | 606 | 4 |
| ZBTB3 | IL2RA | AACTCCATAGTACCCCACGA | 159 | TGGGAACTCCATAGTACCCCACGAAGGAAA | 607 | 2 |
| ZBTB3 | IL2RA | GTTTGTAGGAATGGTCTCAG | 160 | AGTAGTTTGTAGGAATGGTCTCAGTGGAGC | 608 | 2 |
| ZBTB3 | IL2RA | TCGTCAAGGTGTGTAAGCGG | 161 | GACATCGTCAAGGTGTGTAAGCGGCGGCTT | 609 | 2 |
| ZBTB3 | IL2RA | TGAGCCACCAATGTCTAGTG | 162 | CAGATGAGCCACCAATGTCTAGTGGGGCTG | 610 | 2 |
| REST | IL2RA | AACTCGAAAATCAGTAACAG | 163 | CCAGAACTCGAAAATCAGTAACAGAGGTGA | 611 | 4 |
| REST | IL2RA | AATAGTCAGGAAGTGCCAAA | 164 | AAATAATAGTCAGGAAGTGCCAAAGGGTGA | 612 | 4 |
| REST | IL2RA | CAGGGCAATTGAACTGCCGT | 165 | CATACAGGGCAATTGAACTGCCGTGGGTTC | 613 | 4 |
| REST | IL2RA | GATTAGTATTGTAGCCGCAG | 166 | TATCGATTAGTATTGTAGCCGCAGCGGTCA | 614 | 2 |
| ELP2 | IL2RA | AATTTCATGCCAAGTCACCT | 167 | TTGCAATTTCATGCCAAGTCACCTGGGTAA | 615 | 13 |
| ELP2 | IL2RA | CCAGTACCAATATTAGCATG | 168 | TCCCCCAGTACCAATATTAGCATGTGGCAA | 616 | 6 |
| ELP2 | IL2RA | GTTATTGTACAGGTTCGAGT | 169 | GTCTGTTATTGTACAGGTTCGAGTAGGTGA | 617 | 11 |
| ELP2 | IL2RA | TGATAATCAAGTGATTCACT | 170 | GATCTGATAATCAAGTGATTCACTGGGAAA | 618 | 3 |
| STAT6 | IL2RA | ATCAAGCGGTGTGAGCGGAA | 171 | GAAGATCAAGCGGTGTGAGCGGAAGGGCAC | 619 | 11 |
| STAT6 | IL2RA | CAGCCACCACAAAGGGCACG | 172 | CGCTCAGCCACCACAAAGGGCACGCGGTCC | 620 | 13 |
| STAT6 | IL2RA | GCTGGAAGGCCTCCATACTG | 173 | CGGTGCTGGAAGGCCTCCATACTGAGGCTG | 621 | 13 |
| STAT6 | IL2RA | GCTGGGAATAAATGTCCACC | 174 | TGTAGCTGGGAATAAATGTCCACCAGGCTT | 622 | 8 |
| ZMYND8 | IL2RA | AGATGTATTCCGCATAGTCA | 175 | TGGAAGATGTATTCCGCATAGTCAGGGTGC | 623 | 6 |
| ZMYND8 | IL2RA | CACTTAGCGTGATAAACCCG | 176 | CAGACACTTAGCGTGATAAACCCGGGGACA | 624 | 4 |
| ZMYND8 | IL2RA | CTCTTCCGCCCAAACTTCCG | 177 | CCCGCTCTTCCGCCCAAACTTCCGCGGCTG | 625 | 15 |
| ZMYND8 | IL2RA | GGAGCGCGGCATATCCGACA | 178 | TGGGGGAGCGCGGCATATCCGACAAGGAAA | 626 | 11 |
| ZBTB14 | IL2RA | CTAAATGAACAACGCCTGGA | 179 | AACACTAAATGAACAACGCCTGGAAGGAGA | 627 | 4 |
| ZBTB14 | IL2RA | GTGAAAGATGAACAGACACC | 180 | TGAAGTGAAAGATGAACAGACACCAGGCTG | 628 | 4 |
| ZBTB14 | IL2RA | TGATGATGTAGAGGAAATCG | 181 | AGGATGATGATGTAGAGGAAATCGGGGATC | 629 | 4 |
| ZBTB14 | IL2RA | TTCCTGAACCCTGAGCGTTG | 182 | TCGCTTCCTGAACCCTGAGCGTTGTGGTGG | 630 | 4 |
| GFI1 | IL2RA | ACGTGCACCTCGAGCCCGTG | 183 | GCGCACGTGCACCTCGAGCCCGTGCGGCGT | 631 | 5 |
| GFI1 | IL2RA | CCGGTACATTCTCTAAACGG | 184 | GGCGCCGGTACATTCTCTAAACGGAGGGAA | 632 | 2 |
| GFI1 | IL2RA | TGGGCACATTGACTTCTCCG | 185 | GCGATGGGCACATTGACTTCTCCGAGGCTG | 633 | 4 |
| GFI1 | IL2RA | TTCAAACCGTACTCATGGAG | 186 | GCCTTTCAAACCGTACTCATGGAGCGGCCT | 634 | 4 |
| IFNGR2 | IL2RA | GGCCGACATCATGTCCATAG | 187 | TCACGGCCGACATCATGTCCATAGGGGTGA | 635 | 3 |
| IFNGR2 | IL2RA | TGGCCCTGAGCAATAGCACG | 188 | CCAGTGGCCCTGAGCAATAGCACGAGGCCT | 636 | 2 |
| IFNGR2 | IL2RA | TTCCGATAGTGTTGAAACCA | 189 | ACCATTCCGATAGTGTTGAAACCAAGGCAT | 637 | 3 |
| IFNGR2 | IL2RA | TTGTAAACAGTACACTCTGG | 190 | GGACTTGTAAACAGTACACTCTGGAGGGTT | 638 | 5 |
| IL4R | IL2RA | AAGGGATGAAGATCCTCACA | 191 | TGAAAAGGGATGAAGATCCTCACAAGGCTG | 639 | 11 |
| IL4R | IL2RA | CAGTGCGGATAACTATACAC | 192 | TGGTCAGTGCGGATAACTATACACTGGACC | 640 | 5 |
| IL4R | IL2RA | CCTGGATTATTATAGCCACG | 193 | GCATCCTGGATTATTATAGCCACGAGGCGG | 641 | 9 |
| IL4R | IL2RA | GGGTTGCCTGCGATGACGAG | 194 | AGCAGGGTTGCCTGCGATGACGAGGGGCGT | 642 | 11 |
| IL2RB | IL2RA | CAGGGTGACGATGTCAACTG | 195 | CCCTCAGGGTGACGATGTCAACTGTGGTCA | 643 | 5 |
| IL2RB | IL2RA | CCACAGATGCAACATAAGCT | 196 | AGACCCACAGATGCAACATAAGCTGGGAAA | 644 | 6 |
| IL2RB | IL2RA | GCTGGGAAAAGAACTTCGAG | 197 | CTCAGCTGGGAAAAGAACTTCGAGGGGTCT | 645 | 9 |
| IL2RB | IL2RA | TTGGGAAGGACACCATTCCG | 198 | GCCCTTGGGAAGGACACCATTCCGTGGCTC | 646 | 8 |
| NR4A3 | IL2RA | CCTGCGTGTACCAAATGCAG | 199 | CCTTCCTGCGTGTACCAAATGCAGCGGCCC | 647 | 4 |
| NR4A3 | IL2RA | CGGGTGGCTCTCAAGCGCGG | 200 | CGTACGGGTGGCTCTCAAGCGCGGCGGCCT | 648 | 4 |
| NR4A3 | IL2RA | GAAATCGACAGTACTGACAT | 201 | TTCTGAAATCGACAGTACTGACATCGGTTT | 649 | 5 |
| NR4A3 | IL2RA | TGGTCAGCTTGGTGTAGTCG | 202 | TCCATGGTCAGCTTGGTGTAGTCGGGGTTC | 650 | 4 |
| LEF1 | IL2RA | CCTGTATATCCCATCACGGG | 203 | TCAGCCTGTATATCCCATCACGGGTGGATT | 651 | 6 |
| LEF1 | IL2RA | GCAGTCAAATAAAGTGCCCG | 204 | CTTTGCAGTCAAATAAAGTGCCCGTGGTGC | 652 | 4 |
| LEF1 | IL2RA | GTACCCGGAATAACTCGAGT | 205 | TTATGTACCCGGAATAACTCGAGTAGGAGG | 653 | 3 |
| LEF1 | IL2RA | GTGTTACAATAGCTGGATGA | 206 | TGAGGTGTTACAATAGCTGGATGAGGGATG | 654 | 7 |
| ZBTB32 | IL2RA | CACAAGCACTCGCCACCAAG | 207 | GTTGCACAAGCACTCGCCACCAAGAGGCAG | 655 | 2 |
| ZBTB32 | IL2RA | CGTGGTTGAGGGAAAATCCA | 208 | CTCACGTGGTTGAGGGAAAATCCAGGGGGC | 656 | 2 |
| ZBTB32 | IL2RA | GTGATACTCTGATCACCGTA | 209 | CTCTGTGATACTCTGATCACCGTAGGGAGC | 657 | 2 |
| ZBTB32 | IL2RA | TGTCCCTAAATGCCCCCAAA | 210 | CCACTGTCCCTAAATGCCCCCAAAGGGCTC | 658 | 3 |
| ZKSCAN2 | IL2RA | AGATGCCCGAGAGTTAATCA | 211 | GAGCAGATGCCCGAGAGTTAATCAGGGCAT | 659 | 6 |
| ZKSCAN2 | IL2RA | CATAAAACCGAGTCTCACGG | 212 | GCTTCATAAAACCGAGTCTCACGGAGGATA | 660 | 6 |
| ZKSCAN2 | IL2RA | CCATACACTTGGCTATTTCG | 213 | AGCACCATACACTTGGCTATTTCGGGGACA | 661 | 5 |
| ZKSCAN2 | IL2RA | TGAACCGAAAGCGAGAACGT | 214 | CAGCTGAACCGAAAGCGAGAACGTCGGCCC | 662 | 2 |
| ARID5A | IL2RA | CCCCGCCGTACCTCTCGTAG | 215 | CCCGCCCCGCCGTACCTCTCGTAGTGGCGG | 663 | 5 |
| ARID5A | IL2RA | CTTCTCTAGACTTAAAGATG | 216 | ACTTCTTCTCTAGACTTAAAGATGGGGTGC | 664 | 7 |
| ARID5A | IL2RA | GCGACACACGCCCATCGAGA | 217 | AGGAGCGACACACGCCCATCGAGAGGGTGC | 665 | 3 |
| ARID5A | IL2RA | GCTGTCCTGAGGCCTACAAG | 218 | AGCGGCTGTCCTGAGGCCTACAAGCGGCTC | 666 | 7 |
| ZNF114 | IL2RA | ACGAAGACGATGGAGTCTTG | 219 | AACAACGAAGACGATGGAGTCTTGGGGTGG | 667 | 6 |
| ZNF114 | IL2RA | CCAAAACAGAGGAACCACAC | 220 | TGCCCCAAAACAGAGGAACCACACAGGCAG | 668 | 6 |
| ZNF114 | IL2RA | CTTATGGAATCTCCCTGTGA | 221 | TTGTCTTATGGAATCTCCCTGTGAAGGCAC | 669 | 6 |
| ZNF114 | IL2RA | TACGTGATGAAATTGATACG | 222 | CATATACGTGATGAAATTGATACGGGGGCC | 670 | 6 |
| E4F1 | IL2RA | AGGTGGTGGGCACATCAAAG | 223 | TCCTAGGTGGTGGGCACATCAAAGAGGTCA | 671 | 4 |
| E4F1 | IL2RA | CTGAACAAAATCCTCCAAGG | 224 | TGTGCTGAACAAAATCCTCCAAGGCGGTGA | 672 | 2 |
| E4F1 | IL2RA | GGCACAGCGCACACACATAG | 225 | TTGTGGCACAGCGCACACACATAGCGGCCA | 673 | 4 |
| E4F1 | IL2RA | GGGAAGGTCTCACTGCACTG | 226 | TGTCGGGAAGGTCTCACTGCACTGAGGACA | 674 | 9 |
| GLIS2 | IL2RA | ACACCATCCAAATAGCGCAG | 227 | GGGGACACCATCCAAATAGCGCAGTGGCTG | 675 | 3 |
| GLIS2 | IL2RA | GACAGGCTGAGGTCCACGAG | 228 | TGGTGACAGGCTGAGGTCCACGAGAGGGGC | 676 | 2 |
| GLIS2 | IL2RA | GGGTGCGCGTGTGCTTAAAG | 229 | TAGTGGGTGCGCGTGTGCTTAAAGCGGTCA | 677 | 6 |
| GLIS2 | IL2RA | TGACATGGTAATCGTTGACA | 230 | GGCTTGACATGGTAATCGTTGACATGGTCC | 678 | 4 |
| JUNB | IL2RA | CACAGCTACGGGATACGGCC | 231 | CATACACAGCTACGGGATACGGCCGGGCCC | 679 | 1 |
| JUNB | IL2RA | CCGGAGTCTCAAAGCGCCTG | 232 | CCTACCGGAGTCTCAAAGCGCCTGGGGCTC | 680 | 1 |
| JUNB | IL2RA | CTGAGGTTGGTGTAAACGGG | 233 | GCTGCTGAGGTTGGTGTAAACGGGAGGTGG | 681 | 1 |
| JUNB | IL2RA | GGGTAAAAGTACTGTCCCGG | 234 | GCGGGGGTAAAAGTACTGTCCCGGGGGTGT | 682 | 1 |
| NR2F6 | IL2RA | AAGTCGAGCGGCAAGCATTA | 235 | GGACAAGTCGAGCGGCAAGCATTACGGTGT | 683 | 1 |
| NR2F6 | IL2RA | CGAGCTCTTCGTGCTGAACG | 236 | GGAGCGAGCTCTTCGTGCTGAACGCGGCGC | 684 | 3 |
| NR2F6 | IL2RA | GCGGCGGAGACCTCTTCCCG | 237 | GCGAGCGGCGGAGACCTCTTCCCGGGGCAG | 685 | 3 |
| NR2F6 | IL2RA | GCTGGTCGATCTGGCAGTCA | 238 | TGGTGCTGGTCGATCTGGCAGTCACGGTTG | 686 | 2 |
| ZZZ3 | IL2RA | ACAGACGATTGCTGTACTCG | 239 | TATTACAGACGATTGCTGTACTCGAGGCTC | 687 | 6 |
| ZZZ3 | IL2RA | AGAGTATAACGATGTTCTTG | 240 | TCTCAGAGTATAACGATGTTCTTGAGGTTC | 688 | 5 |
| ZZZ3 | IL2RA | GCAGAGTACCCGAGAATCAT | 241 | AACAGCAGAGTACCCGAGAATCATGGGTAA | 689 | 5 |
| ZZZ3 | IL2RA | TCAATTGCCTGAGATCGTAT | 242 | TTGTTCAATTGCCTGAGATCGTATGGGACC | 690 | 7 |
| ETV3 | IL2RA | AGGACACGGGATCCACACCC | 243 | CTGGAGGACACGGGATCCACACCCCGGCGC | 691 | 5 |
| ETV3 | IL2RA | ATCCCTGGCCTAGCAAACAG | 244 | GTACATCCCTGGCCTAGCAAACAGAGGAAG | 692 | 5 |
| ETV3 | IL2RA | GAGAATGGGTGTCCAGAGGT | 245 | GTTGGAGAATGGGTGTCCAGAGGTGGGAAA | 693 | 5 |
| ETV3 | IL2RA | GGGTAACGGGGAAAAGTCCG | 246 | CCCTGGGTAACGGGGAAAAGTCCGAGGACT | 694 | 5 |
| ERF | IL2RA | CGGAAGTGGCTACCACCCGA | 247 | GAAGCGGAAGTGGCTACCACCCGACGGCAC | 695 | 4 |
| ERF | IL2RA | CTGAGCCCGATGTACCCCAG | 248 | CACGCTGAGCCCGATGTACCCCAGTGGTGG | 696 | 4 |
| ERF | IL2RA | GACCGGCCAGAGGCGACACA | 249 | CCAGGACCGGCCAGAGGCGACACAGGGAAG | 697 | 4 |
| ERF | IL2RA | GGAAGGCACCCAGATCCGGA | 250 | CCTCGGAAGGCACCCAGATCCGGAGGGCCG | 698 | 4 |
| RUNX1 | IL2RA | CACTTCGACCGACAAACCTG | 251 | CTTCCACTTCGACCGACAAACCTGAGGTCA | 699 | 5 |
| RUNX1 | IL2RA | CTGATCGTAGGACCACGGTG | 252 | AGGACTGATCGTAGGACCACGGTGGGGATG | 700 | 8 |
| RUNX1 | IL2RA | GGCAGTGGAGTGGTTCAGGG | 253 | TAAAGGCAGTGGAGTGGTTCAGGGAGGCAC | 701 | 7 |
| RUNX1 | IL2RA | TAGATGATCAGACCAAGCCC | 254 | AAACTAGATGATCAGACCAAGCCCGGGAGC | 702 | 7 |
| TGIF2 | IL2RA | ACAGGTTGGTCTGTCCAGAA | 255 | ACTGACAGGTTGGTCTGTCCAGAAAGGCTC | 703 | 2 |
| TGIF2 | IL2RA | GCAAGTACAGCCAGTCCCGG | 256 | CGGTGCAAGTACAGCCAGTCCCGGAGGATC | 704 | 2 |
| TGIF2 | IL2RA | GGAGTCGGTGAAGATCCTCC | 257 | CCAAGGAGTCGGTGAAGATCCTCCGGGACT | 705 | 2 |
| TGIF2 | IL2RA | GGCGGGCAAAAGGAAGCGCA | 258 | CCCTGGCGGGCAAAAGGAAGCGCAGGGGGA | 706 | 2 |
| ZNF574 | IL2RA | ACATGAATGCAGAGGATTCG | 259 | ATGGACATGAATGCAGAGGATTCGGGGTGT | 707 | 2 |
| ZNF574 | IL2RA | AGGGCCCGGAGGAACAACAG | 260 | GCGCAGGGCCCGGAGGAACAACAGTGGAGA | 708 | 2 |
| ZNF574 | IL2RA | CCAGCCGATGCACAAAACGT | 261 | CGCTCCAGCCGATGCACAAAACGTTGGTGC | 709 | 2 |
| ZNF574 | IL2RA | TGTGGAGCACTCATACCGAA | 262 | TGGCTGTGGAGCACTCATACCGAAAGGCAG | 710 | 2 |
| PTEN | IL2RA | AGAGCGTGCAGATAATGACA | 263 | GTATAGAGCGTGCAGATAATGACAAGGAAT | 711 | 8 |
| PTEN | IL2RA | AGCTGGCAGACCACAAACTG | 264 | CTTTAGCTGGCAGACCACAAACTGAGGATC | 712 | 7 |
| PTEN | IL2RA | ATTCTTCATACCAGGACCAG | 265 | ATACATTCTTCATACCAGGACCAGAGGAAA | 713 | 8 |
| PTEN | IL2RA | CCAATTCAGGACCCACACGA | 266 | TCCTCCAATTCAGGACCCACACGACGGGAA | 714 | 7 |
| IL2RA | IL2RA | CTGCAGGGAACCTCCACCAT | 267 | GTCACTGCAGGGAACCTCCACCATGGGAAA | 715 | 4 |
| IL2RA | IL2RA | GGATACAGGGCTCTACACAG | 268 | CCAGGGATACAGGGCTCTACACAGAGGTCC | 716 | 4 |
| IL2RA | IL2RA | TGGCTTTGAATGTGGCGTGT | 269 | GCCATGGCTTTGAATGTGGCGTGTGGGATC | 717 | 2 |
| IL2RA | IL2RA | TTGTTTCGTTGTGTTCCGAG | 270 | TCACTTGTTTCGTTGTGTTCCGAGTGGCTA | 718 | 3 |
| FOXO1 | IL2RA | ACAGGTTGCCCCACGCGTTG | 271 | TAGGACAGGTTGCCCCACGCGTTGCGGCGG | 719 | 1 |
| FOXO1 | IL2RA | GGAGTTTAGCCAGTCCAACT | 272 | GGCCGGAGTTTAGCCAGTCCAACTCGGCCA | 720 | 1 |
| FOXO1 | IL2RA | GGTGGCGCAAACGAGTAGCA | 273 | GTTTGGTGGCGCAAACGAGTAGCACGGCGT | 721 | 2 |
| FOXO1 | IL2RA | TAGCATTTGAGCTAGTTCGA | 274 | GTACTAGCATTTGAGCTAGTTCGAGGGCGA | 722 | 2 |
| FOXP1 | IL2RA | AGAGGAGGAGACACATGTCG | 275 | GTGCAGAGGAGGAGACACATGTCGTGGTCA | 723 | 11 |
| FOXP1 | IL2RA | CATACACCATGTCCATAGAG | 276 | CTTGCATACACCATGTCCATAGAGAGGATG | 724 | 12 |
| FOXP1 | IL2RA | GCCTTCTGACAATTCAGCCC | 277 | CAAGGCCTTCTGACAATTCAGCCCGGGCAG | 725 | 10 |
| FOXP1 | IL2RA | GTTCTGTAGACTTCACATGC | 278 | TTGGGTTCTGTAGACTTCACATGCAGGTGG | 726 | 14 |
| STAT5B | IL2RA | CAGCCAGGACAACAATGCGA | 279 | ATGGCAGCCAGGACAACAATGCGACGGCCA | 727 | 12 |
| STAT5B | IL2RA | GTGGCCTTAATGTTCTCCTG | 280 | CTGGGTGGCCTTAATGTTCTCCTGTGGATT | 728 | 3 |
| STAT5B | IL2RA | GTTCATTGTACAATATATGG | 281 | CTCTGTTCATTGTACAATATATGGCGGATG | 729 | 4 |
| STAT5B | IL2RA | TAAGAGGTCAGACCGTCGTG | 282 | GAATTAAGAGGTCAGACCGTCGTGGGGCAG | 730 | 11 |
| STAT5A | IL2RA | ACATTCTGTACAATGAACAG | 283 | CGGCACATTCTGTACAATGAACAGAGGCTG | 731 | 5 |
| STAT5A | IL2RA | ATCAAGCGTGCTGACCGGCG | 284 | GAGGATCAAGCGTGCTGACCGGCGGGGTGC | 732 | 12 |
| STAT5A | IL2RA | CAGCCAGGACCACAATGCCA | 285 | ACGGCAGCCAGGACCACAATGCCACGGCTA | 733 | 13 |
| STATSA | IL2RA | CGTGCACATGAATCCCCCCC | 286 | TGAACGTGCACATGAATCCCCCCCAGGTGA | 734 | 10 |
| MED12 | IL2RA | ACATCGACTGCTGGACAATG | 287 | ATCCACATCGACTGCTGGACAATGAGGATG | 735 | 28 |
| MED12 | IL2RA | CAGTGAGTAGTGCCAAACCA | 288 | CAGTCAGTGAGTAGTGCCAAACCAAGGCAC | 736 | 8 |
| MED12 | IL2RA | GTGGCGTACTGCACGTGTCG | 289 | ATGGGTGGCGTACTGCACGTGTCGTGGCTG | 737 | 15 |
| MED12 | IL2RA | TTCACATTATGACCAACACC | 290 | ACCTTTCACATTATGACCAACACCAGGTCA | 738 | 18 |
| FOXP3 | IL2RA | ACCCAGGCATCATCCGACAA | 291 | CCTCACCCAGGCATCATCCGACAAGGGCTC | 739 | 9 |
| FOXP3 | IL2RA | CCCACCCACAGGGATCAACG | 292 | TGTCCCCACCCACAGGGATCAACGTGGCCA | 740 | 5 |
| FOXP3 | IL2RA | CCTACTTAGGCACTGCCAGG | 293 | TCTCCCTACTTAGGCACTGCCAGGGGACC | 741 | 7 |
| FOXP3 | IL2RA | GAGGGTGCCACCATGACTAG | 294 | CCCGGAGGGTGCCACCATGACTAGGGGCAG | 742 | 3 |
| FLI1 | IL2RA | ACTCAATCGTGAGGATTGGT | 295 | TGACACTCAATCGTGAGGATTGGTCGGTGT | 743 | 5 |
| FLI1 | IL2RA | ACTGTGTAAAATGAACAAGG | 296 | AGGAACTGTGTAAAATGAACAAGGAGGACT | 744 | 4 |
| FLI1 | IL2RA | CCAACATGACCACCAACGAG | 297 | CCTCCCAACATGACCACCAACGAGAGGAGA | 745 | 3 |
| FLI1 | IL2RA | CTTACCTGGATCCATTCATG | 298 | TGAGCTTACCTGGATCCATTCATGTGGTCA | 746 | 2 |
| ETS1 | IL2RA | CTTACTAATGAAGTAATCCG | 299 | TGAACTTACTAATGAAGTAATCCGAGGTAT | 747 | 4 |
| ETS1 | IL2RA | GAGAAAGCAGTCTTTACCCA | 300 | GCTCGAGAAAGCAGTCTTTACCCAGGGCGC | 748 | 3 |
| ETS1 | IL2RA | GGTCTCGGAGAATGACCGAG | 301 | AGAGGGTCTCGGAGAATGACCGAGGGGTAG | 749 | 5 |
| ETS1 | IL2RA | TGCATGGGGAGGACCAGTCG | 302 | CATGTGCATGGGGAGGACCAGTCGTGGTAG | 750 | 5 |
| ATXN7L3 | IL2RA | CACGGACCCTGATAGCATGA | 303 | ACGACACGGACCCTGATAGCATGAAGGATT | 751 | 2 |
| ATXN7L3 | IL2RA | CATCGCTCAGGAGATATACG | 304 | AGGCCATCGCTCAGGAGATATACGCGGACC | 752 | 2 |
| ATXN7L3 | IL2RA | GCAGCCGAATCGCCAACCGC | 305 | AACAGCAGCCGAATCGCCAACCGCCGGTGA | 753 | 3 |
| ATXN7L3 | IL2RA | GCTTCGCAGCCTGCTAACCA | 306 | AGGAGCTTCGCAGCCTGCTAACCACGGTGA | 754 | 8 |
| KMT2A | IL2RA | AAGATCAGTAGCGGTCCCGG | 307 | ATTCAAGATCAGTAGCGGTCCCGGTGGTGG | 755 | 27 |
| KMT2A | IL2RA | AGAAAGGACGTCGATCGAGG | 308 | GTAAAGAAAGGACGTCGATCGAGGCGGTGT | 756 | 5 |
| KMT2A | IL2RA | AGGGGTCTTAATGATCCGCG | 309 | GCCGÅGGGGTCTTAATGATCCGCGAGGAGA | 757 | 3 |
| KMT2A | IL2RA | TTGACCATAATTATGCTCAG | 310 | TCACTTGACCATAATTATGCTCAGTGGCAG | 758 | 19 |
| ZEB1 | IL2RA | AATGCTTCACCCATACAACA | 311 | AATCAATGCTTCACCCATACAACAAGGTGG | 759 | 7 |
| ZEB1 | IL2RA | CAGACCAGACAGTGTTACCA | 312 | CCAACAGACCAGACAGTGTTACCAGGGAGG | 760 | 2 |
| ZEB1 | IL2RA | GAAGGACAAAAGCTTTGAAG | 313 | CTGAGAAGGACAAAAGCTTTGAAGGGGGGG | 761 | 7 |
| ZEB1 | IL2RA | GGTTACTTGTACACAGCTGA | 314 | ACAGGGTTACTTGTACACAGCTGAGGGTGC | 762 | 7 |
| MBD2 | IL2RA | AGCCGGTCCCTTTCCCGTCG | 315 | CGGGAGCCGGTCCCTTTCCCGTCGGGGAGC | 763 | 1 |
| MBD2 | IL2RA | CCTCAGTTGGCAAGGTACCT | 316 | CAAGCCTCAGTTGGCAAGGTACCTGGGAAA | 764 | 2 |
| MBD2 | IL2RA | CCTCTCAATCAAAATAAGGT | 317 | CGATCCTCTCAATCAAAATAAGGTTGGTTA | 765 | 2 |
| MBD2 | IL2RA | CGAAAATCTGGGCTAAGTGC | 318 | GATCCGAAAATCTGGGCTAAGTGCTGGCAA | 766 | 1 |
| CIC | IL2RA | ACTGTCACTAACCTACTGGT | 319 | TGCCACTGTCACTAACCTACTGGTGGGCAC | 767 | 10 |
| CIC | IL2RA | CCCGCCCGCTGACTGCACAT | 320 | CGTGCCCGCCCGCTGACTGCACATAGGTGA | 768 | 13 |
| CIC | IL2RA | CTCTACCGCCCGGAAAACGT | 321 | TTGCCTCTACCGCCCGGAAAACGTCGGACC | 769 | 4 |
| CIC | IL2RA | TTGGGCCAGAGTACGATGCA | 322 | GGCTTTGGGCCAGAGTACGATGCAGGGCCA | 770 | 8 |
| TAF5L | IL2RA | CGGGACACGTCTACTTGGTG | 323 | GATGCGGGACACGTCTACTTGGTGGGGCTC | 771 | 4 |
| TAF5L | IL2RA | GCAGAACGAGGCTGCCCTAG | 324 | TTCTGCAGAACGAGGCTGCCCTAGAGGTCT | 772 | 4 |
| TAF5L | IL2RA | GCGGACCAGTGTACAGCACG | 325 | CACTGCGGACCAGTGTACAGCACGAGGTTC | 773 | 5 |
| TAF5L | IL2RA | TAAGGTGAGGACTTTGCACA | 326 | TATGTAAGGTGAGGACTTTGCACAGGGCAG | 774 | 4 |
| USP22 | IL2RA | ACCTGGTGTGGACCCACGCG | 327 | CTGCACCTGGTGTGGACCCACGCGAGGCAC | 775 | 6 |
| USP22 | IL2RA | CCTCGAACTGCACCATAGGT | 328 | ATCACCTCGAACTGCACCATAGGTGGGTGG | 776 | 4 |
| USP22 | IL2RA | GCCATTGATCTGATGTACGG | 329 | CTCAGCCATTGATCTGATGTACGGAGGCAT | 777 | 3 |
| USP22 | IL2RA | TGGGGCTCTGCATCTCACAG | 330 | GAGCTGGGGCTCTGCATCTCACAGCGGTGC | 778 | 5 |
| MED30 | IL2RA | ACACTGGAACATATCAAGAC | 331 | TACCACACTGGAACATATCAAGACCGGTTA | 779 | 2 |
| MED30 | IL2RA | GACAAATGCAATGAAAACTG | 332 | ATATGACAAATGCAATGAAAACTGTGGTGG | 780 | 2 |
| MED30 | IL2RA | GGACATCGTGTACCGCACCA | 333 | TGCAGGACATCGTGTACCGCACCATGGAGA | 781 | 1 |
| MED30 | IL2RA | GGCCGCCCGGGAAGTCAACA | 334 | AGCAGGCCGCCCGGGAAGTCAACACGGCGT | 782 | 1 |
| IKZF1 | IL2RA | GAAAATGAATGGCTCCCACA | 335 | GAGAGAAAATGAATGGCTCCCACAGGGACC | 783 | 4 |
| IKZF1 | IL2RA | GATGGCTTGGTCCATCACGT | 336 | TGTTGATGGCTTGGTCCATCACGTGGGACT | 784 | 8 |
| IKZF1 | IL2RA | GGGGCCTCATTCACCCAGAA | 337 | GTGCGGGGCCTCATTCACCCAGAAGGGCAA | 785 | 5 |
| IKZF1 | IL2RA | TCCAAGAGTGACAGAGTCGT | 338 | AAGCTCCAAGAGTGACAGAGTCGTGGGTAA | 786 | 3 |
| MED11 | IL2RA | ATCCCGAACCTGCATTCTGA | 339 | TCGCATCCCGAACCTGCATTCTGAAGGATG | 787 | 1 |
| MED11 | IL2RA | CACCGCTTCAGTGCAACACG | 340 | CCTTCACCGCTTCAGTGCAACACGTGGAGG | 788 | 2 |
| MED11 | IL2RA | CCAAGGAAAAAACTAACGAG | 341 | TTGTCCAAGGAAAAAACTAACGAGCGGCTC | 789 | 2 |
| MED11 | IL2RA | GCGGCTCCTAGACCGGCAGG | 342 | ACGAGCGGCTCCTAGACCGGCAGGCGGCGG | 790 | 2 |
| RXRB | IL2RA | ACGGCTATGTGCAATCTGCG | 343 | GCAAACGGCTATGTGCAATCTGCGGGGACA | 791 | 3 |
| RXRB | IL2RA | GCCCTGGCTGGATCCCGCAG | 344 | GACGGCCCTGGCTGGATCCCGCAGCGGCGG | 792 | 1 |
| RXRB | IL2RA | GGACAACAAAGACTGCACAG | 345 | GCCGGGACAACAAAGACTGCACAGTGGACA | 793 | 4 |
| RXRB | IL2RA | GTGGCTTCACATCTTCAGGG | 346 | ACTGGTGGCTTCACATCTTCAGGGGGGCCA | 794 | 3 |
| IKZF3 | IL2RA | AAGATGAACTGCGATGTGTG | 347 | TGGAAAGATGAACTGCGATGTGTGTGGATT | 795 | 4 |
| IKZF3 | IL2RA | CAAGCAGAGAAGTTCCCTTG | 348 | GTTACAAGCAGAGAAGTTCCCTTGAGGAGC | 796 | 6 |
| IKZF3 | IL2RA | GCTCATACAGACCCGCATGA | 349 | GTGAGCTCATACAGACCCGCATGATGGACC | 797 | 8 |
| IKZF3 | IL2RA | GGACAGATTAGCAAGCAATG | 350 | TACTGGACAGATTAGCAAGCAATGTGGCAA | 798 | 7 |
| PURA | IL2RA | CCTTACTCTCTCCATGTCAG | 351 | GCCGCCTTACTCTCTCCATGTCAGTGGCCG | 799 | 1 |
| PURA | IL2RA | GAACTCGATGAGCCCCTGCG | 352 | CACGGAACTCGATGAGCCCCTGCGCGGGCA | 800 | 1 |
| PURA | IL2RA | GCTCATCGACGACTACGGAG | 353 | CCAAGCTCATCGACGACTACGGAGTGGAGG | 801 | 1 |
| PURA | IL2RA | TCCGCCAGACGGTCAACCGG | 354 | CGCATCCGCCAGACGGTCAACCGGGGGCCT | 802 | 1 |
| SETDB1 | IL2RA | AAGGAAAGAGTCTACTGTCG | 355 | AACAAAGGAAAGAGTCTACTGTCGGGGAAC | 803 | 7 |
| SETDB1 | IL2RA | AGATGTGAGTGGATCTATCG | 356 | CAAAAGATGTGAGTGGATCTATCGAGGCTC | 804 | 10 |
| SETDB1 | IL2RA | CCTTACCTGAATCAATACTG | 357 | CATCCCTTACCTGAATCAATACTGAGGACA | 805 | 3 |
| SETDB1 | IL2RA | GTTATCTATAAGACACCTTG | 358 | TCATGTTATCTATAAGACACCTTGTGGTCT | 806 | 13 |
| RELA | IL2RA | ACTACGACCTGAATGCTGTG | 359 | GGGGACTACGACCTGAATGCTGTGCGGCTC | 807 | 6 |
| RELA | IL2RA | GCTTCCGCTACAAGTGCGAG | 360 | ATGCGCTTCCGCTACAAGTGCGAGGGGCGC | 808 | 3 |
| RELA | IL2RA | GGAAGATCTCATCCCCACCG | 361 | AGTAGGAAGATCTCATCCCCACCGAGGCAG | 809 | 7 |
| RELA | IL2RA | TCAATGGCTACACAGGACCA | 362 | CAGATCAATGGCTACACAGGACCAGGGACA | 810 | 4 |
| SS18 | IL2RA | AATCAGATGACAATGAGTCA | 363 | ACAGAATCAGATGACAATGAGTCAGGGACA | 811 | 5 |
| SS18 | IL2RA | CAATACAATATGCCACAGGG | 364 | TCAGCAATACAATATGCCACAGGGAGGCGG | 812 | 6 |
| SS18 | IL2RA | CCTAACCATATGCCTATGCA | 365 | AGGGCCTAACCATATGCCTATGCAGGGACC | 813 | 5 |
| SS18 | IL2RA | GGCATGTTGTGAGAGCGTGG | 366 | TGAAGGCATGTTGTGAGAGCGTGGAGGTGG | 814 | 4 |
| SRF | IL2RA | AGGTTGGTGACTGTGAACGC | 367 | CGGCAGGTTGGTGACTGTGAACGCCGGCTT | 815 | 3 |
| SRF | IL2RA | AGTTCATCGACAACAAGCTG | 368 | ATGGAGTTCATCGACAACAAGCTGCGGCGC | 816 | 1 |
| SRF | IL2RA | GGGCTGACACTAGCAGACAC | 369 | ACTGGGGCTGACACTAGCAGACACTGGTGC | 817 | 3 |
| SRF | IL2RA | TCTGTTGTGGGGTCTGAACG | 370 | CTGGTCTGTTGTGGGGTCTGAACGGGGTGG | 818 | 2 |
| GATA3 | IL2RA | AGGTACCCTCCGACCCACCA | 371 | GCAGAGGTACCCTCCGACCCACCACGGTGA | 819 | 2 |
| GATA3 | IL2RA | CAGGGAGTGTGTGAACTGTG | 372 | AAGGCAGGGAGTGTGTGAACTGTGGGGCAA | 820 | 4 |
| GATA3 | IL2RA | GGAGCTGTACTCGGGCACGT | 373 | GTCCGGAGCTGTACTCGGGCACGTAGGGCG | 821 | 3 |
| GATA3 | IL2RA | TCCAAGACGTCCATCCACCA | 374 | CTTCTCCAAGACGTCCATCCACCACGGCTC | 822 | 3 |
| ZNF384 | IL2RA | CGTTACCCAGAATATCACGG | 375 | CGTCCGTTACCCAGAATATCACGGTGGTCC | 823 | 4 |
| ZNF384 | IL2RA | GCACATCCGTATACACTCAG | 376 | CCCAGCACATCCGTATACACTCAGGGGCTA | 824 | 7 |
| ZNF384 | IL2RA | GGTAGCATCGACCCTAACCG | 377 | AGAAGGTAGCATCGACCCTAACCGAGGAAG | 825 | 6 |
| ZNF384 | IL2RA | TGGCCCCCGAGTGGATACGG | 378 | GGCTTGGCCCCCGAGTGGATACGGAGGTGC | 826 | 8 |
| ZNF148 | IL2RA | AGATCGAAGTATGCCTCACC | 379 | TTCAAGATCGAAGTATGCCTCACCAGGAGA | 827 | 4 |
| ZNF148 | IL2RA | AGTGCATACTGTAGTCCTTG | 380 | ATTAAGTGCATACTGTAGTCCTTGAGGAAG | 828 | 4 |
| ZNF148 | IL2RA | GAGCCCCCAACTGACGAATG | 381 | ATGCGAGCCCCCAACTGACGAATGTGGCAT | 829 | 9 |
| ZNF148 | IL2RA | TAATTAGTACTACTATGCAC | 382 | ATCATAATTAGTACTACTATGCACAGGTTT | 830 | 9 |
| JAK3 | IL2RA | ACTCTCCAGGCTTAACACAG | 383 | ACTTACTCTCCAGGCTTAACACAGCGGGGC | 831 | 15 |
| JAK3 | IL2RA | AGCTCTCGAAGACTGCTGTG | 384 | CAGGAGCTCTCGAAGACTGCTGTGGGGTCG | 832 | 10 |
| JAK3 | IL2RA | GTGTACAAATTCCTGCACCA | 385 | CCAGGTGTACAAATTCCTGCACCATGGTGC | 833 | 14 |
| JAK3 | IL2RA | TGACGCGGAGGCGTATTCGG | 386 | TTCGTGACGCGGAGGCGTATTCGGAGGACG | 834 | 6 |
| MED14 | IL2RA | ATCACACATAGCGACGAAGT | 387 | TTGTATCACACATAGCGACGAAGTGGGCTA | 835 | 15 |
| MED14 | IL2RA | CAGAGCATCTCTAGCTAACG | 388 | GGACCAGAGCATCTCTAGCTAACGAGGCCA | 836 | 4 |
| MED14 | IL2RA | CTAACTCTGCTACCCAAGTG | 389 | AACACTAACTCTGCTACCCAAGTGCGGTTA | 837 | 17 |
| MED14 | IL2RA | TAATGTTAATCCGAGAACGG | 390 | ACTCTAATGTTAATCCGAGAACGGTGGGGA | 838 | 8 |
| PKNOX1 | IL2RA | AACAGTGAAACTCTGTTGAG | 391 | AATGAACAGTGAAACTCTGTTGAGTGGAGA | 839 | 5 |
| PKNOX1 | IL2RA | GGGAAACGTAGCCATGGCGA | 392 | AGCAGGGAAACGTAGCCATGGCGACGGTGG | 840 | 6 |
| PKNOX1 | IL2RA | GGTTCATCTAAGAACAAGAG | 393 | TGATGGTTCATCTAAGAACAAGAGGGGCGT | 841 | 8 |
| PKNOX1 | IL2RA | TGCTCTGAACCCGATGCAGA | 394 | AAACTGCTCTGAACCCGATGCAGAAGGAGT | 842 | 3 |
| USF2 | IL2RA | AGCCGTAGTATCTCCCACAC | 395 | ACACAGCCGTAGTATCTCCCACACTGGACG | 843 | 5 |
| USF2 | IL2RA | CCACGACAAGGGACCCGAGG | 396 | GCAGCCACGACAAGGGACCCGAGGCGGAGG | 844 | 2 |
| USF2 | IL2RA | CCTGCACATACGGAGAGTAA | 397 | GTCCCCTGCACATACGGAGAGTAAGGGTGT | 845 | 6 |
| USF2 | IL2RA | TTCCGCACAGAGACAAATGG | 398 | CCAGTTCCGCACAGAGACAAATGGAGGACA | 846 | 3 |
| KLF6 | IL2RA | TCGGAGGTAAACTTGGCCGT | 399 | GGGGTCGGAGGTAAACTTGGCCGTGGGAGA | 847 | 2 |
| KLF6 | IL2RA | TCTAAGTTGTAACAAAAGCT | 400 | GGTCTCTAAGTTGTAACAAAAGCTCGGGCT | 848 | 2 |
| KLF6 | IL2RA | TCTGAGGCTGAAACATAGCA | 401 | GATTTCTGAGGCTGAAACATAGCAGGGCTC | 849 | 2 |
| KLF6 | IL2RA | TGCCAGTACTCCTCCAGAGA | 402 | CTGTTGCCAGTACTCCTCCAGAGACGGCAG | 850 | 1 |
| DNMT1 | IL2RA | GAGGCAAAAAGAAATCCCCA | 403 | AGATGAGGCAAAAAGAAATCCCCAGGGTCC | 851 | 27 |
| DNMT1 | IL2RA | GATTTCTGATGAAAAAGACG | 404 | CACAGATTTCTGATGAAAAAGACGAGGATG | 852 | 12 |
| DNMT1 | IL2RA | GCTCTACTGGAGCGACGAGG | 405 | ACCTGCTCTACTGGAGCGACGAGGAGGCCG | 853 | 29 |
| DNMT1 | IL2RA | TCACCCAAAAAAATGCACCA | 406 | GCCGTCACCCAAAAAAATGCACCAGGGGAA | 854 | 23 |
| DDX39B | IL2RA | CCTGCCAAGAAGGATGTCAA | 407 | GGCCCCTGCCAAGAAGGATGTCAAGGGCTC | 855 | 2 |
| DDX39B | IL2RA | CTCAAAGCCACAGTCGACAA | 408 | GATGCTCAAAGCCACAGTCGACAATGGCCC | 856 | 2 |
| DDX39B | IL2RA | GAGGCTCTTATTTCGAGCCA | 409 | GGTTGAGGCTCTTATTTCGAGCCAGGGCTA | 857 | 5 |
| DDX39B | IL2RA | GATAAGATGCTTGAACAGCT | 410 | ATGTGATAAGATGCTTGAACAGCTCGGTGA | 858 | 5 |
| ZNF236 | IL2RA | ATCACGGAAGTCTCGTCCTG | 411 | ATGCATCACGGAAGTCTCGTCCTGAGGTCA | 859 | 20 |
| ZNF236 | IL2RA | GCATGTGCGGTCGCACACCG | 412 | AGCAGCATGTGCGGTCGCACACCGGGGAAA | 860 | 18 |
| ZNF236 | IL2RA | GGCAGCTTACGGCGACACAT | 413 | TGGTGGCAGCTTACGGCGACACATGGGTAT | 861 | 13 |
| ZNF236 | IL2RA | TGGTCTACGTGCCCTCGCAG | 414 | AAACTGGTCTACGTGCCCTCGCAGAGGGTC | 862 | 15 |
| SOCS3 | IL2RA | ACCTACTGAACCCTCCTOCG | 415 | CGCCACCTACTGAACCCTCCTCCGAGGTGC | 863 | 2 |
| SOCS3 | IL2RA | GCGGATCAGAAAGGTGCCGG | 416 | TGTCGCGGATCAGAAAGGTGCCGGGGGGCT | 864 | 2 |
| SOCS3 | IL2RA | TCAGCGTCAAGACCCAGTCT | 417 | ACGCTCAGCGTCAAGACCCAGTCTGGGACC | 865 | 2 |
| SOCS3 | IL2RA | TTGAGCACGCAGTCGAAGCG | 418 | CAGCTTGAGCACGCAGTCGAAGCGGGGCAC | 866 | 2 |
| GABPA | IL2RA | AAGCAGAGTGCACAGAAGAA | 419 | GAGAAAGCAGAGTGCACAGAAGAAAGGTTG | 867 | 2 |
| GABPA | IL2RA | CCGAAATGTTGAGTGTGGTG | 420 | CTCCCCGAAATGTTGAGTGTGGTGAGGTCT | 868 | 6 |
| GABPA | IL2RA | CTCCAGAGAATTTCTCCCCG | 421 | ATGACTCCAGAGAATTTCTCCCCGAGGAAC | 869 | 6 |
| GABPA | IL2RA | TCTTTCTAGATCCCATACAG | 422 | TTTTTCTTTCTAGATCCCATACAGTGGTCC | 870 | 6 |
| RBPJ | IL2RA | AAAGAACAAATGGAACGCGA | 423 | AAAAAAAGAACAAATGGAACGCGATGGTTG | 871 | 5 |
| RBPJ | IL2RA | CACCTAGTAAGTCGTTTAGG | 424 | TACTCACCTAGTAAGTCGTTTAGGTGGAGG | 872 | 3 |
| RBPJ | IL2RA | CATGCCAGTTCACAGCAGTG | 425 | TTTTCATGCCAGTTCACAGCAGTGGGGAGC | 873 | 7 |
| RBPJ | IL2RA | CATTGCCTCAGGAACAAAGG | 426 | TATGCATTGCCTCAGGAACAAAGGTGGCTC | 874 | 7 |
| STAT3 | IL2RA | AACATGGAAGAATCCAACAA | 427 | GATGAACATGGAAGAATCCAACAACGGCAG | 875 | 13 |
| STAT3 | IL2RA | ACGCCGGTCTTGATGACGAG | 428 | CTGGACGCCGGTCTTGATGACGAGGGGCCG | 876 | 10 |
| STAT3 | IL2RA | GAGACCGAGGTGTATCACCA | 429 | CTTTGAGACCGAGGTGTATCACCAAGGCCT | 877 | 15 |
| STAT3 | IL2RA | TCGGCCGGTGCTGTACAATG | 430 | AGCATCGGCCGGTGCTGTACAATGGGGTCC | 878 | 9 |
| VPS52 | IL2RA | CAATGAACGAGCAACAGCAA | 431 | TGGGCAATGAACGAGCAACAGCAAAGGAGA | 879 | 8 |
| VPS52 | IL2RA | CCGTACACTCAGCATGACCC | 432 | TGGGCCGTACACTCAGCATGACCCTGGTAA | 880 | 11 |
| VPS52 | IL2RA | GAAATCGCCAGGCAGTTCGG | 433 | CTTCGAAATCGCCAGGCAGTTCGGGGGAAA | 881 | 5 |
| VPS52 | IL2RA | TCCAGGATCAGTTCAAACCG | 434 | CATCTCCAGGATCAGTTCAAACCGTGGCCA | 882 | 13 |
| IL2 | IL2RA | AAACTTAAATGTGAGCATCC | 435 | TGTAAAACTTAAATGTGAGCATCCTGGTGA | 883 | 2 |
| IL2 | IL2RA | ACAACTGGAGCATTTACTGC | 436 | AGCTACAACTGGAGCATTTACTGCTGGATT | 884 | 1 |
| IL2 | IL2RA | AGAAGAAGAACTCAAACCTC | 437 | GTCTAGAAGAAGAACTCAAACCTCTGGAGG | 885 | 3 |
| IL2 | IL2RA | TTCTTTGTAGAACTTGAAGT | 438 | TGTTTTCTTTGTAGAACTTGAAGTAGGTGC | 886 | 1 |
| BCL11B | IL2RA | AGCAAGTCGTGCGAGTTCTG | 439 | CAAGAGCAAGTCGTGCGAGTTCTGCGGCAA | 887 | 4 |
| BCL11B | IL2RA | CCAGCAGCTCGCTCACGCCG | 440 | CCGGCCAGCAGCTCGCTCACGCCGCGGCTC | 888 | 4 |
| BCL11B | IL2RA | CCGCCATGGACTTCTCGCGG | 441 | TCGCCCGCCATGGACTTCTCGCGGCGGCTC | 889 | 4 |
| BCL11B | IL2RA | TCAGGGTGAGGGTCAGACGG | 442 | GGACTCAGGGTGAGGGTCAGACGGAGGCTC | 890 | 3 |
| RAD21 | IL2RA | AAGTGTTGTTTGATCAGTCA | 443 | GAACAAGTGTTGTTTGATCAGTCATGGTTG | 891 | 8 |
| RAD21 | IL2RA | ACATACTCTAAGTCAGGCAG | 444 | AGACACATACTCTAAGTCAGGCAGTGGCTG | 892 | 4 |
| RAD21 | IL2RA | GTGTAATTTAGAGAGCAGCG | 445 | TCGAGTGTAATTTAGAGAGCAGCGTGGAGA | 893 | 2 |
| RAD21 | IL2RA | TCTGTTCAGACTCTAATAGG | 446 | GTGCTCTGTTCAGACTCTAATAGGAGGTTA | 894 | 6 |
| HIVEP2 | IL2RA | CCCTGGATAGAATACATCGT | 447 | CGGTCCCTGGATAGAATACATCGTCGGAAC | 895 | 5 |
| HIVEP2 | IL2RA | GACAAGATGTCAGACCTAGG | 448 | TTCAGACAAGATGTCAGACCTAGGGGGCAG | 896 | 5 |
| HIVEP2 | IL2RA | GAGGTGGAAGGTAAACACAA | 449 | GTCAGAGGTGGAAGGTAAACACAAGGGGAT | 897 | 5 |
| HIVEP2 | IL2RA | TTCTAGGATAACCACCACTG | 450 | TTTTTTCTAGGATAACCACCACTGTGGCCA | 898 | 5 |
| IRF2 | IL2RA | ACCTGATCCCAAAACATGGA | 451 | ATAAACCTGATCCCAAAACATGGAAGGCGA | 899 | 4 |
| IRF2 | IL2RA | CAGCATTCGGTAGACCCTGA | 452 | GGGGCAGCATTCGGTAGACCCTGAAGGCAT | 900 | 4 |
| IRF2 | IL2RA | GGATGCATGCGGCTAGACAT | 453 | CCCTGGATGCATGCGGCTAGACATGGGTGG | 901 | 3 |
| IRF2 | IL2RA | TACCTGCATAGGAAGACACG | 454 | CGCTTACCTGCATAGGAAGACACGGGGGAG | 902 | 7 |
| TFAP4 | IL2RA | ACAGCTCAAGCGCTTCATCC | 455 | ACACACAGCTCAAGCGCTTCATCCAGGTGC | 903 | 3 |
| TFAP4 | IL2RA | AGGCTCCCCGGACATCTGGG | 456 | GCATAGGCTCCCCGGACATCTGGGAGGACG | 904 | 4 |
| TFAP4 | IL2RA | CACTAACCCCCGAGACTCAG | 457 | ATTCCACTAACCCCCGAGACTCAGGGGAC | 905 | 2 |
| TFAP4 | IL2RA | CGCATGCAGAGCATCAACGC | 458 | GAGACGCATGCAGAGCATCAACGCGGGATT | 906 | 2 |
| PRDM1 | IL2RA | AGGATGCGGATATGACTCTG | 459 | ATGGAGGATGCGGATATGACTCTGTGGACA | 907 | 2 |
| PRDM1 | IL2RA | GGACGCGTTCAAGTAAGCGT | 460 | CGTAGGACGCGTTCAAGTAAGCGTAGGAGT | 908 | 5 |
| PRDM1 | IL2RA | GGGGAGCGAGTGATGTACGT | 461 | AATGGGGGAGCGAGTGATGTACGTGGGTCT | 909 | 5 |
| PRDM1 | IL2RA | TTTGGACAGATCTATTCCAG | 462 | CTCTTTTGGACAGATCTATTCCAGAGGGGA | 910 | 4 |
| TNFAIP3 | IL2RA | CCACTTGTTAACAGAGACCG | 463 | TGTTCCACTTGTTAACAGAGACCGGGGAAG | 911 | 6 |
| TNFAIP3 | IL2RA | CTTGTGGCGCTGAAAACGAA | 464 | GAAGCTTGTGGCGCTGAAAACGAACGGTAA | 912 | 2 |
| TNFAIP3 | IL2RA | TATGCCATGAGTGCTCAGAG | 465 | CCTTTATGCCATGAGTGCTCAGAGAGGCGG | 913 | 7 |
| TNFAIP3 | IL2RA | TGAGAGACTCCAGTTGCCAG | 466 | GATTTGAGAGACTCCAGTTGCCAGCGGAAT | 914 | 3 |
| MEF2D | IL2RA | CAAGTACCGACGCGCCAGCG | 467 | AGGACAAGTACCGACGCGCCAGCGAGGAGC | 915 | 4 |
| MEF2D | IL2RA | CATCATCCCTCACGGACCCG | 468 | GTGACATCATCCCTCACGGACCCGCGGCTC | 916 | 5 |
| MEF2D | IL2RA | GATGACTGCACTCACCAACA | 469 | GAAGGATGACTGCACTCACCAACAGGGCTG | 917 | 6 |
| MEF2D | IL2RA | GGTGAGCGAATGAGTAGACT | 470 | GGGTGGTGAGCGAATGAGTAGACTGGGAGA | 918 | 9 |
| SMARCB1 | IL2RA | GAGAACCTCGGAACATACGG | 471 | TACAGAGAACCTCGGAACATACGGAGGTÅG | 919 | 2 |
| SMARCB1 | IL2RA | GCAGATCGAGTCCTACCCCA | 472 | GACAGCAGATCGAGTCCTACCCCACGGACA | 920 | 6 |
| SMARCB1 | IL2RA | TCTTCTTGTCTCGGCCCATG | 473 | GTTCTCTTCTTGTCTCGGCCCATGCGGTTC | 921 | 4 |
| SMARCB1 | IL2RA | TGAGAACGCATCTCAGCCCG | 474 | TCCATGAGAACGCATCTCAGCCCGAGGTGC | 922 | 5 |
| NFKB2 | IL2RA | ACTCGACTACGGCGTCACCG | 475 | CCCTACTCGACTACGGCGTCACCGCGGACG | 923 | 14 |
| NFKB2 | IL2RA | CCCACTCCATAGAATCTCCG | 476 | ATATCCCACTCCATAGAATCTOCGGGGGCA | 924 | 9 |
| NFKB2 | IL2RA | CTGCAACTGAAACGCAAGCG | 477 | GTTTCTGCAACTGAAACGCAAGCGAGGAGG | 925 | 11 |
| NFKB2 | IL2RA | GGGACCAGCCAAGATCGAGG | 478 | ACGAGGGACCAGCCAAGATCGAGGTGGACC | 926 | 6 |
| HNRNPK | IL2RA | ATGATGTTTGATGACCGTCG | 479 | TACAATGATGTTTGATGACCGTCGCGGACG | 927 | 11 |
| HNRNPK | IL2RA | CTGTTGGGACATACCGCTCG | 480 | TAAACTGTTGGGACATACCGCTCGGGGCCA | 928 | 6 |
| HNRNPK | IL2RA | GATGATATGAGCCCTCGTCG | 481 | TTATGATGATATGAGCCCTCGTCGAGGACC | 929 | 11 |
| HNRNPK | IL2RA | TAAAATCAAAGAACTTCGAG | 482 | GTGCTAAAATCAAAGAACTTCGAGAGGTAA | 930 | 9 |
| MTF1 | IL2RA | AATGCACTTCCACAACACAA | 483 | ATACAATGCACTTCCACAACACAATGGATC | 931 | 6 |
| MTF1 | IL2RA | AATGTGCTGCACATAACCCT | 484 | AGATAATGTGCTGCACATAACCCTGGGACA | 932 | 2 |
| MTF1 | IL2RA | CCACGTGCGAGTGCACACGA | 485 | GGATCCACGTGCGAGTGCACACGAAGGAGA | 933 | 3 |
| MTF1 | IL2RA | GCACATTCGAACTCATACAG | 486 | GGAAGCACATTCGAACTCATACAGGGGAAA | 934 | 4 |
| ABCF1 | IL2RA | CTGCAGAGGCCAAAGCACGG | 487 | GCAGCTGCAGAGGCCAAAGCACGGOGGATC | 935 | 14 |
| ABCF1 | IL2RA | GATGGAGTATGAGCGCCAAG | 488 | CTCAGATGGAGTATGAGCGCCAAGTGGCTT | 936 | 10 |
| ABCF1 | IL2RA | GCAACACATCAATGTTGGGA | 489 | CACAGCAACACATCAATGTTGGGAGGGATG | 937 | 12 |
| ABCF1 | IL2RA | TGTAATTGCCCCTATAGTAG | 490 | TTACTGTAATTGCCCCTATAGTAGTGGAGC | 938 | 16 |
| TBP | IL2RA | ACGTCCCAGCAGGCAACACA | 491 | GTCAACGTCCCAGCAGGCAACACAGGGAAC | 939 | 3 |
| TBP | IL2RA | CCAATGATGCCTTATGGCAC | 492 | TAGTCCAATGATGCCTTATGGCACTGGACT | 940 | 3 |
| TBP | IL2RA | GATAAGAGAGCCACGAACCA | 493 | TGAGGATAAGAGAGCCACGAACCACGGCAC | 941 | 5 |
| TBP | IL2RA | GTTTCGGGCACGAAGTGCAA | 494 | CGGCGTTTCGGGCACGAAGTGCAATGGTCT | 942 | 4 |
| YBX1 | IL2RA | AGACGCTATCCACGTCGTAG | 495 | TTATAGACGCTATCCACGTCGTAGGGGTCC | 943 | 5 |
| YBX1 | IL2RA | GCAAATGTTACAGGTCCTGG | 496 | GGCAGCAAATGTTACAGGTCCTGGTGGTGT | 944 | 5 |
| YBX1 | IL2RA | GGCGGGGACAAGAAGGTCAT | 497 | TGCCGGCGGGGACAAGAAGGTCATCGGTGA | 945 | 1 |
| YBX1 | IL2RA | GTCTTGCAGGAATGACACCA | 498 | TTCTGTCTTGCAGGAATGACACCAAGGAAG | 946 | 3 |
| IRF4 | IL2RA | CAGACCCGTACAAAGTGTAC | 499 | ATCTCAGACCCGTACAAAGTGTACAGGATT | 947 | 3 |
| IRF4 | IL2RA | CCCATGACGTTTGGACCCCG | 500 | ATGTCCCATGACGTTTGGACCCCGCGGCCA | 948 | 5 |
| IRF4 | IL2RA | CTACCGGGAAATCCTCGTGA | 501 | TGTACTACCGGGAAATCCTCGTGAAGGAGC | 949 | 7 |
| IRF4 | IL2RA | CTGATCGACCAGATCGACAG | 502 | GTGGCTGATCGACCAGATCGACAGCGGCAA | 950 | 2 |
| SATB1 | IL2RA | ATGCTAAGTACCTGTGAAAG | 503 | TTCTATGCTAAGTACCTGTGAAAGGGGGCA | 951 | 5 |
| SATB1 | IL2RA | CATTGAATATGATTGCAAGG | 504 | ACGCCATTGAATATGATTGCAAGGAGGAGC | 952 | 3 |
| SATB1 | IL2RA | TAGGTGTTGATACGAGCCCA | 505 | CTGATAGGTGTTGATACGAGCCCAGGGTGC | 953 | 7 |
| SATB1 | IL2RA | TATTCATAGATCTACTGACA | 506 | GGCTTATTCATAGATCTACTGACAGGGGGA | 954 | 7 |
| CREB1 | IL2RA | AGCTGTACTAGAGTTACGGT | 507 | GGGCAGCTGTACTAGAGTTACGGTGGGAGC | 955 | 3 |
| CREB1 | IL2RA | GGCTAACAATGGTACCGATG | 508 | AGCTGGCTAACAATGGTACCGATGGGGTAC | 956 | 6 |
| CREB1 | IL2RA | TGGAGTTGGCACCGTTACAG | 509 | AAATTGGAGTTGGCACCGTTACAGTGGTGA | 957 | 5 |
| CREB1 | IL2RA | TGTGGAGACTGAATAACTGA | 510 | GACTTGTGGAGACTGAATAACTGATGGCTG | 958 | 3 |
| BATF | IL2RA | ACAGAACGCGGCTCTACGCA | 511 | AGAAACAGAACGCGGCTCTACGCAAGGAGA | 959 | 3 |
| BATF | IL2RA | AGGACTCTACCTGTTTGCCA | 512 | AAGGAGGACTCTACCTGTTTGCCAGGGGGA | 960 | 1 |
| BATF | IL2RA | ATGTGAGAAGAGTTCAGAGG | 513 | GATGATGTGAGAAGAGTTCAGAGGAGGGAG | 961 | 2 |
| BATH | IL2RA | GGAGATCAAGCAGCTCACAG | 514 | GCAAGGAGATCAAGCAGCTCACAGAGGAAC | 962 | 3 |
| IRF1 | IL2RA | GAACTCCCTGCCAGATATCG | 515 | CCATGAACTCCCTGCCAGATATCGAGGAGG | 963 | 4 |
| IRF1 | IL2RA | TCTAGGCCGATACAAAGCAG | 516 | GCCCTCTAGGCCGATACAAAGCAGGGGAAA | 964 | 4 |
| IRF1 | IL2RA | TCTCCCTCGACAGTCATGTG | 517 | GCTTTCTCCCTCGACAGTCATGTGGGGATT | 965 | 6 |
| IRF1 | IL2RA | TTAATTCCAACCAAATCCCG | 518 | CAGATTAATTCCAACCAAATCCCGGGGCTC | 966 | 2 |
| CBFB | IL2RA | AAGTCGACATACTCTCGGCT | 519 | TTCTAAGTCGACATACTCTCGGCTAGGTGT | 967 | 3 |
| CBFB | IL2RA | CCTGCCTCACCTCACACTCG | 520 | CCCGCCTGCCTCACCTCACACTCGCGGCTC | 968 | 1 |
| CBFB | IL2RA | GCCGACTTACGATTTCCGAG | 521 | GCCAGCCGACTTACGATTTCCGAGCGGCCG | 969 | 2 |
| CBFB | IL2RA | GGAGTCTGTGTTATCTGGAA | 522 | GAATGGAGTCTGTGTTATCTGGAAAGGCTG | 970 | 4 |
| RNF20 | IL2RA | ACTTCGGCAAGACTTTGAGG | 523 | AGAAACTTCGGCAAGACTTTGAGGAGGTCA | 971 | 9 |
| RNF20 | IL2RA | GCATCGCACCATGTCTCAGG | 524 | AAAAGCATCGCACCATGTCTCAGGAGGTAC | 972 | 6 |
| RNF20 | IL2RA | GGAGGGCACTACCACTACGC | 525 | TGCAGGAGGGCACTACCACTACGCAGGCGT | 973 | 13 |
| RNF20 | IL2RA | TCGGTTGACAATCAATAGTG | 526 | AGTATCGGTTGACAATCAATAGTGAGGCAT | 974 | 3 |
| NR2C2 | IL2RA | CCAGTCGACACCCATCATTG | 527 | GAGACCAGTCGACACCCATCATTGAGGTTG | 975 | 10 |
| NR2C2 | IL2RA | CCCCAGTAAACGCTCCACAG | 528 | TCTTCCCCAGTAAACGCTCCACAGAGGCAG | 976 | 5 |
| NR2C2 | IL2RA | GAACGTCACCTTAGAATCCG | 529 | TAGAGAACGTCACCTTAGAATCCGTGGCCA | 977 | 8 |
| NR2C2 | IL2RA | TCTTTGTCTGCCACAAACGT | 530 | TCCATCTTTGTCTGCCACAAACGTGGGAGT | 978 | 7 |
| FOXK1 | IL2RA | AACAGGCATTCCGGAAACGG | 531 | GTGGAACAGGCATTCCGGAAACGGAGGCAG | 979 | 5 |
| FOXK1 | IL2RA | AGGTCACGTTCTGCACAAAG | 532 | TCCGAGGTCACGTTCTGCACAAAGCGGTAA | 980 | 3 |
| FOXK1 | IL2RA | CTAACTTGGACCCAAACTCA | 533 | GAAGCTAACTTGGACCCAAACTCAGGGTCG | 981 | 6 |
| FOXK1 | IL2RA | GCATTACCCCTACTACCGGA | 534 | CCAAGCATTACCCCTACTACCGGACGGCCG | 982 | 4 |
| MYC | IL2RA | AGAGTGCATCGACCCCTOGG | 535 | CCTCAGAGTGCATCGACCCCTCGGTGGTCT | 983 | 2 |
| MYC | IL2RA | CTGCGGGGAGGACTCCGTCG | 536 | TGCCCTGCGGGGAGGACTCCGTCGAGGAGA | 984 | 2 |
| MYC | IL2RA | CTTCGGGGAGACAACGACGG | 537 | CTCCCTTCGGGGAGACAACGACGGCGGTGG | 985 | 2 |
| MYC | IL2RA | GCTGCACCGAGTCGTAGTCG | 538 | TACGGCTGCACCGAGTCGTAGTCGAGGTCA | 986 | 2 |
| KLF2 | IL2RA | AAACCAGGGCCACCGAAAGG | 539 | GCCGAAACCAGGGCCACCGAAAGGCGGCGG | 987 | 2 |
| KLF2 | IL2RA | CCCTCGCGCTTGAGGCCGCG | 540 | GGCGCCCTCGCGCTTGAGGCCGCGCGGTCC | 988 | 2 |
| KLF2 | IL2RA | CTTCGGTCTCTTCGACGACG | 541 | CAGCCTTCGGTCTCTTCGACGACGCGGCCG | 989 | 2 |
| KLF2 | IL2RA | TCGGGGTAATAGAACGCAGG | 542 | GGGTTCGGGGTAATAGAACGCAGGCGGCGG | 990 | 2 |
| TFDP1 | IL2RA | ACCGGCAGCGTCAAACACCC | 543 | AGAGACCGGCAGCGTCAAACACCCTGGTGG | 991 | 5 |
| TFDP1 | IL2RA | ATGACCAGAAAAACATAAGA | 544 | GCTTATGACCAGAAAAACATAAGACGGCGC | 992 | 7 |
| TFDP1 | IL2RA | CCTTCATGGAGAAATGCCGT | 545 | CAGACCTTCATGGAGAAATGCCGTAGGCCC | 993 | 6 |
| TFDP1 | IL2RA | GGTGCAGAGAAACCGGCATG | 546 | ACCTGGTGCAGAGAAACCGGCATGCGGAGC | 994 | 9 |
| BPTF | IL2RA | AAAGGAGCAGAGCAAAACCG | 547 | CTGTAAAGGAGCAGAGCAAAACCGTGGTCA | 995 | 11 |
| BPTF | IL2RA | ACTGCTATCCTGAATAGACA | 548 | CTTCACTGCTATCCTGAATAGACACGGTAT | 996 | 11 |
| BPTF | IL2RA | GAGGACTTGGGAAATTCAAG | 549 | CTCAGAGGACTTGGGAAATTCAAGGGGCGG | 997 | 2 |
| BPTF | IL2RA | GCCTTCGATCAAGTGCACTG | 550 | AAAGGCCTTCGATCAAGTGCACTGCGGCCA | 998 | 14 |
| KLF13 | IL2RA | CCGACCTCGAGTCCCCGCAG | 551 | CGCGCCGACCTCGAGTCCCCGCAGAGGAAG | 999 | 1 |
| KLF13 | IL2RA | CGTGGTGGCGCGGATCCTAG | 552 | TCTTCGTGGTGGCGCGGATCCTAGCGGACC | 1000 | 1 |
| KLF13 | IL2RA | CGTGTCCATGTCGAGCCGCG | 553 | GCCTCGTGTCCATGTCGAGCCGCGCGGTCG | 1001 | 1 |
| KLF13 | IL2RA | GAGTTCTCAGGTGCGCCTTG | 554 | GTGTGAGTTCTCAGGTGCGCCTTGAGGTGC | 1002 | 1 |
| MYB | IL2RA | ACCAGGCACACAAGAGACTG | 555 | ATTTACCAGGCACACAAGAGACTGGGGAAC | 1003 | 5 |
| MYB | IL2RA | AGAAATACGGTCCGAAACGT | 556 | GTACAGAAATACGGTCCGAAACGTTGGTCT | 1004 | 5 |
| MYB | IL2RA | AGTCTGGAAAGCGTCACTTG | 557 | CCCAAGTCTGGAAAGCGTCACTTGGGGAAA | 1005 | 2 |
| MYB | IL2RA | TATTTACATGTAACGCTACA | 558 | ACTATATTTACATGTAACGCTACAGGGTAT | 1006 | 7 |
| ZNF217 | IL2RA | CAAAATCTCACCCTGAAACG | 559 | AGACCAAAATCTCACCCTGAAACGGGGAAG | 1007 | 1 |
| ZNF217 | IL2RA | CCACGGCGAAGCGCCCTCCG | 560 | ACTCCCACGGCGAAGCGCCCTCCGTGGACG | 1008 | 1 |
| ZNF217 | IL2RA | GGACACATAATGGCAAATCG | 561 | ATGCGGACACATAATGGCAAATCGGGGGCC | 1009 | 1 |
| ZNF217 | IL2RA | TGGGTGGTACTGCCATCCGG | 562 | GTTATGGGTGGTACTGCCATCCGGAGGAGG | 1010 | 3 |

In some embodiments, the T cell comprises (a) a genetic modification or heterologous polynucleotide that inhibits expression of FOXN2, ZNF25, THRA, ZBTB2, CREM. PTF1A. IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574; and/or (b) a heterologous polynucleotide that encodes FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574

In some embodiments, the T cell comprises (a) a genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is increased in the T cell relative to expression of IL2RA in a T cell not comprising the genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1. TGIF2, or ZNF574; and/or (b) a heterologous polynucleotide that encodes FOXN2, ZNF25, THRA, ZBTB2, CREM, PTFIA, IKZF2, MYNN, BACH2, IL15RA, SP3, ZBTB3, REST, ELP2, ZBTB14, GFI1, IL2RB, NR4A3, or ZBTB32, wherein expression of IL2RA is increased in the T cell relative to expression of IL2RA in a T cell not comprising the heterologous polynucleotide that encodes FOXN2, ZNF25, THRA, ZBTB2, CREM, PTFIA, IKZF2, MYNN, BACH2, IL15RA, SP3, ZBTB3, REST, ELP2, ZBTB14, GFI1, IL2RB, NR4A3, or ZBTB32.

In some embodiments, the T cell comprises (a) a genetic modification or heterologous polynucleotide that inhibits expression of FOXN2, ZNF25, THRA, ZBTB2, CREM, PTFIA, IKZF2, MYNN, BACH2, IL15RA, SP3, ZBTB3, REST, ELP2, ZBTB14, GFI1, IL2RB, NR4A3, or ZBTB32, wherein expression of IL2RA is decreased in the T cell relative to expression of IL2RA in a T cell not comprising the genetic modification or heterologous polynucleotide that inhibits expression of FOXN2, ZNF25, THRA, ZBTB2, CREM, PTFIA, IKZF2. MYNN, BACH2, IL15RA, SP3, ZBTB3, REST, ELP2, ZBTB14, GFI1, IL2RB. NR4A3, or ZBTB32; and/or (b) a heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the T cell relative to expression of IL2RA in a T cell not comprising heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574.

In some embodiments, the T cell is a human T cell. In some embodiments, the T cell is a regulatory T cell (Treg), an effector T cell (e.g., a CD4+ T cell) or a stimulated T cell (e.g., a stimulated CD4+ T cell). In some embodiments, the T cell is a resting T cell. Populations comprising any of the genetically modified T cells described herein are also provided.

In some embodiments, the regulatory T cell comprises: (a) a genetic modification or heterologous polynucleotide that inhibits expression ZKSCAN2, ZNF114, E4F1, GLIS2, JUNB, NR2F6, ZZZ3, ETV3, ERF, RUNX1, TGIF2, or ZNF574, wherein expression of IL2RA is increased in the Treg cell relative to expression of IL2RA in a Treg cell not comprising the genetic modification or heterologous polynucleotide that inhibits expression of; and/or (b) a heterologous polynucleotide that encodes FOXN2, ZNF25, THRA, ZBTB2, CREM, PTFIA, IKZF2, MYNN, BACH2, IL15RA, SP3, or ZBTB3, wherein expression of IL2RA is increased in the Treg cell relative to expression of IL2RA in a Treg cell not comprising the heterologous polynucleotide that encodes FOXN2, ZNF25, THRA, ZBTB2, CREM, PTFIA, IKZF2, MYNN, BACH2, IL15RA, SP3, or ZBTB3.

In some embodiments, the Treg cell comprises (a) a genetic modification or heterologous polynucleotide that inhibits expression FOXN2, ZNF25, THRA, ZBTB2, CREM, PTF1A, IKZF2, MYNN, BACH2, IL15RA, SP3, or ZBTB3, wherein expression of IL2RA is decreased in the Treg cell relative to expression of IL2RA in a Treg cell not comprising the genetic modification or heterologous polynucleotide that inhibits expression of FOXN2, ZNF25, THRA, ZBTB2, CREM, PTF1A, IKZF2, MYNN, BACH2, IL15RA, S3, or ZBTB3; and/or (b) a heterologous polynucleotide that encodes ZKSCAN2, ARID5A, ZNF114. E4F1, GLIS2, JUNB, NR2F6, ZZZ3, ETV3, ERF, RUNX1, TGIF2, or ZNF574, wherein expression of IL2RA is decreased in the Treg cell relative to expression of IL2RA in a Treg cell not comprising the heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574.

In some embodiments, the CD4+ T cell comprises: (a) a genetic modification or heterologous polynucleotide that inhibits expression of BACH2, ZBTB3, ABTB14, GFI1, or IL2RB, wherein expression of IL2RA is decreased in the CD4+ T cell relative to expression of IL2RA in a CD4+ T cell not comprising the genetic modification or heterologous polynucleotide that inhibits expression of BACH2, REST, ZBTB3. ABTB14, GFI1, or IL2RB.

In some embodiments, the stimulated CD4+ T cell comprises: (a) a genetic modification or heterologous polynucleotide that inhibits expression of REST, ELP2, IL2RB, NR4A3, or ZBTB32, wherein expression of IL2RA is decreased in the stimulated CD4+ T cell relative to expression of IL2RA in a stimulated CD4+ T cell not comprising the genetic modification or heterologous polynucleotide that inhibits expression of REST, ELP2, IL2RB. NR4A3, or ZBTB32; and/or (b) a heterologous polynucleotide that encodes BACH2, STAT6, ZMYND8, IFNGR2, IL4R, LEF1, or ARID5A, wherein expression of IL2RA is decreased in the stimulated CD4+ T cell relative to expression of IL2RA in a stimulated CD4+ T cell not comprising the heterologous polynucleotide that encodes BACH2, STAT6, ZMYND8, IFNGR2, IL4R, LEF1, or ARID5A.

In some embodiments, the stimulated CD4+ T cell comprises: (a) a genetic modification or heterologous polynucleotide that inhibits expression of BACH2, STAT6, ZMYND8, IFNGR2, IL4R, LEF1, or ARID5A, wherein expression of IL2RA is increased in the stimulated CD4+ T cell relative to expression of IL2RA in a stimulated CD4+ T cell not comprising the genetic modification or heterologous polynucleotide that inhibits expression of BACH2, STAT6, ZMYND8, IFNGR2, IL4R, LEF1, or ARID5A; and/or (b) a heterologous polynucleotide that encodes REST, ELP2, IL2RB, NR4A3, or ZBTB32, wherein expression of IL2RA is increased in the stimulated CD4+ T cell relative to expression of IL2RA in a stimulated CD4+ T cell not comprising the heterologous polynucleotide that encodes REST, ELP2, IL2RB, NR4A3, or ZBTB32.

In some embodiments, expression of an amino acid sequence having at least about 80%, 85%, 90%, 95% or 99% identity to an amino acid sequence set forth in Table 1 is inhibited. In some embodiments, an amino acid sequence having at least about 80%, 85%, 90%, 95% or 99% identity to an amino acid sequence set forth in Table 1 is overexpressed. It is understood that, when referring to one or more nuclear factors set forth in Table 1 this can be the protein, i.e., the nuclear factor, or the polynucleotide encoding the nuclear factor.

In some embodiments of the methods described herein, inhibiting the expression of a nuclear factor set forth in Table 1 may comprise reducing expression of the nuclear factor or reducing expression of a polynucleotide, for example, an mRNA, encoding the nuclear factor in the T cell. In some embodiments expression of one or more nuclear factors set forth in Table 1, is inhibited in the T cell. As described in detail further herein, one or more available methods may be used to inhibit the expression of one or more nuclear factors set forth in Table 1.

In some embodiments of the methods described herein, overexpressing a nuclear factor set forth in Table 1 may comprise introducing a polynucleotide encoding the nuclear factor into the T cell. In other embodiments of the methods described herein, overexpressing a nuclear factor set forth in Table 1 may comprise introducing an agent that induces expression of the endogenous gene encoding the nuclear factor in the T cell. For example, RNA activation, where short double-stranded RNAs induce endogenous gene expression by targeting promoter sequences, can be used to induce endogenous gene expression (See, for example, Wang et al. “Inducing gene expression by targeting promoter sequences using small activating RNAs,” J. Biol. Methods 2(1): e14 (2015). In another example, artificial transcription factors containing zinc-finger binding domains can be used to activate or repress expression of endogenous genes. See, for example, Dent et al., “Regulation of endogenous gene expressing using small molecule-controlled engineered zinc-finger protein transcription factors,” Gene Ther. 14(18): 1362-9 (2007).

In some embodiments, inhibiting expression may comprise contacting a polynucleotide encoding the nuclear factor, with a target nuclease, a guide RNA (gRNA), an siRNA, an antisense RNA, microRNA (miRNA), or short hairpin RNA (shRNA). In particular embodiments, if a gRNA and a target nuclease (e.g., Cas9) are used to inhibit the expression of a polynucleotide encoding a human nuclear factor set forth in Table 1, the gRNA may comprise a sequence set forth in Tables 2, a sequence complementary to a sequence set forth in Table 2, or a portion thereof. Table 1 provides the Gene ID number, Genbank Accession No. for mRNA, genomic sequence, and amino acid sequence for each target (e.g., nuclear factor). Exemplary sgRNA target sequences, target context sequence, and the exon targeted by the sgRNA for each nuclear factor are set forth in Table 2.

As described herein. T cells may be modified by inhibiting the expression of the one or more nuclear factors set forth in Table 1. For example, one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, IKZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217 can be inhibited in a T cell.

T cells may also be modified by overexpressing one or more nuclear factors set forth in Table 1. For example, one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, 1KZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217, can be overexpressed in a T cell.

In some embodiments, one or more nuclear factors set forth in Table 1 can be inhibited to maintain the resting state (e.g., suppress activation) of a T cell, for example, a regulatory T cell or an effector T cell. In some embodiments, one or more nuclear factors set forth in Table 1 can be overexpressed to maintain the resting state (e.g., suppress activation) of a T cell, for example, a regulatory T cell or an effector T cell. In some embodiments, one or more nuclear factors set forth in Table 1 can be inhibited to activate a T cell (e.g., transition from resting state to activated state), for example, a regulatory T cell or an effector T cell. In some embodiments, one or more nuclear factors set forth in Table 1 can be overexpressed to activate a T cell (e.g., transition from resting state to activated state), for example, a regulatory T cell or an effector T cell.

Subsequently, once modified T cells, for example, human T cells, are created, the modified T cells may be administered to a human. Depending on the modification, the modified T cells may be used to treat different indications. For example, T cells may be isolated from a whole blood sample of a human and expanded ex vivo. The expanded T cells may then be treated to inhibit the expression of one or more nuclear factors set forth in Table 1. For example, one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574, can be inhibited in the T cell. In some embodiments, expanded T cells may be treated to overexpress one or more nuclear factors set forth in Table 1. For example, one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574, can be overexpressed in the T cell.

The modified T cells may be reintroduced to the human to treat certain indications. In some embodiments, T cells having less immunosuppressive effects or enhanced cytotoxic or cell-killing effects may be used to treat cancer. In some embodiments, T cells having improved immunosuppressive effects may be used to treat autoimmune diseases.

In other cases, T cells in a subject can be modified in vivo, for example, by using a targeted vector, such as, a lentiviral vector, a retroviral vector an adenoviral or adeno-associated viral vector. In vivo delivery of targeted nucleases that modify the genome of a T cell can also be used. See for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017).

Also provided is a T cell wherein expression of one or more nuclear factors set forth in Table 1 is inhibited. In some embodiments, expression of one or more nuclear factors selected from the group consisting of FOXN2, ZNF25, THRA, ZBTB2. CREM, PTF1A. IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, IKZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B. RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217, is inhibited in a T cell.

Further provided is a T cell wherein one or more nuclear factors set forth in Table 1, are overexpressed. In some embodiments, one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, IKZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217, is overexpressed in a T cell.

The disclosure also features a T cell comprising a genetic modification or heterologous polynucleotide that inhibits expression of one or more nuclear factors set forth in Table 1 and/or a heterologous polynucleotide that encodes a nuclear factor set forth in Table 1.

In some embodiments, the T cell comprises (a) a genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574; and/or (b) a heterologous polynucleotide that encodes FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574.

It is understood that one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574, can be inhibited and/or overexpressed in the T cells provided herein.

In some embodiments, the T cells is a regulatory T cell comprising (a) a genetic modification or heterologous polynucleotide that inhibits expression ZKSCAN2, ZNF114, E4F1, GLIS2, JUNB, NR2F6, ZZZ3, ETV3, ERF, RUNX1, TGIF2, or ZNF574, wherein expression of IL2RA is increased in the Treg cell relative to expression of IL2RA in a Treg cell not comprising the genetic modification or heterologous polynucleotide that inhibits expression of ZKSCAN2, ZNF114, E4F1, GLIS2, JUNB, NR2F6, ZZZ3, ETV3, ERF, RUNX1, TGIF2, or ZNF574 and/or (b) a heterologous polynucleotide that encodes FOXN2, ZNF25, THRA, ZBTB2, CREM, PTFIA, IKZF2, MYNN, BACH2, IL15RA, SP3, or ZBTB3, wherein expression of IL2RA is increased in the Treg cell relative to expression of IL2RA in a Treg cell not comprising the heterologous polynucleotide that encodes FOXN2, ZNF25, THRA. ZBTB2, CREM, PTFIA, IKZF2, MYNN, BACH2, IL15RA, SP3, or ZBTB3. Any of the regulatory T cells provided herein having increased expression of IL2RA can be used to treat an autoimmune disorder in a subject.

In some embodiments, the T cell is a regulatory T cell comprising (a) a genetic modification or heterologous polynucleotide that inhibits expression FOXN2, ZNF25, THRA, ZBTB2, CREM, PTF1A, IKZF2, MYNN, BACH2, IL15RA, SP3, or ZBTB3, wherein expression of IL2RA is decreased in the Treg cell relative to expression of IL2RA in a Treg cell not comprising the genetic modification or heterologous polynucleotide that inhibits expression of FOXN2, ZNF25, THRA, ZBTB2, CREM, PTFIA, IKZF2, MYNN, BACH2, IL15RA, S3, or ZBTB3; and/or (b) a heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the Treg cell relative to expression of IL2RA in a Treg cell not comprising the heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574. Any of the regulatory T cells provided herein having decreased expression of IL2RA can be used to treat cancer in a subject.

In some embodiments, the T cell is an effector cell (e.g., a CD4+ T cell) comprising: (a) a genetic modification or heterologous polynucleotide that inhibits expression of BACH2, ZBTB3, ABTB14, GFI1, or IL2RB, wherein expression of IL2RA is decreased in the CD4+ T cell relative to expression of IL2RA in a CD4+ T cell not comprising the genetic modification or heterologous polynucleotide that inhibits expression of BACH2, REST, ZBTB3, ABTB14, GFI1, or IL2RB.

In some embodiments, the T cell is an effector cell (e.g., a stimulated CD4+ T cell) comprising: (a) a genetic modification or heterologous polynucleotide that inhibits expression of REST, ELP2, IL2RB, NR4A3, or ZBTB32, wherein expression of IL2RA is decreased in the stimulated CD4+ T cell relative to expression of IL2RA in a stimulated CD4+ T cell not comprising the genetic modification or heterologous polynucleotide that inhibits expression of REST, ELP2, IL2RB, NR4A3, or ZBTB32; and/or (b) a heterologous polynucleotide that encodes BACH2, STAT6, ZMYND8, IFNGR2, IL4R, LEF1, or ARID5A, wherein expression of IL2RA is decreased in the stimulated CD4+ T cell relative to expression of IL2RA in a stimulated CD4+ T cell not comprising the heterologous polynucleotide that encodes BACH2, STAT6, ZMYND8, IFNGR2, IL4R, LEF1, or ARID5A. Any of the effector T cells provided herein having decreased expression of IL2RA can be used to treat an autoimmune disorder in a subject.

In some embodiments, the T cell is an effector cell (e.g., a stimulated CD4+ T cell) comprising: (a) (a) a genetic modification or heterologous polynucleotide that inhibits expression of BACH2, STAT6, ZMYND8, IFNGR2, IL4R, LEF1, or ARID5A, wherein expression of IL2RA is increased in the stimulated CD4+ T cell relative to expression of IL2RA in a stimulated CD4+ T cell not comprising the genetic modification or heterologous polynucleotide that inhibits expression of BACH2, STAT6, ZMYND8, IFNGR2, IL4R, LEF1, or ARID5A; and/or (b) a heterologous polynucleotide that encodes REST, ELP2, IL2RB, NR4A3, or ZBTB32, wherein expression of IL2RA is increased in the stimulated CD4+ T cell relative to expression of IL2RA in a stimulated CD4+ T cell not comprising the heterologous polynucleotide that encodes REST, ELP2, IL2RB, NR4A3, or ZBTB32. Any of the effector T cells provided herein having increased expression of IL2RA can be used to treat an cancer in a subject.

In some embodiments, the T cell is a Treg cell. In some embodiments, the T cell is a CD8+, a CD4+ or a CD8+CD4+ T cell. In some embodiments, the T cell is a stimulated T cell (e.g., a CD4+ T cell). Also provided, are populations of cells comprising any of the genetically modified T cells described herein.

In some examples, a Treg cell having decreased IL2RA expression can be used to treat cancer. In some embodiments, the T cell is a conventional T cell, for example, CD4+ or CD8+ T cell, with decreased IL2RA expression. In some examples, a conventional T cell having decreased IL2RA expression can be used to treat autoimmune disease.

In some examples, a Treg cell having increased IL2RA expression can be used to treat an autoimmune disorder. In some embodiments, the T cell is a conventional T cell, for example, CD4+ or CD8+ T cell, with increased IL2RA expression. In some examples, a conventional T cell having increased IL2RA expression can be used to treat cancer.

A genetic modification may be a nucleotide mutation or any sequence alteration in the polynucleotide encoding the nuclear factor that results in the inhibition of the expression of the nuclear factor. A heterologous polynucleotide may refer to a polynucleotide originally encoding the nuclear factor but is altered, i.e., comprising one or more nucleotide mutations or sequence alterations. In some embodiments, the heterologous polynucleotide is inserted into the genome of the T cell by introducing a vector, for example, a viral vector, comprising the polynucleotide. Examples of viral vectors include, but are not limited to adeno-associated viral (AAV) vectors, retroviral vectors or lentiviral vectors. In some embodiments, the lentiviral vector is an integrase-deficient lentiviral vector.

Also disclosed herein are T cells comprising at least one guide RNA (gRNA) comprising a sequence selected from Table 1. The expression of one or more nuclear factors set forth in Table 1 in the T cells comprising the gRNAs, may be reduced in the T cells relative to the expression of the one or more nuclear factors in T cells not comprising the gRNAs. In other examples, an endogenous nuclear factor set forth in Table 1 can be inhibited by targeting a deactivated targeted nuclease, for example dCAs9, fused to a transcriptional repressor, to the promoter region of the endogenous nuclear factor gene. In other examples, an endogenous nuclear factor set forth in Table 1 can be upregulated or overexpressed by targeting a deactivated targeted nuclease, for example dCAs9, fused to a transcriptional activator, to the promoter region of the endogenous nuclear factor gene. See, for example, Qi et al. “The New State of the Art: Cas9 for Gene Activation and Repression,” Mol. and Cell. Biol., 35(22): 3800-3809 (2015).

### II. Methods of Inhibiting Expression

### CRISPR/Cas Genome Editing

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “guide RNA” or “gRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments of the methods described herein, CRISPR/Cas genome editing may be used to inhibit the expression of one or more nuclear factors set forth in Table 1. For example, CRISPR/Cas genome editing may be used to inhibit expression of one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32. ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, IKZF3, PURA, SETDB1, RELA, SS18, SRF, GATA3, ZNF384, ZNF148, JAK3, MED14, PKNOX1. USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2. HNRNPK, MTF1, ABCF1, TBP, YBX1, IRF4, SATB1, CREB1. BATF, IRF1, CBFB, RNF20, NR2C2, FOXK1, MYC, KLF2, TFDP1, BPTF, KLF13, MYB, and ZNF217.

In another example, CRISPR/Cas genome editing may be used to inhibit expression of one or more nuclear factors selected from the group consisting of FOXN2, ZNF25, THRA, ZBTB2, CREM. PTF1A, IKZF2, MYNN, BACH2, IL15RA, SP3. ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574.

In some embodiments, the Cas nuclease has DNA cleavage activity. The Cas nuclease can direct cleavage of one or both strands at a location in a target DNA sequence, i.e., a location in a polynucleotide encoding a nuclear factor set forth in Table 1. In some embodiments, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Some CRISPR-related endonucleases that may be used in methods described herein are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus cotus, Treponema denticola, Peptoniphilus duerdenit, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifdum, Lactobacillus rhamnosus, Iactobacillus gasseri, Finegoldia magna, MyWoplasma mobile, Mycoplasma gallisepticum, Mycoplasma ovipneumoniae, Mycoplasma canis, Mycoplasma synoviae, Eubactenum rectale, Streptococcus thermophilus, Eubacterium dolichum, Lactobacillus coryniformis subsp, Torquens, Ilyobacter polytropus, Ruminococcus albus, Akkermansia muciniphila, Acidothermus cellulolvticus, Bifidobacterium longum, Biftdobacterium dentium, Corynebacterium diphtheria, Elusimicrobium minutum, Nitratifractor salsuginis, Sphaerochaeta globus, Fibrobacter succinogenes subsp, Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzgii, Dinoroseobacter shibae, Azospirillum, Nitrobacter hamburgensis, Bradyrhizobium, Wolinella succinogenes, Campylobacter jejuni subsp, Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp, Multocida, Sutterella wadsworthensis, proteobacterium, Legionella pneumophila, Parasutterella excrementihominis, Wolinella succinogenes, and Francisella novicida.

Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or mor catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 may be a fusion protein. e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease may be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break may be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme may contain a mutation at D10, E762, H983, or D986, as well as a mutation at H840 or N863. In some instances, the dCas9 enzyme may contain a D10A or D10N mutation. Also, the dCas9 enzyme may contain a H840A, H840Y, or H840N. In some embodiments, the dCas9 enzyme may contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

As described above, a gRNA may comprise a crRNA and a tracrRNAs. The gRNA can be configured to form a stable and active complex with a gRNA-mediated nuclease (e.g., Cas9 or dCas9). The gRNA contains a binding region that provides specific binding to the target genetic element. Exemplary gRNAs that may be used to target a region in a polynucleotide encoding a nuclear factor described herein are set forth in Table 1. A gRNA used to target a region in a polynucleotide encoding a nuclear factor set forth in Table 1, or a portion thereof.

In some embodiments, the targeted nuclease, for example, a Cpf1 nuclease or a Cas9 nuclease and the gRNA are introduced into the T cell as a ribonucleoprotein (RNP) complex. In some embodiments, the RNP complex may be introduced into about 1×10⁵ to about 2×10⁶ cells (e.g., 1×10⁵ cells to about 5×10⁵ cells, about 1×10⁵ cells to about ×10⁶ cells, 1×10⁶ cells to about 1.5×10⁶ cells, 1×10⁵ cells to about 2=10⁶ cells, about 1×10⁶ cells to about 1.5×10⁶ cells, or about 1=10⁶ cells to about 2×10⁶ cells). In some embodiments, the T cells are cultured under conditions effective for expanding the population of modified T cells. Also disclosed herein is a population of T cells, in which the genome of at least 20%, 30%, 40%, 50%, 60%, 70%, 80%, 90%, 95%, 99% or greater of the cells comprises a genetic modification or heterologous polynucleotide that inhibits expression of one or more nuclear factors set forth in Table 1.

In some embodiments, the RNP complex is introduced into the T cells by electroporation. Methods, compositions, and devices for electroporating cells to introduce a RNP complex are available in the art, see, e.g., WO 2016/123578, WO/2006/001614, and Kim, J. A. et al. Biosens. Bioelectron. 23, 1353-1360 (2008). Additional or alternative methods, compositions, and devices for electroporating cells to introduce a RNP complex can include those described in U.S. Patent Appl. Pub. Nos. 2006/0094095; 2005/0064596; or 2006/0087522; Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010).

In some embodiments, the sequence of the gRNA or a portion thereof is designed to complement (e.g., perfectly complement) or substantially complement (e.g., 50%, 55%, 60%, 65%, 70%, 75%, 80%, 85%, 90%, 91%, 92%, 93%, 94% 95%, 96%, 97%, 98%, or 99% complement) the target region in the polynucleotide encoding the protein. In some embodiments, the portion of the gRNA that complements and binds the targeting region in the polynucleotide is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more nucleotides in length. In some cases, the portion of the gRNA that complements and binds the targeting region in the polynucleotide is between about 19 and about 21 nucleotides in length. In some cases, the gRNA may incorporate wobble or degenerate bases to bind target regions. In some cases, the gRNA can be altered to increase stability. For example, non-natural nucleotides, can be incorporated to increase RNA resistance to degradation. In some cases, the gRNA can be altered or designed to avoid or reduce secondary structure formation. In some cases, the gRNA can be designed to optimize G-C content. In some cases, G-C content is between about 40% and about 60% (e.g., 40%, 45%, 50%, 55%, 60%). In some cases, the binding region can contain modified nucleotides such as, without limitation, methylated or phosphorylated nucleotides

In some embodiments, the gRNA can be optimized for expression by substituting, deleting, or adding one or more nucleotides. In some cases, a nucleotide sequence that provides inefficient transcription from an encoding template nucleic acid can be deleted or substituted. For example, in some cases, the gRNA is transcribed from a nucleic acid operably linked to an RNA polymerase III promoter. In such cases, gRNA sequences that result in inefficient transcription by RNA polymerase III, such as those described in Nielsen et al., Science. 2013 Jun. 28:340(6140):1577-80, can be deleted or substituted. For example, one or more consecutive uracils can be deleted or substituted from the gRNA sequence. In some cases, if the uracil is hydrogen bonded to a corresponding adenine, the gRNA sequence can be altered to exchange the adenine and uracil. This “A-U flip” can retain the overall structure and function of the gRNA molecule while improving expression by reducing the number of consecutive uracil nucleotides.

In some embodiments, the gRNA can be optimized for stability. Stability can be enhanced by optimizing the stability of the gRNA:nuclease interaction, optimizing assembly of the gRNA:nuclease complex, removing or altering RNA destabilizing sequence elements, or adding RNA stabilizing sequence elements. In some embodiments, the gRNA contains a 5′ stem-loop structure promixal to, or adjacent to, the region that interacts with the gRNA-mediated nuclease. Optimization of the 5′ stem-loop structure can provide enhanced stability or assembly of the gRNA:nuclease complex. In some cases, the 5′ stem-loop structure is optimized by increasing the length of the stem portion of the stem-loop structure.

gRNAs can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Also described herein are expression cassettes and vectors for producing gRNAs in a host cell. The expression cassettes can contain a promoter (e.g., a heterologous promoter) operably linked to a polynucleotide encoding a gRNA. The promoter can be inducible or constitutive. The promoter can be tissue specific. In some cases, the promoter is a U6, HI, or spleen focus-forming virus (SFFV) long terminal repeat promoter. In some cases, the promoter is a weak mammalian promoter as compared to the human elongation factor 1 promoter (EF1A). In some cases, the weak mammalian promoter is a ubiquitin C promoter or a phosphoglycerate kinase 1 promoter (PGK). In some cases, the weak mammalian promoter is a TetOn promoter in the absence of an inducer. In some cases, when a TetOn promoter is utilized, the host cell is also contacted with a tetracycline transactivator. In some embodiments, the strength of the selected gRNA promoter is selected to express an amount of gRNA that is proportional to the amount of Cas9 or dCas9. The expression cassette can be in a vector, such as a plasmid, a viral vector, a lentiviral vector, etc. In some cases, the expression cassette is in a host cell. The gRNA expression cassette can be episomal or integrated in the host cell.

### Zinc-Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some embodiments of the methods described herein, ZFNs may be used to inhibit the expression of one or more nuclear factors set forth in Table 1 or Table 2, i.e., by cleaving the polynucleotide encoding the protein.

In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that may be used in methods described herein include, but are not limited to, those described in Umov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903.185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

### TALENs

TALENS may also be used to inhibit the expression of one or more nuclear factors set forth in Table 1. “TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to FokI have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49.

### Meganucleases

Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments of the methods described herein, meganucleases may be used to inhibit the expression of one or more nuclear factors set forth in Table 1, i.e., by cleaving in a target region within the polynucleotide encoding the nuclear factor. In some instances, the meganuclease is naturally-occurring (found in nature) or wild-type, and in other instances, the meganuclease is non-natural, artificial, engineered, synthetic, or rationally designed. In certain embodiments, the meganucleases that may be used in methods described herein include, but are not limited to, an 1-CreI meganuclease, I-CeuI meganuclease, I-MsoI meganuclease, 1-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

### RNA-Based Technologies

Various RNA-based technologies may also be used in methods described herein to inhibit the expression of one or more nuclear factors set forth in Table 1. Examples of RNA-based technologies include, but are not limited to, small interfering RNA (siRNA), antisense RNA, microRNA (miRNA), and short hairpin RNA (shRNA).

RNA-based technologies may use an siRNA, an antisense RNA, a miRNA, or a shRNA to target a sequence, or a portion thereof, that encodes a transcription factor. In some embodiments, one or more genes regulated by a transcription factor may also be targeted by an siRNA, an antisense RNA, a miRNA, or a shRNA. An siRNA, an antisense RNA, a miRNA, or a shRNA may target a sequence comprising at least 10, at least 20, at least 30, at least 40, at least 50, at least 60, at least 70, at least 80, at least 90, or at least 100 contiguous nucleotides.

An siRNA may be produced from a short hairpin RNA (shRNA). A shRNA is an artificial RNA molecule with a hairpin turn that can be used to silence target gene expression via the siRNA it produces in cells. See, e.g., Fire et al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by a protein called Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

The shRNA or siRNA may be encoded in a vector. In some embodiments, the vector further comprises appropriate expression control elements known in the art, including, e.g., promoters (e.g., inducible promoters or tissue specific promoters), enhancers, and transcription terminators.

### III. Methods of Treatment

Any of the methods described herein may be used to modify T cells in a human subject or obtained from a human subject. Any of the methods and compositions described herein may be used to modify T cells obtained from a human subject to treat or prevent a disease (e.g., cancer, an autoimmune disease, an infectious disease, transplantation rejection, graft vs. host disease or other inflammatory disorder in a subject).

In some embodiments, the T cells obtained from a human subject can be modified to inhibit expression of one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, 1KZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217.

In some embodiments, the T cells obtained from a human subject can be modified to overexpress one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, ZNF574, PTEN, IL2RA, FOXO1, FOXP1, STAT5B, STAT5A, MED12, FOXP3, FLI1, ETS1, ATXN7L3, KMT2A, ZEB1, MBD2, CIC, TAF5L, USP22, MED30, IKZF1, MED11, RXRB, IKZF3, PURA, SETDB1, RELA, SS18, SRF, GATA3, ZNF384, ZNF148, JAK3, MED14, PKNOX1, USF2, KLF6, DNMT1, DDX39B, ZNF236, SOCS3, GABPA, RBPJ, STAT3, VPS52, IL2, BCL11B, RAD21, HIVEP2, IRF2, TFAP4, PRDM1, TNFAIP3, MEF2D, SMARCB1, NFKB2, HNRNPK, MTF1, ABCF1, TBP, YBX1, IRF4, SATB1, CREB1, BATF, IRF1, CBFB, RNF20, NR2C2, FOXK1, MYC, KLF2, TFDP1, BPTF, KLF13, MYB, and ZNF217.

Provided herein is a method of treating an autoimmune disorder in a subject, the method comprising administering a population of regulatory T cells having increased IL2RA expression comprising: (a) a genetic modification or heterologous polynucleotide that inhibits expression of a nuclear factor set forth in Table 3, and/or a (b) a heterologous polynucleotides that encodes a nuclear factor set forth in Table 4, to a subject that has an autoimmune disorder.

TABLE 3
Nuclear Factors that can be inhibited to increase
IL2RA expression in regulatory T cells

| Gene Name | Regulator Type |
|---|---|
| ZKSCAN2 | Negative Regulator |
| ZNF114 | Negative Regulator |
| E4F1 | Negative Regulator |
| GLIS2 | Negative Regulator |
| JUNB | Negative Regulator |
| NR2F6 | Negative Regulator |
| ZZZ3 | Negative Regulator |
| ETV3 | Negative Regulator |
| ERF | Negative Regulator |
| RUNX1 | Negative Regulator |
| TGIF2 | Negative Regulator |
| ZNF574 | Negative Regulator |

TABLE 4
Nuclear Factors that can be overexpressed to increase
IL2RA expression in regulatory T cells

| Gene Name | Regulator Type |
|---|---|
| FOXN2 | Positive Regulator |
| ZNF25 | Positive Regulator |
| THRA | Positive Regulator |
| ZBTB2 | Positive Regulator |
| CREM | Positive Regulator |
| PTF1A | Positive Regulator |
| IKZF2 | Positive Regulator |
| MYNN | Positive Regulator |
| BACH2 | Positive Regulator |
| IL15RA | Positive Regulator |
| SP3 | Positive Regulator |
| ZBTB3 | Positive Regulator |

Also provided herein is a method of treating an autoimmune disorder in a subject, the method comprising administering a population of effector T cells having decreased IL2RA expression comprising: (a) a genetic modification or heterologous polynucleotide that inhibits expression of a nuclear factor set forth in Table 5 to a subject that has an autoimmune disorder. In some embodiments, the effector T cell is a CD4+ T cell.

TABLE 5
Nuclear Factors that can be inhibited to decrease
IL2RA expression in effector T cells

| Gene Name | Regulator Type |
|---|---|
| BACH2 | Positive Regulator |
| REST | Positive Regulator |
| ZBTB14 | Positive Regulator |
| GFI1 | Positive Regulator |
| IL2RB | Positive Regulator |

Also provided herein is a method of treating an autoimmune disorder in a subject, the method comprising administering a population of stimulated effector T cells having decreased IL2Ra expression comprising: (a) a genetic modification or heterologous polynucleotide that inhibits expression of a nuclear factor set forth in Table 6 and/or a (b) heterologous polynucleotide that encodes a nuclear factor set forth in Table 7, to a subject that has an autoimmune disorder. In some embodiments, the stimulated effector T cell is a CD4+ T cell.

TABLE 6
Nuclear Factors that can be inhibited to decrease
IL2RA expression in stimulated effector T cells

| Gene Name | Regulator Type |
|---|---|
| REST | Positive Regulator |
| ELP2 | Positive Regulator |
| IL2RB | Positive Regulator |
| NR4A3 | Positive Regulator |
| ZBTB32 | Positive Regulator |

TABLE 7
Nuclear Factors that can be overexpressed to decrease
IL2RA expression in stimulated effector T cells.

| Gene Name | Regulator Type |
|---|---|
| BACH2 | Negative Regulator |
| STAT6 | Negative Regulator |
| ZMYND8 | Negative Regulator |
| IFNGR2 | Negative Regulator |
| IL4R | Negative Regulator |
| LEF1 | Negative Regulator |
| ARIDSA | Negative Regulator |

Also provided herein is a method of treating cancer in a subject, the method comprising administering a population of regulatory T cells having decreased IL2RA expression comprising: (a) a genetic modification or heterologous polynucleotide that inhibits expression of a nuclear factor set forth in Table 4, and/or a (b) heterologous polynucleotide that encodes a nuclear factor set forth in Table 3, to a subject that has cancer.

Also provided herein is a method of treating an autoimmune disorder in a subject, the method comprising administering a population of stimulated effector T cells having increased IL2RA expression comprising: (a) a genetic modification or heterologous polynucleotide that inhibits expression of a nuclear factor set forth in Table 7 and/or a (b) heterologous polynucleotide that encodes a nuclear factor set forth in Table 6, to a subject that has an autoimmune disorder. In some embodiments, the stimulated effector T cell is a CD4+ T cell.

It is understood that in any of the methods of treatment described herein, for example, methods of treating cancer, or methods of treating autoimmune disorders, one or more nuclear factors set forth in Table 1. Table 2, Table 3. Table 4, Table 5, Table 6 or Table 7 can be inhibited in T cells. Similarly, one or more nuclear factors set forth in Table 1, Table 2, Table 3. Table 4, Table 5, Table 6, or Table 7 can be overexpressed in T cells.

In some embodiments, T cells obtained from a cancer subject may be expanded ex vivo. The characteristics of the subject's cancer may determine a set of tailored cellular modifications (i.e., which nuclear factors from Table 1, Table 2, Table 3, Table 4. Table 5, Table 6 or Table 7 to target), and these modifications may be applied to the T cells using any of the methods described herein. Modified T cells may then be reintroduced to the subject. This strategy capitalizes on and enhances the function of the subject's natural repertoire of cancer specific T cells, providing a diverse arsenal to eliminate mutagenic cancer cells quickly. Similar strategies may be applicable for the treatment of autoimmune diseases.

In other cases, T cells in a subject can be targeted for in vivo modification. See, for example. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Cell Isolation

Primary human regulatory T cells (Treg) and conventional CD4+ T cells (Teff) were isolated from full, fresh Human Peripheral Blood Leukopaks (STEMCELL Technologies. #70500) from healthy donors. Leukopaks were washed with 1.5× volume of FACS Buffer (DPBS without Ca⁺⁺Mg⁺⁺, 2% fetal bovine serum, 1 mM pH 8.0 EDTA) and centrifuged at 500 g for 10 minutes. Pellets were combined and resuspended in FACS Buffer, centrifuged at 500 g for 10 minutes, and resuspended at 200E6 cells/mL in FACS Buffer. CD4+ Treg and Teff (also referred to as T responders) were isolated using the EasySep™ Human CD4+CD127lowCD25+ Regulatory T Cell Isolation Kit (STEMCELL Technologies, #18063) according to the manufacturer's protocol. To enhance Treg purity, kit selected cells were then isolated by sorting. Isolated Treg were pelleted by centrifugation for 10 minutes at 300 g and stained with 1:25 Alexa Fluor 647 anti-human CD25 Antibody (Biolegend, #302618), 1:50 BD Pharmingen™ PE Mouse Anti-Human CD127 (Beckon Dickinson, #557938), and 1:50 Pacific Blue™ anti-human CD4 Antibody (Biolegend, #344620) diluted in FACS Buffer for 30 minutes. Treg were washed with FACS Buffer, pelleted by centrifugation at 300 g for 10 minutes, and resuspended in FACS Buffer at 30E⁶ cells/mL for fluorescence activated cell sorting (FACS). CD4+CD25_{high}CD127_{low} Tregs were sorted into 15 mL centrifuge tubes coated with fetal bovine serum and containing 3 mL XVIVO media. Following FACS, Tregs were pelleted by centrifugation at 300 g for 10 minutes and resuspended in cXVIVO supplemented with 200 U/mL IL2 (R&D Systems, #202-GMP-01M). Teff were resuspended in RPMI with 50 U/mL IL2. Cells were seeded at 1E6 cells/mL and stimulated with 6.25 uL/mL ImmunoCult™ Human CD3/CD28/CD2 T Cell Activator (STEMCELL Technologies, #10990) for Teff and 25 ul/ml for Treg. For Teff screens, 3 donors were used. For Treg screens, 2 donors were used.

### Lentiviral Transduction

Twenty-four hours after stimulation, the trans regulator lentiviral library (1) was transduced by pipetting into the flasks containing Treg or Teff. Cells were counted prior to the addition of virus, which was added with an intended multiplicity of infection (MOI) of 0.8. After adding the virus, the flasks were tilted to disperse the viral media. The cells were then incubated at 37 °C for an additional 24 hours, pelleted by centrifugation, and viral media was replaced with fresh media supplemented with IL2.

### Cas9-Ribonucleoprotein (RNP) Preparation for Pooled CRISPR KO

Cas9 RNPs were generated as previously described in Freimer, J. W. et al (Nat Genet 54, 1133-1144 (2022)). On the day of electroporation, lyophylized Dharmacon Edit-R crRNA Non-targeting Control #3 (Dharmacon, #U-007503-01-05) was resuspended in 10 mL Tris-HCL to a concentration of 160 mM. The crRNA was mixed at a 1:1 molar ratio with Dharmacon Edit-R CRISPR-Cas9 Synthetic tracrRNA (Dharmacon, #U-002005-20), which was previously frozen in Nuclease Free Duplex Buffer (IDT, #114)1-03-01) at 160 uM stock concentration and stored at −80C. The mixture was incubated at 37 °C for 30 minutes. Single-stranded donor oligonucleotides (ssODN; sequence: TTAGCTCTGTTTACGTCCCAGCGGGCATGAGAGTAACAAGAGGGTGTGGTAATAT TACGGTACCGAGCACTATCGATACAATATGTGTCATACGGACACG (SEQ ID NO: 1011), 100 uM stock) was added to complexed gRNAs at a 1:1 molar ratio and incubated at 37 °C for 5 minutes. Cas9 protein (MacroLab. Berkeley, 40 μM stock) was added to complexed gRNAs at a 1:2 molar ratio of Cas9 to complexed gRNAs and incubated at 37 °C for 15 minutes.

### Electroporation

24 hours after viral media removal, Treg and Teff were pelleted by centrifugation at 150 g for 10 minutes, resuspended at 1.5E⁶ cells per 17.8 μL supplemented P3 Primary Cell Nucleofector Solution (Lonza, component of #V4SP-3960) and combined with 7.2 uL RNP/1.5E6 cells in a sterile 10 mL reservoir. After mixing, 25 μL of the cell-RNP mixture was distributed to the wells of a 96-well Nucleocuvette Plate (Lonza, component of #V4SP-3960), ensuring no bubbles. Cells were nucleofected using code EO-115 for Treg and EH-115 for Teff on the Lonza 4D-Nucleofector System with the 96-well Shuttle. Immediately after nucleofection, 90 μL pre-warmed cell appropriate media was added to each well, and cells were incubated at 37 °C for 15 minutes. Following incubation, cells were plated at 1E⁶ cells/mL in appropriate media supplemented with IL2.

### Cell Expansion

With the exception of the days noted, cells were split every other day at a 1:2 ratio as needed with their respective IL2 supplemented media to maintain a density of approximately 1e⁶ cells/mL.

### Restimulation of Teff

For the restimulated Teff screen, 9 days after cell isolation. Teff were restimulated with 6.25 L/mL immunoCult™ Human CD3/CD28/CD2 T Cell Activator in RPMI supplemented with IL2.

### Cell Sorting

For resting Treg and Teff screens, cell sorting was performed 10 days following isolation. For the restimulated Teff screen, sorting was performed 3 days after restimulation. Cells were counted, washed once with FACS Buffer, and stained in 1:25 diluted Alexa FluorX 647 anti-human CD25 Antibody at 4C for 20 minutes. Cells were then washed and resuspended in FACS buffer for sorting. During sorting, cells were gated on the GFP+ population (lentiviral guide library marker) and the top and bottom 20% of CD25 expressing cells were sorted into 15 mL conical tubes coated with FCS. Isolated cells were pelleted, counted, and lysed. For each population and cell type, gDNA was extracted and guides were amplified and sequenced.

### Screen Analysis

All screens were analyzed with Mageck. Mageck count was performed on all donors followed by Mageck test to identify genes that resulted in a statistically significant change in CD25 expression.

### F

Arrayed Validation gRNA Selection, Cell Isolation, Culture, Electroporation, and Phenotyping
gRNA Selection

Nineteen genes were selected from the SLICE screen for validation in an arrayed format. For each gene, the top sgRNAs with the largest LFC in the screen were selected and these sgRNAs were ordered as custom crRNAs from Dharmacon. Custom crRNAs were resuspended in Nuclease Free Duplex Buffer (IDT, #11-01-03-01) at 160 uM, frozen, and stored at −80 °C.

### Cas9-Ribonucleoprotein (RNP) Preparation for Arrayed KO

Cas9 RNPs were generated as previously described in Freimer, et al. Briefly, custom follow-up crRNAs (Dharmacon) and Dharmacon Edit-R CRISPR-Cas9 Synthetic tracrRNA (Dharmacon, #U-002005-20) were resuspended in Nuclease Free Duplex Buffer (IDT, #11-01-03-01) at 160 μM stock concentration, frozen, and stored at −80 °C. Prior to nucleofection, each crRNA and tracrRNA were combined at a 1:1 molar ratio and incubated at 37 °C for 30 minutes. Single-stranded donor oligonucleotides (ssODN; sequence: TTAGCTCTGTITACGTCCCAGCGGGCATGAGAGTAACAAGAGGGTGTGGTAATAT TACGGTACCGAGCACTATCGATACAATATGTGTCATACGGACACG (SEQ ID NO: 1011), 100 uM stock) was added to complexed gRNAs at a 1:1 molar ratio and incubated at 37 °C for 5 minutes. Cas9 protein (MacroLab, Berkeley, 40 μM stock) was added to complexed gRNAs at a 1:2 molar ratio of Cas9 to complexed gRNAs and incubated at 37 °C for 15 minutes. Resulting RNPs were frozen and stored at −80 °C.

### Cell Isolation

Cell isolation was performed according to the prior cell isolation section. Teff validation was performed in 3 donors and Treg validation in 2 donors.

### Electroporation

Forty-eight hours post-stimulation, Treg and Teff were pelleted by centrifugation at 150 g for 10 minutes, resuspended at ≤1.5E6 cells per 21.4 uL supplemented P3 Primary Cell Nucleofector Solution (Lonza, component of #V4SP-3960), and distributed to wells of a PCR plate. 3.6 uL of arrayed thawed RNPs were added to each well and mixed by pipetting (50 pmol total RNP per condition). 25 μL of the cell-RNP mixture was transferred to the wells of a 96-well Nucleocuvette Plate (Lonza, component of #V4SP-3960), ensuring no bubbles. Cells were nucleofected using code DS-137 on the Lonza 4D-Nuclcofector System with the 96-well Shuttle. Immediately after nucleofection, 90 uL pre-warmed cell appropriate media was added to each well, and cells were incubated at 37 °C for 15 minutes. Following incubation, cells were plated at 1E⁶ cells/mL in appropriate media supplemented with IL2. Cells were maintained in U-bottom 96 well plates for the duration of the experiment.

### Cell Expansion and Restimulation

With the exception of the days noted, cells were split every other day at a 1:2 ratio as needed with their respective IL2 supplemented media to maintain a density of approximately 1E⁶ cells/mL. After the resting time point was collected on day 9, cells were restimulated with 6.25 uL/mL ImmunoCult™ Human CD3/CD28/CD2 T Cell Activator (STEMCELL Technologies, #10990) for Teff and 25 μl/ml for Treg.

### Phenotyping

On days 9, 11, and 13 after isolation, cells were phenotyped using flow cytometry. The Biolegend FoxP3 Fix/Perm kit (Biolegend, #421403) was used for staining according to the manufacturer's protocol. Cells were washed in FACS buffer prior to extracellular staining. Cells were stained with Alexa FluorK 647 anti-human CD25 Antibody diluted 1:25 (Biolegend, #302618), Ghost Dye™ Red 780 diluted 1:1000 (Tonobo, #13-0865-T500) and BV711 anti-human CD4 diluted 1:50 (Biolegend, #344648) for 20 minutes at 4C and then washed once with FACS buffer. After fixing and permeabilizing according to the kit, intracellular staining was performed with PE anti-mouse/human Helios Antibody (Biolegend #137216), KIRAVIA Blue 520™ anti-human CD152 (CTLA-4) Antibody (Biolegend #349938), Pacific Blue™ anti-human FOXP3 Antibody (Biolegend, #320116), and PE/Dazzle™ 594 anti-human/mouse Granzyme B Recombinant Antibody (Biolegend, #372216) all diluted 1:50 in perm buffer for 30 minutes at room temperature. Cells were subsequently washed and resuspended in FACS buffer before running on the ThermoFisher Attune flow cytometer.

### Data Analysis

FCS files were analyzed in FlowJo and exported for compilation. Statistical analysis was performed in R. Log 2 fold change for each marker as compared to the average across the two AAVS1 control guide wells per condition. Heatmaps from the validation data are the average across all donors.

### Cell Staining Stimulation Timecourse

For stimulation timecourse experiments, cells were restimulated with ImmunoCult™ Human CD3/CD28/CD2 T Cell Activator (STEMCELL Technologies, #10990). The Biolegend FoxP3 Fix/Perm kit (Biolegend, #421403) was used for staining according to the manufacturer protocol. Cells were washed in EasySep buffer prior to staining with Ghost Dye™ Red 780 (Tonobo, #13-0865-T500). After fixing and permeabilizing according to the kit, intracellular staining was performed with PE anti-mouse/human Helios Antibody (Biolegend #137216), KIRAVIA Blue 520™ anti-human CD152 (CTLA-4) Antibody (Biolegend #349938), Pacific Blue™ anti-human FOXP3 Antibody (Biolegend, #320116), and PE/Dazzle™ 594 anti-human/mouse Granzyme B Recombinant Antibody (Biolegend, #372216) for 30 minutes at room temperature. Cells were subsequently washed in permeabilization buffer and resuspended in EasySep buffer before running on the ThermoFisher Attune NxT flow cytometer.

### Results

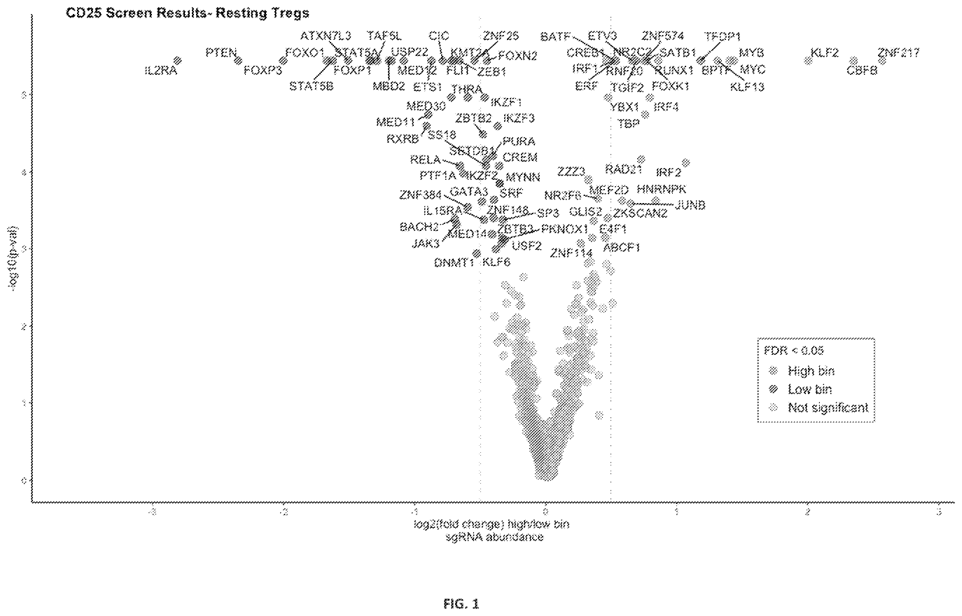

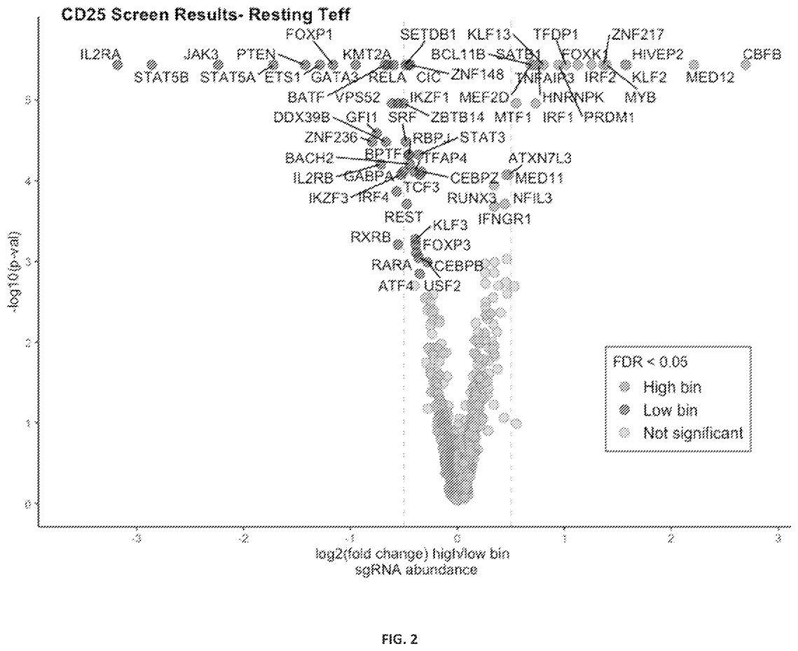

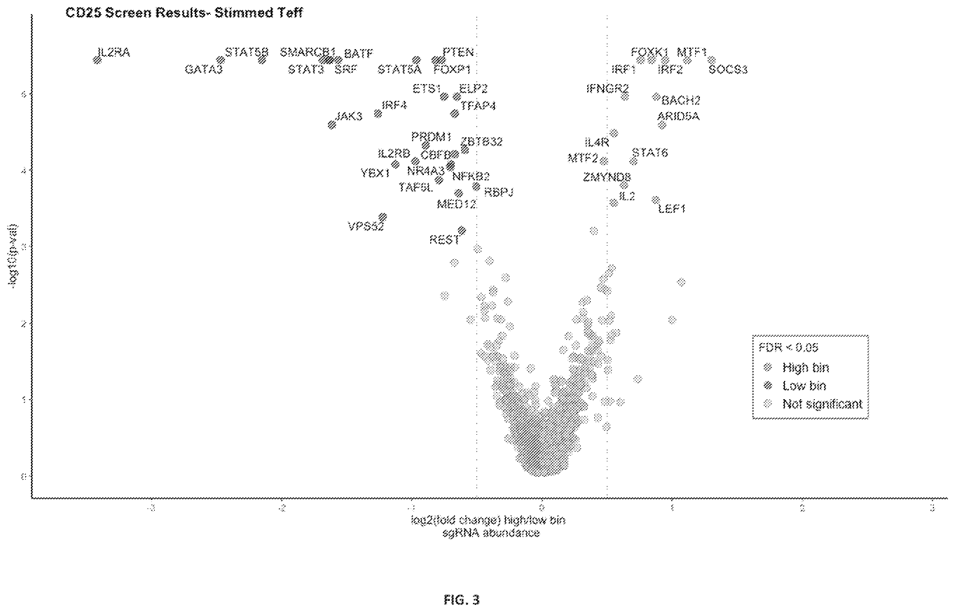

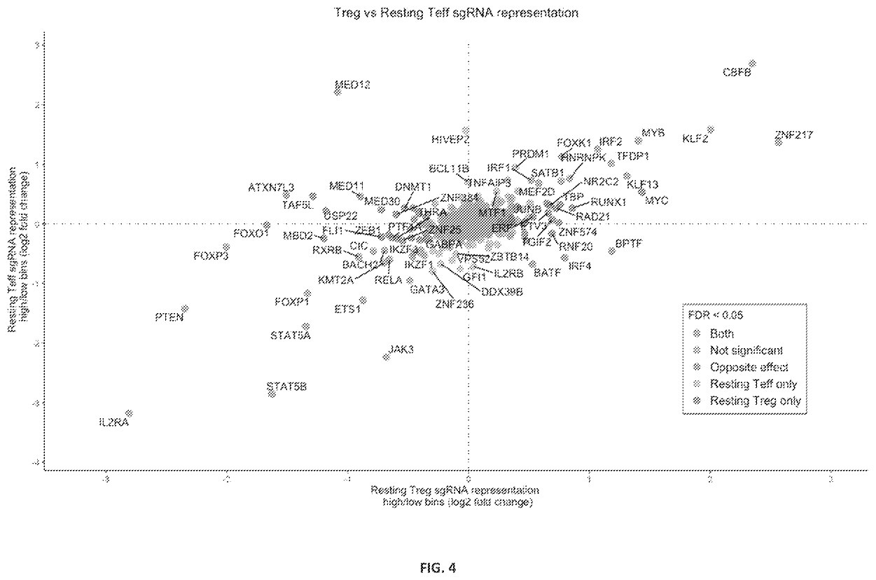

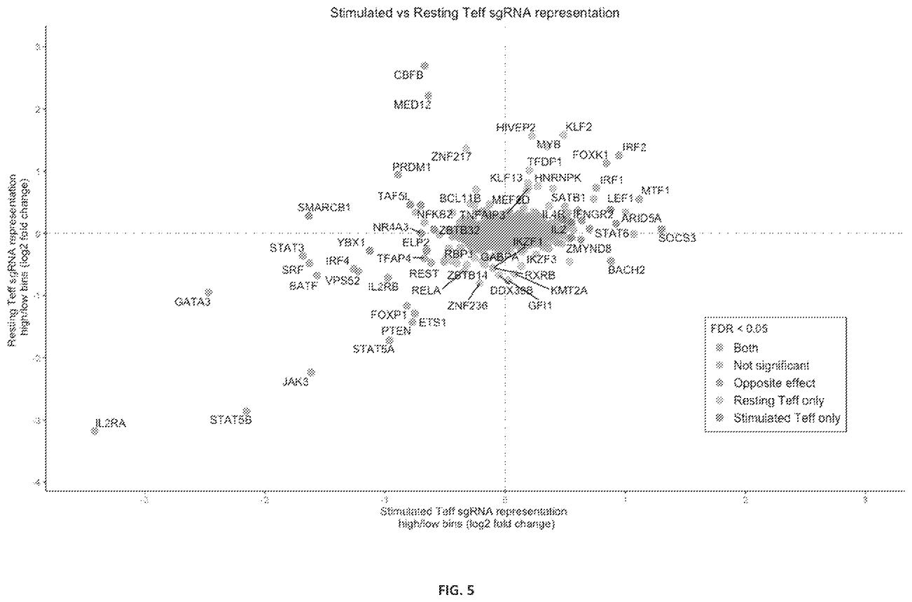

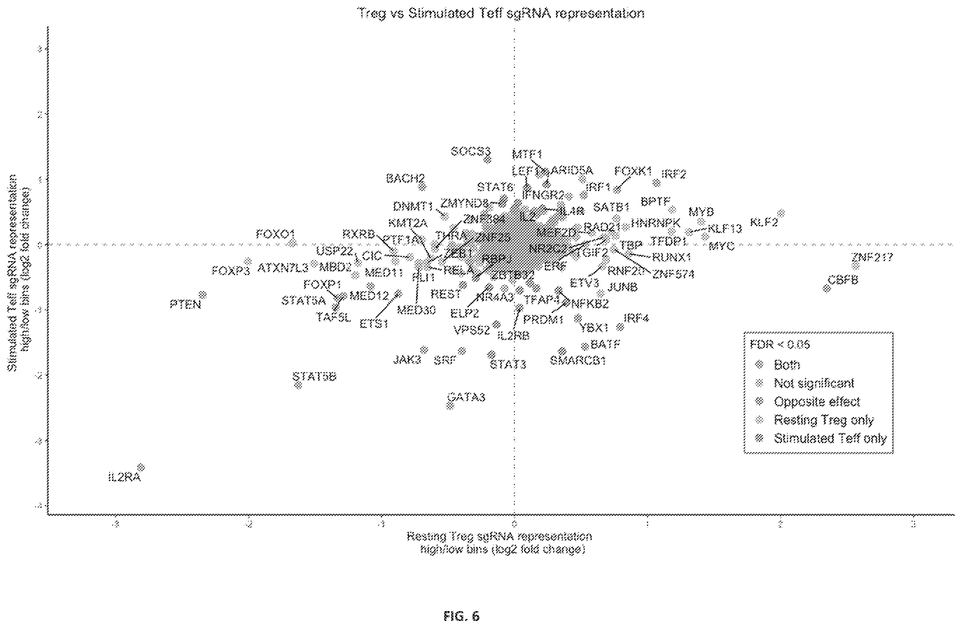

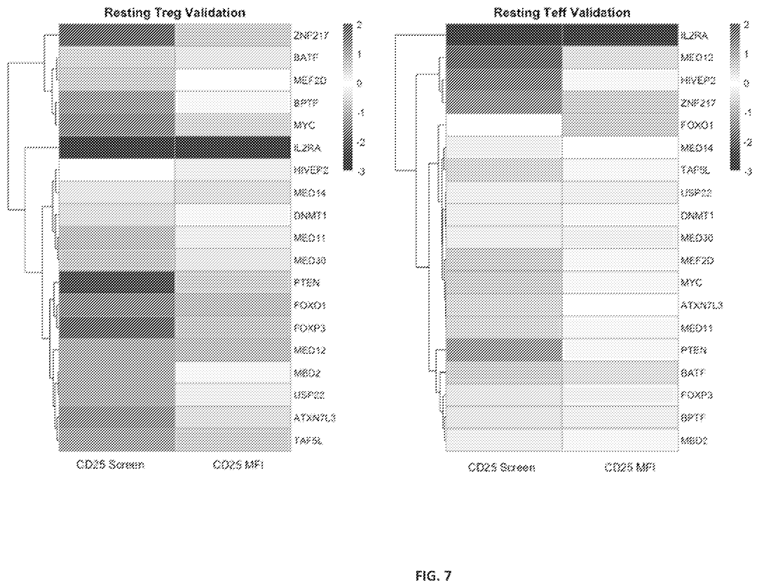

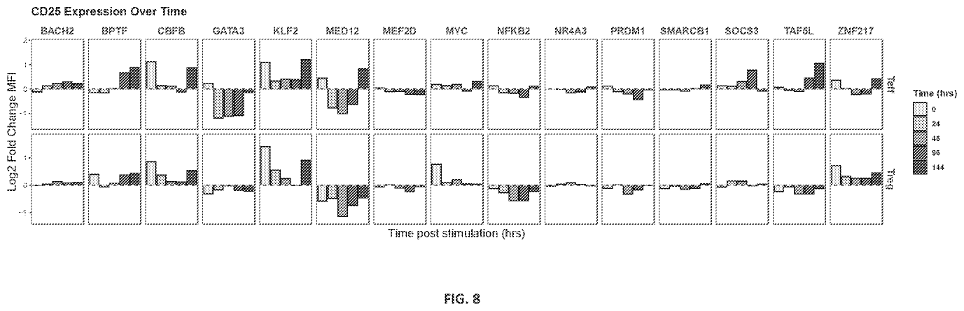

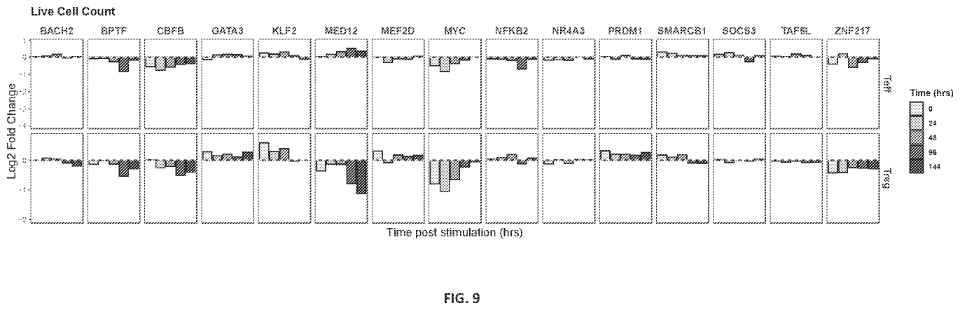

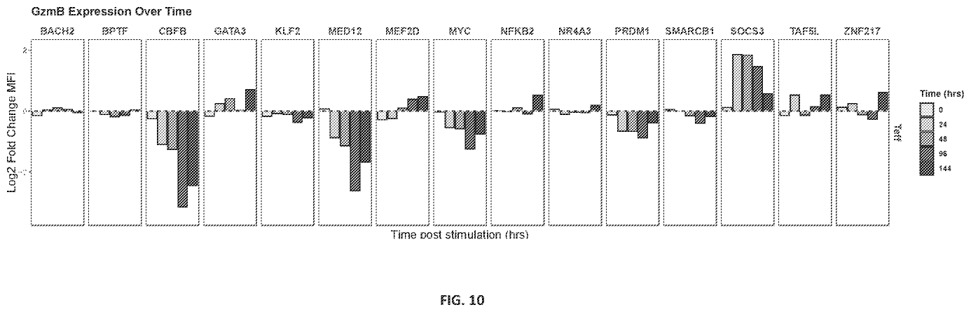

IL2RA is expressed by a wide range of immune cells, including CD4+ T cell subsets to increase affinity for IL2. Although all CD4+ T cells are capable of expressing IL2RA, it is differentially regulated across cell subsets as a means of selectively controlling subset specific proliferation and activity. A series of CRISPR KO screens were performed in primary human CD4+ T cell subsets to identify context dependent upstream regulators of IL2RA. Tens of positive and negative regulators of IL2RA were identified in CD4+ regulatory T cells (FIG. 1) and CD4+ effector T cells (FIGS. 2-3). For effector T cells, which differentially express IL2RA across resting and activated states, a screen was performed in both resting and stimulated cells (FIG. 2, FIG. 3 respectively). Comparative analysis of the screen results revealed regulators of IL2RA in regulatory T cells that do not appear to control IL2RA expression in effector T cells when resting (FIG. 4) or stimulated (FIG. 6). The alternative was also true with several effector T cell specific regulators being identified in comparison to regulatory T cells (FIGS. 4, 6). Additionally, genes that appear to only contribute to the regulation of IL2RA in resting or stimulated effector T cells were also identified by comparison (FIG. 5). Across the conditions there were a number of shared regulators with the same direction of effect between two or all three of the screens (FIG. 4-6), suggesting they are persistent regulators of IL2RA. Surprisingly, a handful of genes that had different directions of effect on the expression of IL2RA depending on the cell subset or stimulation condition (FIG. 4-6), which act as negative regulators of IL2RA in one context and positive regulators in another, were identified. A subset of the genes identified in the screens were also knocked out in an arrayed format which largely validated the regulatory direction of effect for both regulatory T cells and effector T cells (FIG. 7). Further, several regulators affected CD25 expression (FIG. 8), cell count (FIG. 9) and Granzyme B expression (FIG. 10), as compared to adeno-associated virus site 1 (AAVS1) control knockout guides, over the course of stimulation (columns, from left to right representing 0, 24, 48, 96 and 144 hours, for each regulator) in effector T cells and regulatory T cells. These time courses highlight cell type and stimulation specific regulatory roles of these factors. For example, ablation of TAF5L increased CD25 expression in stimulated effector T cells returning to rest at later timepoints but consistently decreased CD25 expression in regulatory T cells. SOCS3 had a similar role in prolonging CD25 expression in an effector T cell specific manner while BPTF prolonged elevated levels of CD25 in both cell subsets. Ablation of GATA3 and NFKB2 prevented cells from reaching peak stimulation levels of CD25 in the middle of the time course with GATA3 having an effect in effectors and NFKB2 having a larger effect in regulatory T cells. KLF2, CBFB, and ZNF217 all had much larger roles in regulation of CD25 in resting T cells compared to stimulated T cells and ablation of these proteins resulted in increased resting levels of CD25 in both effector T cells and regulatory T cells. MED12 had a dynamic effect across timepoints and ablation of the protein increased CD25 levels in resting Teffs, but decreased expression in Tregs at all timepoints and in stimulated Teffs. This also resulted in a decrease of regulatory T cells (FIG. 9) but an increase in effector T cells over time. Granzyme B, a functional marker for effector T cells was affected by many of the perturbations. Ablation of SOCS3, TAF5L, and ZNF217 resulted in increased Granzyme B expression in addition to prolonged CD25 expression, suggesting that perturbation of these regulators may increase effector function and IL-2 sensitivity (FIG. 10). Targeting any of these regulators with cell type or stimulation specific effects in a bulk T cell therapy product may increase purity or functionality of a specific cell subset, increasing the efficacy of the product. Preventing peak stimulation may also prevent activation induced cell death via apoptosis or exhaustion. Comprehensively, these results encompass a network of trans regulators governing expression of IL2RA in a cell subset and stimulation dependent manner within the CD4+ T cell lineage.

### Example 2

### Primary Human T Cell Isolation and Expansion

CD4+ regulatory and effector T cells were isolated from fresh Peripheral Blood Leukopaks (STEMCELL Technologies, #70500) from healthy human donors, after institutional review board-approved informed written consent (STEMCELL Technologies). The contents of the Leukopaks were washed twice with a 1× volume of EasySep buffer (DPBS, 2% fetal Bovine Serum (FBS), 1 mM pH 8.0 EDTA) using centrifugation. The washed cells were resuspended at 200E6 cells/mL in EasySep buffer and isolated with the EasySep™ Human CD4+CD127lowCD25+ Regulatory T Cell Isolation Kit (STEMCELL Technologies, #18063), according to the manufacturer's protocol. Following isolation with the kit, Tregs were stained Alexa Fluor®647 anti-human CD25 Antibody (Biolegend. #302618). PE anti-Human CD127 (Beckon Dickinson, #557938), and Pacific Blue™ anti-human CD4 Antibody (Biolegend, #344620) and isolated using FACS to ensure a pure population without contaminating effector cells. After sorting pure CD4+CD127lowCD25+ Regulatory T Cells, the cells were seeded at 1×10⁶ cells/mL in XVIVO-15 (Lonza, #02-053Q) supplemented with 55 uM 2-mercaptoethanol, 4 mM N-acetyl L-cysteine, and 200 U/mL IL-2 (Amerisource Bergen, #10101641). Teffs were seeded at 1×10⁶ cells/mL in RPMI-1640 supplemented with 10% FCS, 2 mM L-Glutamine (Fisher Scientific #25030081), 10 mM HEPES (Sigma, #H0887-100ML), 1×MEM Non-essential Amino Acids (Fisher, #11140050), 1 mM Sodium Pyruvate (Fisher Scientific #11360070), 100 U/mL Penicillin-Streptomycin (Sigma, #P4333-100ML), and 50 U/mL IL-2 (Amerisource Bergen, #10101641). Both cell subsets were then stimulated with ImmunoCult™ Human CD3/CD28/CD2 T Cell Activator (STEMCELL Technologies, #10990) at 25 uL/mL for Tregs and 6.25 uL/mL for Teff. Following activation and electroporation, cells were split 1:2 every 48 hours to maintain an approximate density of 1×10⁶ cells/mL and supplemented with respective doses of IL-2.

### Pooled CRISPR Knock-Out Screen Trans Regulator Editing

Pooled screens were performed following the protocol described in Freimer et al. (Nat Genet 54, 1133-1144 (2022)). In brief, 24 hours after stimulating and plating the T cells, the trans regulator lentiviral library was added to each culture. The cells were counted prior to transduction, and virus was added at a multiplicity of infection (MOI) of 0.8, using gentle mixing to disperse the viral media without disrupting cell bundling. The cells were then incubated at 37 °C for an additional 24 hours, pelleted by centrifugation, and viral media was replaced with fresh media supplemented with IL-2.

24 hours after washing, the cells were pelleted by centrifugation at 150 g for 10 minutes, resuspended at 1.5E⁶ cells per 17.8 μL supplemented P3 Primary Cell Nucleofector Buffer (Lonza, component of #V4SP-3960) and combined with 7.2 uL RNP/1.5E⁶ cells in a sterile 10 mL reservoir. After mixing the cells and RNPs, 25 μL of the mixture was distributed to the wells of a 96-well Nucleocuvette Plate (Lonza, component of #V4SP-3960). Cells were nucleofected using code EO-115 for Tregs and EH-115 for Teffs on the Lonza 4D-Nucleofector System with the 96-well Shuttle. Immediately after nucleofection, 90 μL pre-warmed cell appropriate media was added to each well, and the cells were incubated at 37 °C for 15 minutes. Following incubation, cells were seeded at 1E6 cells/mL in media supplemented with IL-2.

### IL2RA Screen Sorting and Library Preparation

Transduced and electroporated cells were expanded for a minimum of 6 days following editing prior to sorting. Cell sorting was performed 10 days following isolation for the resting screens. For the stimulated Teff screen, cells were restimulated with ImmunoCult™ Human CD3/CD28/CD2 T Cell Activator (STEMCELL Technologies, #10990) 9 days following initial isolation and sorting was performed 72 hours after restimulation, at the time of peak CD25 expression. Prior to sorting, cells were counted, washed once with EasySep buffer, and stained with Alexa Fluor® 647 anti-human CD25 Antibody (Biolegend, #302618). Cells were then washed and resuspended in EasySep buffer. During sorting, cells were gated on the GFP+ population (lentiviral guide library marker) and the top and bottom 20% of CD25 expressing cells were sorted into 15 mL conical tubes coated with FCS. Isolated cells were pelleted, counted and lysed. gDNA extraction was performed using phenol-chloroform extractions and guide libraries were amplified and prepared for sequencing using custom primers. Libraries were sequenced on an Illumina HiSeq 4000 at the UCSF CAT.

### Screen Analysis

All pooled screens were analyzed with Mageck (v0.5.9.5). Mageck count was performed on all donors using --norm-method none followed by Mageck test --sort-criteria pos to identify genes that resulted in a statistically significant change in CD25 expression. All genes with an FDR-adjusted P<0.05 were considered significant.

### Arrayed CRISPR KO of Select Regulators

Guide loaded Cas9 RNPs were assembled with custom crRNAs (Dharmacon) which were resuspended in IDT duplex buffer (IDT, #11-01-03-01) at 160 μM. Dharmacon Edit-R CRISPR-Cas9 Synthetic tracrRNA (Dharmacon, #U-002005-20) also resuspended in Nuclease Free Duplex Buffer at 160 uM was combined at a 1:1 molar ratio in a 96 well plate and incubated at 37 °C for 30 minutes. Single-stranded donor oligonucleotides (ssODN; sequence: TTAGCTCTGTfTACGTCCCAGCGGGCATGAGAGTAACAAGAGGGTGTGGTAATAT TACGGTACCGAGCACTATCGATACAATATGTGTCATACGGACACG (SEQ ID NO: 1011), 100 uM stock) was added to the complex at a 1:1 molar ratio and incubated at 37 °C for 5 minutes. Finally. Cas9 protein (MacroLab, Berkeley, 40 μM stock) was added at a 1:2 molar ratio and incubated at 37 °C for 15 minutes. The resulting RNPs were frozen at −80 °C until the day of electroporation and were thawed to room temperature prior to use. 48 hours following T cell activation, the cells were pelleted at 100×g for 10 minutes and resuspended in room temperature P3 Primary Cell Nucleofector Buffer (Lonza, catalog no. V4XP-3032) at 1.5E6 cells per 17.8 uL. 1.5E6 cells were transferred to each RNP containing well and mixed gently. 25 ul of the combined RNP cell solution was transferred to a 96-well electroporation cuvette plate (Lonza, #VVPA-1002) and nucleofected with pulse code EH-115. Immediately following electroporation, the cells were gently resuspended in 90 ul warmed media and incubated at 37 °C for 15 minutes. After recovery, the cells were cultured in 96 well round-bottom plates at 1×10⁶ cells/mL for the duration of the experiment. To prevent edge effects, the guides were randomly distributed across each plate and the first and last columns and rows of each plate was filled with PBS to prevent evaporation.

### Flow Cytometry Analysis of Arrayed Knockouts

The Biolegend FoxP3 Fix/Perm kit (Biolegend, #421403) was used for staining according to the manufacturer protocol. Cells were washed in EasySep buffer prior to extracellular staining. Cells were stained with Alexa Fluor® 647 anti-human CD25 Antibody diluted 1:25 (Biolegend, #302618). Ghost Dye™ Red 780 diluted 1:1000 (Tonobo, #13-0865-T500) and BV711 anti-human CD4 diluted 1:50 (Biolegend, #344648) for 20 minutes at 4C and then washed once with EasySep buffer. After fixing and permeabilizing according to the kit, intracellular staining was performed with PE anti-mouse/human Helios Antibody (Biolegend #137216), KIRAVIA Blue 520™ anti-human CD152 (CTLA-4) Antibody (Biolegend #349938). Pacific Blue™ anti-human FOXP3 Antibody (Biolegend, #320116), and PE/Dazzle™ 594 anti-human/mouse Granzyme B Recombinant Antibody (Biolegend, #372216) diluted 1:50 in permeabilization buffer for 30 minutes at room temperature. Cells were subsequently washed in permeabilization buffer and resuspended in EasySep buffer before running on the ThermoFisher Attune NxT flow cytometer. Analysis of flow data was performed in FlowJo (v10.8.1). Gating was performed to select for lymphocytes, singlets, live cells (Ghost Dye negative), and CD4+ cells in the specified order. This population was then used to calculate the median fluorescence intensity (MFI) for CD25, CTLA-4, or Granzyme B. Visualization was performed in R using ggplot2 (v3.4.1).

### Bulk RNAseq

8 days after T cell isolation and activation, the cells were pelleted and resuspended at 1×10⁶ cells per 300 ul of RNA lysis buffer (Zymo, #R1060-1-100). Cells were pipette mixed and vortexed to lyse and frozen at −80 until RNA isolation was performed. RNA was isolated using the Zymo-Quick RNA micro prep kit (#R1051) according to the manufacturer's protocol with the following modifications: After thawing the samples, each sample was vortexed vigorously to ensure total lysis prior to loading into the extraction columns. The optional kit provided DNAse step was skipped, and instead RNA was eluted from the isolation column after the recommended washes and digested with Turbo-DNAse (Fisher Scientific, AM2238) at 37 C for 20 minutes. Following digestion, RNA was purified using the RNA Clean & Concentrator-5 kit (Zymo, #R1016) according to the manufacturer's protocol. The purified RNA was submitted to the UC Davis DNA Technologies and Expression Analysis Core to generate 3′ Tag-seq libraries with unique molecular indices (UMIs). Barcoded sequencing libraries were prepared using the QuantSeq FWD kit (Lexogen) for multiplexed sequencing on an Hiseq 4000 (Illumina).

### Bulk RNAseq Analysis

RNAseq data was processed using the pipeline described in Freimer et al. In brief, fastq adapter trimming was performed with cutadapt (v2.10). Low-quality bases were trimmed with seqtk (v0.5.0). Reads were then aligned with STAR (v 2.7.10a) (Bioinformatics 29, 15-21 (2013)) and mapped to GRCh38. UMI counting and deduplication was performed with umi_tools (v1.0.1) and gene counts were generated from the deduplicated reads using featureCounts (subread v2.0.1) using Gencode v41 basic transcriptome annotation. Quality control metrics were generated for each sample with Fastqc (v0.11.9), rseqc (v3.0.1), and Multiqc (v1.9). Differentially expressed genes between Mediator KOs and AAVS1 KO samples as well as stimulated and resting AAVS1 KO samples were identified from the deduplicated count matrix using Deseq (v1.32.0) in R (v4.1.0) (Genome Biol 15, 1-21 (2014)). Comparisons were made within each cell type and stimulation condition across 3 donors, using donor ID as a covariate in the model. Differentially expressed genes were defined by a cut off of padj<0.05. Pathway analysis was performed using PathfindR (v1.6.4). Pathway visualization was performed using Cytoscape (v3.8.2).

### CUT&RUN

CUT&RUN from isolated nuclei was performed according to the manufacturer's protocol with the EpiCypher CUTANA™ ChIC/CUT&RUN Kit and provided reagents. In brief, 5E′ T cells per reaction were washed with PBS before nuclear isolation using the EpiCypher recommended lysis buffer consisting of 20 mM HEPES pH 7.9 (Sigma-Aldrich), 10 mM KCl (Sigma-Aldrich), 0.1% Triton X-100 (Sigma-Aldrich), 20% Glycerol (Sigma-Aldrich), 1 mM MnCl2 (Sigma-Aldrich), 1× cOmplete Mini-Tablet (Roche), and 0.5 mM Spermidine (Sigma-Aldrich). The cells were resuspended in 100 ul per reaction cold nuclear extraction buffer and incubated on ice for 10 minutes. Following lysis, nuclei were pelleted and resuspended in 100 ul per reaction of nuclear extraction buffer. The isolated nuclei were then frozen at −80 in extraction buffer until DNA isolation. After thawing the samples at 37C, the nuclei were bound to activated conA beads. After adsorption of nuclei to beads, permeabilization was performed with 0.01% digitonin containing buffer. Antibodies for H3K27ac (EpiCypher) and IgG (EpiCypher) were added at 500 ng per reaction. Following overnight antibody binding, pAG-MNase addition, and chromatin cleavage, 0.5 ng of the provided E. coli DNA was added to each sample following chromatin cleavage by MNase. The provided spin columns and buffers were used for DNA isolation and purification. The resulting DNA was prepared for sequencing using the CUTANA™ CUT&RUN Library Prep Kit (Cat 14-1002) according to the manufacturer's protocol.

### CUT&RUN Analysis

Pooled libraries were sequenced on a NextSeq 500/550 with Mid and High Output v2.5 150 cycle kits (Illumina) and paired end sequencing. Bcl2fastq (v2.19) with the settings --minimum-trimmed-read-length 8 was used to generate fastqs. CUT&RUN data analysis was performed according Zheng et al, with the recommended settings unless otherwise specified below (https://yezhengstat.github.io/CUTTag_tutorial/). In brief, the fastqs were trimmed with cutadapt (v1.18) prior to merging of technical replicates. Bowtie2 (v2.2.5) was used to align the trimmed fastqs to GRCh38 using settings --local --very-sensitive --no-mixed --no-discordant --phred33 --dovetail -I 10 -X 700 -p 8 -q and E. coli (EMBL accession U00096.2) with settings --local --very-sensitive --no-overlap --no-dovetail --no-mixed --no-discordant --phred33 -I 10 -X 700 -p 8 -q. Barn files were generated with samtools (version 1.9) view -bS -F 0x04 and bam to bed conversion performed with bedtools (v2.30.0) bamtobed -bedpe. Bedfiles were filtered to include only paired reads of less than 1000 bp with the command awk ‘$1=$4 && $6-$2<1000 {print $0}’ samplename.bed before generating bedgraph files using bedtools (version 2.30.0) genomecov -bg. Peak calling was performed using the bedgraph files as input with SEACRClick or tap here to enter text (v1.3). Each H3K27ac bed file was compared to the respective donor and KO condition IgG file to identify peaks about the background using the norm and stringent options.

Prior to generating a peak by sample matrix, ChIP-seq blacklist regions were removed from the ban files. The sample matrix was reduced across all peaks within the dataset and segmented into regions of 5000 bps. Regions of differential acetylation between the regulator KOs and AAVS1 KO samples were identified for the 5000 bp peaks called across any of the samples from bam files using Deseq (v1.32.0) in R (v4.1.0) (Genome Biol 15, 1-21 (2014)). Comparisons were made within each cell type and stimulation condition using AAVS1s prepared in the same batch of samples. Super enhancer calling was performed using ROSE³² with blacklist filtered bam files for each KO and the respective IgG sample as input. Super enhancer genes were annotated using the gene with the nearest transcription start site to each super enhancer region with the GenomicRanges (v1.44.0) nearest function.

### CUT&RUN Visualization

Bedgraph scaling was performed based on peak coverage across all samples and conditions using Deseq2 (v1.32.0). Visualization of the scaled peaks was performed with ggplot2 (v3.4.1) and gggenes (v0.5.0) in R (v4.1.0). CD4+ CBP ChIPseq data was accessed from ChIP Atlas-SRX017698, GSM393945 and generated by Wang et al. (Cell 138, 1019-1031 (2009)). CD4+ Treg STAT5 ChIPseq data was accessed from ChIP Atlas-SRX212432, GSM1056923 and generated by Hoffmann et al. (Eur J Immunol 39, 1088-1097 (2009)).

### Cloning and Lentivirus Preparation

CRISPRi guides for perturb-seq were selected from the Dolcetto library (Nat Commun 9, 1-15 (2018)) and cloned into the LGR2.1 plasmid backbone (Addgene #108098). A lenti EF1a-Zim-3-dCas9-P2A-BSD with Blasticidin resistance plasmid was generated using Gibson assembly. Lentivirus was prepared according to the protocol in Schmidt et al. Science (1979) 375, (2022)).

### Perturb-Seq

24 hours after stimulation of isolated human Tregs and Teffs from 2 donors, the cells were transduced with Zim3-dCas9 lentivirus at 3% v/v. The following day, perturb-seq guide library lentivirus was added at 0.75% v/v. 48 hours after transduction with Zim3-dCas9, 10 mg/ml blasticidin (Gibco, #A1113903) was added to each sample to select for dCas9+ cells. Blasticidin n was replenished every 48 hours until the cells were processed for sequencing. 8 days after initial isolation and stimulation of cells, half of the Treg and Teff culture was restimulated with ImmunoCult™ Human CD3/CD28/CD2 T Cell Activator (STEMCELL Technologies, #10990). On the 10^{th} day after initial isolation, the samples were collected for 10× single cell sequencing. First, cells from each donor within the same stimulation and cell type condition were pooled at equal concentrations. Sorting was performed to isolate live GFP+ cells from each condition. Sorted cells were processed according to the Chromium Next GEM Single Cell 5′ HT Reagent Kits v2 (Dual Index) with Feature Barcode technology for CRISPR Screening and Cell Surface Protein guide User Guide, CG000513. In brief, sorted cells were pelleted and washed once with Cell Staining Buffer (Biolegend, #420201). Next, the samples were blocked with Human TruStain FcX™ Fc Blocking reagent (Biolegend, #422302). Meanwhile, TotalSeq™-C Human Universal Cocktail V1.0 (Biolegend, #399905) was prepared using Cell Staining Buffer (Biolegend, #420201) and TotalSeq™-C0251 anti-human Hashtag Antibodies 1-4 (Biolegend, #394661) were added to aliquots of the cocktail. After blocking, cells were stained with TotalSeq-C cocktail including one Hashtag per cell and stimulation condition. After staining, the cells were washed three times in Cell Staining Buffer. The samples were then resuspended in PBS with 1% BSA (Gibco) for final counting. The resulting samples were pooled across conditions and approximately 65,000 cells per well were loaded into 8 wells of a Chromium Next GEM Chip N Single Cell Kit (10× Genomics, #1000375) for GEM generation. The samples were prepared for sequencing using the Chromium Next GEM Single Cell 5′ HT Kit v2 (#1000374), 5′ Feature Barcode Kit, (#1000256), and 5′ CRISPR Kit (#1000451) according to the manufacturer's protocol. GEM generation and library preparation was performed by the Gladstone Genomics Core. The resulting libraries were sequenced using a NovaSeqX Series 10B flowcell (Illumina, #20085595) at the UCSF CAT.

### Perturb-Seq Analysis

Fastqs for each 10× well were concatenated across lanes and flow cells. Alignment of perturb-seq data and count aggregation for the gene expression, CRISPR guide, and Antibody Derived Tag (ADT) libraries was performed with cellranger (Nat Commun 8, (2017)) count (v7.1.0) using the default settings and -expect-cells=45000 -chemistry=SC5P-R2. Gene expression fastqs were aligned to “refdata-gex-GRCh38- 2020-A” human transcriptome reference acquired from 10× Genomics. Guide sequences were aligned to a custom reference file using the pattern TAGCTCTTAAAC(BC) while ADTs were aligned to the TotalSeq-C-Human-Universal-Cocktail-399905-Antibody-reference-UMI-counting.csv provided by Biolegend, also including the hashtag oligo (HTO) sequences which were used to distinguish each cell type and stimulation condition. Counts for each respective library were aggregated across wells with cellranger aggr using the default settings. Cells were assigned to a donor using genetic demultiplexing with Souporcell (Nat Methods 17, 615-620 (2020) (https://github.com/wheaton5/souporcell). For each well, souporcell_pipeline.py was run using the ban file and cellranger count output barcodes.tsv as input in addition to the reference fasta. Donor calls shared across wells were identified using shared_samples.py using the vcf file outputs from Souporcell.

Perturb-seq analysis was performed in R (v4.3.1) using Seurat (Nat Biotechnol 36, 411-420 (2018)) (v4.3.0.1). Count matrices were imported into R using the Seurat Read10× function. After creating a Seurat object with CreateSeuratObject, quality filtering was performed to retain cells with more than 1000 RNA features identified and less than 7.5% mitochondrial RNA. Cells without a singular donor assignment were also excluded from the object as well as cells with more than one HTO assignment as determined after running HTODemux. Low abundance transcripts were filtered using the threshold of 10 cells per feature and TCR genes were removed from the primary RNA assay as they were found to be a major source of variance in the dataset. No sgRNA targets were removed as the number of cells in each condition exceeded the threshold set of 150 cells. After filtering, gene-expression counts were normalized and transformed using the Seurat SCTransform function with regression of both S-phase score and G2/M-phase score, as described on the Satija website (https://satijalab.org/scurat/articles/cell cycle_vignette.html). ADT counts were normalized using the CLR normalization method of NormalizeData. After generating PCAs of both normalized and transformed RNA and ADT data. Harmony (v0.1.1) was used to correct for donor associated variability in the dataset (Nat Methods 16, 1289-1296 (2019). The resulting normalized and transformed counts were used for downstream analysis unless otherwise specified.

Activation scoring was performed according to Schmidt et al. (Science (1979) 375, (2022)). In brief, Seurat FindMarkers was used to identify differentially expressed genes between stimulated and resting non-targeting control cells within the Teffs and Tregs individually. Genes that had a log 2-fold change >0.25 and were detected in 10% of restimulated or resting cells were used to generate gene weights for the score calculated as sum (GE×GW/GM), where GE is a gene's normalized/transformed expression count, GW is the genes weight, and GM is the gene's mean expression in non-target control cells of the respective cell type. Wilcox tests were performed to determine significance compared to non-targeting control cells with Bonferroni correction for multiple hypothesis testing. To observe the effect of each guide within independent cell and stimulation conditions, the cells were subset by HTO. RNA and ADT normalization, transformation, and donor variability correction was repeated for each subset as described above for the combined dataset. UMAPs were generated using the transformed and corrected RNA and ADT counts with Seurat function FindMultiModalNeighbors followed by RunUMAP using weighted.nn. Cell cycle quantification for each subset was performed using cycle assignments generated using the Satija cell cycle vignette referenced above.

Pseudobulking of resting and stimulated Treg and Teff samples was performed using Seurat AggregateExpression grouped by HTO, target Gene, and donor pulling from the counts slot (sgRNAs targeting the same gene were collapsed within the same donor). Differential expression analysis was performed with the resulting pseudobulked raw counts for both RNA and ADTs. DeSeq2 (v1.32.0) was used to identify differentially expressed genes and proteins between each sgrna and non-targeting control sample within each cell type and stimulation condition, using donor information as a covariate. Network plots of differentially expressed gene connections were visualized in R using influential (v2.2.7) and ggraph (v2.1.0), including only genes with an adjusted p-value <0.05. Other visualization of differentially expressed genes and surface proteins was performed using ggplot2 (v3.4.1).

### MEL12 CAR Activation Scoring

MED12 CAR RNAseq data from Freitas et al. (Science (1979) 378, (2022)) was accessed from GEO, using the downloader to retrieve raw counts file GSE174279_raw_counts_GRCh38.p13_NCBI.tsv.gz. First, DeSeq2 (v1.32.0) was used to identify differentially expressed genes between AAVS1 KO stimulated and resting samples. The top upregulated genes were defined using the following criteria: padj<0.01, Log 2FoldChange >2, baseMean >10. The resulting 797 genes were used to generate a gene signature of activation. Normalized counts for the MED12 KO and AAVS1 KO resting and stimulated samples were generated with DeSeq2 vst and converted to a summarized experiment with SummarizedExperiment (v1.22.0). The normalized count matrix and activation score were used as input for GSVA⁶¹ (v1.40.1) using the gsva function with min.sz=10, max.sz=6000, kcdf=“Poisson”. Visualization of the resulting gene scores was performed with ggplot2(v3.4.1) and adjusted P values generated using rstatix (v0.7.2).

### Activation Induced Cell Death Assays

Activation induced cell death assays were performed using titrated amounts of ImmunoCult™ Human CD3/CD28/CD2 T Cell Activator (STEMCELL Technologies, #10990) in addition to 50 U/mL of IL-2. Active caspase-3/7 staining was performed 72 hours following addition of stimulus using the CellEvent™ Caspase-3/7 Green Flow Cytometry Assay Kit (Invitrogen, #C10427) according to manufacturer's protocol. FAS staining was performed using PE anti-human CD95 (Fas) Antibody (Biolegend, #305608).

### Results

### Identification of Context Dependent Trans-Regulators of IL2RA in CD4+ T Cell Subsets

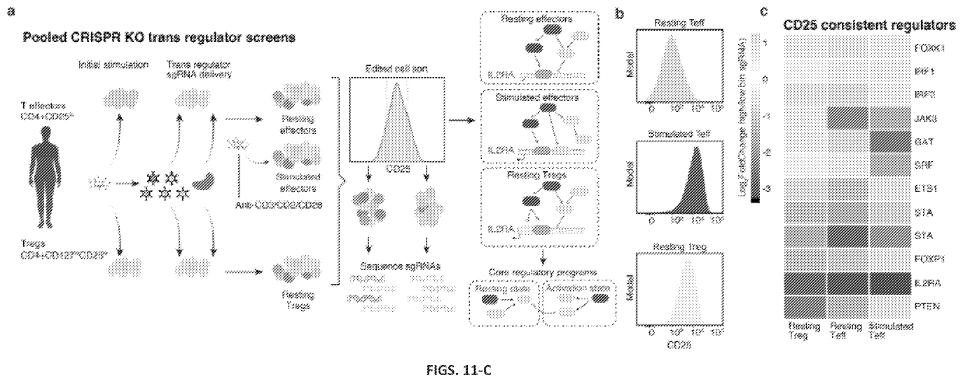

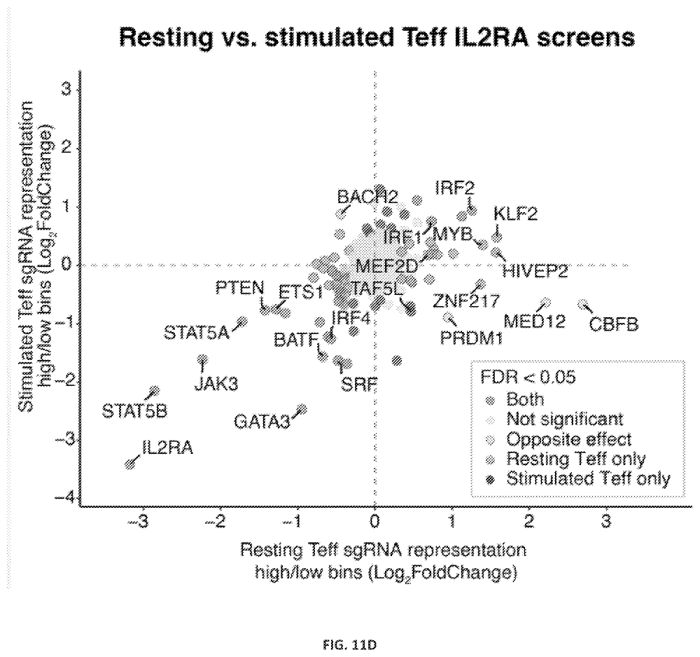

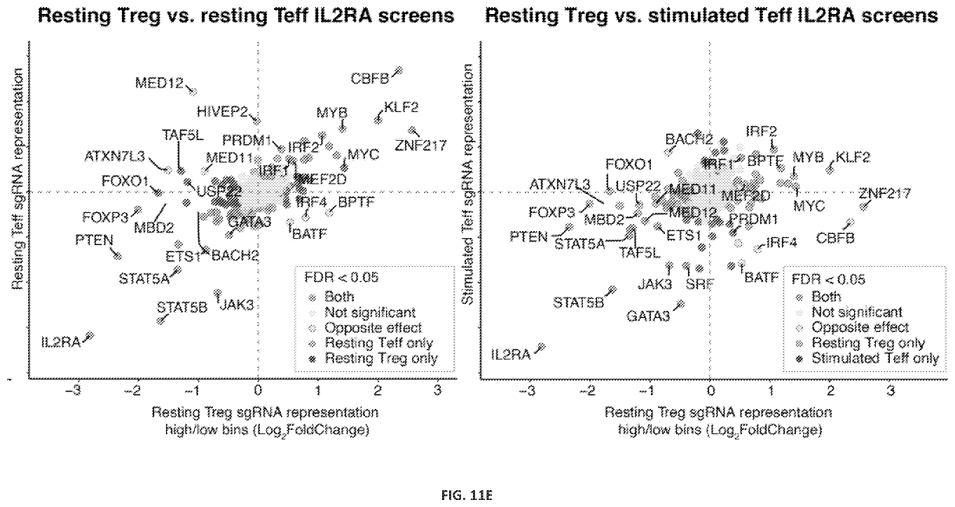

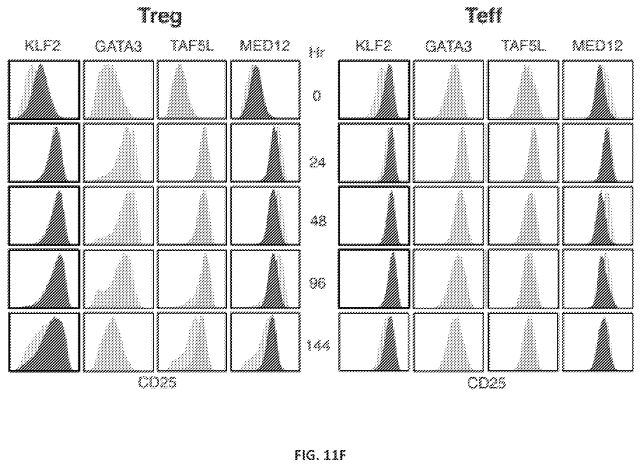

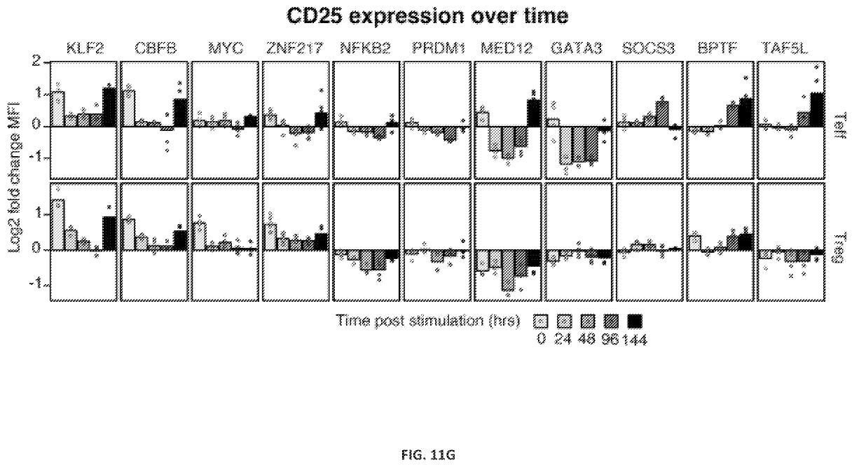

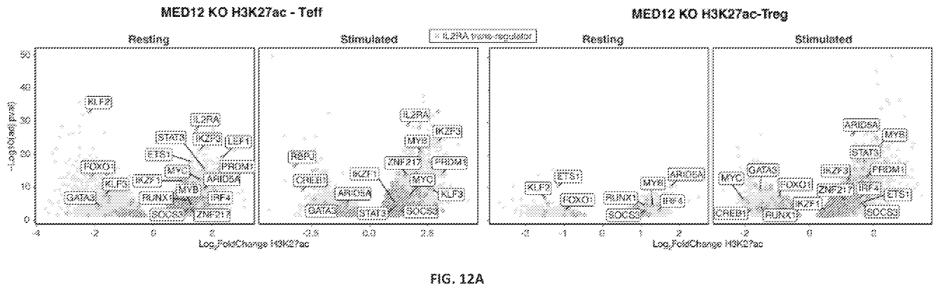

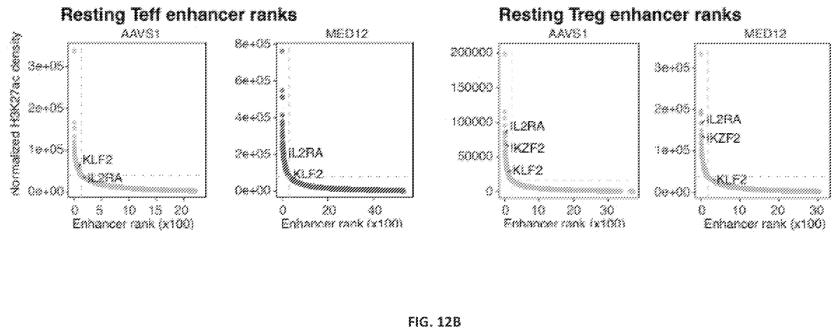

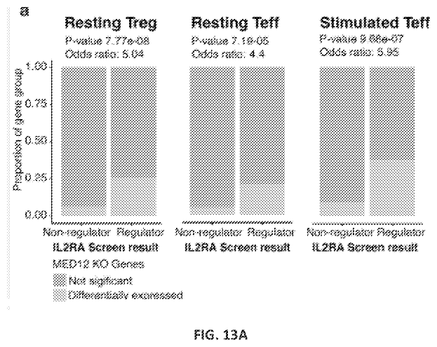

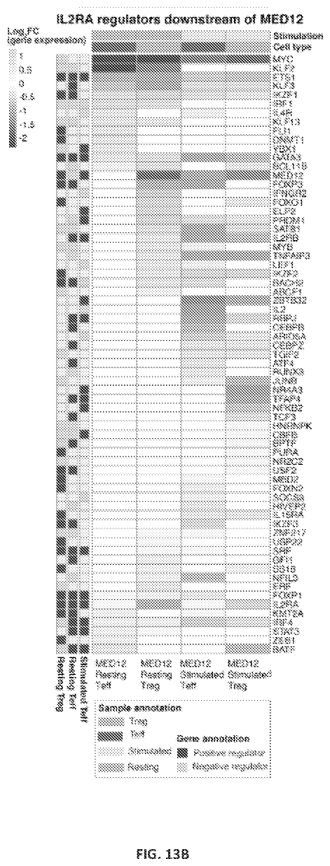

To probe systems of context-dependent gene expression, pooled CRISPR KO screens were used to identify upstream trans-regulators of IL2RA across cell type and stimulation conditions. A 6000 sgRNA library was used to target 1350 nuclear factors (TFs and chromatin regulators) in CD4+ T cell subsets (FIG. 11A; see also, Freimer et al., and Schumann et al. (Nat Immunol 21, 1456-1466 (2020)). We isolated, edited and expanded primary human Tregs (CD4+CD25hiCD127low) and Teffs (CD4+CD25low) to screen for regulators of IL2RA in resting Teffs (IL2RA low) and restimulated Teffs near their highest expression levels, 72 hours following stimulation (IL2RA high) (FIG. 11B), as well as in resting Tregs (IL2RA high). This screening approach was previously utilized to identify upstream trans regulators of IL2RA in resting Teffs (Freimer et al.). The published results were replicated with increased coverage and expanded to the additional cellular contexts to define regulators with context-dependent functions.

Collectively, the screens resulted in the detection of over 100 trans regulators (FDR<0.05) required for proper IL2RA expression in at least one context (see, for example, Table 1). Interestingly, only 16 regulators were hits in all three screens and, of these, only 12 shared the same direction of effect across conditions (FIG. 11C). These 12 regulators (FOXK1, IRF1, IRF2, JAK3, GATA3, SRF, ETS1, STA, FOXP1, IL2RA, and PTEN) ¬—deemed “consistent regulators” of IL2RA—included members of the JAK-STAT pathway and interferon regulatory family (IRF) members. Even among the consistent regulators, the effect sizes of several trans factors were often much larger in a specific condition compared to other screens. GATA3, for example, appeared as a particularly potent positive regulator of IL2RA in stimulated Teffs, with a decrease in guide abundance in the IL2RA high bin/IL2RA low bin of 5.5-fold compared to 1.96-fold and 1.39-fold in resting Teffs and resting Tregs, respectively.

The majority of identified IL2RA regulators appeared as a significant hit in only one or two screens, demonstrating cell type or stimulation specific effects. The direction and magnitude of effect of the perturbations was compared across the three screens to categorize context-dependent regulators of IL2RA. The overall distribution of positive and negative regulators across screens distinguished the resting and activated Teff screen results, with few negative regulators identified in the stimulated state compared to rest (FIG. 11D, Table 1). These results suggest broad structural differences in the upstream regulatory network across conditions, where stimulated Teffs relieve negative regulatory forces to increase levels of IL2RA. KLF2 was one of a few negative regulators in resting Teffs without an effect in stimulated Teffs that was also expressed at much higher levels in resting cells. Conversely, many of the other conditional regulators had relatively similar expression levels across states. It was also observed that, although Tregs also express high levels of IL2RA, a number of negative regulators were shared across the resting Treg and resting Teff screens while there was greater discordance between the resting Treg and stimulated Teff screens (FIG. 11E). Overall, the screen performed in Tregs yielded a particularly large number of significant hits, including both positive and negative regulators, despite IL2RA being constitutively expressed. Because IL2RA is required for the fitness of Tregs, this large network of regulators could act as a buffering system to prevent large fluctuations in expression, unlike Teffs, which rapidly upregulate expression after stimulation.

Not only was the set of regulators condition-specific, but a few factors even exerted effects in different directions across conditions. Notably, MED12, CBFB, and PRDM1 were identified as strong positive regulators of IL2RA in stimulated Teffs but negative regulators in resting Teffs (FIG. 11D). MED12 and to a lesser extent MED11, components of the Mediator of RNA Pol II Complex (Mediator), were both identified as positive regulators of IL2RA in resting Tregs but negative regulators of IL2RA in resting Teff (FIG. 11E). Additionally, BATF and IRF4, which co-bind genomic sites in T cells, were identified as differential regulators with a negative effect in resting Tregs and a positive effect on IL2RA expression in both resting and stimulated Teffs. Comprehensively, this screening approach led to the identification of cell type- and stimulation-specific regulators upstream of IL2RA, as well as the unexpected class of differential regulators with the ability to up and downregulate expression of IL2RA across contexts.

### Distinct Regulators Shape Dynamic Expression of IL2RA Following Stimulation

In order to validate the cell type-specific regulators and hits from our Treg screen, we ablated select regulators and examined the effect on IL2RA protein expression in both Tregs and Teffs. We found a strong correlation between the arrayed KO effect and screen effect of each perturbation, which affirmed the conditional regulator role of many factors. To more precisely dissect the stimulation-responsive regulatory mechanisms of each cell subset, we performed an additional arrayed KO with an extended series of collection time points following restimulation. Much like the screen, we found that during peak expression of IL2RA, approximately 48-72 hours after stimulation, Teffs specifically lacked many negative regulators, with some even temporarily functioning as positive regulators (ZNF217, MED12. PRDM1) (FIG. 11G). Interestingly, the direction of effect was generally more consistent in Tregs, although the effect size of particular regulators was modulated by activation. We also identified differences in the prominent drivers of IL2RA expression after stimulation across subsets where GATA3 was most impactful in Teffs, but NFKB2 had a larger effect in Tregs. Surprisingly, MYC also had negative regulatory effects in the resting state, with particularly large effects in Tregs, despite its characterized role in T cell activation. Despite differences in stimulation response, both Tregs and Teffs both appeared reliant on KLF2 and CBFB to actively suppress IL2RA expression at resting timepoints (FIGS. 11F-G).

Notably, both the cell type and stimulation specific effects of MED12 observed in the screen replicated in an arrayed format. Additional subunits of Mediator (MED14, MED11, MED30) also had directionally similar, albeit more modest, effects on IL2RA expression. Because Mediator has 30 subunits but only four were targeted in the screen, we CRISPR ablated each Mediator subunit in an arrayed format. Many subunit KOs drove expression of IL2RA in the same direction as the MED12 KO, with select subunits demonstrating distinct patterns of conditional regulation and MED12 generally having the largest effect.

While these screens were particularly useful for capturing regulators that enable maintenance of maximum and minimum levels of IL2RA expression, the arrayed time course also revealed regulators that promote transition between states. We identified several factors that enable the transition from activation to rest (~96-144 hrs), with particularly large effects in Teffs, which undergo the greatest fluctuations in IL2RA expression (FIG. 11G). TAF5L, BPTF, and SOCS3 appear to contribute to this “breaking mechanism” required for appropriate loss of cell surface IL2RA as cells return to rest. To investigate whether stimulation responsive regulators participated in similar processes for other receptors, we looked at expression of CTLA-4, which is also upregulated in response to stimulation in Teffs and expressed constitutively on the surface of Tregs. We identified similar patterns of temporal regulation by the perturbed genes across the stimulation time course, especially in Teffs, suggesting that these regulatory mechanisms control a broader network of key genes in CD4+ T cell subsets. Comprehensively, it was a found that many regulators contribute to activation and rest associated gene regulation in temporally defined stages, with some regulatory systems specific to T cell subset.

### Core Conditional Regulators Shape the Cell Type and Stimulation Specific Enhancer Landscape

We speculated that some of the identified context-dependent regulators are required to establish and maintain chromatin features that distinguish each condition. H3K27ac distribution varies considerably between Tregs and Teffs and across activation states at associated enhancers and has been established as a prerequisite for gene expression. IL2RA in particular has been prominently categorized as a super enhancer gene in Tregs with a large stretch of H3K27ac surrounding the transcription start site. However, in resting Teffs, there is significantly less acetylation of the region. Using H3K27ac CUT&RUN, we assessed alterations to the enhancer landscape of resting and stimulated Tregs and Teffs following KO of select regulators. We prioritized subunits of Mediator (MED12, MED11, MED24) and SAGA (TAF5L, ATXN7L3, USP22), as well as BATF and ZNF217 due to their cell type and stimulation differential phenotypes.

We discovered that perturbation of several regulators, but predominantly MED12, resulted in significant changes to the chromatin landscape when compared to control samples. Thousands of differentially acetylated regions were identified across each condition in the MED12 KO samples, including at the loci of numerous IL2RA regulators (FIG. 12A). In resting T cells, an extended region of chromatin at the KLF2 locus was marked with H3K27ac, consistent with KLF2 playing a core role in regulation of the resting state. MED12 KO caused significantly deacetylation within the gene body and at a distal enhancer (~16,370 Kb) in resting Teffs and Tregs. These KLF2 regions of decreased acetylation were occupied by CBP, an H3K27ac acetyltransferase (FIG. 12B). MED12 is a known binding partner of CBP and may recruit the enzyme in a state specific manner. Interestingly, MED12 KO samples also resulted in a number of regions of increased acetylation. At the SOCS3 locus, a negative regulator of IL2RA required to suppress expression after stimulation, both the gene body as well as two distal enhancers had significantly higher levels of H3K27ac, with all three sites being affected in the stimulated cells. Taken together, MED12 is required for normal chromatin landscape transitions between the resting and active state, especially at genomic sites encoding key regulators of IL2RA expression.

MED12 ablation also altered chromatin at the IL2RA locus. Most prominently, in MED12 KO Teffs, the region upstream of the transcription start site (TSS) that is generally more acetylated in Tregs had significantly higher levels of acetylation. The most striking increase was localized within the CaRE3 enhancer, which were previously characterized as a Treg specific element using tiled CRISPR activation screens and in vivo murine models. An additional region located directly downstream of the IL2RA TSS, showed significantly less acetylation specifically in Tregs as the result of MED12 KO. To investigate what signaling changes might result from increased acetylation of CaRE3, we looked at Treg ChIP-seq data for regulators that bind this region. STAT5A binds sites within the region, suggesting that increased acetylation in Teffs may predispose the cells to increased STAT5 sensitivity and more Treg-like gene expression. While it is difficult to disentangle IL2RA and STAT5 signaling as the two components participate in a feed-forward loop, there is apparent dysregulation of the signaling pathway that prevents cell type characteristic regulation. Collectively, these changes at the locus of IL2RA and state specific regulators such as KLF2 and SOCS3 demonstrate a loss of context specific chromatin features required for activation state transitions and maintenance.

While the other perturbations' effects on H3K27ac were less extensive than MED12, we observed that regions of differential acetylation in the TAF5L KO were highly correlated with MED12, particularly in stimulated Teffs. Interestingly, loci affected by both MED12 and TAF5L KOs were generally at distal enhancers. While the respective complexes of TAF5L and MED12, SAGA and Mediator, are capable of interacting within the pre-initiation complex, these results suggest a potential interaction or shared downstream regulator that modulates chromatin farther from the transcription start site.

Having defined MED12 as an orchestrator of T cell state and identity features, we asked how its ablation affects maintenance of “super enhancers,” extended active enhancer regions at genes associated with cell-specific gene expression. Using the ROSE algorithm (Cell 153, 307-319 (2013)), we ranked enhancers from each sample based on H3K27ac and identified contiguous stretches of high acetylation considered to be super enhancers. Consistent with previous reports, IL2RA was annotated as a super enhancer in resting Tregs, but not in resting Teffs in control cells (FIG. 12B). Upon stimulation, IL2RA achieved super enhancer status in both Teffs and Tregs, demonstrating that super enhancers vary across activation states Strikingly, in the MED12 KO resting Teffs. IL2RA rose in enhancer rank to the status of super enhancer, whereas KLF2 fell in rank in the resting Tregs and Teffs (FIG. 12B). Collectively, we found that MED12 is required for the maintenance and establishment of context specific super enhancers, including state dependent regulators of IL2RA identified within our screens.

### MED12 is Required for Conditional Expression of Core Regulators of IL2RA

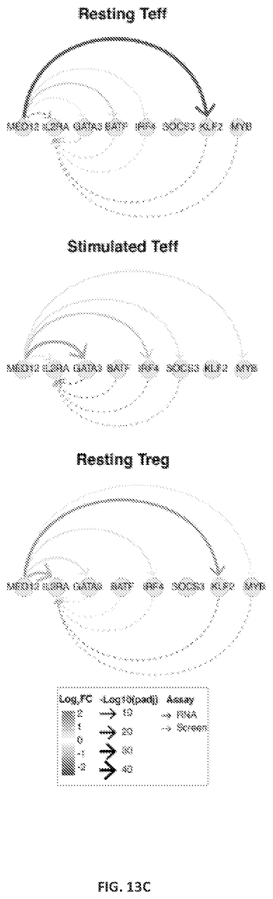

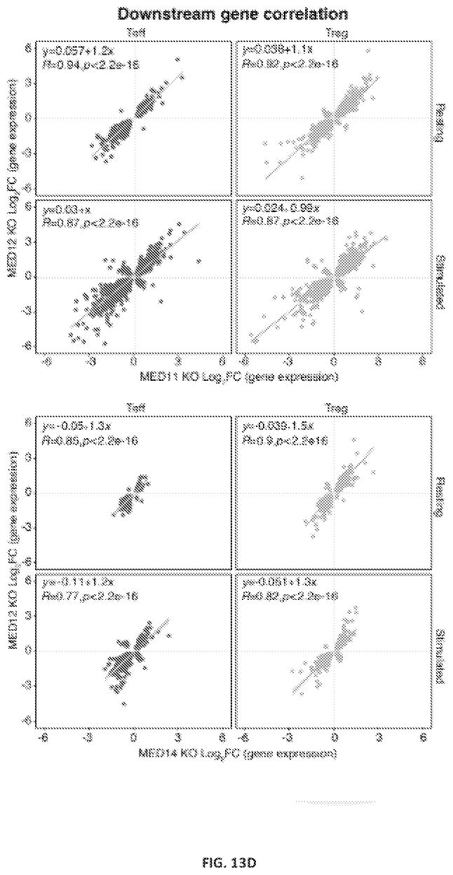

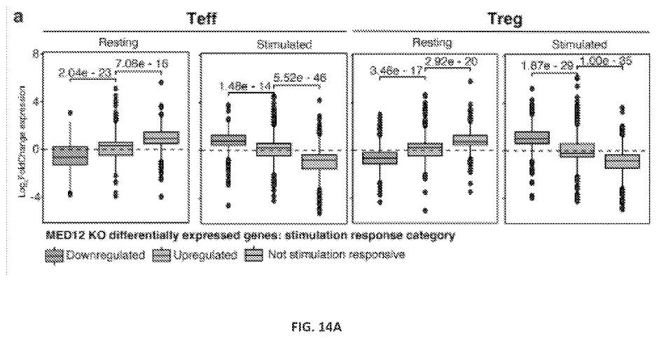

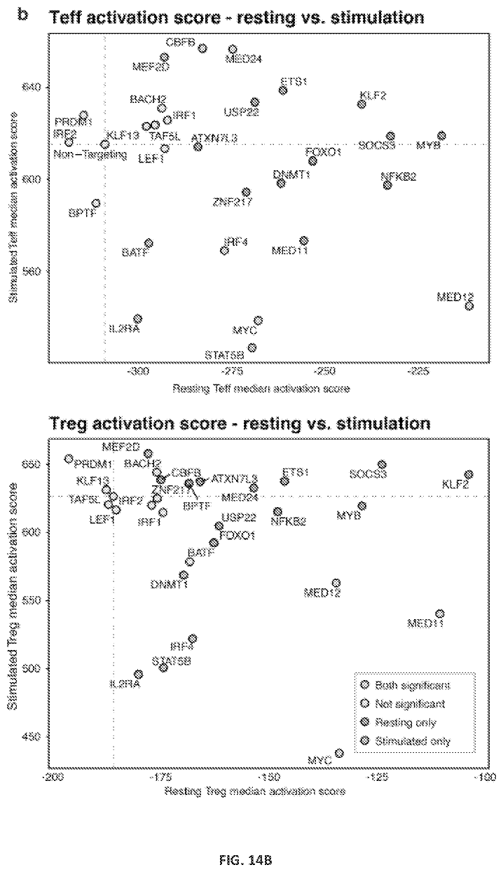

We continued to directly probe the mechanism by which MED12 enables context-dependent expression, with the hypothesis that MED12 specifically affects expression of IL2RA differentially by controlling expression of key conditional regulators identified in our screens. We perturbed MED12 and performed bulk RNAseq to reveal the downstream network in resting and stimulated Tregs and Teffs. Regulators of IL2RA were enriched in the differentially expressed genes downstream of MED12 across all conditions (OR=5.04—resting Tregs, 4.4—resting Teffs, and 5.95—stimulated Teffs) (FIG. 13A). MED12 therefore governs expression of a large subset of IL2RA regulators across both cell subsets and stimulation conditions.

Closer examination of the IL2RA regulators downstream of MED12 revealed cell type and stimulation specific circuits. Following MED12 KO, expression of IRF4 was increased in resting Teffs and Tregs, but decreased in the stimulated cell conditions (FIG. 13B). The common partner of IRF4 BATF, also shared a portion of these effects in resting Teffs and stimulated Tregs. Having characterized BATF-IRF4 as a positive regulator of IL2RA in Teffs and negative regulator in Tregs within our screen data, we were able to place them in cell type and stimulation responsive circuits downstream of MED12 (FIG. 13C). Additional resting and stimulation state regulators were affected as the result of MED12 ablation including positive regulator GATA3, which was most significantly decreased in stimulated Teffs, whereas expression of SOCS3, a negative regulator required to transition back to the resting state, was increased (FIG. 13C). KLF2, a potent negative regulator of IL2RA in the resting state, underwent one of the largest decreases in expression within the regulators and was selectively lost in MED12 KO resting cells. Both the loss of KLF2 and GATA3 as well as increase of SOCS3 corresponded with altered acetylation of the respective enhancer, as described above. Collectively, these results reveal that MED12 directs a network composed of cell type and stimulation specific of regulators, in order to achieve context dependent expression.

After defining the downstream network of MED12, we asked if MED12 might be functioning through the Mediator complex. MED12 is a part of the kinase domain, which transiently associates with the complex to regulate transcriptional initiation. The kinase domain is generally viewed as an inhibitory component because its presence prevents binding of the complex to RNA Pol 116. To directly compare the effect of MED12 and core Mediator, we perturbed one subunit from each functional module and performed bulk RNAseq. We observed that much like the screen, MED12 and core Mediator KOs often shared the same direction of effect, reflected by a strong positive correlation between MED12 and core subunits MED11 and MED14 (FIG. 13D). Additionally, MED12 often had a larger effect on regulator expression than the other probed subunits, which highlights a broader role in T cell gene regulation and argues against the notion that MED12 is working through inhibition of core Mediator in these contexts. Instead, MED12 may be a particularly critical component of Mediator in T cells and function as a scaffold for numerous cell type and state specific proteins.

### Activation State Specific Gene Expression is Coordinated by MED12 and Core Regulators

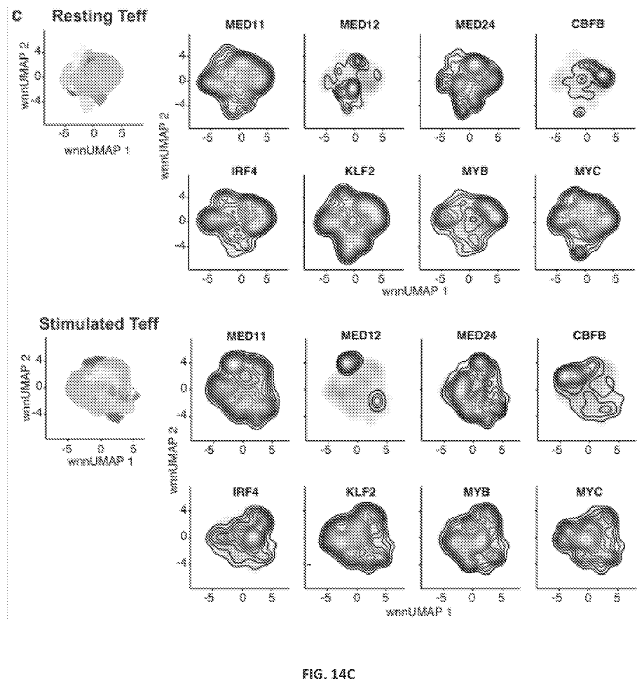

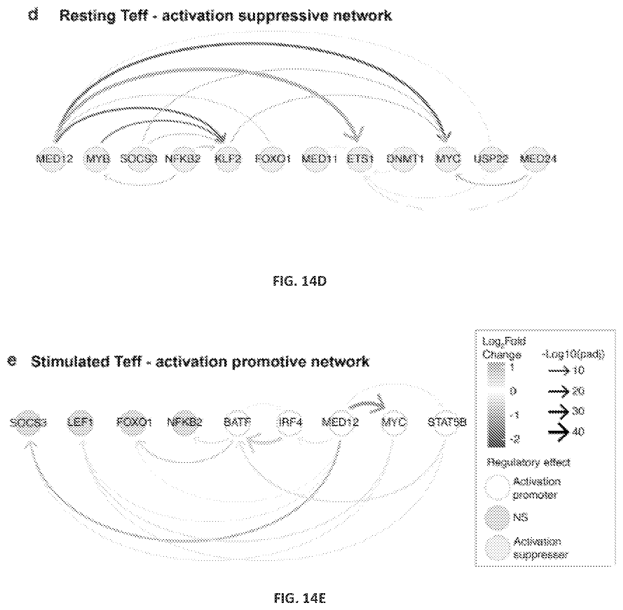

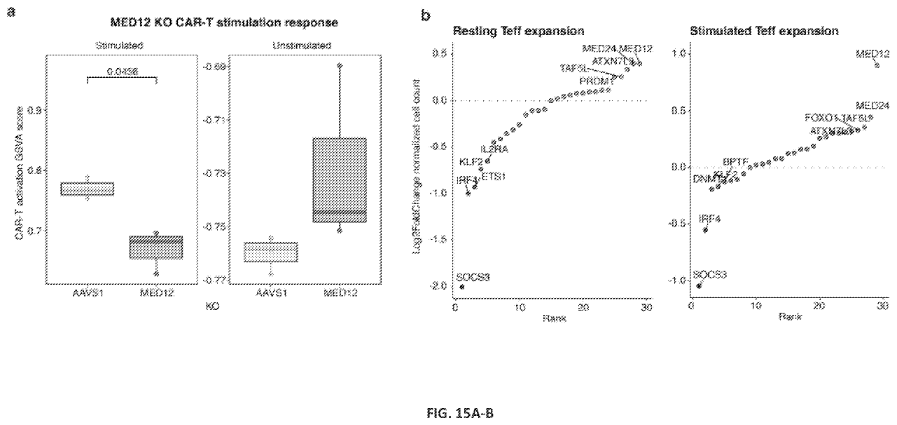

Induction of IL2RA in response to stimulation is a canonical marker of T cell activation. We suspected that many of the stimulation specific differences in expression of IL2RA were a reflection of alterations to the overall activation state of the perturbed cells. To test this, we identified genes within the control RNAseq samples with altered expression in response to stimulation. We then separated the differentially expressed genes downstream of MED12 based on their respective stimulation response category. We found that across both cell subsets, genes differentially expressed in response to stimulation were prematurely up or downregulated in the resting MED12 KO samples (FIG. 14A). However, the opposite was true for the stimulated samples, where stimulation responsive genes appeared differentially expressed in the opposite direction, suggesting that the MED12 KO cells failed to increase or decrease expression to the same degree as stimulated control cells. We performed an additional binomial test to determine that aberrant expression of stimulation specific genes was significant in all MED12 KO conditions, suggesting dysregulation of both the resting and activated cell state. More specifically, it appears that without MED12, the cells are unable to reach a full rested state or achieve peak levels of activation and instead lie in a moderate middle ground.

To characterize the global effects on cell state of regulators identified in our screens, we performed perturb-CITE-seq (pooled CRISPR perturbations coupled with scRNA-seq and single cell surface proteomics) in resting and stimulated Tregs and Teffs. We used a CRISPRi library to knock down 28 regulators of IL2RA in a pooled format, prioritizing trans factors with state specific effects. We then scored each of the perturbations to assess changes in the transcriptional activation state of the cells, using the algorithm described in Schmidt et a137. This process revealed many additional context-specific regulators of IL2RA as modulators of overall resting or activation states. In resting T cells, MYB. KLF2, and SOCS3 stood out as strong suppressors of activation whereas STAT5B, BATF and IRF4 appeared particularly important to promote activation in stimulated cells (FIG. 14B). Once again, MED12-targeted resting cells had significantly higher activation scores than non-targeting control cells, while stimulated MED12-targeted cells had significantly lower activation scores (FIG. 14B). In Tregs, this trend was even stronger for MED11, a core Mediator subunit. Surprisingly, MYC also appeared as a significant regulator of both the resting and activation state although its effect size was greater in stimulated cells. Although MYC is known to participate in T cell stimulation induced signaling, it also consistently emerged as a negative regulator of IL2RA in resting Tregs within the screens and arrayed assays described herein. The CITE-seq data also demonstrated significant increases in CD25, CD69, and CD82—all markers of T cell activation—in resting MYC KO Tregs compared to non-targeting cells with less substantial changes in resting Teff. Collectively, these results affirmed the role of MED12 as a regulator of both rest and activation and identified additional core regulators that enable global state specific gene expression.

To assess the effect of each perturbation across conditions, we subset the data into resting and stimulated Tregs and Teffs. Subsequent clustering of each group of cells revealed strong perturbation driven effects (FIG. 14C). Pseudo-bulking of cells with each gene perturbation and cellular condition enabled the construction of state specific regulatory networks. Amongst suppressors of activation in the resting state, the network converged on core resting state regulator KLF2 as well as the dynamic regulator MYC (FIG. 14D). This structure suggests that numerous TFs promote high levels of these core regulators to maintain the resting state. In contrast, we found that in stimulated Teffs there were few instances of positive regulators of activation promoting other positive regulators, with the exception of MED12 driving the expression of MYC (FIG. 14E). Instead, these regulators suppressed expression of several resting state maintenance factors. Interestingly, this model mimics the general structure observed in our stimulated Teff IL2RA screen results, which were specifically depleted of many negative regulators that we now see are repressed by factors that promote activation. Stimulated Tregs had a similar network distribution, albeit with greater convergence on MYC. These results lead to a model whereby the resting state—instead of being a state of regulatory inactivity—is actively reinforced by a self-promoting network of regulators and the transition to activation state is achieved through suppression of resting state factors.

### Perturbation of Activation State Regulators Alters T Cell Fitness and Durability

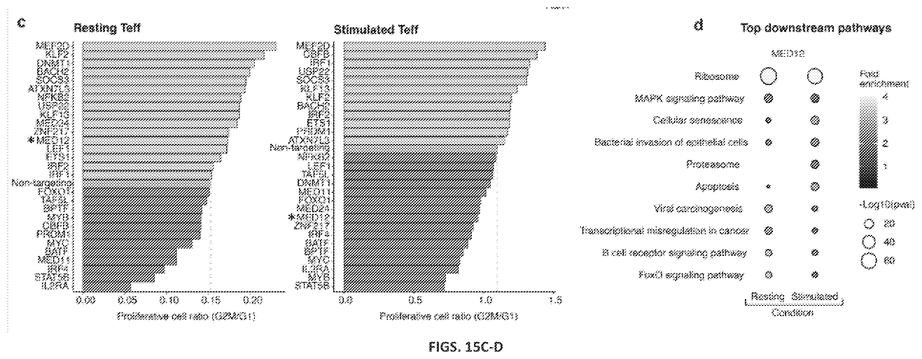

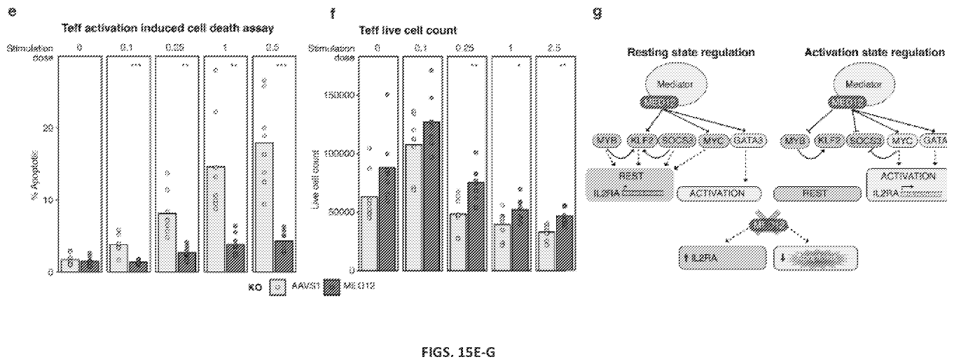

Synthetic perturbation of key regulators is a promising strategy to improve adoptive T cell therapies. Recently MED12 KO was nominated by a genome-wide CRISPR screen in CAR T cells to promote durable cell fitness. Ablation of MED12 resulted in an improved CAR-T product with sustained expansion and improved tumor killing in preclinical models, in congruence with increased STAT5 activation and IL2RA expression. An additional critical part of the therapeutic success of the MED12 KO CAR-T in vivo and in vitro could be mediated by broader changes in cell state transitions—avoiding complete rest and a state of peak stimulation. Using bulk RNAseq data from Freitas et al. (Science (1979) 378, (2022)) we generated an activation score using genes differentially expressed between the control resting and restimulated CARs. We then applied this score to the control and MED12 KO CAR-T samples and found a significant decrease in activation for the stimulated MED12 KO CARs compared to the control samples (FIG. 15A). Despite the MED12 CAR RNAseq data having been collected only 3 hours after stimulation—much earlier than our 48-hour collection point—blunted stimulation appears as a consistent feature of MED12 ablation including in the CAR T cell setting.

Consistent with the reported findings of increased expansion of MED12 KO CAR T cells, we found that within our Perturb-seq pool, MED12-targeted cells experienced the largest increase in total Teff cell counts, especially stimulated cells (FIG. 15B). We wondered why MED12-targeted cells with reduced capacity to achieve full activation would be more abundant than controls. We compared the ratio of proliferative cells in each Perturb-seq condition to those in a non-proliferative state using a ratio of G2M/G1 cells. Interestingly, MED12 targeted cells showed a slight increase in proliferative cells in the resting condition and a substantial decrease in proliferative cells in the stimulated condition compared to non-targeting cells (FIG. 15C). Overall, across the perturbed T cell pool, the percentage of proliferative cells and total cell abundance were not well correlated. Perturbation of regulators such as KLF2 and SOCS3 (which both suppress activation) resulted in pronounced increases in the percentage of proliferative cells, but also resulted in decreased cell abundances (FIG. 14B, 14D). Comprehensively these results suggest that increased cell cycling does not necessarily lead to greater overall numbers, possibly due to decreased viability.

We reasoned that the reduced stimulation responses, especially in MED12-targeted cells, may improve cell durability and therefore abundance by limiting activation-induced cell death (AICD). Consistent with this hypothesis, pathway analysis in our bulk RNAseq data showed apoptosis amongst the most enriched pathways for MED12 KO cells (FIG. 15D), which was driven by a mix of both up and downregulated genes. Similarly, examination of stimulatory and inhibitory surface proteins revealed increased expression of apoptosis initiating receptor FAS40, but a large decrease in an essential co-stimulatory receptor CD241. To determine the functional effect of this complex dysregulation, we performed a titration of stimulation strength using anti-CD3/CD28/CD2 soluble tetramers and quantified apoptosis via caspase-3/7 activation. We found that in control Teffs, apoptosis increased relative to the strength of stimulation (FIG. 19E). In marked contrast, MED12 KO cells underwent minimal apoptosis in response to stimulation. Overall MED12 KO resulted in significantly decreased apoptosis compared to control samples at higher levels of stimulation. The MED12 KO associated reductions in apoptosis translated to improved live cell abundance within the assay, providing an explanation for improved T cell and CAR-T durability following MED12 ablation, despite reduced activation following stimulation (FIG. 19F). This avoidance of apoptosis is likely complemented by higher levels of IL2RA in resting Teff, which we observed in our screens and arrayed assays as a state specific phenotype as well as in the MED12 KO CAR product.

In total, perturbation of context specific regulators revealed a network of nuclear regulators across the resting and activation states orchestrated by MED12 (FIG. 15G). We found the effects of MED12 on activation state to be stimulation-dependent and observed that reduced stimulation sensitivity and downstream signaling deficiencies protected MED12 from apoptosis induced cell death (AICD). In the resting state however, increased activation was observed with marked increase in CD25 expression, also corresponding with increased levels of enhancer acetylation in Teffs. Comprehensively, these altered features of the MED12 KO cells increase the sensitivity of Teffs to local IL-2 and STAT5 signaling, providing a proliferative advantage in the absence of TCR stimulation (FIG. 15G). In contrast, MED12 ablation in Tregs resulted in decreased CD25 expression across conditions and did not provide an obvious proliferative advantage in the resting state pool. These distinctions highlight the importance of cell type and state specific regulation of both IL2RA and activation states.

Context-specific regulation of gene expression by nuclear proteins is essential for cell identity maintenance and response to environmental stimulus but is challenging to study in high throughput. By performing CRISPR screens across conditions, a dynamic network of trans regulators that enable cell type and stimulation specific expression of IL2RA was defined. This approach highlighted broad structural differences in the upstream network of IL2RA across the T cell compartment. Most notably, the difference in regulation between Tregs with constitutive high levels of IL2RA and stimulated Teffs with transient high levels was large and indicative that expression of IL2RA is not governed by a simple on-off switch. Instead. Teffs utilize waves of regulators to maintain rest (KLF2 and MED12), achieve peak expression (GATA3 and MED12), and return to a resting state (TAF5L and SOCS3); while Tregs appear to utilize a more static but expansive network of regulators. Ultimately, these studies demonstrated a simple but effective means to dissect complex regulatory systems, including cell identity maintenance and stimulus response. Beyond single gene regulation, by reading out an activation marker, these screens also yielded rich insight into the regulation of T cell activation and rest. Within the resting state we defined suppressors of activation, including KLF2, MYB and SOCS3 (FIG. 15G), with intertwined downstream connections. In stimulated cells, both GATA3 and MYC were defined as potent positive regulators of activation (FIG. 15G). Most strikingly, MED12 appeared consistently as a hierarchical conductor of each network, required to establish both resting and activated T cell states.

This characterization of MED12 showed that deletion of MED12 resulted in increased cell abundance without evidence of significantly improved cell cycling. Instead, MED12 ablation prevents activation induced death, likely from the inability of these cells to reach a peak stimulated state. Complementary to this avoidance of death, we also observed indications of increase IL2 sensitivity and STAT5 signaling in resting state Teffs, which can be attributed to significantly higher levels of IL2RA. These findings provide evidence of an ideal activation range to improve engineered T cell product efficacy and avoid exhaustion.

What is claimed is:

1. A T cell comprising:
(a) a genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574; and/or
(b) a heterologous polynucleotide that encodes FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, or ZNF574.

2. The T cell of claim 1, wherein the T cell comprises:
(a) a genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is increased in the T cell relative to expression of IL2RA in a T cell not comprising the genetic modification or heterologous polynucleotide that inhibits expression of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574; and/or
(b) a heterologous polynucleotide that encodes FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32, wherein expression of IL2RA is increased in the T cell relative to expression of IL2RA in a T cell not comprising the heterologous polynucleotide that encodes FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32.

3. The T cell of claim 1, wherein the T cell comprises:
(a) a genetic modification or heterologous polynucleotide that inhibits expression of FOXN2, ZNF25, THRA, ZBTB2, CREM, PTFIA, IKZF2, MYNN, BACH2, IL15RA, SP3, ZBTB3, REST, ELP2, ZBTB14, GFI1, IL2RB, NR4A3, or ZBTB32, wherein expression of IL2RA is decreased in the T cell relative to expression of IL2RA in a T cell not comprising the genetic modification or heterologous polynucleotide that inhibits expression of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32; and/or
(b) a heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the T cell relative to expression of IL2RA in a T cell not comprising heterologous polynucleotide that encodes BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574.

4. The T cell of any one of claims 1-3, wherein the T cell is a regulatory T cell.

5. The Treg cell of claim 4, wherein the regulatory T cell comprises:
(a) a genetic modification or heterologous polynucleotide that inhibits expression ZKSCAN2, ZNF114, E4F1, GLIS2, JUNB, NR2F6, ZZZ3, ETV3, ERF, RUNX1, TGIF2, or ZNF574, wherein expression of IL2RA is increased in the Treg cell relative to expression of IL2RA in a Treg cell not comprising the genetic modification or heterologous polynucleotide that inhibits expression of ZKSCAN2, ZNF114, E4F1, GLIS2, JUNB, NR2F6, ZZZ3, ETV3, ERF, RUNX1, TGIF2, or ZNF574; and/or
(b) a heterologous polynucleotide that encodes FOXN2, ZNF25, THRA, ZBTB2, CREM, PTFIA, IKZF2, MYNN, BACH2, IL15RA, SP3, or ZBTB3, wherein expression of IL2RA is increased in the Treg cell relative to expression of IL2RA in a Treg cell not comprising the heterologous polynucleotide that encodes FOXN2, ZNF25, THRA, ZBTB2, CREM, PTF1A, IKZF2, MYNN, BACH2, IL15RA, SP3, or ZBTB3.

6. The Treg cell of claim 4, wherein the Treg cell comprises:
(a) a genetic modification or heterologous polynucleotide that inhibits expression FOXN2, ZNF25, THRA, ZBTB2, CREM, PTF1A, IKZF2, MYNN, BACH2, IL15RA, SP3, or ZBTB3, wherein expression of IL2RA is decreased in the Treg cell relative to expression of IL2RA in a Treg cell not comprising the genetic modification or heterologous polynucleotide that inhibits expression of FOXN2, ZNF25, THRA, ZBTB2, CREM, PTF1A, IKZF2, MYNN, BACH2, IL15RA, S3, or ZBTB3; and/or
(b) a heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574, wherein expression of IL2RA is decreased in the Treg cell relative to expression of IL2RA in a Treg cell not comprising the heterologous polynucleotide that encodes ZKSCAN2, ARID5A, ZNF114, E4F1, GLIS2, JUNB, NR2F6, ZZZ3, ETV3, ERF, RUNX1, TGIF2, or ZNF574.

7. The T cell of any one of claims 1-3, wherein the T cell is a CD8+ or a CD4+ T cell.

8. The CD4+ T cell of claim 7, wherein the CD4+ T cell comprises:
(a) a genetic modification or heterologous polynucleotide that inhibits expression of BACH2, ZBTB3, ABTB14, GFI1, or IL2RB, wherein expression of IL2RA is decreased in the CD4+ T cell relative to expression of IL2RA in a CD4+ T cell not comprising the genetic modification or heterologous polynucleotide that inhibits expression of BACH2, REST, ZBTB3, ABTB14, GFI1, or IL2RB.

9. The T cell of any one of claims 1-3, wherein the T cell is a stimulated CD4+ T cell.

10. The T cell of claim 9, wherein the stimulated CD4+ T cell comprises:
(a) a genetic modification or heterologous polynucleotide that inhibits expression of REST, ELP2, IL2RB, NR4A3, or ZBTB32, wherein expression of IL2RA is decreased in the stimulated CD4+ T cell relative to expression of IL2RA in a stimulated CD4+ T cell not comprising the genetic modification or heterologous polynucleotide that inhibits expression of REST, ELP2, IL2RB, NR4A3, or ZBTB32; and/or
(b) a heterologous polynucleotide that encodes BACH2, STAT6, ZMYND8, IFNGR2, IL4R, LEF1, or ARID5A, wherein expression of IL2RA is decreased in the stimulated CD4+ T cell relative to expression of IL2RA in a stimulated CD4+ T cell not comprising the heterologous polynucleotide that encodes BACH2, STAT6, ZMYND8, IFNGR2, IL4R, LEF1, or ARID5A.

11. The T cell of claim 9, wherein the stimulated CD4+ T cell comprises:
(a) a genetic modification or heterologous polynucleotide that inhibits expression of BACH2, STAT6, ZMYND8, IFNGR2, IL4R, LEF1, or ARID5A, wherein expression of IL2RA is increased in the stimulated CD4+ T cell relative to expression of IL2RA in a stimulated CD4+ T cell not comprising the genetic modification or heterologous polynucleotide that inhibits expression of BACH2, STAT6, ZMYND8, IFNGR2, IL4R, LEF1, or ARID5A; and/or
(b) a heterologous polynucleotide that encodes REST, ELP2, IL2RB, NR4A3, or ZBTB32, wherein expression of IL2RA is increased in the stimulated CD4+ T cell relative to expression of IL2RA in a stimulated CD4+ T cell not comprising the heterologous polynucleotide that encodes REST, ELP2, IL2RB, NR4A3, or ZBTB32.

12. The T cell of any one of claims 1-11, wherein the T cell is a human T cell.

13. A population of cells comprising the genetically modified T cell of any one of claims 1-12.

14. A method of making a modified T cell, the method comprising:
(a) inhibiting expression of one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2 and ZNF574; and/or
(b) overexpressing one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574.

15. The method of claim 14, wherein the inhibiting comprises reducing expression of the nuclear factor, or reducing expression of a polynucleotide encoding the nuclear factor.

16. The method of claim 15, wherein the inhibiting comprises contacting a polynucleotide encoding the nuclear factor with a targeted nuclease, a guide RNA (gRNA), an siRNA, an antisense RNA, microRNA (miRNA), or short hairpin RNA (shRNA).

17. The method of any one of claims 14-16, wherein the inhibiting comprises mutating the polynucleotide encoding the nuclear factor.

18. The method of claim 17, wherein the inhibiting comprises contacting the polynucleotide with a targeted nuclease.

19. The method of claim 18, wherein the targeted nuclease introduces a double-stranded break in a target region in the polynucleotide.

20. The method of claim 18 or 19, wherein the targeted nuclease is an RNA-guided nuclease.

21. The method of claim 20, wherein the RNA-guided nuclease is a Cpf1 nuclease or a Cas9 nuclease and the method further comprises introducing into a T cell a gRNA that specifically hybridizes to a target region in the polynucleotide.

22. The method of claim 21, wherein the Cpf1 nuclease or the Cas9 nuclease and the gRNA are introduced into the T cell as a ribonucleoprotein (RNP) complex.

23. The method of any one of claims 19-21, wherein the inhibiting comprises performing clustered regularly interspaced short palindromic repeats (CRISPR)/Cas genome editing.

24. The method of any one of claims 14-23, wherein the T cell is administered to a human following the inhibiting.

25. The method of any one of claims 14-24, wherein the T cell is obtained from a human prior to treating the T cell to inhibit expression of the nuclear factor, and the treated T cell is reintroduced into a human.

26. The method of claim 25, wherein the T cell is a Treg cell.

27. The method of claim 25, wherein the T cell is a CD8+ or a CD4+ T cell.

28. The method of claim 27, wherein the T cell is a stimulated CD4+ T cell.

29. The method of any one of claims 25-28, wherein expression of one or more nuclear factors selected from the group consisting of BACH2, STAT6, ZMYND8, IFNGR2, IL4R, LEF1, ZKSCAN2, ARID5A, ZNF114, E4F1, GLIS2, JUNB, NR2F6, ZZZ3, ETV3, ERF, RUNX1, TGIF2, or ZNF574 is inhibited in the T cell.

30. The method of any one of claims 25-28, wherein expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32 is inhibited in the T cell.

31. The method of any one of claims 25-28, wherein the T cell is a regulatory T cell and expression of one or more nuclear factors selected from the group consisting of ZKSCAN2, ARID5A, ZNF114, E4F1, GLIS2, JUNB, NR2F6, ZZZ3, ETV3, ERF, RUNX1, TGIF2, or ZNF574 is inhibited in the Treg cell.

32. The method of any one of claims 25-28, wherein the T cell is a regulatory T cell and expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, ZBTB14, GFI1, IL2RB, NR4A3, or ZBTB32 is inhibited to decrease IL2RA expression in the regulatory T cell, and the subject has cancer.

33. The method of any one of claims 25-28, wherein the T cell is a CD4+ T cell and expression of one or more nuclear factors selected from the group consisting of BACH2, ZBTB3, ABTB14, GFI1, IL2RB, REST, ELP2, IL2RB, NR4A3, or ZBTB32 is inhibited to decrease IL2RA expression in the CD4+ T cell, and wherein the subject has an autoimmune disorder.

34. The method of any one of claims 25-28, wherein the T cell is a CD4+ T cell and expression of one or more nuclear factors selected from the group consisting of BACH2, STAT6, ZMYND8, IFNGR2, IL4R, LEF1, or ARID5A is inhibited to increase IL2RA expression in the CD4+ T cell, and wherein the subject has cancer.

35. A T cell made by the method of any one of claims 25-34.

36. A method of modifying T cells in a subject in need thereof, comprising inhibiting expression of a one or more nuclear factors or overexpressing one or more factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574 in the human T cells of the subject.

37. The method of claim 36, wherein inhibiting expression of one or more nuclear factors or overexpression of one or more nuclear factors occurs in vivo.

38. The method of claim 36, wherein the method comprises:
a) obtaining T cells from the subject;
b) modifying the T cells by inhibiting expression of one or more nuclear factors selected from the group consisting of FOXN2, ZNF25, THRA, ZBTB2, CREM, PTF1A, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574; and
c) administering the T cells to the subject.

39. The method of claim 36, wherein the method comprises:
a) obtaining T cells from the subject;
b) modifying the T cells by overexpressing one or more nuclear factors selected from the group consisting of FOXN2, ZNF25, THRA, ZBTB2, CREM, PTFIA, IKZF2, MYNN, BACH2, IL15RA, SP3, ZBTB3, REST, ELP2, STAT6, ZMYND8, ZBTB14, GFI1, IFNGR2, IL4R, IL2RB, NR4A3, LEF1, ZBTB32, ZKSCAN2, ARID5A, ZNF114, E4F1, GLIS2, JUNB, NR2F6, ZZZ3, ETV3, ERF, RUNX1, TGIF2, and ZNF574; and
c) administering the T cells to the subject.

40. The method of claim 38 or 39, wherein the subject has cancer or an autoimmune disorder.

41. A method of treating an autoimmune disorder in a subject, the method comprising administering a population of the T cells of claim 5, 8 or 10 to a subject that has an autoimmune disorder.

42. A method of treating cancer in a subject, the method comprising administering a population of the T cells of claim 6 or 11 to a subject that has cancer.
